(19) United States
(12) Patent Application Publication
(10) Pub. No.: US 2007/0112035 A1
(43) Pub. Date: May 17, 2007

(54) Substituted oxyarenes

(75) Inventors: Peter Jeschke, Bergisch Gladbach (DE); Michael Muller, Leverkusen (DE); Iris Escher, Ebsdorfergrund (DE); Olga Malsam, Rosrath (DE); Karl-Josef Haack, Heiligenhaus (DE); Ralf Braun, Ramberg (DE); Christian Arnold, Langenfeld (DE)

Correspondence Address: STERNE, KESSLER, GOLDSTEIN & FOX P.L.L.C., 1100 NEW YORK AVENUE, N.W., WASHINGTON, DC, 20005, US

(73) Assignee: BAYER CROPSCIENCE AKTIENGESELLSCHAFT, Monheim (DE)

(21) Appl. No.: 10/556,426

(22) PCT Filed: Apr. 27, 2004
(86) PCT No.: PCT/EP04/04415

§ 371 Date: Nov. 27, 2006

(30) Foreign Application Priority Data
May 9, 2003 (DE) 103 20 782.1

(51) Int. Cl. A01N 43/40; A01N 43/80; C07D 413/02; C07D 261/06

(52) U.S. Cl. 514/340; 514/378; 546/272.1; 548/247

(57) ABSTRACT

The invention relates to compounds of formula (I), wherein A¹, A², R¹, R², R³, R⁴, R⁵, and Y have the meaning indicated in the description, methods and intermediate compounds for the production thereof, and the use thereof for controlling pests.

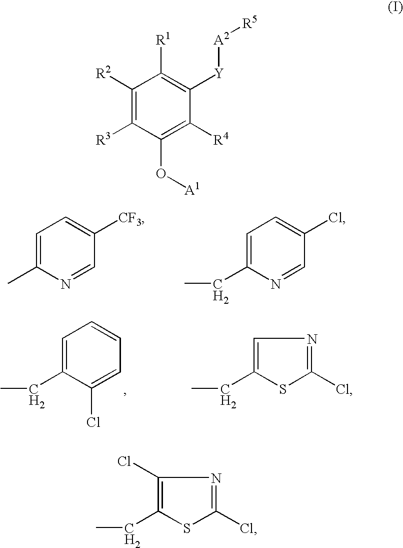

The present invention relates to new substituted oxyarenes, methods for their production and their use as pest control agents.

Substituted 5-benzyloxymethyl-4,5-dihydro-isoxazoles have already been known for use as herbicides in rice crops (compare WO 02/19825, U.S. Pat. No. 4,983,210, U.S. Pat. No. 5,262,388, JP 09-143171), but have so far achieved no significance as a result of an effect that is not always satisfactory.

Now new substituted oxyarenes of the general formula (I) were found, 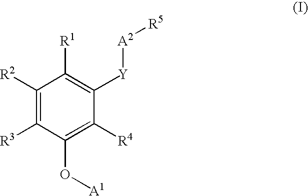
wherein
A¹ stands for one of the groupings —CH₂—CH═CCl₂, —CH₂—CH═CBr₂, —CH₂—CH═CClF, —CH₂—CF═CCl₂, —(CH₂)₂—CH═CF₂, —CH₂—CH═CBrCl, —CH₂—CH═CBrF, —CF═CH—CH═CH₂, —CH₂—CF═CF—CH═CH₂, —CH₂—CH═CClCF₃, —(CH₂)₂—CX₃ and —CH₂—CH═CClCH₃, whereby X stands for halogen,
   or stands for one of the groupings, 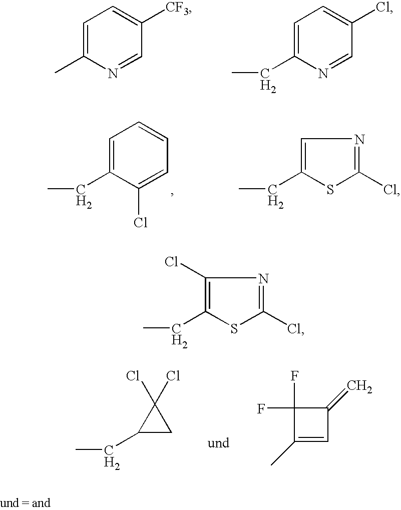
A² in each case stand for straight-chain or branched alkanediyl or alkenediyl with up to 8 carbon atoms in each case, which optionally contain an oxygen atom, a sulphur atom or a grouping selected from SO, SO₂, NH or N(C₁-C₄-alkyl) at the beginning of, at the end of or within the carbon chain,
R¹ stands for hydrogen, nitro, hydroxy, amino, cyano, halogen, for alkyl, alkoxy, alkylthio, alkylamino, dialkylamino, alkylcarbonylamino or alkoximinoalkyl with 1 to 10 carbon atoms in the alkyl groups, optionally substituted in each case by cyano, halogen, C₁-C₆-alkylsulfinyl, C₁-C₆-alkylsulfonyl or C₁-C₆-alkoxy, for C₁-C₆-alkylcarbonyloxy, for C₁-C₆-alkoxycarbonyloxy, for C₃-C₆-cycloalkoxycarbonyloxy, for C₁-C₆-dialkyaminocarbonyloxy, for aryloxy, arylthio or arylalkyl with 6 or 10 carbon atoms in each case in the aryl groups and optionally 1 to 4 carbon atoms in the alkyl part, optionally substituted in each case by nitro, hydroxy, amino, cyano, halogen, C₁-C₆-alkyl, C₁-C₆-halogenalkyl, C₁-C₆-alkoxy or C₁-C₆-halogenalkoxy, for heterocyclyloxy or heterocyclylthio with up to 10 carbon atoms, up to 4 nitrogen atoms and optionally an oxygen or sulphur atom in each case, optionally substituted in each case by nitro, hydroxy, amino, cyano, halogen, C₁-C₆-alkyl, C₁-C₆-halogenalkyl, C₁-C₆-alkoxy or C₁-C₆-halogenalkoxy, or stands for the grouping —O-A¹, whereby the A¹ has the meaning given above, or stands for the grouping —N(R,R′), whereby R und R′ together stand for straight-chain or branched alkanediyl with up to 8 carbon atoms, which optionally contains an oxygen atom, a sulphur atom or a grouping selected from SO, SO₂, NH or N(C₁-C₄-alkyl) at the beginning of, at the end of or within the carbon chain,
R² stands for hydrogen, nitro, hydroxy, amino, cyano, cyanato, thiocyanato, formyl, halogen, for alkyl, alkoxy, alkylthio, alkylsulphinyl, alkylsulphonyl, alkylamino, dialkylamino or alkylcarbonylamino with 1 to 6 carbon atoms in each case in the alkyl groups, optionally substituted in each case by cyano, halogen or C₁-C₆-alkoxy, for C₁-C₆-alkyl-carbonyl, C₁-C₆-alkoxy-carbonyl, C₁-C₆-alkoximinoformyl, C₁-C₆-alkoximino-acetyl, or for C₂-C₆-alkenyl or C₂-C₆-alkinyl,
R³ stands for hydrogen, nitro, hydroxy, amino, cyano, halogen, for alkyl, alkoxy, alkylthio, alkylamino, dialkylamino or alkylcarbonylamino with 1 to 6 carbon atoms in each case in the alkyl groups, optionally substituted by cyano, halogen or C₁-C₆-alkoxy,
R⁴ stands for hydrogen, nitro, hydroxy, amino, cyano, halogen, for alkyl, alkoxy, alkylthio, alkylamino, dialkylamino or alkylcarbonylamino with 1 to 6 carbon atoms in each case in the alkyl groups, optionally substituted by cyano, halogen or C₁-C₆-alkoxy,
R⁵ stands for hydrogen, for aryl with 6 or 10 carbon atoms in the aryl group optionally substituted in each case by nitro, hydroxy, amino, cyano, halogen, C₁-C₆-alkyl, C₁-C₆-halogenalkyl, C₁-C₆-alkoxy, C₁-C₆-halogenalkoxy, C₁-C₂-alkylendioxy, C₁-C₂-haloalkylendioxy, C₁-C₆-alkylthio, C₁-C₆-halogenalkylthio, C₁-C₆-alkoxyimino-C₁-C₆-alkyl, or for heteroaryl with up to 10 carbon atoms, up to 4 nitrogen atoms and optionally an oxygen or sulphur atom, optionally substituted the same or differently one to three times, whereby the substituents can be selected from the following group of substituents:
   Nitro, hydroxy, amino, cyano, halogen, C₁-C₆-alkyl, C₁-C₆-halogenalkyl, C₁-C₆-alkoxy, C₁-C₆-halogenalkoxy, C₁-C₆-alkylcarbonyl, C₂-C₆-alkoxycarbonyl, C₂-C₆-alkenyl, C₂-C₆-alkenyloxy, C₂-C₆-halogenalkenyl, C₂-C₆-halogenalkenyloxy, C₂-C₆-alkinyl, C₂-C₆-alkinyloxy, C₁-C₂-alkylendioxy, C₁-C₂-haloalkylendioxy, C₁-C₆-alkylthio, C₁-C₆-halogenalkylthio, C₁-C₆-alkoxyimino-C₁-C₆-alkyl and the grouping 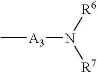
   wherein
   A³ stands for a single bond, or stands for C₁-C₆-alkanediyl, which is optionally substituted by one to six equivalent or different substituents from the group C₁-C₃-halogenalkyl, C₁-C₈-cycloalkyl and C₃-C₈-cycloalkyl-C₁-C₆-alkyl,
   R⁶ stands for hydrogen, cyano, hydroxy, C₁-C₆-alkyl, C₃-C₈-cycloalkyl, C₃-C₈-cycloalkyl-C₁-C₆-alkyl, C₁-C₆-halogenalkyl, C₁-C₆-alkoxy, C₁-C₆-halogenalkoxy, C₂-C₆-alkenyloxy, C₂-C₆-halogenalkenyloxy, C₂-C₆-alkinyloxy, —C(═O)R⁸, —C(═O)R⁸, or optionally for phenyl or benzyl substituted one to five times, the same or differently, in each case in the aryl part by halogen, C₁-C₆-alkyl, C₁-C₆-halogenalkyl, C₁-C₆-alkoxy, C₁-C₆-halogenalkoxy, hydroxy, cyano or nitro,
   R⁷ stands for hydrogen, cyano, hydroxy, C₁-C₆-alkyl, C₃-C₈-cycloalkyl, C₃-C₈-cycloalkyl-C₁-C₆-alkyl, C₁-C₆-halogenalkyl, C₁-C₆-alkoxy, C₁-C₆-halogenalkoxy, C2-C₆-alkenyloxy, C2-C₆-halogenalkenyloxy, C2-C₆-alkinyloxy, —C(═O)R⁸, —C(═O)R⁸, or for phenyl or benzyl optionally substituted one to five times, the same or differently, in each case in the aryl part by halogen, C₁-C₆-alkyl, C₁-C₆-halogenalkyl, C₁-C₆-alkoxy, C₁-C₆-halogenalkoxy, hydroxy, cyano or nitro, or
   R⁷ together with R⁶ stands for C₄-C₈-alkanediyl or C₄-C₈-alkylenediyl optionally substituted in each case one to four times, the same or differently, by C₁-C₆-alkyl, C₃-C₈-Cycloalkyl-C₁-C₆-alkyl, C₁-C₆-halogenalkyl, cyano or C₁-C₆-alkylcarbonyl, whereby a CH₂ group can be optionally replaced by O, S or NR⁹, or
   R⁷ stands for —C(═O)R⁸ or —C(═S)R⁸, whereby R⁶ und R⁸ then stand together in each case for C₂-C₈-alkanediyl or C₂-C₈-alkylenediyl optionally substituted one to four times, the same or differently, by C₁-C₆-alkyl, C₃-C₈-cycloalkyl-C₁-C₆-alkyl, C₁-C₆-halogenalkyl, cyano or C₁-C₆-alkylcarbonyl, whereby a CH₂ group can be optionally replaced by O, S or NR⁹, or
   R⁶ and R⁷ independently from one another stand for —C(═O)R⁸ or —C(═S)R⁸, and both of the moieties R⁸ together stand in each case for straight-chain or branched C₂-C₈-alkanediyl or C₂-C₈-alkylenediyl optionally substituted one to four-times, the same or differently, by C₁-C₆-alkyl, C₃-C₈-cycloalkyl-C₁-C₆-alkyl, C₁-C₆-halogenalkyl, cyano or C₁-C₆-alkylcarbonyl, and wherein a CH₂ group can be optionally replaced by O, S or NR⁹,
   R⁸ stands for C₁-C₆-alkyl, C₁-C₆-halogenalkyl, C₂-C₆-alkenyl, C₂-C₆-halogenalkenyl, C₂-C₆-alkinyl, C₁-C₆-alkoxy, C₁-C₆-halogenalkoxy, C₂-C₆-alkenyloxy, C₂-C₆-halogenalkenyloxy, C₂-C₆-alkinyloxy, C₃-C₆-cycloalkyl, for phenyl or benzyl optionally substituted in each case one to three times, the same or differently, in the aryl part by halogen, cyano, nitro, C₁-C₆-alkyl, C₁-C₆-halogenalkyl, C₁-C₆-alkylcarbonyl, C₂-C₆-alkenyl, C₂-C₆-halogenalkenyl, C₂-C₆-alkynyl, C₁-C₆-alkoxy, C₁-C₆-halogenalkoxy, C₁-C₆-alkoxycarbonyl, C₁-C₃-halogenalkoxycarbonyl or C₂-C₆-halogenalkenyloxy, and
   R⁹ stands for hydrogen, C₁-C₆-alkyl, C₁-C₃-halogenalkyl, C₁-C₃-halogenalkylcarbonyl, C₁-C₆-alkoxyalkyl, C₁-C₆-alkylcarbonyl or C₃-C₈-cycloalkyl, and
Y stands for a five or six-membered heterocyclic grouping connected with the adjacent groupings at two different positions with at least 2 carbon atoms, at least one nitrogen atom and optionally an oxygen or sulphur atom, in particular for a heterocyclic grouping selected from the following list (in this respect, the exocyclic dashes indicate the connections with the adjacent groupings in each case according to the order in formula (I)), 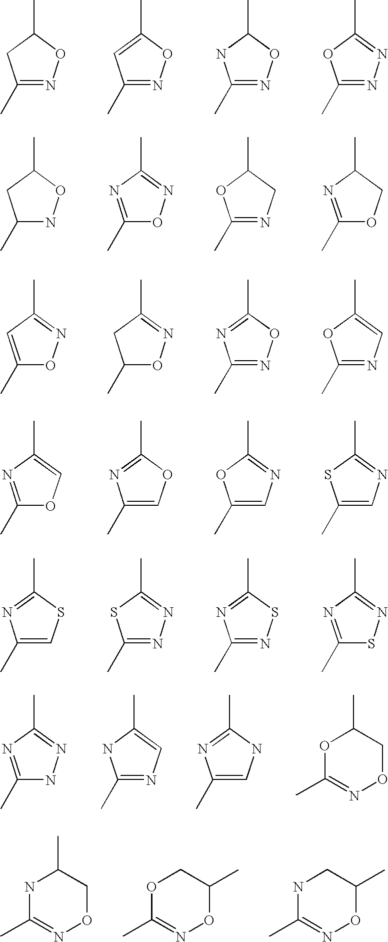
   whereby these heterocyclic groupings can be optionally substituted in each case by one or two substituents from the series nitro, hydroxy, amino, cyano, halogen, C₁-C₆-alkyl, C₁-C₆-halogenalkyl, C₁-C₆-alkoxy, C₁-C₆-halogenalkoxy, C₁-C₆-alkylthio, C₁-C₆-halogenalkythio.

Depending on the type of the substituents, the compounds of the formula (I) can also optionally exist as stereoisomers, i.e. as geometric and/or as optical isomers or mixtures of isomers in different compounds. The pure stereoisomers as well as any mixtures of these isomers are the subject of this invention, even if only compounds of the formula (I) are mentioned here in general.

The invention also relates to saline derivatives formed from compounds of the formula (I) by reaction with basic or acidic compounds.

Preferred substituents and preferred areas of the moieties present in the formulas listed above and below are defined below.
A¹ preferably stands for one of the following groupings:
   —CH₂—CH═CCl₂, —CH₂—CH═CBr₂, —CH₂—CH═CClF, —CH₂—CF═CCl₂, —(CH₂)₂—CH═CF₂, —CH₂—CH═CBrCl, —CH₂—CH═CBrF, —CF═CH—CH═CH₂, —CH₂—CF═CF—CH═CH₂, —CH₂—CH═CClCF₃ and —CH₂—CH═CClCH₃ or for the grouping
A² preferably stands in each case for straight-chain or branched alkanediyl or alkenediyl with up to 4 carbon atoms in each case, which optionally contain an oxygen atom, a sulphur atom or a grouping selected from SO, SO₂, NH or N(C₁-C₃-alkyl) at the end or within the carbon chain.
R¹ preferably stands for hydrogen, nitro, hydroxy, amino, cyano, halogen, for alkyl, alkoxy, alkylthio, alkylamino, dialkylamino, alkylcarbonylamino or alkoximinoalkyl with 1 to 8 carbon atoms in the alkyl groups optionally substituted in each case by cyano, halogen, C₁-C₃-alkylsulphinyl, C₁-C₃-alkylsulphonyl or C₁-C₅-alkoxy, for C₁-C₃-alkylcarbonyloxy, for C₁-C₃-alkoxycarbonyloxy, for C₃-C₅-cycloalkoxycarbonyloxy, for C₁-C₆-dialkyaminocarbonyloxy, for aryloxy, arylthio or arylalkyl with 6 or 10 carbon atoms in each case in the aryl groups and optionally 1 to 3 carbon atoms in the alkyl part, optionally substituted in each case by nitro, hydroxy, amino, cyano, halogen, C₁-C₅-alkyl, C₁-C₅-halogenalkyl, C₁-C₅-alkoxy or C₁-C₅-halogenalkoxy, or for the grouping —O-A¹ whereby A¹ has the as meaning given above, or stands for the grouping —N(R,R′), whereby R und R′ together stand for straight-chain or branched alkanediyl with up to 6 carbon atoms, which optionally contains an oxygen atom, a sulphur atom or a grouping selected from SO, SO₂, NH or N(C₁-C₃-alkyl) at the beginning of, at the end of or within the carbon chain.
R² preferably stands for hydrogen, nitro, cyano, cyanato, thiocyanato, formyl, halogen, for alkyl, alkoxy, alkylthio, alkylamino, dialkylamino or alkylcarbonylamino with 1 to 5 carbon atoms in each case in the alkyl groups, optionally substituted in each case by cyano, halogen or C₁-C₅-alkoxy, for C₁-C₅-alkyl-carbonyl, C₁-C₅-alkoxy-carbonyl, C₁-C₅-alkoximinoformyl, C₁-C₅-alkoximino-acetyl, or for C₂-C₅-alkenyl or C₂-C₅-alkinyl.
R³ preferably stands for hydrogen, nitro, halogen, for alkyl, alkoxy, alkylthio or alkylamino with 1 to 5 carbon atoms in each case in the alkyl groups, optionally substituted by cyano, halogen or C₁-C₅-alkoxy.
R⁴ preferably stands for hydrogen, nitro, halogen, for alkyl, alkoxy, alkylthio or alkylamino with 1 to 5 carbon atoms in each case in the alkyl groups, optionally substituted by cyano, halogen or C₁-C₅-alkoxy.
R⁵ preferably stands for hydrogen, for aryl with 6 or 10 carbon atoms in the aryl group optionally substituted in each case by nitro, hydroxy, amino, cyano, halogen, C₁-C₅-alkyl, C₁-C₅-halogenalkyl, C₁-C₅-alkoxy, C₁-C₅-halogenalkoxy, C₁-C₂-alkylendioxy, C₁-C₂-haloalkylendioxy, C₁-C₅-alkylthio, C₁-C₅-halogenalkylthio, C₁-C₅-alkoxyiminoC₁-C₅-alkyl, or for heteroaryl with up to 9 carbon atoms, 1 to 3 nitrogen atoms and/or an oxygen or sulphur atoms optionally substituted the same or differently one to three times, whereby the substituents can be selected from the following group of substituents:
   nitro, hydroxy, amino, cyano, halogen, C₁-C₅-alkyl, C₁-C₅-halogenalkyl, C₁-C₅-alkoxy, C₁-C₅-halogenalkoxy, C₁-C₅-alkylcarbonyl, C₂-C₅-alkoxycarbonyl, C₂-C₅-alkenyl, C₂-C₅-alkenyloxy, C₂-C₅-halogenalkenyl, C₂-C₅-halogenalkenyloxy, C₂-C₅-alkinyl, C₂-C₅-alkinyloxy, C₁-C₅-alkylendioxy, C₁-C₂-haloalkylendioxy, C₁-C₅-alkylthio, C₁-C₅-halogenalkylthio, C₁-C₅-alkoxyimino-C₁-C₅-alkyl and the grouping
A³ preferably stands for a single bond or for C₁-C₆-alkanediyl, which is optionally substituted by one to six equivalent or different substituents from the group C₁-C₃-halogenalkyl, C₃-C₈-cycloalkyl and C₃-C₈-cycloalkyl-C₁-C₆-alkyl,
R⁶ preferably stands for hydrogen, cyano, hydroxy, C₁-C₅-alkyl, C₃-C₆-cycloalkyl, C₃-C₆-cycloalkyl-C₁-C₅-alkyl, C₁-C₅-halogenalkyl, C₁-C₅-alkoxy, C₁-C₅-halogenalkoxy, C₂-C₅-alkenyloxy, C₂-C₅-halogenalkenyloxy, C₂-C₅-alkinyloxy, —C(═O)R⁸, —C(═O)R⁸, or optionally for phenyl or benzyl substituted one to five times, the same or differently, in each case in the aryl part by halogen, C₁-C₅-alkyl, C₁-C₅-halogenalkyl, C₁-C₅-alkoxy, C₁-C₅-halogenalkoxy, hydroxy, cyano or nitro.
R⁷ preferably stands for hydrogen, cyano, C₁-C₅-alkyl, C₃-C₆-cycloalkyl, C₃-C₆-cycloalkyl-C₁-C₅-alkyl, C₁-C₅-halogenalkyl, —C(═O)R⁸, —C(═S)R⁸, or for phenyl or benzyl optionally substituted one to five times, the same or differently, in each case in the aryl part by halogen, C₁-C₅-alkyl, C₁-C₅-halogenalkyl, C₁-C₅-alkoxy, C₁-C₅-halogenalkoxy, hydroxy, cyano or nitro.
R⁷ together with R⁶ likewise preferably stands for C₄-C₆-alkanediyl or C₄-C₆-alkylenediyl optionally substituted in each case one to four times, the same or differently, by C₁-C₅-alkyl, C₃-C₆-cycloalkyl-C₁-C₅-alkyl, C₁-C₅-halogenalkyl, cyano or C₁-C₅-alkylcarbonyl, whereby a CH₂ group can be optionally replaced by O, S or NR⁹.
R⁷ likewise preferably stands for —C(═O)R⁸ or —C(═S)R⁸, whereby R⁶ und R⁸ then stand together in each case for C₂-C₄-alkanediyl or C₂-C₄-alkylenediyl optionally substituted one to four times, the same or differently, by C₁-C₅-alkyl, C₃-C₆-cycloalkyl-C₁-C₅-alkyl, C₁-C₅-halogenalkyl, cyano or C₁-C₅-alkylcarbonyl, whereby a CH₂ group can be optionally replaced by O, S or NR⁹, or
R⁶ and R⁷ likewise independently from one another preferably stand for —C(═O)R⁸ or —C(═S)R⁸, whereby both of the moieties R⁸ together stand in each case for straight-chain or branched C₂-C₄-alkanediyl or C₂-C₄-alkylenediyl optionally substituted one to four times, the same or differently, by C₁-C₅-alkyl, C₃-C₆-cycloalkyl-C₁-C₅-alkyl, C₁-C₅-halogenalkyl, cyano or C₁-C₅-alkylcarbonyl, and wherein a CH₂ group can be optionally replaced by O, S or NR⁹.
R⁸ preferably stands for C₁-C₅-alkyl, C₁-C₅-halogenalkyl, C₂-C₅-alkenyl, C₂-C₅-halogenalkenyl, C₂-C₅-alkinyl, C₁-C₅-alkoxy, C₁-C₅-halogenalkoxy, C₂-C₅-alkenyloxy, C₂-C₅-halogenalkenyloxy, C₂-C₅-alkinyloxy, C₃-C₅-Cycloalkyl, for phenyl or benzyl optionally substituted in each case one to three times, the same or differently, in the aryl part by halogen, cyano, nitro, C₁-C₅-alkyl, C₁-C₅-halogenalkyl, C₁-C₅-alkylcarbonyl, C₂-C₅-alkenyl, C₂-C₅-halogenalkenyl, C₂-C₅-alkinyl, C₁-C₅-alkoxy, C₁-C₅-halogenalkoxy, C₁-C₅-alkoxycarbonyl, C₁-C₃-halogenalkoxycarbonyl or C₂-C₅-halogenalkenyloxy.
R⁹ preferably stands for hydrogen, C₁-C₅-alkyl, C₁-C₃-halogenalkyl, C₁-C₃-halogenalkylcarbonyl, C₁-C₅-alkoxyalkyl, C₁-C₅-alkylcarbonyl or C₃-C₆Cycloalkyl.
Y preferably stands for a heterocyclic grouping connected with the adjacent groupings at two different positions selected from the following list (in this respect, the exocyclic dashes indicate the connections with the adjacent groupings in each case according to the order in formula (I)), 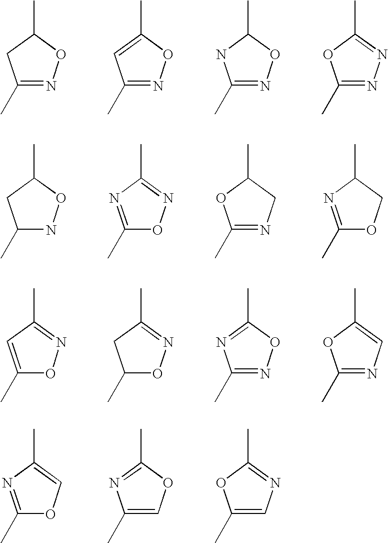
   whereby these heterocyclic groupings can be optionally substituted in each case by one or two substituents from the series nitro, hydroxy, amino, cyano, halogen, C₁-C₅-alkyl, C₁-C₅-halogenalkyl, C₁-C₅-alkoxy, C₁-C₅-halogenalkoxy, C₁-C₅-alkylthio, C₁-C₅-halogenalkythio.
A¹ particularly preferably stands for one of the following groupings:
   —CH₂—CH═CCl₂, —CH₂—CH═CBr₂, —CH₂—CH═CClF, —CH₂—CH═CBrCl.
A² particularly preferably stands for one of the following listed alkanediyl groupings:
   —CH₂—, —CH₂CH₂—, —CH(CH₃)—CH₂—, —CH₂CH(CH₃)—, —CH₂CH₂CH₂—, —CH(CH₃)CH₂CH₂—, —CH₂CH(CH₃)CH₂—, —CH₂CH₂CH(CH₃)—, —CH₂CH₂CH₂CH₂—, —CH₂CH₂CH₂CH₂CH₂—
   which likewise in each case contains an oxygen atom, a sulphur atom or a grouping selected from SO, SO₂, NH or N(methyl) at the beginning of, at the end of or within the carbon chain.
R¹ particularly preferably stands for hydrogen, nitro, hydroxy, amino, cyano, fluorine, chlorine, bromine, iodine, for methyl, ethyl, n- or i-propyl, n-, i-, s- or t-butyl, methoxy, ethoxy, n- or i-propoxy, n-, i-, s- or t-butoxy, methylthio, ethylthio, n- or i-propylthio, n-, i-, s- or t-butylthio, methylamino, ethylamino, n- or i-propylamino, n-, i-, s- or t-butylamino, dimethylamino, diethylamino, dipropylamino, acetylamino, propionylamino, n- or i-butyroylamino, methoximinomethyl, ethoximinomethyl, methoximinoethyl or ethoximinoethyl optionally substituted in each case by cyano, fluorine, chlorine, methylsulphinyl, methylsulphonyl, methoxy, ethoxy, n- or i-propoxy, for methylcarbonyloxy, ethylcarbonyloxy, n- or i-propylcarbonyloxy, methoxycarbonyloxy, ethoxycarbonyloxy, n- or i-propoxycarbonyloxy, cyclopropoxycarbonyloxy, cyclobutoxycarbonyloxy, cyclopentoxycarbonyloxy, cyclohexoxycarbonyloxy, for phenoxy, naphthyloxy, phenylthio, naphthylthio, benzyl or phenylethyl optionally substituted in each case by nitro, hydroxy, amino, cyano, fluorine, chlorine, bromine, iodine, methyl, ethyl, n- or i-propyl, n-, i-, s- or t-butyl, difluoromethyl, trifluoromethyl, chlorodifluoromethyl, fluoroethyl, difluoroethyl, trifluoroethyl, chloroethyl, dichloroethyl, trichloroethyl, methoxy, ethoxy, n- or i-propoxy, n-, i-, s- or t-butoxy, fluoromethoxy, difluoromethoxy, trifluoromethoxy, chlorodifluoromethoxy, fluoroethoxy, difluoroethoxy, trifluoroethoxy, chloroethoxy or dichloroethoxy, for heterocyclyloxy or heterocyclylthio with up to 9 carbon atoms, 1 to 4 nitrogen atoms and/or an oxygen or sulphur atom in each case, substituted in each case by nitro, hydroxy, amino, cyano, fluorine, chlorine, bromine, iodine, methyl, ethyl, n- or i-propyl, n-, i-, s- or t-butyl, difluoromethyl, trifluoromethyl, chlorodifluoromethyl, fluoroethyl, difluoroethyl, trifluoroethyl, chloroethyl, dichloroethyl, trichloroethyl, methoxy, ethoxy, n- or i-propoxy, n-, i-, s- or t-butoxy, fluoromethoxy, difluoromethoxy, trifluoromethoxy, chlorodifluoromethoxy, fluoroethoxy, difluoroethoxy, trifluoroethoxy, chloroethoxy or dichloroethoxy, or for the grouping —O-A¹, whereby A¹ has the meaning provided above, or for the grouping —N(R,R′), whereby R and R′ together with the N atom to which they are connected stand for pyrrolidinyl, piperidinyl, morpholinyl or piperazinyl optionally substituted in each case once or twice by methyl and/or ethyl.
R² particularly preferably stands for hydrogen, nitro, cyano, cyanato, thiocyanato, formyl, fluorine, chlorine, bromine, iodine, for methyl, ethyl, n- or i-propyl, n-, i-, s- or t-butyl, methoxy, ethoxy, n- or i-propoxy, n-, i-, s- or t-butoxy, methylthio, ethylthio, n- or i-propylthio, n-, i-, s- or t-butylthio, methylamino, ethylamino, n- or i-propylamino, n-, i-, s- or t-butylamino, dimethylamino, diethylamino, acetylamino, propionylamino, n- or i-butyroylamino, acetyl, propionyl, n- or i-butyroyl, methoxycarbonyl, ethoxycarbonyl, n- or i-propoxycarbonyl, methoximinoformyl, ethoximinoformyl, methoximinoacetyl or ethoximinoacetyl optionally substituted in each case by cyano, fluorine, chlorine, methoxy, ethoxy, n- or i-propoxy.
R³ particularly preferably stands for hydrogen, nitro, fluorine, chlorine, bromine, iodine, for methyl, ethyl, n- or i-propyl, n-, i-, s- or t-butyl, methoxy, ethoxy, n- or i-propoxy, n-, i-, s- or t-butoxy, methylthio, ethylthio, n- or i-propylthio, n-, i-, s- or t-butylthio, methylamino, ethylamino, n- or i-propylamino, n-, i-, s- or t-butylamino, optionally substituted in each case by cyano, fluorine, chlorine, methoxy, ethoxy, n- or i-propoxy.
R⁴ particularly preferably stands for hydrogen, nitro, fluorine, chlorine, bromine, iodine, for methyl, ethyl, n- or i-propyl, n-, i-, s- or t-butyl, methoxy, ethoxy, n- or i-propoxy, n-, i-, s- or t-butoxy, methylthio, ethylthio, n- or i-propylthio, n-, i-, s- or t-butylthio, methylamino, ethylamino, n- or i-propylamino, n-, i-, s- or t-butylamino, optionally substituted in each case by cyano, fluorine, chlorine, methoxy, ethoxy, n- or i-propoxy.
R⁵ particularly preferably stands for hydrogen, for phenyl or naphthyl substituted by nitro, hydroxy, amino, cyano, fluorine, chlorine, bromine, iodine, methyl, ethyl, n- or i-propyl, n-, i-, s- or t-butyl, difluoromethyl, trifluoromethyl, chlorodifluoromethyl, fluoroethyl, difluoroethyl, trifluoroethyl, chloroethyl, dichloroethyl, trichloroethyl, methoxy, ethoxy, n- or i-propoxy, n-, i-, s- or t-butoxy, fluoromethoxy, difluoromethoxy, trifluoromethoxy, chlorodifluoromethoxy, fluoroethoxy, difluoroethoxy, trifluoroethoxy, chloroethoxy or dichloroethoxy, C₁-C₂-alkylendioxy, C₁-C₂-fluoroalkylendioxy, methylthio, ethylthio, n- or i-propylthio, n-, i-, s- or t-butylthio, difluoromethylthio, trifluoromethylthio, chlorodifluoromethylthio, methoximinomethyl, ethoximinomethyl, methoximinoethyl or ethoximinoethyl, or for optionally substituted heteroaryl from the series furyl, thienyl, pyrrolyl, pyrazolyl, oxazolyl, isoxazolyl, thiazolyl, isothiazolyl, oxadiazolyl, thiadiazolyl, triazolyl, pyridinyl and pyrimidinyl, whereby the substituents can be selected from the following group of substituents:
   nitro, hydroxy, amino, cyano, fluorine, chlorine, bromine, iodine, methyl, ethyl, n- or i-propyl, n-, i-, s- or t-butyl, difluoromethyl, trifluoromethyl, chlorodifluoromethyl, fluoroethyl, difluoroethyl, trifluoroethyl, chloroethyl, dichloroethyl, trichloroethyl, methoxy, ethoxy, n- or i-propoxy, n-, i-, s- or t-butoxy, fluoromethoxy, difluoromethoxy, trifluoromethoxy, chlorodifluoromethoxy, fluoroethoxy, difluoroethoxy, trifluoroethoxy, chloroethoxy or dichloroethoxy, methylcarbonyl, ethylcarbonyl, n- or i-propylcarbonyl, n-, i-, s- or t-butylcarbonyl, ethoxycarbonyl, n- or i-propoxycarbonyl, n-, i-, s- or t-butoxycarbonyl, ethenyl, 2-propenyl, 1-butenyl, 2-butenyl, 3-butenyl, 1-pentenyl, 2-pentenyl, 3-pentenyl, ethenyloxy, 2-propenyloxy, 1-butenyloxy, 2-butenyloxy, 3-butenyloxy, 1-pentenyloxy, 2-pentenyloxy, 3-pentenyloxy, fluoroethenyl, difluoroethenyl, trifluoroethenyl, chloroethenyl, dichloroethenyl, trichloroethenyl, fluoroethenyloxy, difluoroethenyloxy, trifluoroethenyloxy, chloroethenyloxy, dichloroethenyloxy, trichloroethenyloxy, ethinyl, 1-propinyl, 2-propinyl, 1-butinyl, 2-butinyl, 3-butinyl, 1-pentinyl, 2-pentinyl, 3-pentinyl, C₁-C₂-alkylendioxy, C₁-C₂-fluoroalkylendioxy, methylthio, ethylthio, n- or i-propylthio, n-, i-, s- or t-butylthio, difluoromethylthio, trifluoromethylthio, chlorodifluoromethylthio, methoximinomethyl, ethoximinomethyl, methoximinoethyl or ethoximinoethyl and the grouping
A³ particularly preferably stands for a single bond or for one of the groups —CH₂—, —CH₂CH₂—, —CH₂—CH₂—CH₂—, —CH₂—CH₂—CH₂—CH₂—, —CH₂—CH₂—CH₂—CH₂—CH₂—, —CH(CH₃)—, —CH(CH₃)CH₂—CH₂—, —CH(C₂H₅)—, —C(CH₃)₂—, —CH(CH₃)CH₂—, —CH(CH₃)CH(CH₃)— and —CH₂C(CH₃)₂—CH₂—, which is optionally substituted with one to four identical or different substituents from the group difluoromethyl, trifluoromethyl, chlorodifluoromethyl, fluoroethyl, difluoroethyl, trifluoroethyl, chloroethyl, dichloroethyl, trichloroethyl, cyclopropyl, cyclobutyl, cyclopentyl, cyclohexyl, cyclopropylmethyl, cyclopropylethyl, cyclobutylmethyl, cyclobutylethyl, cyclopentylmethyl, cyclopentylethyl, cyclohexylmethyl and cyclohexylethyl.
R⁶ particularly preferably stands for hydrogen, cyano, hydroxy, methyl, ethyl, n- or i-propyl, n-, i-, s- or t-butyl, cyclopropyl, cyclobutyl, cyclopentyl, cyclohexyl, cyclopropylmethyl, cyclopropylethyl, cyclobutylmethyl, cyclobutylethyl, cyclopentylmethyl, cyclopentylethyl, cyclohexylmethyl and cyclohexylethyl, difluoromethyl, trifluoromethyl, chlorodifluoromethyl, fluoroethyl, difluoroethyl, trifluoroethyl, chloroethyl, dichloroethyl, trichloroethyl, methoxy, ethoxy, n- or i-propoxy, n-, i-, s- or t-butoxy, fluoromethoxy, difluoromethoxy, trifluoromethoxy, chlorodifluoromethoxy, fluoroethoxy, difluoroethoxy, trifluoroethoxy, chloroethoxy or dichloroethoxy, ethenyloxy, 2-propenyloxy, 1-butenyloxy, 2-butenyloxy, 3-butenyloxy, 1-pentenyloxy, 2-pentenyloxy, 3-pentenyloxy, fluoroethenyloxy, difluoroethenyloxy, trifluoroethenyloxy, chloroethenyloxy, dichloroethenyloxy, trichloroethenyloxy, —C(═O)R⁸, —C(═O)R⁸, or for phenyl or benzyl optionally substituted in each case one to five times, the same or differently, in the aryl part by fluorine, chlorine, bromine, methyl, ethyl, n- or i-propyl, n-, i-, s- or t-butyl difluoromethyl, trifluoromethyl, chlorodifluoromethyl, fluoroethyl, difluoroethyl, trifluoroethyl, chloroethyl, dichloroethyl, trichloroethyl, methoxy, ethoxy, n- or i-propoxy, n-, i-, s- or t-butoxy, fluoromethoxy, difluoromethoxy, trifluoromethoxy, chlorodifluoromethoxy, fluoroethoxy, difluoroethoxy, trifluoroethoxy, chloroethoxy or dichloroethoxy, hydroxy, cyano or nitro.
R⁷ particularly preferably stands for hydrogen, cyano, methyl, ethyl, n- or i-propyl, n-, i-, s- or t-butyl, cyclopropyl, cyclobutyl, cyclopentyl, cyclohexyl, cyclopropylmethyl, cyclopropylethyl, cyclobutylmethyl, cyclobutylethyl, cyclopentylmethyl, cyclopentylethyl, cyclohexylmethyl and cyclohexylethyl, difluoromethyl, trifluoromethyl, chlorodifluoromethyl, fluoroethyl, difluoroethyl, trifluoroethyl, chloroethyl, dichloroethyl, trichloroethyl, —C(═O)R⁸, —C(═S)R⁸, or for phenyl or benzyl optionally substituted in each case one to five times, the same or differently, in the aryl part by halogen, methyl, ethyl, n- or i-propyl, n-, i-, s- or t-butyl, difluoromethyl, trifluoromethyl, chlorodifluoromethyl, fluoroethyl, difluoroethyl, trifluoroethyl, chloroethyl, dichloroethyl, trichloroethyl, methoxy, ethoxy, n- or i-propoxy, n-, i-, s- or t-butoxy, fluoromethoxy, difluoromethoxy, trifluoromethoxy, chlorodifluoromethoxy, fluoroethoxy, difluoroethoxy, trifluoroethoxy, chloroethoxy or dichloroethoxy, hydroxy, cyano or nitro.
R⁷ together with R⁶ likewise particularly preferably stands for alkanediyl or alkylenediyl from the series —CH₂—, —CH₂CH₂—, —CH₂—CH₂—CH₂—, —CH₂—CH₂—CH₂—CH₂—, —CH₂—CH₂—CH₂—CH₂—CH₂—, —CH(CH₃)—, —CH(CH₃)CH₂—CH₂—, —CH(C₂H₅)—, —C(CH₃)₂—, —CH(CH₃)CH₂—, —CH(CH₃)CH(CH₃)—, —CH₂C(CH₃)₂—CH₂—, —CH═CH—, —CH═CH—CH₂—, —CH₂—CH═CH—CH₂—, —CH₂—CH═CH—CH₂—CH₂— and —CH(CH₃)CH═CH—, optionally substituted one to four times, the same or differently, by methyl, ethyl, n- or i-propyl, n-, i-, s- or t-butyl, cyclopropylmethyl, cyclopropylethyl, cyclobutylmethyl, cyclobutylethyl, cyclopentylmethyl, cyclopentylethyl, cyclohexylmethyl and cyclohexylethyl, difluoromethyl, trifluoromethyl, chlorodifluoromethyl, fluoroethyl, difluoroethyl, trifluoroethyl, chloroethyl, dichloroethyl, trichloroethyl, cyano or methylcarbonyl, ethylcarbonyl, n- or i-propylcarbonyl, n-, i-, s- or t-butylcarbonyl, whereby a CH₂ group can be optionally replaced by O, S or NR⁹.
R⁷ likewise particularly preferably stands for —C(═O)R⁸ or —C(═S)R⁸, whereby R⁶ and R⁸ together stand for alkanediyl or alkylenediyl from the series —CH₂—, —CH₂CH₂—, —CH₂—CH₂—CH₂—, —CH₂—CH₂—CH₂—CH₂—, —CH(CH₃)—, —CH(CH₃)CH₂—CH₂—, —CH(C₂H₅)—, —C(CH₃)₂—, —CH(CH₃)CH₂—, —CH(CH₃)CH(CH₃)—, —CH═CH—, —CH═CH—CH₂—, —CH₂—CH═CH—CH₂—, and —CH(CH₃)CH═CH—, optionally substituted in each case one to four times, the same or differently, by methyl, ethyl, n- or i-propyl, n-, i-, s- or t-butyl, cyclopropylmethyl, cyclopropylethyl, cyclobutylmethyl, cyclobutylethyl, cyclopentylmethyl, cyclopentylethyl, cyclohexylmethyl and cyclohexylethyl, difluoromethyl, trifluoromethyl, chlorodifluoromethyl, fluoroethyl, difluoroethyl, trifluoroethyl, chloroethyl, dichloroethyl, trichloroethyl, cyano or methylcarbonyl, ethylcarbonyl, n- or i-propylcarbonyl, n-, i-, s- or t-butylcarbonyl, and whereby a CH₂ group can be optionally replaced by O, S or NR⁹.
R⁶ and R⁷ likewise particularly preferably stand independently from one another for —C(═O)R⁸ or —C(═S)R⁸, whereby both of the moieties R⁸ stand for alkanediyl or alkylenediyl from the series —CH₂—, —CH₂CH₂—, —CH₂—CH₂—CH₂—, —CH₂—CH₂—CH₂—CH₂—, —CH(CH₃)—, —CH(CH₃)CH₂—CH₂—, —CH(C₂H₅)—, —C(CH₃)₂—, —CH(CH₃)CH₂—, —CH(CH₃)CH(CH₃)—, —CH═CH—, —CH═CH—CH₂—, —CH₂—CH═CH—CH₂—, and —CH(CH₃)CH═CH—, optionally substituted in each case one to four times, the same or differently, by methyl, ethyl, n- or i-propyl, n-, i-, s- or t-butyl, cyclopropylmethyl, cyclopropylethyl, cyclobutylmethyl, cyclobutylethyl, cyclopentylmethyl, cyclopentylethyl, cyclohexylmethyl and cyclohexylethyl, difluoromethyl, trifluoromethyl, chlorodifluoromethyl, fluoroethyl, difluoroethyl, trifluoroethyl, chloroethyl, dichloroethyl, trichloroethyl, cyano or methylcarbonyl, ethylcarbonyl, n- or i-propylcarbonyl, n-, i-, s- or t-butylcarbonyl, and whereby a CH₂ group can be optionally replaced by O, S or NR⁹.
R⁸ particularly preferably stands for methyl, ethyl, n- or i-propyl, n-, i-, s- or t-butyl, difluoromethyl, trifluoromethyl, chlorodifluoromethyl, fluoroethyl, difluoroethyl, trifluoroethyl, chloroethyl, dichloroethyl, trichloroethyl, ethenyl, 2-propenyl, 1-butenyl, 2-butenyl, 3-butenyl, 1-pentenyl, 2-pentenyl, 3-pentenyl, fluoroethenyl, difluoroethenyl, trifluoroethenyl, chloroethenyl, dichloroethenyl, trichloroethenyl, ethinyl, 1-propinyl, 2-propinyl, 1-butinyl, 2-butinyl, 3-butinyl, 1-pentinyl, 2-pentinyl, 3-pentinyl, methoxy, ethoxy, n- or i-propoxy, n-, i-, s- or t-butoxy, fluoromethoxy, difluoromethoxy, trifluoromethoxy, chlorodifluoromethoxy, fluoroethoxy, difluoroethoxy, trifluoroethoxy, chloroethoxy or dichloroethoxy, ethenyloxy, 2-propenyloxy, 1-butenyloxy, 2-butenyloxy, 3-butenyloxy, 1-pentenyloxy, 2-pentenyloxy, 3-pentenyloxy, fluoroethenyl, difluoroethenyl, trifluoroethenyl, chloroethenyl, dichloroethenyl, trichloroethenyl, ethinyloxy, 1-propinyloxy, 2-propinyloxy, 1-butinyloxy, 2-butinyloxy, 3-butinyloxy, C₃-C₅-cycloalkyl, for phenyl or benzyl optionally substituted in each case one to three times, the same or differently, in the aryl part by halogen, cyano, nitro, methyl, ethyl, n- or i-propyl, n-, i-, s- or t-butyl, difluoromethyl, trifluoromethyl, chlorodifluoromethyl, fluoroethyl, difluoroethyl, trifluoroethyl, chloroethyl, dichloroethyl, trichloroethyl, methylcarbonyl, ethylcarbonyl, n- or i-propylcarbonyl, n-, i-, s- or t-butylcarbonyl, ethenyl, 2-propenyl, 1-butenyl, 2-butenyl, 3-butenyl, 1-pentenyl, 2-pentenyl, 3-pentenyl, fluoroethenyl, difluoroethenyl, trifluoroethenyl, chloroethenyl, dichloroethenyl, trichloroethenyl, ethinyl, 1-propinyl, 2-propinyl, 1-butinyl, 2-butinyl, 3-butinyl, 1-pentinyl, 2-pentinyl, 3-pentinyl, methoxy, ethoxy, n- or i-propoxy, n-, i-, s- or t-butoxy, fluoromethoxy, difluoromethoxy, trifluoromethoxy, chlorodifluoromethoxy, fluoroethoxy, difluoroethoxy, trifluoroethoxy, chloroethoxy or dichloroethoxy, methoxycarbonyl, ethoxycarbonyl, n- or i-propoxycarbonyl, n-, i-, s- or t-butoxycarbonyl, fluoromethoxycarbonyl, difluoromethoxycarbonyl, trifluoromethoxycarbonyl, chlorodifluoromethoxycarbonyl, fluoroethoxycarbonyl, difluoroethoxycarbonyl, trifluoroethoxycarbonyl, chloroethoxycarbonyl or dichloroethoxycarbonyl.
R⁹ particularly preferably stands for hydrogen, methyl, ethyl, n- or i-propyl, n-, i-, s- or t-butyl, difluoromethyl, trifluoromethyl, chlorodifluoromethyl, fluoroethyl, difluoroethyl, trifluoroethyl, chloroethyl, dichloroethyl, trichloroethyl, methoxymethyl, methoxyethyl, ethoxymethyl, ethoxyethyl, n- or i-propoxymethyl, n- or i-propoxyethyl, n-, i-, s- or t-butoxymethyl, n-, i-, s- or t-butoxymethyl, methoxycarbonyl, ethoxycarbonyl, n- or i-propoxycarbonyl, n-, i-, s- or t-butoxycarbonyl, cyclopropyl, cyclobutyl, cyclopentyl or cyclohexyl.
Y particularly preferably stands for a heterocyclic grouping connected with the adjacent groupings at two different positions selected from the following list (in this respect, the exocyclic dashes indicate the connections with the adjacent groupings in each case according to the order in formula (I)), 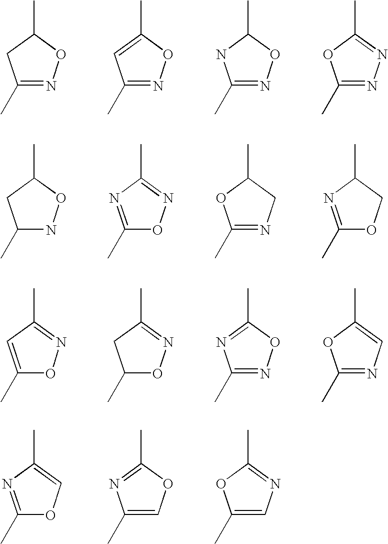
   whereby these heterocyclic groupings can be optionally substituted in each case by one or two substituents from the series nitro, hydroxy, amino, cyano, fluorine, chlorine, bromine, methyl, ethyl, n- or i-propyl, n-, i-, s- or t-butyl, difluoromethyl, trifluoromethyl, chlorodifluoromethyl, fluoroethyl, difluoroethyl, trifluoroethyl, chloroethyl, dichloroethyl, trichloroethyl, methoxy, ethoxy, n- or i-propoxy, n-, i-, s- or t-butoxy, fluoromethoxy, difluoromethoxy, trifluoromethoxy, chlorodifluoromethoxy, fluoroethoxy, difluoroethoxy, trifluoroethoxy, chloroethoxy or dichloroethoxy, methylthio, ethylthio, n- or i-propylthio, n-, i-, s- or t-butylthio, difluoromethylthio, trifluoromethylthio or chlorodifluoromethylthio.
A¹ very particularly preferably stands for the grouping —CH₂—CH═CCl₂.
A² very particularly preferably stands for one of the following listed alkanediyl groupings:
   —CH₂O—, —CH₂CH₂O—, —CH₂CH₂CH₂O—, —CH₂CH₂CH₂CH₂O—.
R¹ very particularly preferably stands for hydrogen, nitro, hydroxy, cyano, fluorine, chlorine, bromine, methyl, ethyl, n- or i-propyl, methoxy, ethoxy, n- or i-propoxy, methylthio, ethylthio, n- or i-propylthio, methylamino, ethylamino, n- or i-propylamino, dimethylamino, for phenoxy, phenylthio, benzyl or phenylethyl optionally substituted in each case by nitro, hydroxy, cyano, fluorine, chlorine, bromine, methyl, ethyl, n- or i-propyl, n-, i-, s- or t-butyl, difluoromethyl, trifluoromethyl, chlorodifluoromethyl, fluoroethyl, difluoroethyl, trifluoroethyl, chloroethyl, dichloroethyl, trichloroethyl, methoxy, ethoxy, n- or i-propoxy, fluoromethoxy, difluoromethoxy, trifluoromethoxy, chlorodifluoromethoxy, fluoroethoxy, difluoroethoxy, trifluoroethoxy, chloroethoxy or dichloroethoxy, or for the grouping —O-A¹, whereby A¹ has one of the meanings provided above.
R² very particularly preferably stands for hydrogen, cyano, fluorine, chlorine, bromine, methyl, ethyl, methoxy or ethoxy.
R³ very particularly preferably stands for hydrogen, cyano, fluorine, chlorine, bromine, methyl, ethyl, methoxy or ethoxy.
R⁴ very particularly preferably stands for hydrogen, cyano, fluorine, chlorine or bromine.
R⁵ very particularly preferably stands for hydrogen, for phenyl optionally substituted by nitro, cyano, fluorine, chlorine, bromine, methyl, ethyl, n- or i-propyl, difluoromethyl, trifluoromethyl, chlorodifluoromethyl, fluoroethyl, difluoroethyl, trifluoroethyl, chloroethyl, dichloroethyl, trichloroethyl, methoxy, ethoxy, n- or i-propoxy, fluoromethoxy, difluoromethoxy, trifluoromethoxy, chlorodifluoromethoxy, fluoroethoxy, difluoroethoxy, trifluoroethoxy, chloroethoxy or dichloroethoxy, C₁-C₂-alkylendioxy, C₁-C₂-fluoroalkylendioxy, methylthio, ethylthio, n- or i-propylthio, difluoromethylthio, trifluoromethylthio, chlorodifluoromethylthio, methoximinomethyl, ethoximinomethyl, methoximinoethyl or ethoximinoethyl, or for optionally substituted pyridinyl, whereby the substituents are selected from the following group of substituents:
   nitro, hydroxy, amino, cyano, fluorine, chlorine, bromine, iodine, methyl, ethyl, n- or i-propyl, n-, i-, s- or t-butyl, difluoromethyl, trifluoromethyl, chlorodifluoromethyl, fluoroethyl, difluoroethyl, trifluoroethyl, chloroethyl, dichloroethyl, trichloroethyl, methoxy, ethoxy, n- or i-propoxy, n-, i-, s- or t-butoxy, fluoromethoxy, difluoromethoxy, trifluoromethoxy, chlorodifluoromethoxy, fluoroethoxy, difluoroethoxy, trifluoroethoxy, chloroethoxy or dichloroethoxy, methylcarbonyl, ethylcarbonyl, n- or i-propylcarbonyl, n-, i-, s- or t-butylcarbonyl, ethoxycarbonyl, n- or i-propoxycarbonyl, n-, i-, s- or t-butoxycarbonyl, ethenyl, 2-propenyl, 1-butenyl, 2-butenyl, 3-butenyl, 1-pentenyl, 2-pentenyl, 3-pentenyl, ethenyloxy, 2-propenyloxy, 1-butenyloxy, 2-butenyloxy, 3-butenyloxy, 1-pentenyloxy, 2-pentenyloxy, 3-pentenyloxy, fluoroethenyl, difluoroethenyl, trifluoroethenyl, chloroethenyl, dichloroethenyl, trichloroethenyl, fluoroethenyloxy, difluoroethenyloxy, trifluoroethenyloxy, chloroethenyloxy, dichloroethenyloxy, trichloroethenyloxy, ethinyl, 1-propinyl, 2-propinyl, 1-butinyl, 2-butinyl, 3-butinyl, 1-pentinyl, 2-pentinyl, 3-pentinyl, C₁-C₂-alkylendioxy, C₁-C₂-fluoroalkylendioxy, methylthio, ethylthio, n- or i-propylthio, n-, i-, s- or t-butylthio, difluoromethylthio, trifluoromethylthio, chlorodifluoromethylthio, methoximinomethyl, ethoximinomethyl, methoximinoethyl or ethoximinoethyl and the grouping 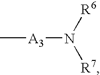
   wherein the moieties A³, R⁶ and R⁷ have one of the meanings provided above.
Y very particularly preferably stands for one of the following heterocyclic groupings (in this respect, the exocyclic dashes indicate the connections with the adjacent groupings in each case according to the order in formula (I)), 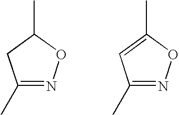
   whereby these heterocyclic groupings can be optionally substituted in each case by one or two substituents from the series nitro, hydroxy, cyano, fluorine, chlorine, bromine, methyl, ethyl, n- or i-propyl, difluoromethyl, trifluoromethyl, chlorodifluoromethyl, fluoroethyl, difluoroethyl, trifluoroethyl, chloroethyl, dichloroethyl, trichloroethyl, methoxy, ethoxy, n- or i-propoxy, fluoromethoxy, difluoromethoxy, trifluoromethoxy, chlorodifluoromethoxy, fluoroethoxy, difluoroethoxy, trifluoroethoxy, chloroethoxy or dichloroethoxy, methylthio, ethylthio, n- or i-propylthio, difluoromethylthio, trifluoromethylthio or chlorodifluoromethylthio.
R¹ most preferably stands for hydrogen, nitro, hydroxy, cyano, fluorine, chlorine, bromine, methyl, ethyl, n- or i-propyl, methoxy, ethoxy, n- or i-propoxy, methylthio, ethylthio, n- or i-propylthio, methylamino, ethylamino, n- or i-propylamino or dimethylamino.
R² most preferably stands for hydrogen, fluorine, chlorine or bromine.
R⁵ most preferably stands for hydrogen or for pyridinyl optionally substituted by fluorine, chlorine, bromine, methyl, ethyl, n- or i-propyl, difluoromethyl, trifluoromethyl, chlorodifluoromethyl, fluoroethyl, difluoroethyl, trifluoroethyl, chloroethyl, dichloroethyl, trichloroethyl, methoxy, ethoxy, n- or i-propoxy, fluoromethoxy, difluoromethoxy, trifluoromethoxy, chlorodifluoromethoxy, fluoroethoxy, difluoroethoxy, trifluoroethoxy, chloroethoxy or dichloroethoxy.
Y most preferably stands for one of the following heterocyclic groupings (in this respect, the exocyclic dashes indicate the connections with the adjacent groupings in each case according to the order in formula (I)) 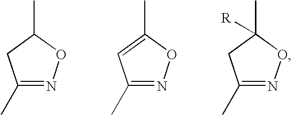
   wherein R stand for C₁-C₄-alkyl and preferably for methyl.

The general moiety definitions given above or given in the preferred groups apply both for the end products of the formula (I) as well as correspondingly for the starting and intermediate products required for production in each case. The moiety definitions can be arbitrarily combined with one another, as well as among the provided preferred groups.

The compounds of the formula (I) in which a combination of the meanings listed above as preferred above exists are preferred according to the invention.

The compounds of the formula (I) in which a combination of the meaning listed as particularly preferred above exist are particularly preferred according to the invention.

The compounds of the formula (I) in which a combination of the meanings listed as very particularly preferred above exists are very particularly preferred according to the invention.

The compounds of the formula (I) in which a combination of the meanings listed above as most preferred above exists are most preferred according to the invention.

In the moiety definitions listed above and below, hydrocarbon moieties such as alkyl—also in compound with heteroatoms such as in alkoxy—are straight-chain or branched to the extent possible in each case.

Examples for the compounds of the general formula (I) according to the invention are listed in the groups below.

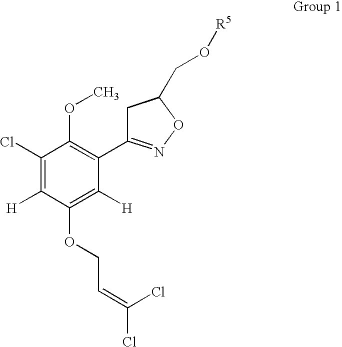

Here, R⁵ has the meanings provided in the list below:
2-trifluoromethyl-phenyl, 3-trifluoromethyl-phenyl, 4-trifluoromethyl-phenyl, 2-chloro-4-trifluoromethyl-phenyl, 2,6-dichloro-4-trifluoromethyl-phenyl, 5-trifluoromethyl-thien-3-yl, pyridin-2-yl, 5-fluoro-pyridin-2-yl, 5-chloro-pyridin-2-yl, 5-bromo-pyridin-2-yl, 5-nitro-pyridin-2-yl, 5-cyano-pyridin-2-yl, 5-methyl-pyridin-2-yl, 5-trifluoromethyl-pyridin-2-yl, 5-chlorodifluoromethyl-pyridin-2-yl, 5-methoxy-pyridin-2-yl, 3-fluoro-pyridin-2-yl, 3-chloro-pyridin-2-yl, 3-bromo-pyridin-2-yl, 3-nitro-pyridin-2-yl, 3-cyano-pyridin-2-yl, 3-methyl-pyridin-2-yl, 3-trifluoromethyl-pyridin-2-yl, 4-trifluoromethyl-pyridin-3-yl, 3-chlorodifluoromethyl-pyridin-2-yl, 3-methoxy-pyridin-2-yl, 3-chloro-5-trifluoromethyl-pyridin-2-yl, 3-bromo-5-trifluoromethyl-pyridin-2-yl, 6-(2,2,2-trifluoroethoxy)-pyridin-3-yl.

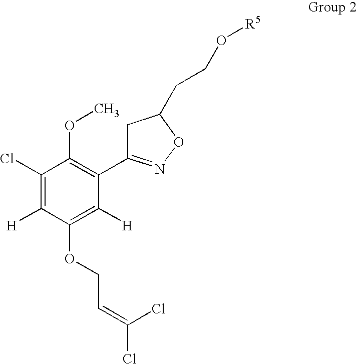

Here, R⁵ has the meanings provided above in Group 1.

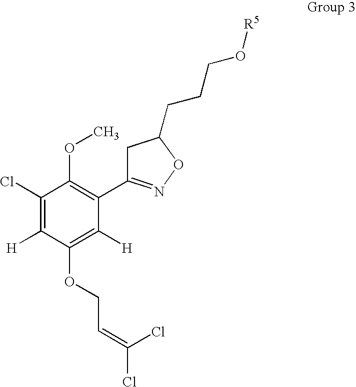

Here, R⁵ has the meanings provided above in Group 1.

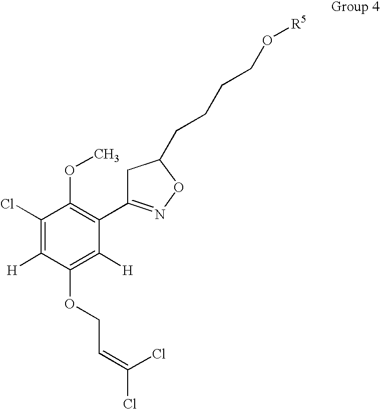

Here, R⁵ has the meanings provided above in Group 1.

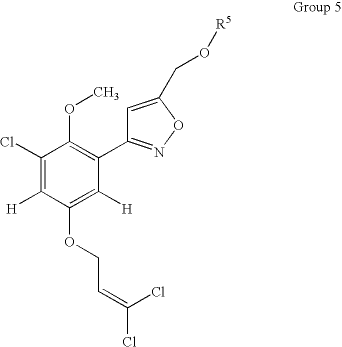

Here, R⁵ has the meanings provided above in Group 1.

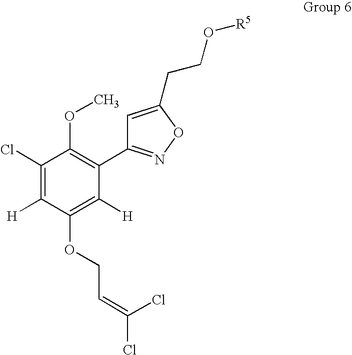

Here, R⁵ has the meanings provided above in Group 1.

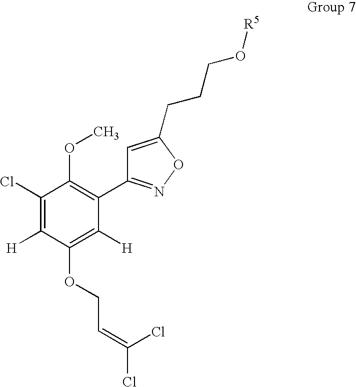

Here, R⁵ has the meanings provided above in Group 1.

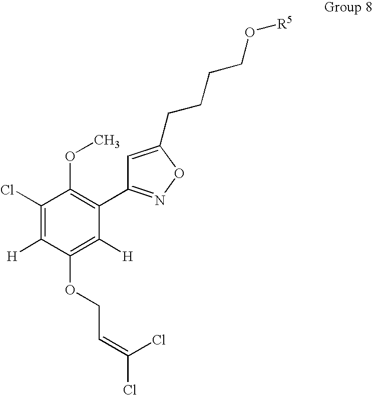

Here, R⁵ has the meanings provided above in Group 1.

The novel substituted oxyarenes of the general formula (I) have interesting biological characteristics. In particular, they distinguish themselves by strong arthropodicidal (insecticidal and acaricidal) as well as nematicidal effectiveness and can be used in agriculture, in forestry, in inventory and material protection as well as in the hygiene field.

One obtains the novel substituted oxyarenes of the general formula (I) when one causes substituted benzaldoximes of the general formula (II), 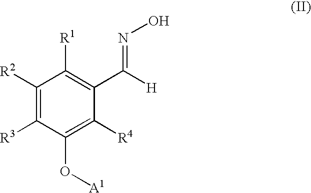
wherein
A¹, R¹, R², R³ and R⁴ have the meaning provided above,
to react with halogenation agents, optionally in the presence of one or more diluents,
here the generated substituted benzhydroxamine acid halogenides of the general formula (III), 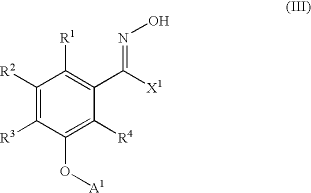
wherein
A¹, R¹, R², R³ and R⁴ have the meaning provided above and
X¹ stands for halogen,
caused to react in situ—i.e. without intermediate isolation—with one or more acid binding agents,
and the substituted arylnitrile-N-oxides of the general formula (IV) thus generated, 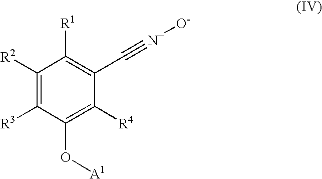
wherein
A¹, R¹, R², R³ and R⁴ have the meaning provided above,
caused to react in situ—i.e. without intermediate isolation—with alkenes of the general formula (V), 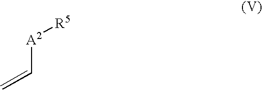
wherein
A² and R⁵ have the meaning provided above and
the carbon atoms of the olefinic double bond are optionally substituted as provided above for Y,
optionally in the presence of one or more diluents and optionally in the presence of one or more reaction aids,
and the compounds of the formula (I) thus obtained optionally converted into other compounds of the formula (I) according to traditional methods.

For example, if one uses 2-chloro-5-[(3,3-dichloro-2-propenyl)-oxy]-benzaldehyd-oxime and N-chloro-succinimide (NCS) in the first conversion step as well as 2-(allyloxy)-5-chloro-pyridine in the last conversion step as a starting substance, then the reaction activity during the process according to the invention can be outlined by the following formula schema:

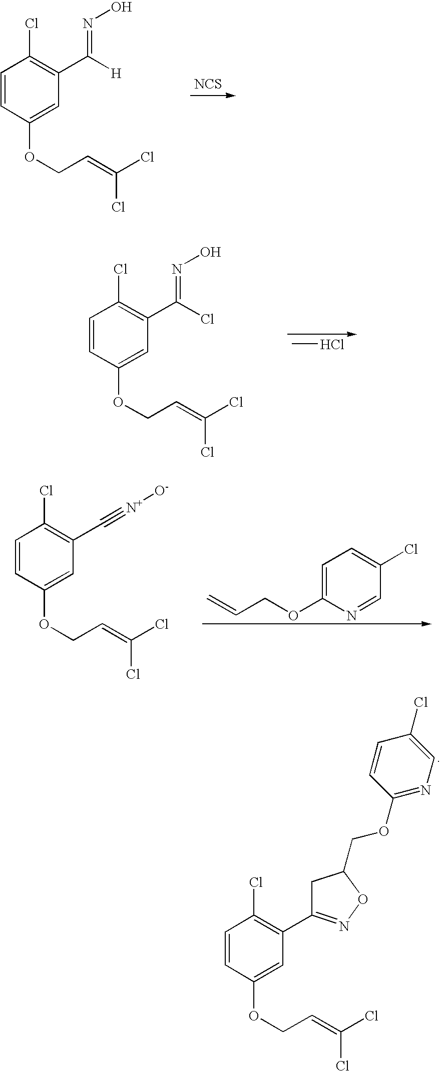

Compounds of the general formula (I) can, for example, also be synthesised as illustrated schematically as follows:
(a) by the conversion of arylnitrile-N-oxides of the general formula (IV) with alkines of the general formula (VI), 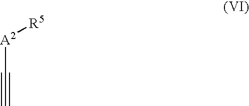
   wherein
   A² and R⁵ have the meaning provided above,
   analogous to the description above (also compare production examples as well as A. R. Kochetkov, S. D. Sokolov: Advances Heterocyclic Chem., A. R. Katritzky, A. J. Boulton (eds.), Vol. 2, New York: Academic Press 1963, p. 365; Houben Weyl, Methoden der Organischen Chemie, Band E8a, p. 45-176, G. Thieme Verlag, Stuttgart New York), whereby this conversion can be outlined as follows, 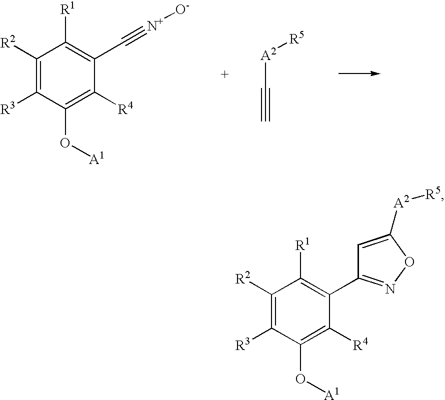
   and wherein R¹, R², R³, R⁴, R⁵, A¹ and A² have the preceding provided meanings;
(b) through the conversion of aryl-N-oxides of the general formula (IV) with nitriles of the general formula (VII), 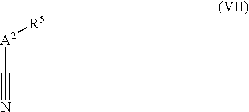
   wherein
   A² and R⁵ have the meaning provided above,
   analogous to the description above (also compare I. J. Turchi, J. S. Dewar: Chem. Reviews 75, (1975) p. 389; R. Lakhan, B. Ternahi: Advances Heterocyclic Chem., A. R. Katritzky, A. J. Boulton (eds.), Vol. 17, New York: Academic Press 1974, p. 99; J. W. Cornforth: Heterocyclic Compounds, R. C. Elderfield (ed), Vol. 5 New York: Wiley & Sons 1957, p. 298), whereby this conversion can be outlined as follows, 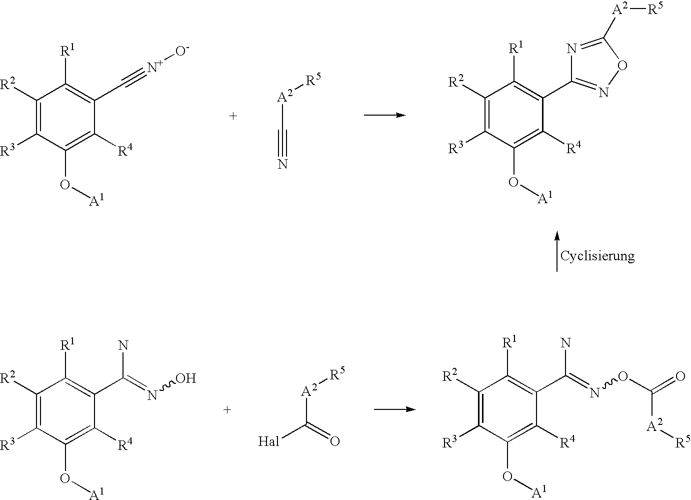
   and wherein R¹, R², R³, R⁴, R⁵, A¹ and A² have the preceding provided meanings.

Alternatively, production of the compounds of the formula (I) is possible from corresponding carboxylic acid derivatives, for example an amidoxime and an activated carboxylic acid derivative, for example a carboxylic acid halogenide, and subsequent cyclisation according to generally known methods, for example
(α) through the conversion of carboxylic acid hydrazides with an activated carboxylic acid derivative, for example a carboxylic acid halogenide and subsequent cyclisation in the presence of dehydrating agents, for example phosphoryl chloride, according to generally known methods (compare A. Hetzheim, K. Möckel, In: Advances Heterocyclic Chem., A. R. Katritzky, A. J. Boulton (eds.), Vol. 7, New York: Academic Press 1974, p. 183; J. H. Boyer: Heterocyclic Compounds, R. C. Elderfield (ed.) Vol. 7, New York, J. Wiley & Sons 1961, p. 462), whereby the conversion can be outlined as follows, 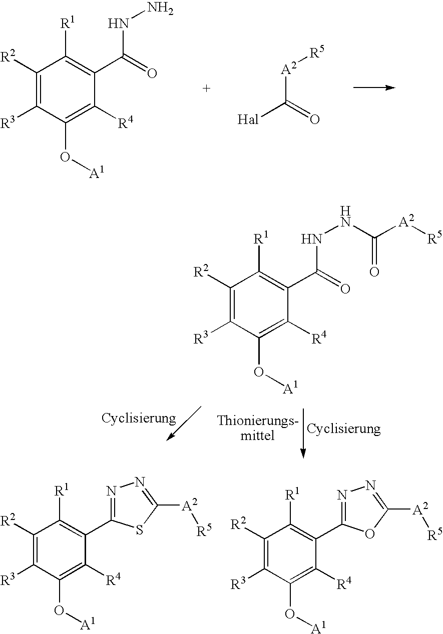
Cyclisierung=cyclisation; Thionierungsmittel=thionation agent
and wherein R¹, R², R³, R⁴, R⁵, A¹ and A² have the preceding provided meanings,
and whereby the use of a suitable thionation agent, for example diphosphorus pentasulfide (P₂S₅) or Lawesson's reagent (compare Review of Lawessons Reagent: R. A. Cherkasov et. al., Tetrahedron 41, 1985, p. 2567) continues the cyclisation in a known manner by incorporating sulphur (also compare J. Sandström: Advances Heterocyclic Chem., A. R. Katritzky, A. J. Boulton (eds.), Vol. 9, New York: Academic Press 1968, p. 165; L. L. Bambas, five-Membered Heterocyclic Compounds with Nitrogen and Sulfur or Nitrogen, Sulfur, and Oxygen, the Chemistry of Heterocyclic Compounds, Vol. 4, A. Weissberger (ed.), New York, Interscience Publ. 1952, p. 81), or
(β) through the conversion of α-halogen keto compounds, for example phenacyl halogenides, with a thioamide according to the generally known Hantzsch method (also compare R. H. Wiley et al., Org. Reactions 6 (1951) 367; J. M. Sprague, A. M. Land, Heterocyclic Compounds, Elderfield, R. C. (ed.) Vol. 5, New York, J. Wiley & Sons 1957, p. 484), which can be outlined as follows, 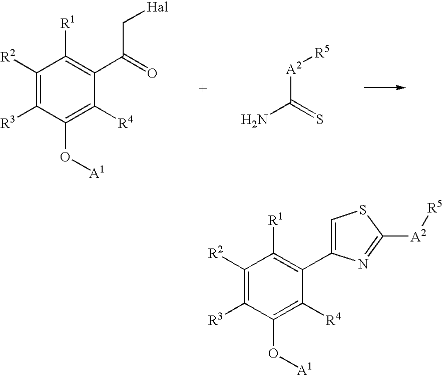
Cyclisierung=cyclisation; Thionierungsmittel=thionation agent
and wherein R¹, R², R³, R⁴, R⁵, A¹ and A² have the preceding provided meanings, or
(γ) through the conversion of α-halogen keto compounds, for example phenacyl halogenides, with a corresponding amidine according to sufficient and generally known methods (compare H. Beyer, Neue Synthesen von Imidazolen und imidazo-Bicyclen [Novel Synthesis of Imidazoles and Imidazo-bicyclics], Z. Chem. 10 (1970) p. 289; Grimmet, M. R., In: Advances Heterocyclic Chem., A. R. Katritzky, A. J. Boulton (eds.), Vol. 12, New York: Academic Press 1970, p. 104; K. Hoffmann, Imidazole and its Derivatives, The Chemistry of Heterocyclic Compounds, A. Weissberger, Taylor E. C. (eds.), New York, Wiley-Interscience 1953; E. S. Schippper, A. R. Day, Heterocyclic Compounds, R. C. Elderfield (ed.), Vol. 5, New York, J. Wiley & Sons 1956, p. 194), whereby the conversion can be outlined as follows, 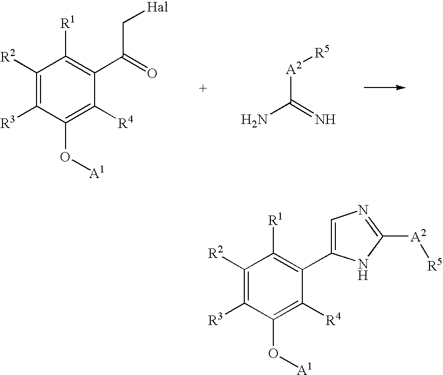
and wherein R¹, R², R³, R⁴, R⁵, A¹ and A² have the preceding provided meanings,
(δ) through the conversion of activated carboxylic acid derivatives with α-amino keto compounds to corresponding acylated α-amino keto compounds and subsequent cyclisation in the presence of dehydrating agents, for example phosphor(V) chloride or thionyl chloride, according to generally known methods (also compare M. R. Grimmet: Advances Heterocyclic Chem., A. R. Katritzky, A. J. Boulton (eds.), Vol. 12, New York: Academic Press 1970, p. 104; R. J. Ferm, J. L. Riebsommer Chem. Review 54 (1954) p. 593), whereby the conversion can be outlined as follows, 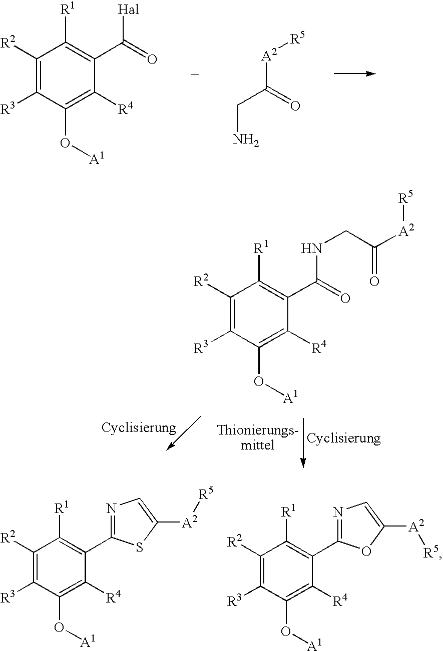
Cyclisierung=cyclisation; Thionierungsmittel=thionation agent
wherein R¹, R², R³, R⁴, R⁵, A¹ and A² have the preceding provided meanings,
and whereby the use of a suitable thionation agent, for example diphosphorus pentasulfide (P₂S₅) or Lawesson's reagent (compare Review of Lawessons Reagent: R. A. Cherkasov et al., Tetrahedron 41, 1985, p. 2567) the cyclisation in known ways by incorporating sulphur (also compare J. M. Sprague, A. M. Land; Heterocyclic Compounds, R. C. Elderfield, Vol. 5, New York, J. Wiley & Sons 1957, p. 484; R. H. Wiley, D. C. England, L. C. Behr, Org. Reactions 6 (1951) 367), or
(ε) through the conversion of activated carboxylic acid derivative with amidehydrazines according to sufficient and generally known methods (compare K. T. Potts, Chem. Reviews 61 (1961) 87; J. H. Boyer, Heterocyclic Compounds, R. C. Elderfield (ed.), Vol. 7, New York, J. Wiley & Sons 1961, p. 384), which can be outlined as follows, 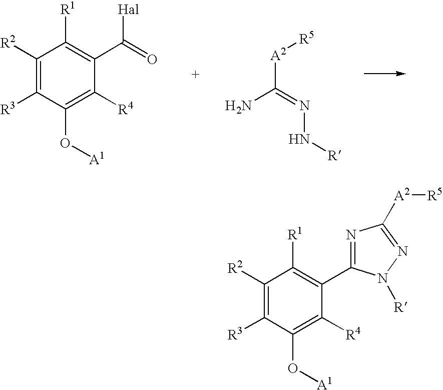
and wherein R¹, R², R³, R⁴, R⁵, A¹ and A² have the preceding provided meanings.

The substituted benzaldoximes to be used as a starting substance for the production of compounds of the general formula (I) by the methods according to the invention are generally defined by the formula (II). In the general formula (II), A¹, R¹, R², R³ and R⁴ preferably have those meanings that have already been provided above in connection with the description of the compounds of the general formula (I) according to the invention as preferred or as particularly, very particularly or most preferred for A¹, R¹, R², R³ and R⁴.

The substituted benzaldoximes of the general formula (II) are not yet known from the literature; as novel substances, they are also related to the present application.

One obtains the novel substituted benzaldoximes of the general formula (II) when one causes substituted benzaldehydes of the general formula (VIII), 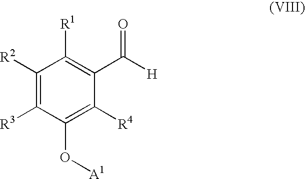
wherein
A¹, R¹, R², R³ and R⁴ have the meaning provided above,
to react with hydroxylamine hydrochloride, optionally in the presence of a diluent such as, for example acetonitrile or N,N-dimethylformamide, and optionally in the presence of a reaction aid such as, for example, potassium carbonate or triethylamine, at temperatures between 0° C. and 100° C. (compare Houben-Weyl, Methoden der Organischen Chemie [Methods of Organic Chemistry], Bd. X/4, 4th edition, 1968, G. Thieme Verlag, Stuttgart New York, p. 55; Bd. 14 b, 4th edition, 1990, G. Thieme Verlag, Stuttgart New York, p. 287; J. P. Freemann Chem. Rev. 73 (1973), p. 283.

The halogenation of compounds for the general formula (III) is carried out by optionally placing compounds of the general formula (II) in a diluent and adding the corresponding halogenation agent that is optionally dissolved in a diluent (also compare Houben-Weyl, Methoder der Organischen Chemie [Methods of Organic Chemistry], 4th edition, 1952, G. Thieme Verlag, Stuttgart New York, p. 691; Bd. X/3, 4th edition. 1965, G. Thieme Verlag, Stuttgart-New York, p. 847, production examples).

The benzaldoximes of the general formula (II) and the compounds of the general formula (III) can naturally be used both in the form of their E or Z isomers as well as in the form of their mixtures of these stereoisomers.

With the exception of the compound 3-[(3,3-dichloro-2-propenyl)-oxy]-benzaldehyde (compare JP-57018658 and JP-57114503), the substituted benzaldehydes of the general formula (VIII) are not yet know from the literature; with the exception of the compound 3-[(3,3-dichloro-2-propenyl)oxy]-benzaldehyde, they are also related to the present application as novel substances.

One obtains the substituted benzaldehydes of the formula (VIII) in a known manner (compare Houben-Weyl, Methoden der Organischen Chemie [Methods of Organic Chemistry], volume 3, pages 3-608, G. Thieme Verlag, Stuttgart New York), for example through the conversion of corresponding hydroxybenzoic acid esters of the general formula (IX) with halogen compounds of the general formula (X), subsequent hydrolysis of the esters of the general formula (XI), reduction of the carboxylic acids of the general formula (XII) thus generated to the benzyl alcohols of the general formula (XII) and oxidation of these compounds, such as can be reproduced through the following reaction schema: 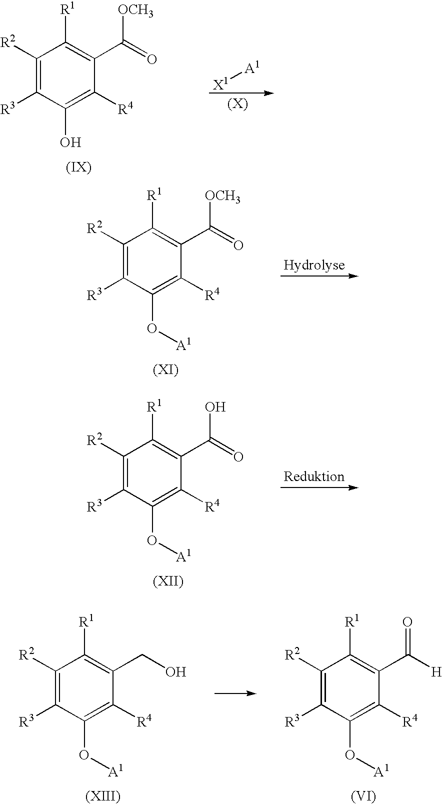
Hydrolyse=hydrolysis; Reduktion=reduction

Here, A¹, R¹, R², R³ and R⁴ have the meanings provided above; X¹ stands for halogen, in particular chlorine, bromine or iodine.

Compounds of the general formula (XIII) can also optionally be obtained directly from compounds of the general formula (XI). For example, this is the case when using lithium alanate (compare production example).

The primary products of the formulas (XI), (XII) and (XIII) are not yet known from the literature.

The substituents of the compounds of the formula (VIII) such as, for example, the substituent R¹, can also be optionally modified in additional reaction steps. For example, in the case that R¹ stands for halogen, and fluorine in particular, a nucleophilic exchange can be carried out in the presence of basic reaction media to be mentioned below with suitable nucleophiles in the context of the substituent definition of R¹ (for example, compare methods from Bioorg. Med. Chem. 9 (2001) for the N,N-dimethylamino moiety, p. 677-694; J. Med. Chem. 45, 25 (2002) S. 5417, for the isopropylthio moiety). Suitable nucleophiles for the exchange reaction are mercapto compounds, hydroxy compounds or amino compounds.

The production of the aldehydes of the general formula (VIII) can also be carried out according to the invention in such a way that one first produces an aldehyde of the general formula (VIIIb) by means of generally known methods and subsequently introduces the moiety A¹ by means of generally known methods: 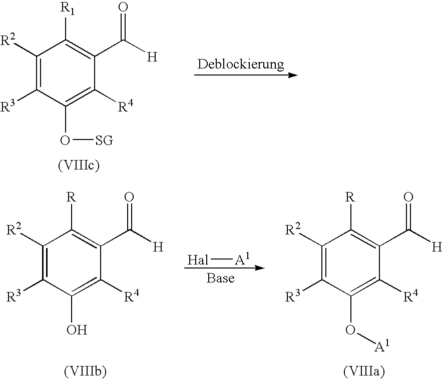
Deblockierung=deblocking

With this approach, compounds of the general formula (VIIIc), which possess a suitable protective group (SG), can also be used as preliminary steps for the production of the compounds of the general formula (VIIIb). For example, hydroxy groups, substituted methyl ether and ether, substituted ethyl ether, substituted benzyl ether, silyl ether, ester, carbonates or sulphonates are known as suitable protective groups (SG) (compare Greene T. W., Wuts P. G. W. in Protective Groups in Organic Synthesis; John Wiley & Sons, Inc. 1999). Possible reaction paths can be outlined as follows:
Protective Group Strategy (Path a) 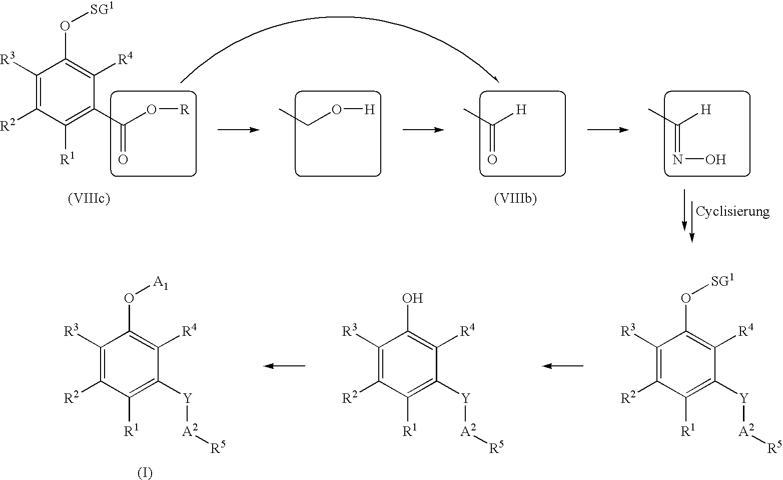
Cyclisierung=cyclisation
wherein
R preferably stands for methyl, ethyl or benzyl and
SG¹ preferably stands for benzyl (Bn), Si(Pr)₃ (TIBS) or SiMe₂-tBu (TBDMS).

The introduction of the group A¹ based on the group SG¹ can be exemplarily outlined as follows: 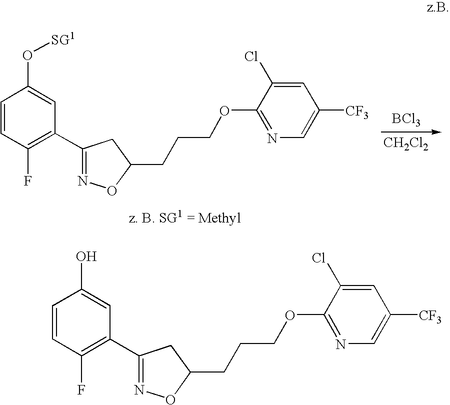
=for example
or 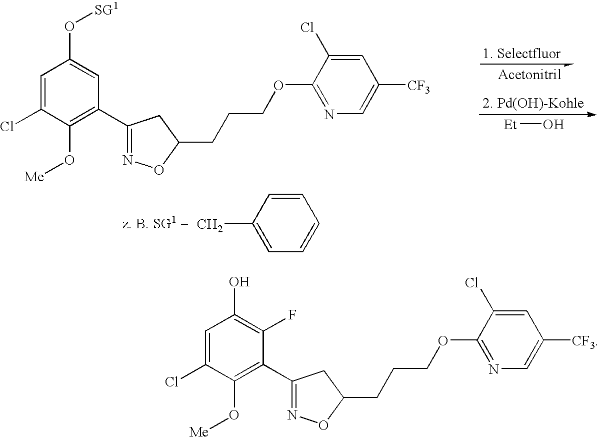
Selectfluor=select fluorine; Acetonitril=acetonitrile; Kohle=carbon
Protective Group Strategy (Path b) 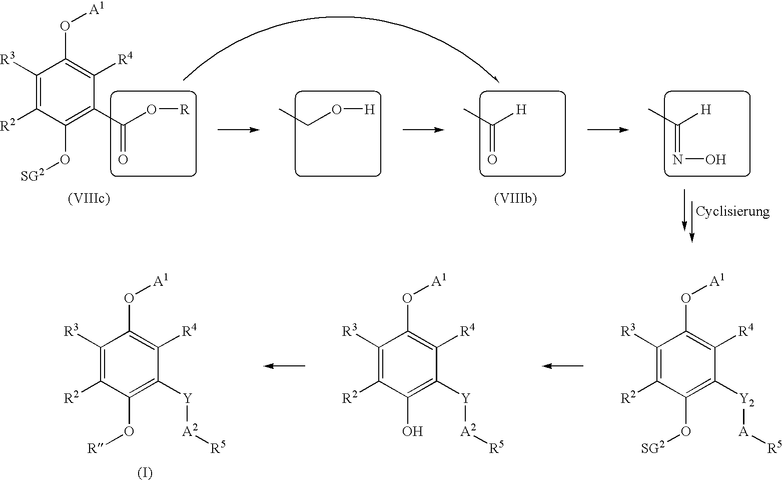
Cylisierung=cyclisation
wherein
R″ stands for the same moiety as provided for R¹ above, and
SG² preferably stands for the group 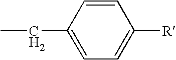
wherein R′ stands for hydrogen, methoxy or phenyl.

The introduction of the group R″ based on the group SG² can be exemplarily outlined as follows:

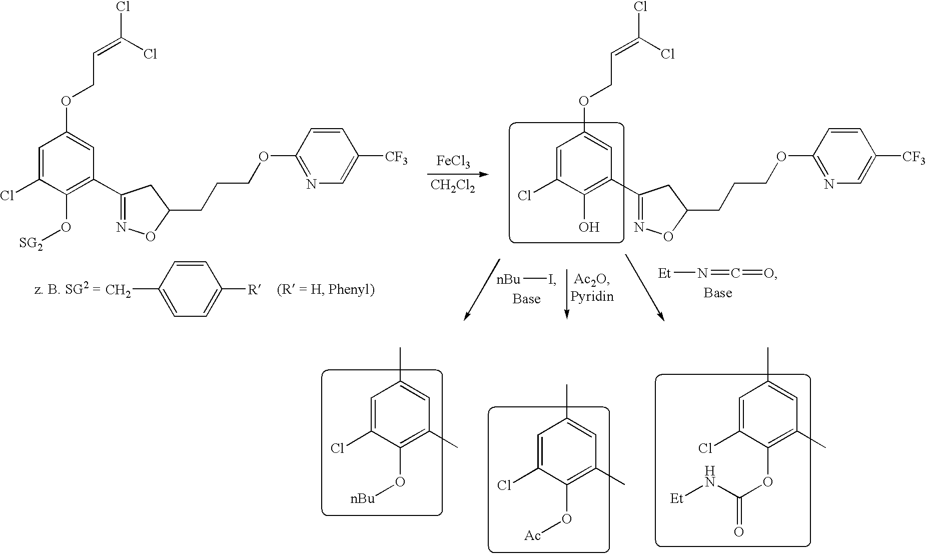

The paths a) and b) can also be combined if R¹=O-SG² and/or O-A¹=O-SG¹.

The alkenes to be used further as a starting substance for the production of compounds of the general formula (I) by the methods according to the invention are generally defined by the formula (V). In the general formula (V), A² and R⁵ preferably have those meanings that have already been provided above in connection with the description of the compounds of the general formula (I) according to the invention as preferred or as particularly, very particularly or most preferred for A² and R⁵.

The starting substances of the general formula (V) are known and/or can be produced according to known methods (compare production examples).

The method according to the invention for the production of the compounds of the general formula (I) is carried out in the first step by using a halogenation agent. Here, suitable halogenation agents are all halogen compounds that are suitable for the conversion of benzaldehyde oximes into corresponding benzhydroxamic acid halogenides. N-bromine-succinimide and N-chloro-succinimide are mentioned exemplarily.

The method according to the invention for the production of the compounds of the general formula (I) is preferably carried out using one or more acid binding agents or reaction agents. In general, the traditional inorganic or organic bases or acid acceptors are suitable as reaction agents for the method according to the invention. Alkaline metal- or alkaline-earth-metal acetates, -amides, -carbonates, -hydrogen carbonates, -hydrides, -hydroxides or -alkanolates such as, for example, sodium-, potassium- or calcium acetate, lithium-, sodium-, potassium- or calcium amide, sodium-, potassium-, cesium- or calcium carbonate, sodium-, potassium- or calcium hydrogen carbonate, lithium-, sodium-, potassium- or calcium hydride, lithium-, sodium-, potassium- or calcium hydroxide, sodium-oder potassium methanolate, -ethanolate, -n- or -i-propanolate, -n-, -i-, -s- or -t-butanolate; furthermore also basic organic nitrogen compounds such as, for example, trimethylamine, triethylamine, tripropylamine, tributylamine, ethyl-diisopropylamine, N,N-dimethylcyclohexylamine, dicyclohexylamine, ethyldicyclohexylamine, N,N-dimethylaniline, N,N-dimethylbenzylamine, pyridine, 2-methyl-, 3-methyl-, 4-methyl-, 2,4-dimethyl-, 2,6-dimethyl-, 3,4-dimethyl- and 3,5-dimethyl-pyridine, 5-ethyl-2-methyl-pyridine, 4-dimethylamino-pyridine, N-methyl-piperidine, 1,4-diazabicyclo[2.2.2]-octane (DABCO), 1,5-diazabicyclo[4.3.0]-non-5-ene (DBN), or 1,8-diazabicyclo[5.4.0]-undec-7-ene (DBU) are preferably included for this purpose.

The method according to the invention for the production of the compounds of the general formula (I) is preferably carried out using one or more diluents. All inert organich solvents are suitable as diluents for carrying out the method according to the invention. Aliphatic, alicyclic or aromatic, optionally halogenated hydrocarbons such as, for example, petrol ether, benzene, toulene, xylol, chlorobenzol, dichlorobenzol, petroleum ether, hexane, cyclohexane, dichloromethane, chloroform, carbon tetrachloride; ethers, such as diethylether, diisopropylether, dioxane, tetrahydrofuran or ethylene glycol dimethyl- or -diethyl ether; ketones, such as acetone, butanone or methyl isobutyl ketone; nitriles, such as acetonitrile, propionitrile or butyronitrile; amides, such as N,N-dimethylformamide, N,N-dimethylacetamide, N-methyl formanilide, N-methylpyrrolidone or hexamethyl phosphoric acid triamide; esters such as acetic acid methyl ester or acetic acid ethyl ester, sulphoxides, such as dimethyl sulphoxide, alcohols, such as methanol, ethanol, n- or i-propanol, ethylene glycol monomethyl ether, ethylene glycol monoethyl ether, diethylene glycol monomethyl ether, diethylene glycol monoethyl ether, whose mixtures with water or pure water are included in particular for this purpose.

The reaction temperatures can be varied within a wide range while carrying out the method according to the invention. In general, one works at temperatures between 0° C. and 150° C., preferably between 10° C. and 120° C.

In general, the method according to the invention is carried out under normal pressure. However, it is also possible to carry out the method according to the invention under increased or decreased pressure—in general between 0.1 bar and 10 bar.

For carrying out the method according to the invention, the starting substances are generally added in approximately equimolar amounts. However, it is also possible to use one of the components in a greater amount. The conversion is generally carried out in a suitable diluent in the presense of a reaction agent, and the reaction mixture is generally stirred several hours at the required temperature. The processing is carried out according to customary methods (compare the production examples).

The compounds according to the invention of the general formula (I) can be converted into other compounds of the general formula (I) according to principally known methods. Some of these possible conversion reactions are outlined exemplarily as follows: 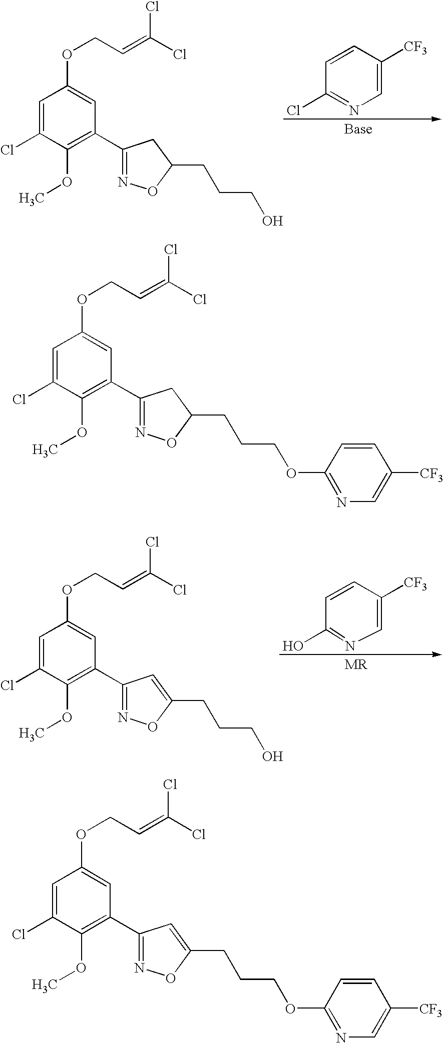
MR=Mitsunobu-Reaktion: vgl. O. Mitsunobu Synthesis (1981), S. 1-28
MR=Mitsunobe reaction; compater O. Mitsunobu synthesis (1981), p. 1-28

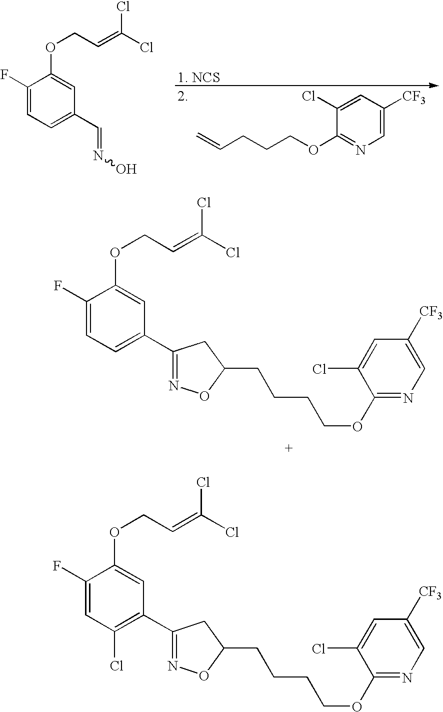

The compounds according to the invention of the general formula (I) can form salts. Traditional non-toxic salts, i.e. salts with bases and salts (“adducts”) with acids, are identified as suitable salts of the compounds of the general formula (I). Preferably mentioned are salts with inorganic bases, such as alkaline metal salts, for example sodium-, potassium- or cesium salts, alkaline earth metal salts, for example calcium- or magnesium salts, ammonium salts, salts with organic bases, in particular with organic amines, for example triethylammonium-, dicyclohexylammonium-, N,N′-dibenzylethylendiammonium-, pyridinium-, picolinium- or ethanolammonium salts, salts with inorganic acids, for example hydrochlorides, hydrobromides, dihydrosulfates, trihydrosulfates, or phosphates, salts with organic carboxylic acids or organic sulphonic acids, for example formiates, acetates, trifluoroacetates, maleates, tartrates, methane sulfonates, benzene sulphonates or paratoluol sulphonates.

Salts are created according to the standard methods for salt production. For example, the compounds according to the invention are caused to react with corresponding acids in order to create acid addition salts. Representative acid addition salts are salts that form through the reaction with inorganic acids such as, for example, sulphuric acid, hydrochloric acid, hydrobromic acid, phosphoric acid or organic carboxylic acids such as acetic acid, trifluoroacetic acid, citric acid, succinic acid, lactic acid, formic acid, maleic acid, camphoric acid, phthalic acid, glycolic acid, glutaric acid, stearic acid, salicylic acid, sorbic acid, cinnamic acid, picric acid, benzoic acid or organic sulphonic acids such as methane sulphonic acids such as methane sulphonic acid and paratoluene sulphonic acid.

The active substances according to the invention are suitable for good botanical compatibility, more favourable endotherm toxicity and good environmental compatibility for the protection of plants and plant organs, for the increase of crop yields, improving the quality of the harvested goods and for combating animal pests, in particular insects, arachnids and nematodes that appear in agriculture, in forestry, in gardens and leisure facilities, in inventory and material protection as well as in the hygiene sector. The can preferably be used as botanical protection agents. They are effective against normally sensitive and resistant types as well as against all or individual development stages. To the pests mentioned above belong:

From the order of Isopoda i.e. Oniscus asellus, Armadillidium vulgare, Porcellio scaber. From the order of the Diplopoda i.e. Blaniulus guttulatus. From the order of the Chilopoda i.e. Geophilus carpophagus, Scutigera spp. From the order of the Symphyla i.e. Scutigerella immaculata. From the order of the Thysanura i.e. Lepisma saccharina. From the order of the Collembola i.e. Onychiurus armatus. From the order of the Orthoptera i.e. Acheta domesticus, Gryllotalpa spp., Locusta migratoria migratorioides, Melanoplus spp., Schistocerca gregaria. From the order of the Blattaria i.e. Blatta orientalis, Periplaneta americana, Leucophaea maderae, Blattella germanica. From the order of the Dermaptera i.e. Forficula auricularia. From the order of the Isoptera i.e. Reticulitermes spp. From the order of the Phthiraptera i.e. Pediculus humanus corporis, Haematopinus spp., Linognathus spp., Trichodectes spp., Damalinia spp. From the order of the Thysanoptera i.e. Hercinothrips femoralis, Thrips tabaci, Thrips palmi, Frankliniella accidentalis. From the order of the Heteroptera i.e. Eurygaster spp., Dysdercus intermedius, Piesma quadrata, Cimex lectularius, Rhodnius prolixus, Triatoma spp. From the order of the Homoptera i.e. Aleurodes brassicae, Bemisia tabaci, Trialeurodes vaporariorum, Aphis gossypii, Brevicoryne brassicae, Cryptomyzus ribis, Aphis fabae, Aphis pomi, Eriosoma lanigerum, Hyalopterus arundinis, Phylloxera vastatrix, Pemphigus spp., Macrosiphum avenae, Myzus spp., Phorodon humuli, Rhopalosiphum padi, Empoasca spp., Euscelis bilobatus, Nephotettix cincticeps, Lecanium corni, Saissetia oleae, Laodelphax striatellus, Nilaparvata lugens, Aonidiella aurantii, Aspidiotus hederae, Pseudococcus spp., Psylla spp. From the order of the Lepidoptera i.e. Pectinophora gossypiella, Bupalus piniarius, Chematobia brumata, Lithocolletis blancardella, Hyponomeuta padella, Plutella xylostella, Malacosoma neustria, Euproctis chrysorrhoea, Lymantria spp., Bucculatrix thurberiella, Phyllocnistis citrella, Agrotis spp., Euxoa spp., Feltia spp., Earias insulana, Heliothis spp., Mamestra brassicae, Panolis flammea, Spodoptera spp., Trichoplusia ni, Carpocapsa pomonella, Pieris spp., Chilo spp., Pyrausta nubilalis, Ephestia kuehniella, Galleria mellonella, Tineola bisselliella, Tinea pellionella, Hofmannophila pseudospretella, Cacoecia podana, Capua reticulana, Choristoneura fumiferana, Clysia ambiguella, Homona magnanima, Tortrix viridana, Cnaphalocerus spp., Oulema oryzae. From the order of the Coleoptera i.e. Anobium punctatum, Rhizopertha dominica, Bruchidius obtectus, Acanthoscelides obtectus, Hylotrupes bajulus, Agelastica alni, Leptinotarsa decemlineata, Phaedon cochleariae, Diabrotica spp., Psylliodes chrysocephala, Epilachna varivestis, Atomaria spp., Oryzaephilus surinamensis, Anthonomus spp., Sitophilus spp., Otiorrhynchus sulcatus, Cosmopolites sordidus, Ceuthorrhynchus assimilis, Hypera postica, Dermestes spp., Trogoderma spp., Anthrenus spp., Attagenus spp., Lyctus spp., Meligethes aeneus, Ptinus spp., Niptus hololeucus, Gibbium psylloides, Tribolium spp., Tenebrio molitor, Agriotes spp., Conoderus spp., Melolontha melolontha, Amphimallon solstitialis, Costelytra zealandica, Lissorhoptrus oryzophilus. From the order of the Hymenoptera i.e. Diprion spp., Hoplocampa spp., Lasius spp., Monomorium pharaonis, Vespa spp. From the order of the Diptera i.e. Aedes spp., Anopheles spp., Culex spp., Drosophila melanogaster, Musca spp., Fannia spp., Calliphora erythrocephala, Lucilia spp., Chrysomyia spp., Cuterebra spp., Gastrophilus spp., Hyppobosca spp., Stomoxys spp., Oestrus spp., Hypoderma spp., Tabanus spp., Tannia spp., Bibio hortulanus, Oscinella frit, Phorbia spp., Pegomyia hyoscyami, Ceratitis capitata, Dacus oleae, Tipula paludosa, Hylemyia spp., Liriomyza spp. Aus der Ordnung der Siphonaptera i.e. Xenopsylla cheopis, Ceratophyllus spp. Aus der Klasse der Arachnida i.e. Scorpio maurus, Latrodectus mactans, Acarus siro, Argas spp., Ornithodoros spp., Dermanyssus gallinae, Eriophyes ribis, Phyllocoptruta oleivora, Boophilus spp., Rhipicephalus spp., Amblyomma spp., Hyalomma spp., Ixodes spp., Psoroptes spp., Chorioptes spp., Sarcoptes spp., Tarsonemus spp., Bryobia praetiosa, Panonychus spp., Tetranychus spp., Hemitarsonemus spp., Brevipalpus spp.

To the plant parasite nematodes belong, for example, Pratylenchus spp., Radopholus similis, Ditylenchus dipsaci, Tylenchulus semipenetrans, Heterodera spp., Globodera spp., Meloidogyne spp., Aphelenchoides spp., Longidorus spp., Xiphinema spp., Trichodorus spp., Bursaphelenchus spp.

The compounds according to the invention can optionally also be used at designated concentrations or application rates as herbicides and microbicides, for example as fungicides, antimycotics and bactericides. The can also optionally be used as intermediate or primary products for the synthesis of additional active substances.

All plants and plant parts can be treated according to the invention. In this respect, all plants and plant populations are included under plants, such as desired and undesired wild plants or crops (including naturally occurring crops). Crops can be plants that can be obtained through conventional breeding and optimisation methods or through methods of biotechnology and genetic technology or combinations of these methods, including the transgenic plants and including the plant species protectable or not protectable by species intellectual property rights. All aboveground and below-ground parts and organs of the plants, such as sprouts, foliage, blooms and roots are included under plants parts, whereby leaves, needles, stalks, stems, blooms, fruit bodies, fruits and seeds as well as roots, bulbs and rhizomes are listed exemplarily. Also included under plant parts are harvested goods as well as vegetative and generative propagation material, for example cuttings, bulbs, rhizomes, scions and seeds.

The treatment according to the invention of the plants and plant parts with the active substances takes place directly or through exposure to their environment, habitat or storage area according to the traditional treatment methods, i.e. by immersion, spraying, vaporising, atomising, scattering, spreading, injecting and for propagation material, in particular for seeds, furthermore by single- or multi-layer envelopment.

The active substances can be transferred in the traditional formulations, such as solutions, emulsions, sprayable powders, suspensions, powders, dusting agents, pastes, soluble powders, granulates, suspension-emulsion concentrates, natural and synthetic materials impregnated with active substance, as well as microencapsulations in polymeric materials.

The formulations are produced in known ways, i.e by mixing the active substances with extenders as liquid solvents and/or solid carrier substances, optionally using surface-active agents as emulsifiers and/or dispersants and/or foaming agents.

In the case that water is used as an extender, organic solvents can also be used as an auxiliary solvent, for example. In essense, suitable as liquid solvents are: Aromates, such as xylol, toluene, or alkylnaphthalines, chlorinated aromates and chlorinated aliphatic hydrocarbons, such as chlorobenzenes, chloroethylenes or methylene chloride, aliphatic hydrocarbons, such as cyclohexane or paraffins, i.e. crude oil fractions, mineral and vegetable oils, alcohols, such as butanol or glycol as well as their ethers and esters, ketones such as acetone, methyl ethyl ketone, methylisobutylketone or cyclohexanone, strong polar solvents, such as dimethylformamide and dimethylsulphoxide, as well as water.

Suitable as solid carrier substances are:

i.e. Ammonium salts and natural rock flours, such as kaolinite, clays, talcum, chalk, quartz, attapulgite, montmorillonite or diatomaceous earth and synthetic rock flours, such as highly dilute silicon dioxide, aluminium oxide and silicates; suitable as solid substances for granulates are: i.e. broken and fractured natural stones such as calcite, marble, pumice, sepiolite, dolomite as well as synthetic granulates from inorganic and organic flours such as granulates from organic material such as saw dust, coconut shells, corn cobs and tobacco stalks; suitable as emulsifying and/or foaming agents are: i.e. non-ionisable and anionic emulsifiers, such as polyoxyethylene fatty acid esters, polyoxyethylene fatty alcohol ethers, i.e. alkylaryl polyglycol ether, alkyl sulphonates, alkyl sulphates, aryl sulphonates such as egg white hydrolysate; suitable as dispersants are: i.e. lignin sulphite waste liquors and methyl cellulose.

Adhesives such as carboxymethylcellulose, natural and synthetic powdered, granular or polymers in the form of latex such as gum arabic, polyvinyl alcohol, polyvinyl acetate, as well as natural phospholipids such as cephaline and lecithin and synthetic phospholipids can be used in the formulations. Additional additives can be mineral and vegetable oils.

Dyestuffs such as inorganic pigments, i.e. iron oxide, titanium oxide, ferrocyan blue and organic dyestuffs such as alizarin-, azo- and metal phthalocyanine dyestuffs and trace nutrients such as salts of iron, manganese, boron, copper, cobalt, molybdenum and zinc can be used.

The formulations generally contain between 0.1 and 95% by weight of active substance, preferably between 0.5 and 90%.

The active substance according to the invention can be present in its traditional commercial formulation as well as in the application forms prepared from these formulations in mixture with other active substances, such as insecticides, attractants, sterilants, bactericides, acaricides, nematicides, fungicides, growth-regulating materials or herbicides. Included with the insecticides are, for example, phosphoric acid esters, carbamates, carboxylic acid esters, chlorinated hydrocarbons, phenylurea, materials produced by microorganisms and others. Particularly favourable mixture partners are the following, for example:

Fungicides:

2-phenylphenol; 8-hydroxyquinoline sulfate; acibenzolar-S-methyl; aldimorph; amidoflumet; ampropylfos; ampropylfos potassium; andoprim; anilazine; azaconazole; azoxystrobin; benalaxyl; benodanil; benomyl; benthiavalicarb-isopropyl; benzamacril; benzamacril-isobutyl; bilanafos; binapacryl; biphenyl; bitertanol; blasticidin-S; bromuconazole; bupirimate; buthiobate; butylamine; calcium polysulfide; capsimycin; captafol; captan; carbendazim; carboxin; carpropamid; carvone; chinomethionate; chlobenthiazone; chlorfenazole; chloroneb; chlorothalonil; chlozolinate; clozylacon; cyazofamid; cyflufenamid; cymoxanil; cyproconazole; cyprodinil; cyprofuram; Dagger G; debacarb; dichlofluanid; dichlone; dichlorophen; diclocymet; diclomezine; dicloran; diethofencarb; difenoconazole; diflumetorim; dimethirimol; dimethomorph; dimoxystrobin; diniconazole; diniconazole-M; dinocap; diphenylamine; dipyrithione; ditalimfos; dithianon; dodine; drazoxolon; edifenphos; epoxiconazole; ethaboxam; ethirimol; etridiazole; famoxadone; fenamidone; fenapanil; fenarimol; fenbuconazole; fenfuram; fenhexamid; fenitropan; fenoxanil; fenpiclonil; fenpropidin; fenpropimorph; ferbam; fluazinam; flubenzimine; fludioxonil; flumetover; flumorph; fluoromide; fluoxastrobin; fluquinconazole; flurprimidol; flusilazole; flusulfamide; flutolanil; flutriafol; folpet; fosetyl-Al; fosetyl-sodium; fuberidazole; furalaxyl; furametpyr; furcarbanil; furmecyclox; guazatine; hexachlorobenzene; hexaconazole; hymexazol; imazalil; imibenconazole; iminoctadine triacetate; iminoctadine tris(albesilate); iodocarb; ipconazole; iprobenfos; iprodione; iprovalicarb; irumamycin; isoprothiolane; isovaledione; kasugamycin; kresoxim-methyl; mancozeb; maneb; meferinizone; mepanipyrim; mepronil; metalaxyl; metalaxyl-M; metconazole; methasulfocarb; methfuroxam; metiram; metominostrobin; metsulfovax; mildiomycin; myclobutanil; myclozolin; natamycin; nicobifen; nitrothal-isopropyl; noviflumuron; nuarimol; ofurace; orysastrobin; oxadixyl; oxolinic acid; oxpoconazole; Oxycarboxin; oxyfenthiin; paclobutrazol; pefurazoate; penconazole; pencycuron; phosdiphen; phthalide; picoxystrobin; piperalin; polyoxins; polyoxorim; probenazole; prochloraz; procymidone; propamocarb; propanosine-sodium; propiconazole; propineb; proquinazid; prothioconazole; pyraclostrobin; pyrazophos; pyrifenox; pyrimethanil; pyroquilon; pyroxyfur; pyrrolnitrine; quinconazole; quinoxyfen; quintozene; simeconazole; spiroxamine; sulphur; tebuconazole; tecloftalam; tecnazene; tetcyclacis; tetraconazole; thiabendazole; thicyofen; thifluzamide; thiophanate-methyl; thiram; tioxymid; tolclofos-methyl; tolylfluanid; triadimefon; triadimenol; triazbutil; triazoxide; tricyclamide; tricyclazole; tridemorph; trifloxystrobin; triflumizole; triforine; triticonazole; uniconazole; validamycin A; vinclozolin; zineb; ziram; zoxamide; (2S)—N-[2-[4-[[3-(4-chlorophenyl)-2-propynyl]oxy]-3-methoxyphenyl]ethyl]-3-methyl-2-[(methylsulfonyl)amino]-butanamide; 1-(1-naphthalenyl)-1H-pyrrole-2,5-dione; 2,3,5,6-tetrachloro-4-(methylsulfonyl)-pyridine; 2-amino-4-methyl-N-phenyl-5-thiazolecarboxamide; 2-chloro-N-(2,3-dihydro-1,1,3-trimethyl-1H-inden-4-yl)-3-pyridincarboxamide; 3,4,5-trichloro-2,6-pyridinedicarbonitrile; Actinovate; cis-1-(4-chlorophenyl)-2-(1H-1,2,4-triazole-1-yl)-cycloheptanol; methyl 1-(2,3-dihydro-2,2-dimethyl-1H-inden-1-yl)-1H-imidazole-5-carboxylate; monopotassium carbonate; N-(6-methoxy-3-pyridinyl)-cyclopropanecarboxamide; N-butyl-8-(1,1-dimethylethyl)-1-oxaspiro[4.5]decan-3-amine; sodium tetrathiocarbonate as well as copper salts and preparations, such as Bordeaux mixture; copper hydroxide; copper naphthenate; copper oxychloride; copper sulfate; cufraneb; cuprous oxide; mancopper; oxine-copper.

Bactericides:

Bronopol, dichlorophen, nitrapyrin, nickel dimethyldithiocarbamate, kasugamycin, octhilinon, furancarboxylic acid, oxytetracycline, probenazole, streptomycin, tecloftalam, copper sulphate and other copper preparations.

Insecticides/Acaricides/Nematicides:

Abamectin, ABG-9008, acephate, acequinocyl, acetamiprid, acetoprole, acrinathrin, AKD-1022, AKD-3059, AKD-3088, alanycarb, aldicarb, aldoxycarb, allethrin, allethrin 1R-isomers, alphacypermethrin (alphamethrin), amidoflumet, aminocarb, amitraz, avermectin, AZ-60541, azadirachtin, azamethiphos, azinphos-methyl, azinphos-ethyl, azocyclotin, Bacillus popilliae, Bacillus sphaericus, Bacillus subtilis, Bacillus thuringiensis, Bacillus thuringiensis strain EG-2348, Bacillus thuringiensis strain GC-91, Bacillus thuringiensis strain NCTC-11821, Baculovirus, Beauveria bassiana, Beauveria tenella, bendiocarb, benfuracarb, bensultap, benzoximate, beta-cyfluthrin, beta-cypermethrin, bifenazate, bifenthrin, binapacryl, bioallethrin, bioallethrin-S-cyclopentyl-isomer, bioethanomethrin, biopermethrin, bioresmethrin, bistrifluron, BPMC, brofenprox, bromophos-ethyl, bromopropylate, bromfenvinfos (-methyl), BTG-504, BTG-505, bufencarb, buprofezin, butathiofos, butocarboxim, butoxycarboxim, butylpyridaben, cadusafos, camphechlor, carbaryl, carbofuran, carbophenothion, carbosulphan, cartap, CGA-50439, chinomethionat, chlordane, chlordimeform, chloethocarb, chlorethoxyfos, chlorfenapyr, chlorfenvinphos, chlorfluazuron, chlormephos, chlorobenzilate, chloropicrin, chlorproxyfen, chlorpyrifos-methyl, chlorpyrifos (-ethyl), chlovaporthrin, chromafenozide, cis-cypermethrin, cis-resmethrin, cis-permethrin, clocythrin, cloethocarb, clofentezine, clothianidin, clothiazoben, codlemone, coumaphos, cyanofenphos, cyanophos, cycloprene, cycloprothrin, Cydia pomonella, cyfluthrin, cyhalothrin, cyhexatin, cypermethrin, cyphenothrin (1R-trans-isomer), cyromazine, DDT, deltamethrin, demeton-S-methyl, demeton-S-methylsulphon, diafenthiuron, dialifos, diazinon, dichlofenthion, dichlorvos, dicofol, dicrotophos, dicyclanil, diflubenzuron, dimethoate, dimethylvinphos, dinobuton, dinocap, dinetofuran, diofenolan, disulphoton, docusate sodium, dofenapyn, DOWCO-439, eflusilanate, emamectin, emamectin benzoate, empenthrin (1R-isomer), endosulphan, Entomopthora spp., EPN, esfenvalerate, ethiofencarb, ethiprole, ethion, ethoprophos, etofenprox, etoxazole, etrimfos, famphur, fenamiphos, fenazaquin, fenbutatin oxide, fenfluthrin, fenitrothion, fenobucarb, fenothiocarb, fenoxacrim, fenoxycarb, fenpropathrin, fenpyrad, fenpyrithrin, fenpyroximate, fensulfothion, fenthion, fentrifanil, fenvalerate, fipronil, flonicamid, fluacrypyrim, fluazuron, flubenzimine, flubrocythrinate, flucycloxuron, flucythrinate, flufenerim, flufenoxuron, flufenprox, flumethrin, flupyrazofos, flutenzin (flufenzine), fluvalinate, fonofos, formetanate, formothion, fosmethilan, fosthiazate, fubfenprox (fluproxyfen), furathiocarb, gamma-HCH, gossyplure, grandlure, Granulose virus, halfenprox, halofenozide, HCH, HCN-801, heptenophos, hexaflumuron, hexythiazox, hydramethylnone, hydroprene, IKA-2002, imidacloprid, imiprothrin, indoxacarb, iodofenphos, iprobenfos, isazofos, isofenphos, isoprocarb, isoxathion, ivermectin, japonilure, kadethrin, nuclear polyhedrosis viruses, kinoprene, lambda-cyhalothrin, lindane, lufenuron, malathion, mecarbam, mesulfenfos, metaldehyde, metam-sodium, methacrifos, methamidophos, metharhizium anisopliae, metharhizium flavoviride, methidathion, methiocarb, methomyl, methoprene, methoxychlor, methoxyfenozide, metolcarb, metoxadiazone, mevinphos, milbemectin, milbemycin, MKI-245, MON-45700, monocrotophos, moxidectin, MTI-800, naled, NC-104, NC-170, NC-184, NC-194, NC-196, niclosamide, nicotine, nitenpyram, nithiazine, NNI-0001, NNI-0101, NNI-0250, NNI-9768, novaluron, noviflumuron, OK-5101, OK-5201, OK-9601, OK-9602, OK-9701, OK-9802, omethoate, oxamyl, oxydemeton-methyl, Paecilomyces fumosoroseus, parathion-methyl, parathion (-ethyl), permethrin (cis-, trans-), petroleum, PH-6045, phenothrin (1R-trans isomer), phenthoate, phorate, phosalone, phosmet, phosphamidon, phosphocarb, phoxim, piperonyl butoxide, pirimicarb, pirimiphos-methyl, pirimiphos-ethyl, prallethrin, profenofos, promecarb, propaphos, propargite, propetamphos, propoxur, prothiofos, prothoate, protrifenbute, pymetrozine, pyraclofos, pyresmethrin, pyrethrum, pyridaben, pyridalyl, pyridaphenthion, pyridathion, pyrimidifen, pyriproxyfen, quinalphos, resmethrin, RH-5849, ribavirin, RU-12457, RU-15525, S-421, S-1833, salithion, sebufos, SI-0009, silafluofen, spinosad, spirodiclofen, spiromesifen, sulfluramid, sulphotep, sulprofos, SZI-121, tau-fluvalinate, tebufenozide, tebufenpyrad, tebupirimfos, teflubenzuron, tefluthrin, temephos, temivinphos, terbam, terbufos, tetrachlorvinphos, tetradifon, tetramethrin, tetramethrin (1R-isomer), tetrasul, theta-cypermethrin, thiacloprid, thiamethoxam, thiapronil, thiatriphos, thiocyclam hydrogen oxalate, thiodicarb, thiofanox, thiometon, thiosultap-sodium, thuringiensin, tolfenpyrad, tralocythrin, tralomethrin, transfluthrin, triarathene, triazamate, triazophos, triazuron, trichlophenidine, trichlorfon, triflumuron, trimethacarb, vamidothion, vaniliprole, verbutin, Verticillium lecanii, WL-108477, WL-40027, YI-5201, YI-5301, YI-5302, XMC, xylylcarb, ZA-3274, zeta-cypermethrin, zolaprofos, ZXI-8901, the compound 3-methylphenyl-propylcarbamate (tsumacide Z), the compound 3-(5-chlor-3-pyridinyl)-8-(2,2,2-trifluorethyl)-8-azabicyclo[3.2.1]octan-3-carbonitrile (CAS-Reg.-No. 185982-80-3) and the corresponding 3-endo-isomers (CAS-Reg.-No. 185984-60-5) (compare WO-96/37494, WO-98/25923) as well as preparations which contain insecticidally-effective plant extracts, nematodes, fungi or viruses.

A mixture with other known active substances, such as herbicides, fertilisers, growth regulators, safeners or semiochemicals is also possible.

The active substances according to the invention can also be present in mixture with synergists during use as insecticides in their traditional commercial formulations as well as in the application forms prepared from these formulations. Synergists are compounds through which the action of the active substances are enhanced without the added synergist itself being required to be active.

The active substances according to the invention can also be present in mixtures with inhibitors during use as insecticides in their traditional commercial formulations as well as in the application forms prepared from these formulations, which inhibit a degradation of the active substance after application to the environment of the plant, on the surface of plant parts or in plant tissues.

The active substance concentration of the application forms prepared from the traditional commercial formulations can vary in wide ranges. The active substance concentration for the application forms can lie between 0.0000001 up to 95% by weight of active substance, preferably between 0.0001 und 1% by weight.

The application occurs in one of the application forms adjusted in the traditional manner.

The active substance is characterised by an excellent residual effect on wood and clay as well as by good alkaline stability on calcareous substrates during use against hygenic and inventory pests.

As already mentioned above, all plants and their parts can be treated according to the invention. Plant species and plant breeds occurring in the wild or obtained through conventional biological breeding methods such as crossing or protoplastic infusion, as well as their parts, are treated in a preferred embodiment. Transgenic plants and plant species that were obtained by means of genetic technology methods optionally in combination with conventional methods (Genetically Modified Organisms) and their parts are treated in an additional preferred embodiment. The term “parts” and “parts of plants” or “plant parts” were explained above.

The respective traditional commercial plant species or those in use are particularly preferably treated according to the invention. Under plant species are included plants with novel characteristics (“traits”) that have been bred both through conventional breeding and by mutagenesis or through recombinant DNA techniques. These can be species, bio and genotypes.

Depending on plant species or plant breeds, their location and growth conditions (soil, climate vegetation period, nutrition), additive (“synergistic”) effects can appear through the treatment according to the invention. Thus possible, for example, are reduced application rates and/or extensions of the spectrum of action and/or a strengthening of the action of the substances and agents that are usable according to the invention, better plant development, increased tolerance to high or low temperatures, increased tolerance to dryness or to water and soil salt content, increased blossom yield, simpler harvest, acceleration of maturation, higher crop yields, higher quality and/or higher nutritional value of the harvested products, greater storability and/or processibility of the harvested products, which exceed the actual effects to be expected.

All plants obtained through genetic modification of genetic material, which imparts particularly favourable useful characteristics (“traits”) to these plants, are included in the transgenic plants and plant species to be treated preferably according to the invention. Examples of such characteristics are better plant growth, increased tolerance to high or low temperatures, increased tolerance to dryness or to water and soil salt content, increased blossom yield, simpler harvest, acceleration of maturation, higher crop yields, higher quality and/or higher nutritional value of the harvested products, greater storability and/or processibility of the harvested products. Additional and particularly emphasised examples for such characteristics are an increased defense by the plants against animal and microbial pests, such as against insects, mites, plant pathogenic fungi, bacteria and/or viruses, as well as an increased tolerance by the plants against particular herbicidal active substances. The important crops such as grains (wheat, rice), maize, soy, potatoes, cotton, tobacco, rapeseed and fruit plants (with the fruits apple, pears, citrus fruits and grapes) are mentioned as examples of transgenic plants, whereby maize, soy, potatoes, cotton, tobacco and rapeseed are particularly emphasised. The increased defense by the plants against insects, arachnids, nematodes and snails by means of toxins originating from the plants, in particular those that are produced in the plants from the genetic material from Bacillus thuringiensis (for example through the genes CryIA(a), CryIA(b), CryIA(c), CryIIA, CryIIIA, CryIIIB2, Cry9c Cry2Ab, Cry3Bb and CryIF as well as their combinations) are particularly emphasised as characteristics (“traits”). The increased defense by the plants against fungi, bacteria and viruses by means of systematically acquired resistance (SAR), systemin, phytoalexines, elicitors and resistance genes and correspondingly expressed proteins and toxins are also particularly emphasised as characteristics (“traits”). Furthermore, the increased tolerance of the plants against particular herbicidal active substances, for example imidazolinones, sulphonylurea, glyphosate or phosphinotricin (i.e. “PAT” gene) are particularly emphasised as characteristics (“traits”). The genes imparting the desired characteristics (“traits”) in each case can also occur in combinations with one another in the transgenic plants. Maize species, cotton species, soy species and potato species that are distributed under the trade names YIELD GARD® (i.e. maize, cotton, soy), KnockOut® (i.e maize), StarLink® (i.e. maize), Bollgard® (cotton), Nucotn® (cotton) and NewLeaf® (potato) are mentioned as examples of “Bt plants”. Maize species, cotton species and soy species that are distributed under the trade names Roundup Ready® (tolerance against glyphosate, i.e. M maize, cotton, soy), LibertyLink® (tolerance against phosphinotricin, i.e. rapeseed), IMI® (tolerance against imidazolinone) and STS® (tolerance against sulphonylurea i.e. maize). The species distributed under the name Clearfield® (i.e. maize) are also mentioned as herbicide-resistant (conventionally bred for herbicide tolerance) plants. Naturally these statements also apply for plant species developed in the future or coming onto the market in the future with these genetic characteristics (“traits”), or with genetic characteristics (“traits”) developed in the future.

The listed plants can be treated particularly favourably according to the invention with the compounds of the general formula I and the active substance mixtures according to the invention. The preferred areas provided above for the active substances and mixtures also apply for the treatment of these plants. Particularly emphasised is the treatment of plants with the compounds and mixtures listed specifically in the preceding text.

The active substances according to the invention are not only effective against plant-, hygienic- and inventory pests, but also in the veterinary medicine sector against animal parasites (ectoparasites) such as hard ticks, soft ticks, scabies mites, harvest mites, flies (stinging and licking), parasitic fly larvae, lice, biting lice, feather lice and fleas. To these parasites belong:

From the order of the Anoplurida z.B. Haematopinus spp., Linognathus spp., Pediculus spp., Phtirus spp., Solenopotes spp. From the order of the Mallophagida and of the suborder Amblycerina and Ischnocerina i.e. Trimenopon spp., Menopon spp., Trinoton spp., Bovicola spp., Werneckiella spp., Lepikentron spp., From the order Diptera and the suborder Nematocerina and Brachycerina i.e. Aedes spp., Anopheles spp., Culex spp., Simulium spp., Eusimulium spp., Phlebotomus spp., Lutzomyia spp., Culicoides spp., Chrysops spp., Hybomitra spp., Atylotus spp., Tabanus spp., Haematopota spp., Philipomyia spp., Braula spp., Musca spp., Hydrotaea spp., Stomoxys spp., Haematobia spp., Morellia spp., Fannia spp., Glossina spp., Calliphora spp., Lucilia spp., Chrysomyia spp., Wohlfahrtia spp., Sarcophaga spp., Oestrus spp., Hypoderma spp., Gasterophilus spp., Hippobosca spp., Lipoptena spp., Melophagus spp. From the order of the Siphonapterida i.e. Pulex spp., Ctenocephalides spp., Xenopsylla spp., Ceratophyllus spp. From the order of the Heteropterida i.e. Cimex spp., Triatoma spp., Rhodnius spp., Panstrongylus spp. From the order of the Blattarida i.e. Blatta orientalis, Periplaneta americana, Blattela germanica, Supella spp. From the subclass of the Acari (Acarina) and of the orders of the Meta- and Mesostigmata i.e. Argas spp., Ornithodorus spp., Otobius spp., Ixodes spp., Amblyomma spp., Boophilus spp., Dermacentor spp., Haemophysalis spp., Hyalomma spp., Rhipicephalus spp., Dermanyssus spp., Raillietia spp., Pneumonyssus spp., Stemostoma spp., Varroa spp. From the order of the Actinedida (Prostigmata) and Acaridida (Astigmata) i.e. Acarapis spp., Cheyletiella spp., Ornithocheyletia spp., Myobia spp., Psorergates spp., Demodex spp., Trombicula spp., Listrophorus spp., Acarus spp., Tyrophagus spp., Caloglyphus spp., Hypodectes spp., Pterolichus spp., Psoroptes spp., Chorioptes spp., Otodectes spp., Sarcoptes spp., Notoedres spp., Knemidocoptes spp., Cytodites spp., Laminosioptes spp.

The active substances according to the invention of the formula (I) are also suitable for combating arthropods that afflict agricultural livestock such as, for example, cattle, sheep, goats, horses, pigs, donkeys, camel, buffalo, rabbits, chickens, turkeys, ducks, geese, bees, other pets such as, for example, dogs, cats, domesticated birds, aquarium fish as well as so-called research animals such as, for example, hamsters, guinea pigs, rats and mice. By combating these arthropods, cases of death and performance losses (for meat, milk, wool, skins, eggs, honey and so forth) are minimised such that more economical and simpler animal husbandry is possible through the use of the active substances according to the invention.

The use of the active substances according to the invention occurs in the veterinary sector in known ways, for example, by means of enteral application in the form of tablets, capsules, drinks, drenches, granulates, pastes, boli, of the feed-through method, of suppositories, through parenteral administration such as, for example, through injections (intramuscular, subcutaneous, intravenous, intraperitoneal and others), implants, through nasal application, through dermal use in the form of immersion or bath (dips), for example, spray, infusion, of wash, of dusting as well as with the aid of molded paddings that contain active substances such as collars, ear markers, tail markers, limb bands, halters, marking devices and so forth.

For the use with livestock, poultry, pets etc., one can apply the active substances of the formula (I) as formulations (for example powders, emulsions, flowing agents) that contain the active substances in an amount from 1 to 80% by weight, directly or after dilution 100 to 10,000 times, or use them as a chemical bath.

Furthermore, it was found that the compounds according to the invention show a good insecticidal action against insects that destroy technical materials.

Exemplarily and preferably—however without limitation—the following insects are identified:

Beetles such as Hylotrupes bajulus, Chlorophorus pilosis, Anobium punctatum, Xestobium rufovillosum, Ptilinus pecticornis, Dendrobium pertinex, Ernobius mollis, Priobium carpini, Lyctus brunneus, Lyctus africanus, Lyctus planicollis, Lyctus linearis, Lyctus pubescens, Trogoxylon aequale, Minthes rugicollis, Xyleborus spec. Tryptodendron spec. Apate monachus, Bostrychus capucins, Heterobostrychus brunneus, Sinoxylon spec. Dinoderus minutus; Hymenoptera such as Sirex juvencus, Urocerus gigas, Urocerus gigas taignus, Urocerus augur; Termites such as Kalotermes flavicollis, Cryptotermes brevis, Heterotermes indicola, Reticulitermes flavipes, Reticulitermes santonensis, Reticulitermes lucifugus, Mastotermes darwiniensis, Zootermopsis nevadensis, Coptotermes formosanus; Silverfish such as Lepisma saccharina.

Non-living materials to be included under technical materials in the preceding context are preferably those such as plastics, adhesives, glues, papers and cartons, leather, wood, wood-working products and coating materials.

The material to be protected prior to insect infestation very particularly preferably involves wood and wood-working products.

Included under wood and wood-working products, which can be protected by the agent according to the invention or mixtures containing it, are exemplarily:

Lumber, wooden beams, railroad ties, bridge parts, boat moorings, wooden vehicles, boxes, pallets, containers, telephone poles, wood paneling, wood windows and doors, plywood, particle board, carpentry work or wood products that are generally found in use for house construction or carpentry.

The active substances can be applied as such in the form of concentrates or generally customary formulations such as powders, granulates, solutions, suspensions, emulsions or pastes.

The formulations identified can be produced in a known manner, i.e. by mixing the active substances with at least one solvent or diluent, emulsifier, dispersing and/or binding or fixing agent, water repellant, optionally siccatives and UV stabilisers and optionally dyestuffs and pigments as well as other treatment resources.

The insecticidal agent or concentrate used for the protection of wood and wood-working materials contains the active substance according to the invention in a concentration from 0.0001 to 95% by weight, in particular 0.001 to 60% by weight.

The amount of agent or concentrate used is dependent on the type and on the appearance of the insects and on the medium. The optimal amount to use for the application can be determined through the use of test rows in each case. However, in general it is sufficient to use 0.0001 to 20% by weight, preferably 0.001 to 10% by weight of the active substance, in terms of the material to be protected.

An organic chemical solvent or solvent mixture and/or an oily or oil-like organic chemical solvent with low volatility or solvent mixture and/or a polar organic chemical solvent or solvent mixture and/or water and optionally an emulsifier and/or wetting agent serve as a solvent or diluent.

Oily or oil-like solvents with an evaporation number over 35 and a flame point over 30° C., preferably over 45° C., are preferably used as organic chemical solvents. Corresponding mineral oils or their aromatic fractions or solvent mixtures containing mineral oils, preferably petroleum spirit, petroleum and/or alkyl benzene are used as water-insoluble, oily and oil-like solvents that are not easily volatised.

Mineral oils with a boiling range of from 170 to 220° C., petroleum spirit with a boiling range from 170 to 220° C., spindle oil with a boiling range from 250 to 350° C., petroleum or aromates of a boiling range from 160 to 280° C., turpentine oil and similar items are used advantageously.

Liquid aliphatic hydrocarbons with a boiling range from 180 to 220° C. or high-boiling mixtures of aromatic and aliphatic hydrocarbons with a boiling range from 180 to 220° C. and/or spindle oil and/or monochloro naphthaline, preferably α-monochloro naphthaline are used a preferred embodiment.

The oily or oil-like organic solvents with an evaporation number over 35 and a flame point above 30° C., preferably above 45° C., that are not easily volatised can be partially replaced by organic chemical solvents of high or intermediate volatility, with the condition that the solvent mixture also has an evaporation number and a flame point above 30° C., preferably above 45° C., and that the insecticide-fungicide mixture is soluble or emulsifiable in this solvent mixture.

According to a preferred embodiment, a portion of the organic chemical solvent or solvent mixture or an aliphatic polar organic chemical solvent or solvent mixture is replaced. Hydroxyl- and/or ester- and/or ether groups containing aliphatic organic chemical solvents such as, for example, glycol ethers, esters or similar are preferably used.

In the context of the present invention, the synthetic resins and/or bonded dried oils, in particular binding agents consisting of or containing an acrylic resin, a vinyl resin, i.e. polyvinyl acetate, polyester resin, polycondensation- or polyaddition resin, polyurethane resin, alkyd resin or modified alkyd resin, phenol resin, hydrocarbon resin such as indene-coumarone resin, silicon resin, dried vegetable and/or dried oils and/or physically dried binding agents on the basis of a natural and/or artificial resin are used as organic chemical binding agents.

The synthetic resin used as a binding agent can be used in the form of an emulsion, dispersion or solution. Bitumen or bituminous substances can be used as binding agents up to 10% by weight. In addition, known dyestuffs, pigments, water-repellent agents, scent markers and inhibitors and corrosion prevention agents and similar items can be used.

According to the invention, at least one alkyd resin or modified alkyd resin and/or a dried vegetable oil is preferably included in the agent or in the concentrate. According to the invention, alkyd resins with an oil content of more than 45% by weight, preferably 50 to 68% by weight, are preferably used.

The binding agent mentioned can be used in whole or in part by means of a fixing agent (mixture) or a plasticiser (mixture). These additives should prevent a volatilisation of the active substances as well as a crystallisation or precipitation. They preferably replace 0.01 to 30% of the binding agent (in terms of 100% of the binding agent used).

The plasticisers originate from the chemical classes of the phthalic acid esters such as dibutyl-, dioctyl- or benzyl butyl phthalate, phosphoric acid esters such as tributyl phosphate, adipic acid esters such as di-(2-ethylhexyl)-adipate, stearates such as butyl stearate or amyl stearate, oleates such as butyloleate, glycerin ethers or high molecular glycol ether, glycerine esters and p-toluene sulphonic acid ester.

Fixing agents are chemically based on polyvinyl alkyl ethers such as, for example, polyvinyl methyl ether or ketones such as benzophenone, ethylene benzophenone.

Water is especially suitable as a solvent or diluent, optionally in mixture with one or more of the organic chemical solvents, diluents, emulsifiers and dispersants mentioned above.

A particularly effective protection of wood is achieved by means of industrial impregnation processes, i.e. vacuum, double vacuum or pressure processes.

The agents that are ready for use can optionally contain yet additional insecticides and optionally yet one or more fungicides.

The insecticides and fungicides mentioned in WO 94/29 268 are preferred suitable mixture partners. The compounds mentioned in this document are an express element of the present application.

Insecticides such as chlorpyriphos, phoxim, silafluofin, alphamethrin, cyfluthrin, dypermethrin, deltamethrin, permethrin, imidacloprid, NI-25, flufenoxuron, hexaflumuron, transfluthrin, thiacloprid, methoxyfenozide, triflumuron, clothianidin, spinosad, tefluthrin and fungicides such as epoxyconazole, hexaconazole, azaconazole, propiconazole, tebuconazole, cyproconazole, metconazole, imazalil, dichlorfluanid, tolylfluanid, 3-iodine-2-propinyl-butylcarbamate, N-octylisothiazolin-3-one and 4,5-dichloro-N-octylisothiazolin-3-one, can be very particularly preferred mixture partners.

At the same time, the compounds according to the invention can be used for the prevention of fouling of objects, in particular of ship hulls, sieves, nets, structures, wharf installations and signaling installations, which come into contact with seawater or brackish water.

Fouling by sessile oligochaetes such as tubificid worms as well as by mussels and species of the group Ledamorpha (goose barnacles), such as various Lepas and Scalpellum species, or by species from the group balanomorpha (sea pox), such as Balanus or Pollicipes species, increases the friction resistance of ships and as a consequence leads to increased energy consumption and furthermore to a clear increase in operating costs through frequent dry-dock layovers.

Alongside the fouling by algaes, for example Ectocarpus sp. and Ceramium sp., of particular importance is the fouling by sessile Entomostraca groups, which are summarised under the name Cirripedia (river crabs).

It was surprisingly found that the compounds according to the invention have an excellent antifouling effect, alone or in combination with other active substances.

Through the use of compounds according to the invention alone or in combination with other active substances, the use of heavy metals such as, for example bis(trialkyltin) sulphides, tri-n-butyl tin laurate, tri-n-butyltin chloride, copper(I) oxide, triethyltin chloride, tri-n-butyl(2-phenyl-4-chlorophenoxy) tin, tributyl tin oxide, molybdenum disulphide, antimony oxide, polymeric butyl titanium, phenyl-(bispyridine) bismuth chloride, tri-n-butyltin fluoride, manganese ethylene bisthiocarbamate, zinc dimethyl dithiocarbamate, zinc ethylene bisthiocarbamate, zinc- and copper salts of 2-pyridinethiol-1-oxide, bisdimethyldithiocarbamoyl zinc ethylene bisthiocarbamate, zinc oxide, copper(I) ethylene bisdithiocarbamate, copper thiocyanate, copper naphthenate and tributyltin halogenides can be avoided, or the concentration of these compounds can be decidedly reduced.

The antifouling paints that are ready for use can optionally contain yet other active substances, preferably algicides, fungicides, herbicides, molluscicides and other antifouling active substances.

Suitable as combination partners for the antifouling agents according to the invention are preferably:

Algicides such as 2-tert.-butylamino-4-cyclopropylamino-6-methylthio-1,3,5-triazine, dichlorophen, diuron, endothal, fentin acetat, isoproturon, methabenzthiazuron, oxyfluorfen, quinoclamine and terbutryn; fungicides such as benzo[b]thiophene carboxylic acid cyclohexylamide-S,S-dioxide, dichlofluanid, fluorfolpet, 3-iodine-2-propinyl-butylcarbamate, tolylfluanid and azoles such as azaconazole, cyproconazole, epoxyconazole, hexaconazole, metconazole, propiconazole and tebuconazole; molluscicides such as fentin acetate, metaldehyde, methiocarb, niclosamide, thiodicarb and trimethacarb, Fe-chelate, or conventional antifouling active substances such as 4,5-dichloro-2-octyl-4-isothiazolin-3-one, diiodine methylparatryl sulphone, 2-(N,N-dimethylthiocarbamoylthio)-5-nitrothiazyl, potassium-, copper-, sodium- and zinc salts of 2-pyridinthiol-1-oxide, pyridine triphenylborane, tetrabutyldistannoxane, 2,3,5,6-tetrachloro-4-(methylsulphonyl)-pyridine, 2,4,5,6-tetrachloroisophthalonitrile, tetramethylthiuramdisulphide and 2,4,6-trichlorphenylmaleinimide.

The antifouling agents used contain the active substances according to the invention in a concentration of 0.001 to 50% by weight, in particular from 0.01 to 20% by weight.

In addition, the antifouling agents according to the invention contain traditional components such as described for example, in Ungerer, Chem. Ind. 1985, 37, 730-732 and Williams, Antifouling Marine Coatings, Noyes, Park Ridge, 1973.

Alongside the algicides, fungicides, molluscicides and insecticidal active substances according to the invention, antifouling coating materials contain binding agents in particular.

Examples of approved binding agents are polyvinyl chloride in a solvent system, chlorinated rubber in a solvent system, acrylic resin in a solvent system, in particular in an aqueous system, vinyl chloride/vinyl acetate copolymer systems in the form of aqueous dispersions or in the form of organic solvent systems, butadiene/styrene/acryl nitrile rubbers, dried oils such as flaxseed oil, resin esters or modified solid resins in combination with tar or bitumen, asphalts such as epoxy compounds, limited amounts of chlorinated rubber, chlorinated polypropylene and vinyl resin.

Coating materials also optionally contain inorganic pigments, organic pigments or dyestuffs, which preferably are insoluble in sea water. In addition, coating materials can contain materials such as colophonium, in order to make a controlled release of the active substances possible. In addition, the coatings can be plasticisers that contain modification agents that affect rheological characteristics as well as other traditional components. The compounds according to the invention or the mixture mentioned above can also incorporated into self-polishing antifouling systems.

The active substances are also suitable for combating animal pests, in particular insects, arachnids and mites that occur in closed areas, for example apartments, factories, offices, vehicle cabins and others. They can be used for combating these pests alone or in combination with other active substances and auxiliary materials in household insecticide products. They are effective against sensitive and resistant species as well as against all development stages. To these pests belong:

From the order of the Scorpionidea i.e. Buthus occitanus. From the order of the Acarina i.e. Argas persicus, Argas reflexus, Bryobia ssp., Dermanyssus gallinae, Glyciphagus domesticus, Ornithodorus moubat, Rhipicephalus sanguineus, Trombicula alfreddugesi, Neutrombicula autumnalis, Dermatophagoides pteronissimus, Dermatophagoides forinae. From the order of the Araneae i.e. Aviculariidae, Araneidae. From the order of the Opiliones i.e. Pseudoscorpiones chelifer, Pseudoscorpiones cheiridium, Opiliones phalangium. From the order of the Isopoda i.e. Oniscus asellus, Porcellio scaber. From the order of the Diplopoda i.e. Blaniulus guttulatus, Polydesmus spp. From the order of the Chilopoda i.e. Geophilus spp. From the order of the Zygentoma i.e. Ctenolepisma spp., Lepisma saccharina, Lepismodes inquilinus. From the order of the Blattaria i.e. Blatta orientalies, Blattella germanica, Blattella asahinai, Leucophaea maderae, Panchlora spp., Parcoblatta spp., Periplaneta australasiae, Periplaneta americana, Periplaneta brunnea, Periplaneta fuliginosa, Supella longipalpa. From the order of the Saltatoria i.e. Acheta domesticus. From the order of the Dermaptera i.e. Forficula auricularia. From the order of the Isoptera i.e. Kalotermes spp., Reticulitermes spp. From the order of the Psocoptera i.e. Lepinatus spp., Liposcelis spp. From the order of the Coleoptera i.e. Anthrenus spp., Attagenus spp., Dermestes spp., Latheticus oryzae, Necrobia spp., Ptinus spp., Rhizopertha dominica, Sitophilus granarius, Sitophilus oryzae, Sitophilus zeamais, Stegobium paniceum. From the order of the Diptera i.e. Aedes aegypti, Aedes albopictus, Aedes taeniorhynchus, Anopheles spp., Calliphora erythrocephala, Chrysozona pluvialis, Culex quinquefasciatus, Culex pipiens, Culex tarsalis, Drosophila spp., Fannia canicularis, Musca domestica, Phlebotomus spp., Sarcophaga carnaria, Simulium spp., Stomoxys calcitrans, Tipula paludosa. From the order of the Lepidoptera i.e. Achroia grisella, Galleria mellonella, Plodia interpunctella, Tinea cloacella, Tinea pellionella, Tineola bisselliella. From the order of the Siphonaptera i.e. Ctenocephalides canis, Ctenocephalides felis, Pulex irritans, Tunga penetrans, Xenopsylla cheopis. From the order of the Hymenoptera i.e. Camponotus herculeanus, Lasius fuliginosus, Lasius niger, Lasius umbratus, Monomorium pharaonis, Paravespula spp., Tetramorium caespitum. From the order of the Anoplura i.e. Pediculus humanus capitis, Pediculus humanus corporis, Phthirus pubis. From the order of the Heteroptera i.e. Cimex hemipterus, Cimex lectularius, Rhodinus prolixus, Triatoma infestans.

The application in the area of household insecticides takes place alone or in combination with other suitable active substances such as phosphoric acid esters, carbamates, pyrethroids, neonicotinoids, growth regulators or active substances from other known insecticide classes.

The application takes place in aerosols, unpressurised spray devices, i.e. pump and atomising sprays, misting machines, foggers, foaming, gelling, vaporiser products with vaporising dies of cellulose or plastic, fluid vaporisers, gel and membrane vaporisers, propeller-driven vaporisers, no-power or passive vaporising systems, moth papers, moth sacks and moth gels, as granulates or dust, in straw lures or lure stations.

### PRODUCTION EXAMPLES

### Example (I-1)

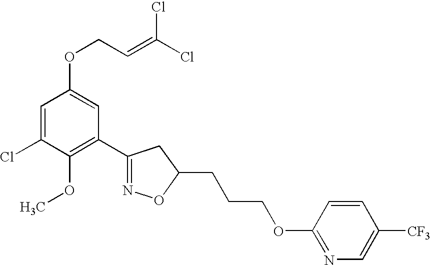

### (R/S)-3-(2-methoxy-3-chloro-5-(1,1-dichloro-1-propen-3-oxy)-phenyl)-5-((5-trifluormethylpyridin-2-yl)-3-(propyl)ether-1-yl)-Δ²-isoxazoline

0.5 g (1.61 mMol) of 3-chloro-5-(3,3-dichlor-allyloxy)-2-methoxy-benzaldehyde oxime are dissolved in 15 ml of N,N-dimethylformamide (DMF) and mixed with 0.24 g (1.77 mMol) of N-chloro succinimide (NCS). The reaction mixture is then stirred for approximately two hours at room temperature (RT, approximately 20° C.). One then adds 0.56 (2.4 mMol) of 2-(n-pent-5-ene-1-yl-oxy)-5-trifluoromethylpyridine and 0.18 g (1.77 mMol) of triethylamine and allows the brown solution to stand for approximately 16 hours at room temperature. To finish, the reaction solution is mixed with approximately 20 ml of water and extracted three times with 50 ml of dichloromethane. After the concentration of the organic phase to dryness, the remaining residue is chromatographed over silica gel.

One obtains 347 mg (40% of the theory) of 3-(2-methoxy-3-chloro-5-(1,1-dichloro-1-propen-3-oxy)-phenyl)-5-((5-trifluormethyl-pyridin-2-yl)-3-(propyl)ether 1-yl)-Δ²-isoxazoline.

Melting point: 62° C., MS (ES+): 541.

¹H-NMR: CDCl₃, δ=1.9 (m, 4H, CH₂—CH₂—CH₂—O-Py); 3.12, 3.5 (2×dd, 2×1H, diastereotopes N═C—CH₂, hetaryl); 3.80 (s, 3H, OCH₃); 4.62 (d, 2H, CH₂—CH═CCl₂); 4.80 (m, 1H, CH—O, hetaryl); 4.9 (m, 2H, CH₂—O-Py); 6.23 (t, 1H, CH═CCl₂); 6.8 (d, 1H, Py); 7.0, 7.18 (2×d, 2×1H, Ar—H); 8.4 (d, 1H, Py); 7.75 (dd, 1H, Py) ppm.

### Example (I-2)

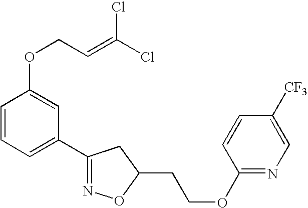

### (R/S)-3-(3-chloro-5-(1,1-dichloro-1-propen-3-oxy)-phenyl)-5-((5-trifluormethyl-pyridin-2-yl)-3-(ethyl)ether-1-yl)-Δ²-isoxazoline

0.2 g (0.81 mMol) of (3,3-dichloro-allyoxy)-benzaldehyde oxime are dissolved in 15 ml of N,N-dimethylformamide (DMF) and mixed with 0.12 g (0.89 mMol) of N-chloro succinimide (NCS), and this reaction solution is stirred over 16 hours at room temperature (RT). One then adds 0.26 (1.22 mMol) of 2-(but-3-en-1-yl-oxy)-5-trifluoromethylpyridine and 0.09 g (0.89 mMol) of triethylamine and stirs the reaction mixture approximately 16 hours at room temperature and then an additional 24 hours at 70° C. To finish, the reaction solution is mixed with approximately 20 ml of water and extracted three times with 50 ml of dichloromethane. After the concentration of the organic phase to dryness, the remaining residue is purified by means of preparative HPLC.

One obtains 24 mg (purity: 100% according to HPLC) and 80 mg (purity: 77% according to HPLC) (23% of the theory) of 3-(3-chloro-5-(1,1-dichloro-1-propen-3-oxy)-phenyl)-5-((5-trifluormethyl-pyridin-2-yl)-3-(ethyl)ether-1-yl)-Δ²-isoxazoline.

LC-MS (ES⁺) m/z (%)=461

¹H-NMR: CDCl₃, δ=2.1-2.3 (m, 2H, CH₂—CH₂—O-Py); 3.10, 3.48 (2×dd, 2×1H, diastereotopes N═C—CH₂, hetaryl); 4.56 (t, 2H, CH₂—O-Py); 4.68 (d, 2H, CH₂—CH═CCl₂); 4.98 (m, 1H, CH—O, hetaryl); 6.17 (t, 1H, CH═CCl₂); 6.8 (d, 1H, Py); 6.95 (dd, 1H, Ar—H); 7.20-7.27 (m, 2H, Ar—H); 7.72 (t, 1H, Ar—H); 8.43 (m, 1H, Py) ppm.

¹³C-NMR (signal selection): CDCl₃, δ=35 (CH₂—CH₂—O-Py); 41 (N═C—CH₂, hetaryl); 63 (CH₂—O-Py); 65 (CH₂—CH═CCl₂); 78 (CH—O, hetaryl); 112 (Py-C); 113 (Ar—C); 117 (Ar—C); 121 (Ar—C); 126 (CH═CCl₂); 130 (Ar—C); 135 (Py-C); 146 (Py-C) ppm.

### Example (I-3)

### 3-(2-methoxy-3-chloro-5-(1,1-dichloro-1-propen-3-oxy)-phenyl)-5-(hydroxymethyl)-isoxazole

The implementation takes place analogously to Example 1 using approximately 400 equivalents of propargyl alcohol. The reaction time amounts to approximately 2 hours for the cycloaddition:

¹H-NMR: δ (CDCl₃)=7.25 and 7.04 (in each case d, 1H, PhH), 6.8 (s, 1H, isoxazole), 6.18 (t, 1H, CHCCl₂), 4.64 (d, 2H, CH₂CHCCl₂), 4.83 (s, 2H, CH₂OH), 3.7 (s, 3H, OCH₃).

### Example (I-4)

### 3-(2-methoxy-3-chloro-5-(1,1-dichloro-1-propen-3-oxy)-phenyl)-5-((5-trifluormethyl-pyridin-2-yl)-3-(propyl)ether-1-yl)-isoxazole

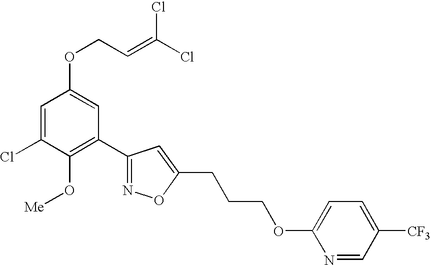

150 mg (0.38 mMol) of 3-(2-methoxy-3-chloro-5-(1,1-dichlor-1-propen-3-oxy)-phenyl)-5-(3-hydroxypropyl)-isoxazole, 70 mg (0.43 mMol) of 5-trifluoromethyl pyridinol and 210 mg (0.8 mMol) of triphenylphosphane are added to approximately 10 mL of tetrahydrofuran (THF) under a protective gas atmosphere at room temperature (approximately 20° C.), then mixed with 140 mg (0.9 mMol) of azodicarboxylic acid diethyl ester and allowed to stand over night. To finish, the mixture is concentrated to dryness and chromatographed over silica gel.

One obtains 114 mg (55% of the theory) of 3-(2-methoxy-3-chloro-5-(1,1-dichloro-1-propen-3-oxy)-phenyl)-5-((5-trifluoromethyl-pyridin-2-yl)-3-(propyl)ether-1-yl)-isoxazole.

¹H-NMR: δ (CDCl₃)=8.41 (d, 1H, Py), 7.79 (dd, 1H, Py), 6.81 (d, 1H, Py), 7.03 and 7.3 (in each case d, 1H, PhH), 6.60 (s, 1H, isoxazole), 6.15 (t, 1H, CHCCl₂), 4.63 (d, 2H, CH₂CHCCl₂), 4.45 (t, 2H, CH₂OPy), 3.0 (t, 2H, CH₂), 2.25 (m, 2H, CH₂), 3.7 (s, 3H, OCH₃).

Compounds of the general formula listed in the following Table 1 can also be produced analogously to the Examples I-1 to I-4 as well as corresponding to the general description of the method according to the invention.

TABLE 1
Examples for the compounds of the formula (I)

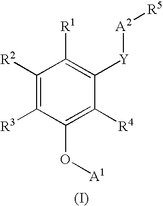

| Ex. | | | | | | | | | |
|---|---|---|---|---|---|---|---|---|---|
| no. | A¹ | A² | R¹ | R² | R³ | R⁴ | R⁵ | Y | Physical data |
| I-5 | 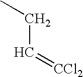 | 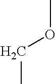 | OCH₃ | Cl | H | H | 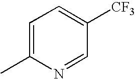 | 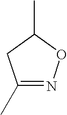 | (see information following this table) |
| I-6 | 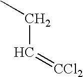 | 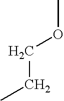 | OCH₃ | Cl | H | H | 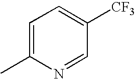 | 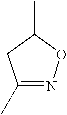 | (see information following this table) |
| I-7 | 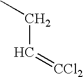 | 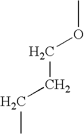 | OCH₃ | Cl | H | H | 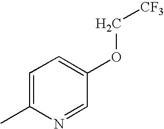 | 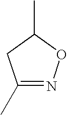 | (see information following this table) |
| I-8 | 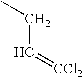 | 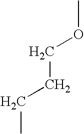 | 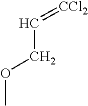 | Cl | H | H | H | 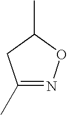 | |
| I-9 | 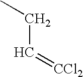 | 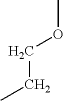 | OCH₃ | Cl | H | H | 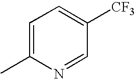 | 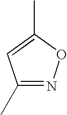 | (see information following this table) |
| I-10 | 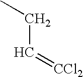 | 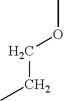 | OCH₃ | Cl | H | H | 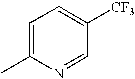 | 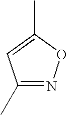 | (see information following this table) |
| I-11 | 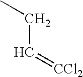 | 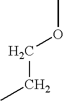 | OCH₃ | Cl | H | H | 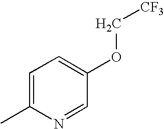 | 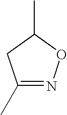 | (see information following this table) |
| I-12 | 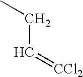 | 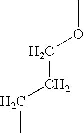 | OCH₃ | Cl | H | H | H | 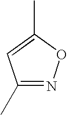 | (see information following this table) |
| I-13 | 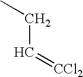 | 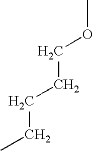 | OCH₃ | Cl | H | H | H | 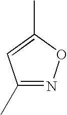 | (see information following this table) |
| I-14 | 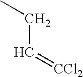 | 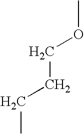 | OCH₃ | Cl | H | H | 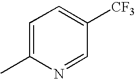 | 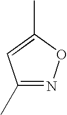 | |
| I-15 | 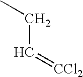 | 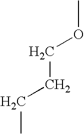 | OCH₃ | Cl | H | H | 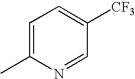 | 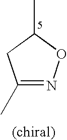 | |
| I-16 | 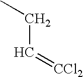 | 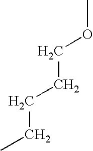 | OCH₃ | Cl | H | H | 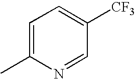 | 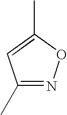 | (see information following this table) |
| I-17 | 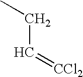 | 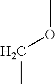 | OCH₃ | Cl | H | H | 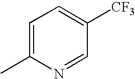 | 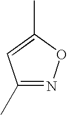 | (see information following this table) |
| I-18 | 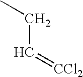 | 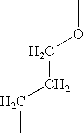 | H | H | OCH₃ | H | 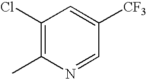 | 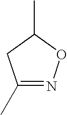 | (see information following this table) |
| I-19 | 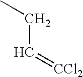 | 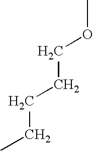 | Cl | H | OCH₃ | H | 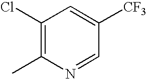 | 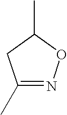 | MS (ES+): 589 |
| I-20 | 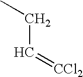 | 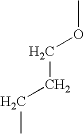 | NO₂ | H | H | H | 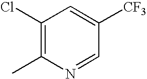 | 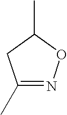 | MS (ES+): 556 |
| I-21 | 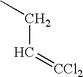 | 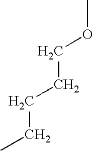 | NO₂ | H | H | H | 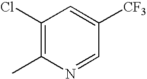 | 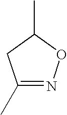 | MS (ES+): 570 |
| I-22 | 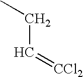 | 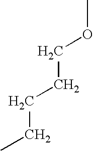 | H | H | OCH₃ | H | 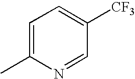 | 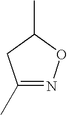 | (see information following this table) |
| I-23 | 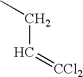 | 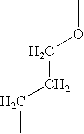 | H | H | OCH₃ | H | H | 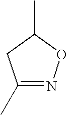 | MS (ES+): 360 |
| I-24 | 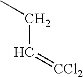 | 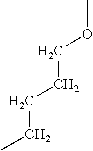 | H | H | OCH₃ | H | H | 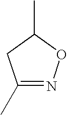 | MS (ES+): 374 |
| I-25 | 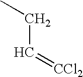 | 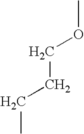 | Cl | H | OCH₃ | H | 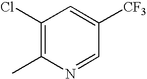 | 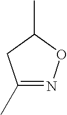 | (see information following this table) |
| I-26 | 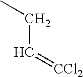 | 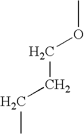 | Cl | H | OCH₃ | H | H | 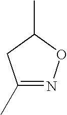 | MS (ES+): 394 |
| I-27 | 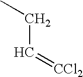 | 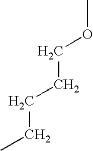 | Cl | H | OCH₃ | H | H | 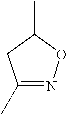 | MS (ES+): 408 |
| I-28 | 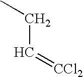 | 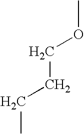 | H | H | OCH₃ | H | 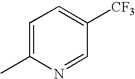 | 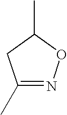 | MS (ES+): 473 |
| I-29 | 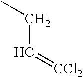 | 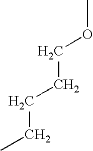 | NO₂ | H | H | H | H | 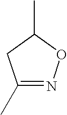 | MS (ES+): 389 |
| I-30 | 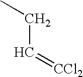 | 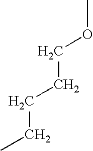 | H | H | F | H | 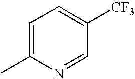 | 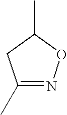 | MS (ES+): 507 |
| I-31 | 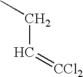 | 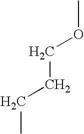 | H | H | F | H | 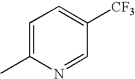 | 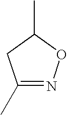 | (see information following this table) |
| I-32 | 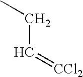 | 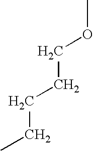 | H | H | F | H | 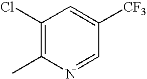 | 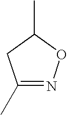 | MS (ES+): 541 |
| I-33 | 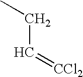 | 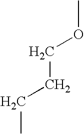 | H | H | F | H | 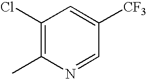 | 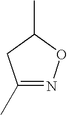 | MS (ES+): 527 |
| I-34 | 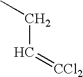 | 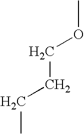 | H | H | F | H | 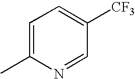 | 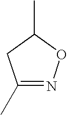 | MS (ES+): 461 |
| I-35 | 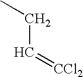 | 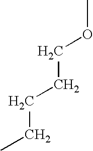 | Cl | H | F | H | 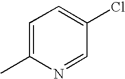 | 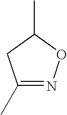 | MS (ES+): 509 |
| I-36 | 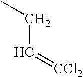 | 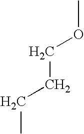 | Br | H | H | H | 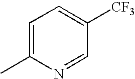 | 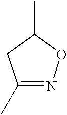 | MS (ES+): 555 |
| I-37 | 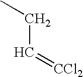 | 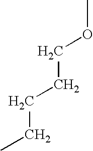 | Br | H | H | H | 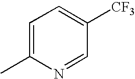 | 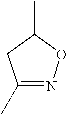 | MS (ES+): 569 |
| I-38 | 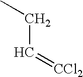 | 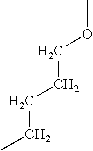 | Br | H | H | H | 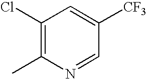 | 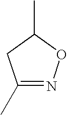 | MS (ES+): 603 |
| I-39 | 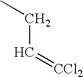 | 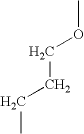 | Br | H | H | H | 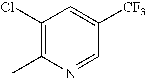 | 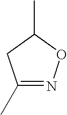 | MS (ES+): 589 |
| I-40 | 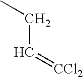 | 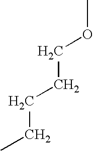 | Br | H | H | H | 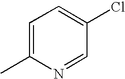 | 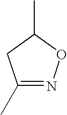 | MS( ES+): 535 |
| I-41 | 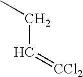 | 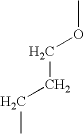 | Br | H | H | H | 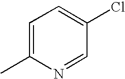 | 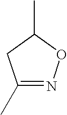 | MS (ES+): 521 |
| I-42 | 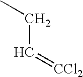 | 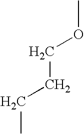 | Cl | H | F | H | 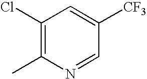 | 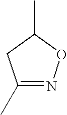 | MS (ES+): 563 |
| I-43 | 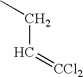 | 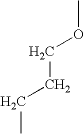 | Cl | H | F | H | 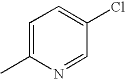 | 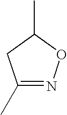 | MS (ES+): 495 |
| I-44 | 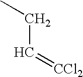 | 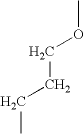 | Cl | H | F | H | 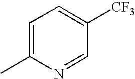 | 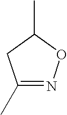 | MS (ES+): 527 |
| I-45 | 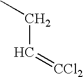 | 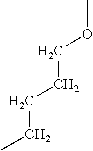 | Cl | H | F | H | 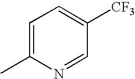 | 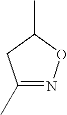 | MS (ES+): 543 |
| I-46 | 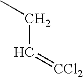 | 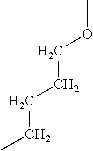 | Cl | H | F | H | 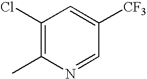 | 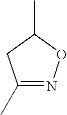 | MS (ES+): 577 |
| I-47 | 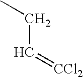 | 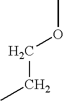 | OC₂H₅ | Cl | H | H | 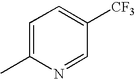 | 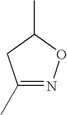 | MS (ES+): 541 |
| I-48 | 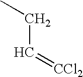 | 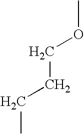 | OC₂H₅ | Cl | H | H | 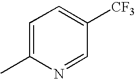 | 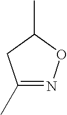 | MS (ES+): 555 |
| I-49 | 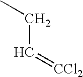 | 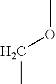 | OCH₃ | H | H | H | 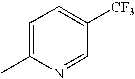 | 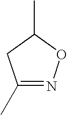 | MS (ES+): 477 |
| I-50 | 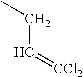 | 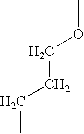 | OCH₃ | H | H | H | 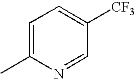 | 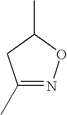 | MS (ES+): 505 |
| I-51 | 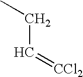 | 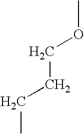 | H | H | H | H | 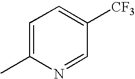 | 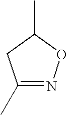 | MS (ES+): 475 |
| I-52 | 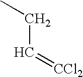 | 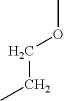 | Cl | H | H | Cl | 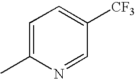 | 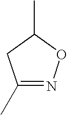 | MS (ES+): 531 |
| I-53 | 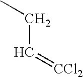 | 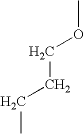 | Cl | H | H | Cl | 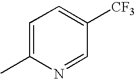 | 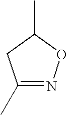 | MS (ES+): 545 |
| I-54 | 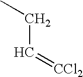 | 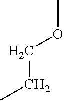 | H | Cl | H | H | 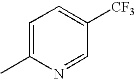 | 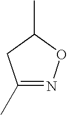 | MS (ES+): 495 |
| I-55 | 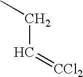 | 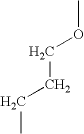 | H | Cl | H | H | 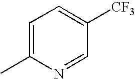 | 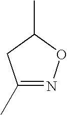 | MS (ES+): 509 |
| I-56 | 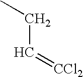 | 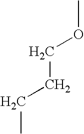 | OCH₃ | Cl | H | H | 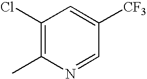 | 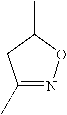 | MS (ES+): 575 |
| I-57 | 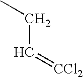 | 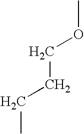 | F | H | H | H | 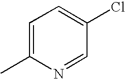 | 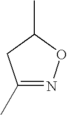 | MS (ES+): 459 |
| I-58 | 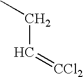 | 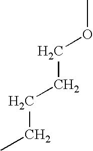 | F | H | H | H | 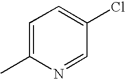 | 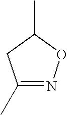 | MS (ES+): 475 |
| I-59 | 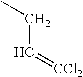 | 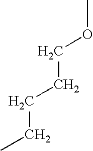 | F | H | H | H | 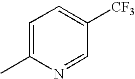 | 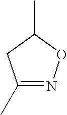 | MS (ES+): 493 |
| I-60 | 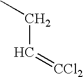 | 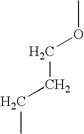 | F | H | H | H | 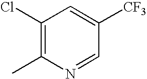 | 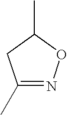 | MS (ES+): 529 |
| I-61 | 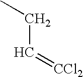 | 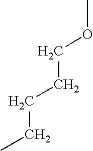 | F | H | H | H | 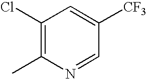 | 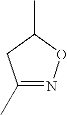 | MS (ES+): 543 |
| I-62 | 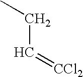 | 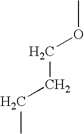 | 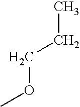 | Cl | H | H | 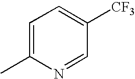 | 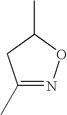 | (see information following this table) |
| I-63 | 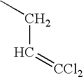 | 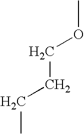 | 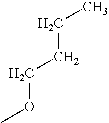 | Cl | H | H | 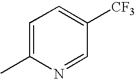 | 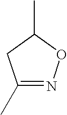 | (see information following this table) |
| I-64 | 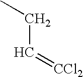 | 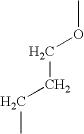 | 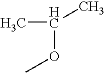 | Cl | H | H | 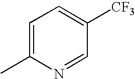 | 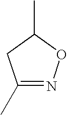 | (see information following this table) |
| I-65 | 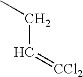 | 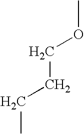 | 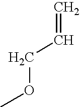 | Cl | H | H | 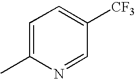 | 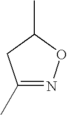 | MS (ES+): 565 |
| I-66 | 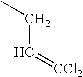 | 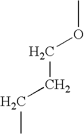 | 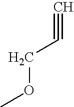 | Cl | H | H | 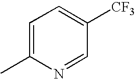 | 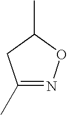 | (see information following this table) |
| I-67 | 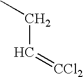 | 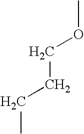 | 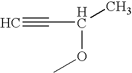 | Cl | H | H | 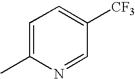 | 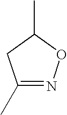 | MS (ES+): 577 |
| I-68 | 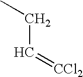 | 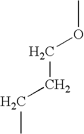 | 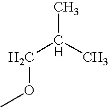 | Cl | H | H | 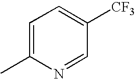 | 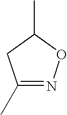 | (see information following this table) |
| I-69 | 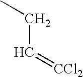 | 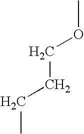 | 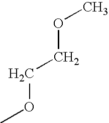 | Cl | H | H | 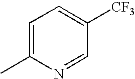 | 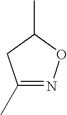 | MS (ES+): 583, M + Na: 605 |
| I-70 | 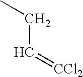 | 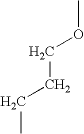 | 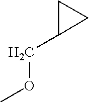 | Cl | H | H | 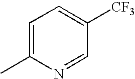 | 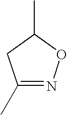 | MS (ES+): 581 |
| I-71 | 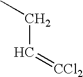 | 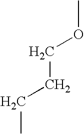 | 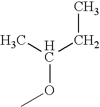 | Cl | H | H | 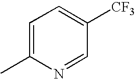 | 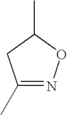 | MS (ES+): 581 |
| I-72 | 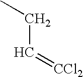 | 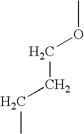 | 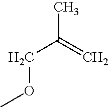 | Cl | H | H | 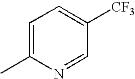 | 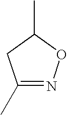 | MS (ES+): 579 |
| I-73 | 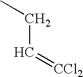 | 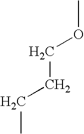 | 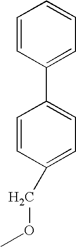 | Cl | H | H | 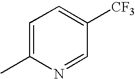 | 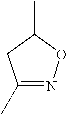 | MS (ES+): 693 |
| I-74 | 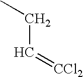 | 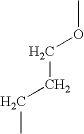 | 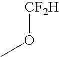 | Cl | H | H | 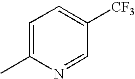 | 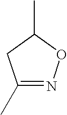 | (see information following this table) |
| I-75 | 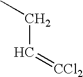 | 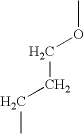 | OH | Cl | H | H | 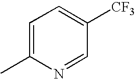 | 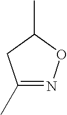 | (see information following this table) |
| I-76 | 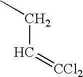 | 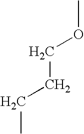 | 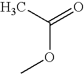 | Cl | H | H | 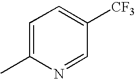 | 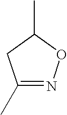 | (see information following this table) |
| I-77 | 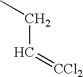 | 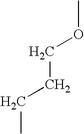 | 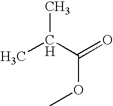 | Cl | H | H | 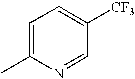 | 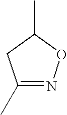 | (see information following this table) |
| I-78 | 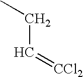 | 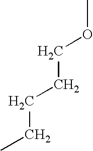 | 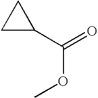 | Cl | H | H | 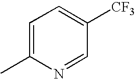 | 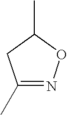 | (see information following this table) |
| I-79 | 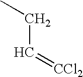 | 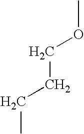 | 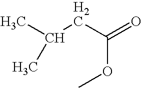 | Cl | H | H | 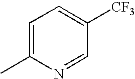 | 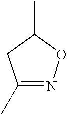 | (see information following this table) |
| I-80 | 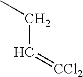 | 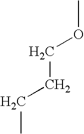 | 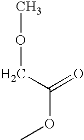 | Cl | H | H | 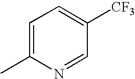 | 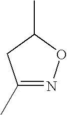 | MS (ES+): 597 |
| I-81 | 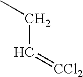 | 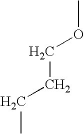 | 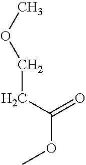 | Cl | H | H | 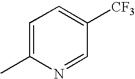 | 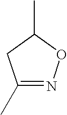 | MS (ES+): 611 |
| I-82 | 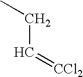 | 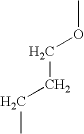 | 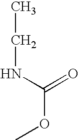 | Cl | H | H | 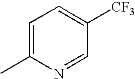 | 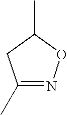 | (see information following this table) |
| I-83 | 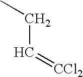 | 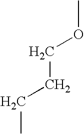 | H | CF₃ | H | H | 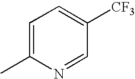 | 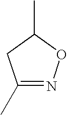 | (see information following this table) |
| I-84 | 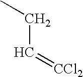 | 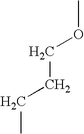 | OC₂H₅ | Cl | H | H | 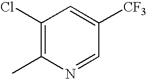 | 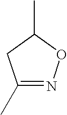 | MS (ES+): 589 |
| I-85 | 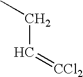 | 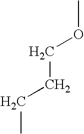 | H | Cl | H | H | 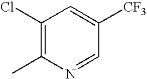 | 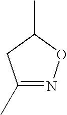 | (see information following this table) |
| I-86 | 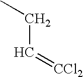 | 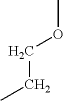 | OCH₃ | Cl | H | H | 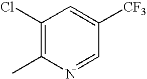 | 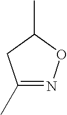 | MS (ES+): 561 |
| I-87 | 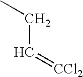 | 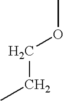 | OCH₃ | Cl | H | H | 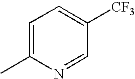 | 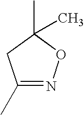 | (see information following this table) |
| I-88 | 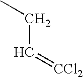 | 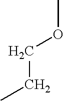 | OCH₃ | Cl | H | H | H | 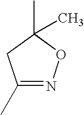 | MS (ES+): 394 |
| I-89 | 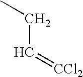 | 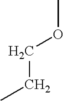 | OCH₃ | Cl | H | H | 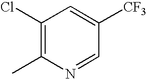 | 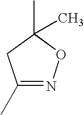 | MS (ES+): 608 |
| I-90 | 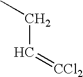 | 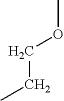 | OCH₃ | Cl | H | H | 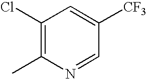 | 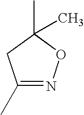 | (see information following this table |
| I-91 | 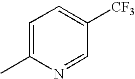 | 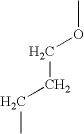 | OCH₃ | Cl | H | H | 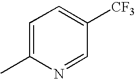 | 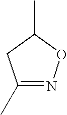 | MS (ES+): 576 |
| I-92 | 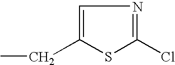 | 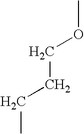 | OCH₃ | Cl | H | H | 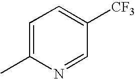 | 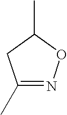 | MS (ES+): 562 |
| I-93 | 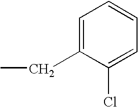 | 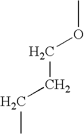 | OCH₃ | Cl | H | H | 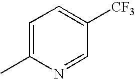 | 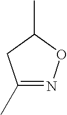 | MS (ES+): 555 |
| I-94 | 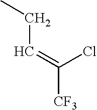 | 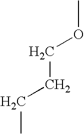 | OCH₃ | Cl | H | H | 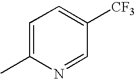 | 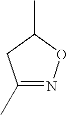 | |
| I-95 | 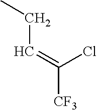 | 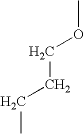 | OCH₃ | Cl | H | H | 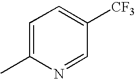 | 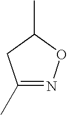 | MS (ES+): 519 |
| I-96 | 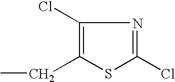 | 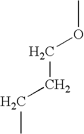 | OCH₃ | Cl | H | H | 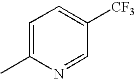 | 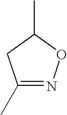 | MS (ES+): 597 |
| I-97 | 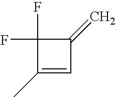 | 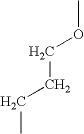 | OCH₃ | Cl | H | H | 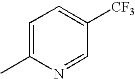 | 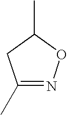 | MS (ES+): 531 |
| I-98 | 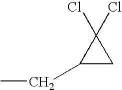 | 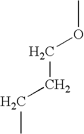 | OCH₃ | Cl | H | H | 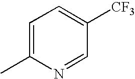 | 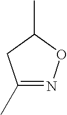 | MS (ES+): 555 |
| I-99 | 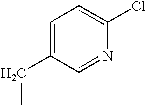 | 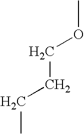 | OCH₃ | Cl | H | H | 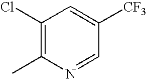 | 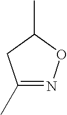 | MS (ES+): 590 |
| I-100 | 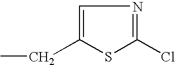 | 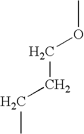 | OCH₃ | Cl | H | H | 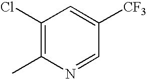 | 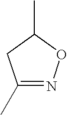 | MS (ES+): 598 |
| I-101 | 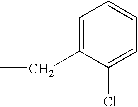 | 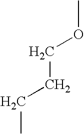 | OCH₃ | Cl | H | H | 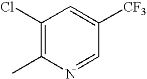 | 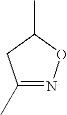 | MS (ES+): 589 |
| I-102 | 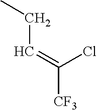 | 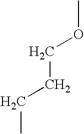 | OCH₃ | Cl | H | H | 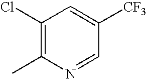 | 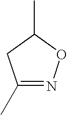 | MS (ES+): 607 |
| I-103 | 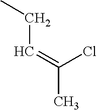 | 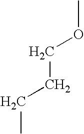 | OCH₃ | Cl | H | H | 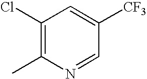 | 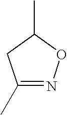 | MS (ES+): 553 |
| I-104 | 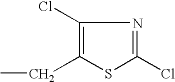 | 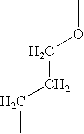 | OCH₃ | Cl | H | H | 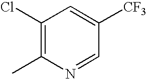 | 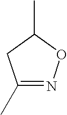 | MS (ES+): 632 |
| I-105 | 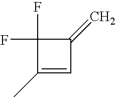 | 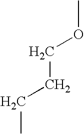 | OCH₃ | Cl | H | H | 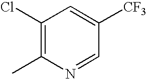 | 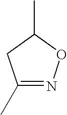 | MS (ES+): 565 |
| I-106 | 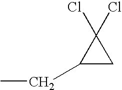 | 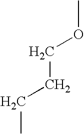 | OCH₃ | Cl | H | H | 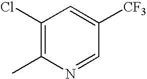 | 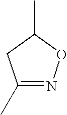 | MS (ES+): 589 |
| I-107 | 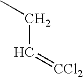 | 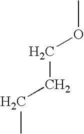 | 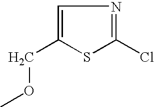 | Cl | H | H | 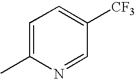 | 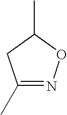 | MS (ES+): 658 |
| I-108 | 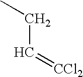 | 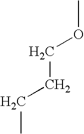 | 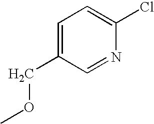 | Cl | H | H | 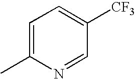 | 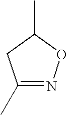 | MS (ES+): 652 |
| I-109 | 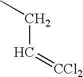 | 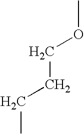 | 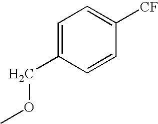 | Cl | H | H | 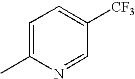 | 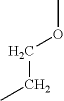 | MS (ES+): 683 |
| I-110 | 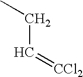 | 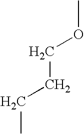 | 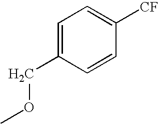 | Cl | H | H | 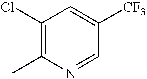 | 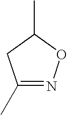 | MS (ES+): 719 |
| I-111 | 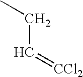 | 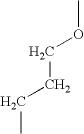 | 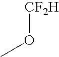 | Cl | H | H | 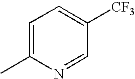 | 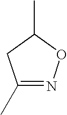 | MS (ES+):683 |
| I-112 | 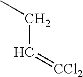 | 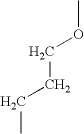 | 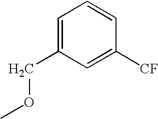 | Cl | H | H | 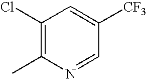 | 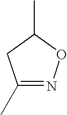 | MS (ES+): 719 |
| I-113 | 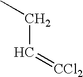 | 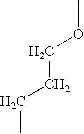 | 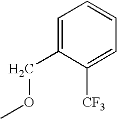 | Cl | H | H | 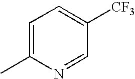 | 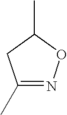 | MS (ES+): 683 |
| I-114 | 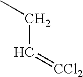 | 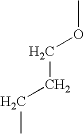 | 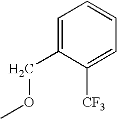 | Cl | H | H | 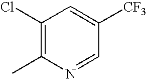 | 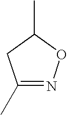 | MS (ES+): 719 |
| I-115 | 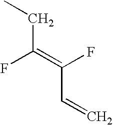 | 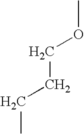 | OCH₃ | Cl | H | H | 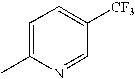 | 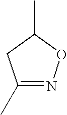 | MS (ES+): 519 |
| I-116 | 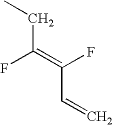 | 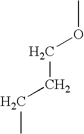 | OCH₃ | Cl | H | H | 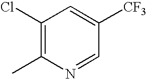 | 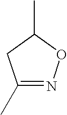 | MS (ES+): 553 |
| I-117 | 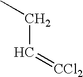 | 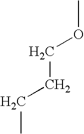 | OCH₃ | Cl | H | F | 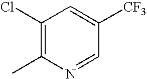 | 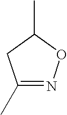 | MS (ES+): 593 |
| I-118 | 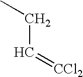 | 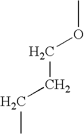 | OCH₃ | Cl | H | H | 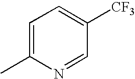 | 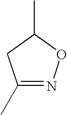 | MS (ES+): 557 |
| I-119 | 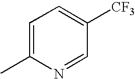 | 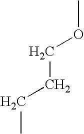 | OCH₃ | Cl | H | H | 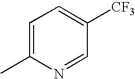 | 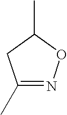 | MS (ES+): 576 |
| I-120 | 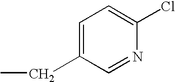 | 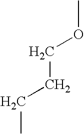 | OCH₃ | Cl | H | H | 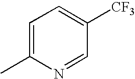 | 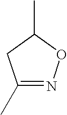 | MS (ES+): 556 |
| I-121 | 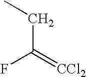 | 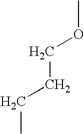 | OCH₃ | Cl | H | H | 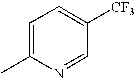 | 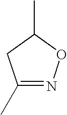 | MS (ES+): 557 |
| I-122 | 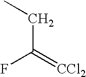 | 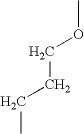 | OCH₃ | Cl | H | H | 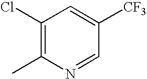 | 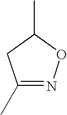 | MS (ES+): 593 |
| I-123 | 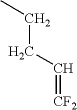 | 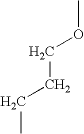 | OCH₃ | Cl | H | H | 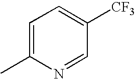 | 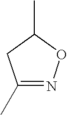 | |
| I-124 | 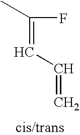 | 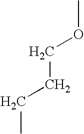 | OCH₃ | Cl | H | H | 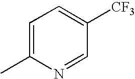 | 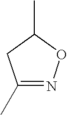 | |
| I-125 | 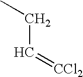 | 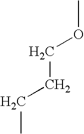 | OCH₃ | H | H | H | 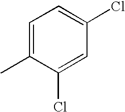 | 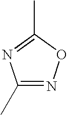 | (see information following the table) |
| I-126 | 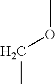 | 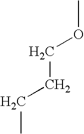 | OCH₃ | H | H | H | 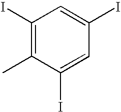 | 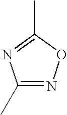 | (see information following the table) |
| I-127 | 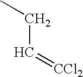 | 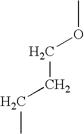 | OCH₃ | H | H | H | 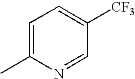 | 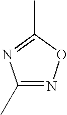 | (see information following the table) |
| I-128 | 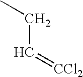 | 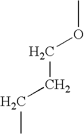 | OCH₃ | Cl | H | H | 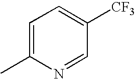 | 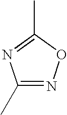 | (see information following the table) |
| I-129 | 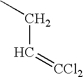 | 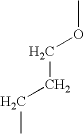 | OCH₃ | Cl | H | H | 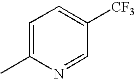 | 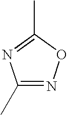 | (see information following the table) |
| I-130 | 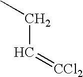 | 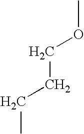 | OCH₃ | Cl | H | H | 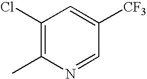 | 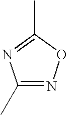 | (see information following the table) |
| I-131 | 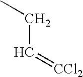 | 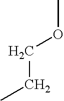 | OCH₃ | Cl | H | H | 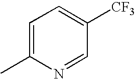 | 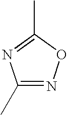 | (see information following the table) |
| I-132 | 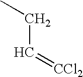 | 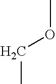 | OCH₃ | Cl | H | H | 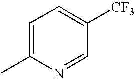 | 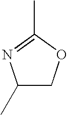 | (see information following the table) |
| I-133 | 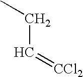 | 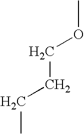 | 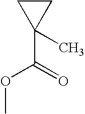 | Cl | H | H | 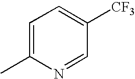 | 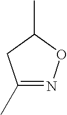 | MS (ES+): 607 |
| I-134 | 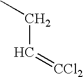 | 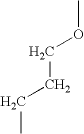 | 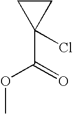 | Cl | H | H | 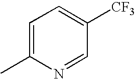 | 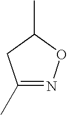 | MS (ES+): 629 |
| I-135 | 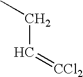 | 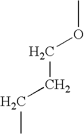 | 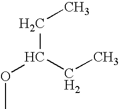 | Cl | H | H | 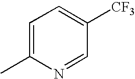 | 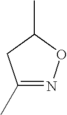 | MS (ES+): 595 |
| I-136 | 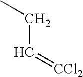 | 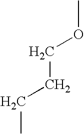 | 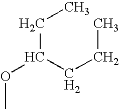 | Cl | H | H | 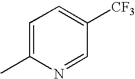 | 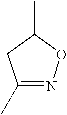 | MS (ES+): 609 |
| I-137 | 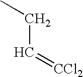 | 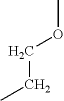 | OCH₃ | Cl | H | H | 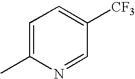 | 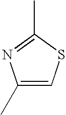 | (see information following the table) |
| I-138 | 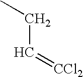 | 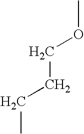 | 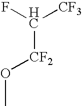 | Cl | H | H | 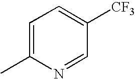 | 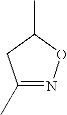 | (see information following the table) |
| I-139 | 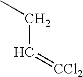 | 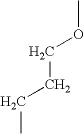 | —N(CH₃)₂ | Cl | H | H | 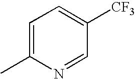 | 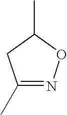 | |

Physical Data and Production Processes for Compounds of Table 1:

### Example (I-5)

### (R/S)-3-(2-methoxy-3-chloro-5-(dichlorpropenoxy)phenyl)-5-((5-trifluormethylpyridin-2-yl)-2-(methyl)ether-1-yl)Δ²-isoxazoline

¹H-NMR: δ (CDCl₃)=8.42 (d, 1H, Py), 7.8 (dd, 1H, Py), 6.83 (d, 1H, Py), 7.05 and 7.2 (in each cash d, 1H, PhH), 6.18 (t, 1H, CHCCl₂), 4.62 (d, 2H, CH₂CHCCl₂), 5.18 (m, 1H, CHO (isoxazoline)), 4.58 (d, 2H, CH₂OPy), 3.8 (s, 3H, CH₃), 3.6 and 3.4 (in each case dd, 1H, diastereotopes N═CCH₂ (isoxazoline)).

### Example (I-6)

### A.I.1.1 (R/S)-3-(2-methoxy-3-chloro-5-(dichloropropenoxy)phenyl)-5-((6-trifluorethoxypyridin-3-yl)-2-(ethyl)ether-1-yl)Δ²-isoxazoline

¹H-NMR: δ (CDCl₃)=7.8 (d, 1H, Py), 7.23 (dd, 1H, Py), 6.8 (d, 1H, Py), 7.0 and 7.16 (in each case d, 1H, PhH), 6.15 (t, 1H, CHCCl₂), 4.6 (d, 2H, CH₂CHCCl₂), 4.7 (q, 2H, CH₂CF₃), 5.0 (m, 1H, CHO (isoxazoline)), 4.18 (m, 2H, CH₂OPy), 3.8 (s, 3H, CH₃), 3.6 and 3.2 (in each case dd, 1H, diastereotopes N═CCH₂ (isoxazoline)), 2.2 (m, 2H, CH₂CH₂OPy).

### Example (I-7)

### (R/S)-3-(2-methoxy-3-chloro-5-(dichloropropenoxy)phenyl)-5-((6-trifluorethoxypyridin-3-yl)-3-(propyl)ether-1-yl)Δ²-isoxazoline

¹H-NMR: δ (CDCl₃)=7.78 (d, 1H, Py), 7.25 (dd, 1H, Py), 6.8 (d, 1H, Py), 7.0 and 7.18 (in each case d, 1H, PhH), 6.15 (t, 1H, CHCCl₂), 4.6 (d, 2H, CH₂CHCCl₂), 4.7 (q, 2H, CH₂CF₃), 4.8 (m, 1H, CHO (isoxazoline)), 4.02 (m, 2H, CH₂OPy), 3.8 (s, 3H, CH₃), 3.5 and 3.1 (in each case dd, 1H, diastereotopes N═CCH₂ (isoxazoline)), 1.8 bis 2.0 (m, altogether 4H, CH₂CH₂CH₂OPy).

### Example (I-9)

### 3-(2-methoxy-3-chloro-5-(dichloropropenoxy)phenyl)-5-((5-trifluormethylpyridin-2-yl)-2-(ethyl)ether-1-yl)isoxazole

¹H-NMR: δ (CDCl₃)=8.41 (d, 1H, Py), 7.79 (dd, 1H, Py), 6.81 (d, 1H, Py), 7.0 and 7.3 (in each case d, 1H, PhH), 6.65 (s, 1H, isoxazole), 6.15 (t, 1H, CHCCl₂), 4.6 (d, 2H, CH₂CHCCl₂), 4.75 (t, 2H, CH₂OPy), 3.3 (t, 2H, CH₂), 3.63 (s, 3H, OCH₃).

### Example (I-10)

### (R/S)-3-(2-methoxy-3-chloro-5-(dichloropropenoxy)phenyl)-5-((5-trifluormethylpyridin-2-yl)-4-(butyl)ether-1-yl)Δ²-isoxazoline

¹H-NMR: δ (CDCl₃)=8.42 (d, 1H, Py), 7.78 (dd, 1H, Py), 6.80 (d, 1H, Py), 7.0 and 7.16 (in each case d, 1H, PhH), 6.15 (t, 1H, CHCCl₂), 4.6 (d, 2H, CH₂CHCCl₂), 4.78 (m, 1H, CHO (isoxazoline)), 4.4 (t, 2H, CH₂OPy), 3.8 (s, 3H, CH₃), 3.45 and 3.05 (in each case dd, 1H, diastereotopes N═CCH₂ (isoxazoline)), 1.5 to 1.9 (m, altogether 4H, CH₂CH₂CH₂OPy).

### Example (I-11)

### (R/S)-3-(2-methoxy-3-chloro-5-(dichloropropenoxy)phenyl)-5-((6-trifluorethoxypyridin-3-yl)-2-(ethyl)ether-1-yl)Δ²-isoxazoline

¹H-NMR: δ (CDCl₃)=8.42 (d, 1H, Py), 7.78 (dd, 1H, Py), 6.83 (d, 1H, Py), 7.0 and 7.16 (in each case d, 1H, PhH), 6.15 (t, 1H, CHCCl₂), 4.6 (d, 2H, CH₂CHCCl₂), 5.0 (m, 1H, CHO (isoxazoline)), 4.58 (m, 2H, CH₂OPy), 3.8 (s, 3H, CH₃), 3.58 and 3.2 (in each case dd, 1H, diastereotopes N═CCH₂ (isoxazoline)), 2.2 (m, 2H, CH₂CH₂OPy).

### Example (I-12)

### 3-(2-methoxy-3-chloro-5-(dichloropropenoxy)phenyl)-5-(3-hydroxypropyl)isoxazole

¹H-NMR: δ (CDCl₃)=7.0 and 7.3 (in each case d, 1H, PhH), 6.58 (s, 1H, isoxazole), 6.18 (t, 1H, CHCCl₂), 4.64 (d, 2H, CH₂CHCCl₂), 3.78 (t, 2H, CH₂OH), 2.95 (t, 2H, CH₂), 2.0 (m, 2H, CH₂), 3.7 (s, 3H, OCH₃).

### Example (I-13)

### 3-(2-methoxy-3-chloro-5-(dichloropropenoxy)phenyl)-5-(4-hydroxybutyl)isoxazole

¹H-NMR: δ (CDCl₃)=7.02 and 7.3 (in each case d, 1H, PhH), 6.58 (s, 1H, isoxazole), 6.18 (t, 1H, CHCCl₂), 4.62 (d, 2H, CH₂CHCCl₂), 3.68 (m, 2H, CH₂OH), 2.83 (t, 2H, CH₂), 1.64, 1.85 (in each case m, 2H, CH₂), 3.68 (s, 3H, OCH₃).

### Example (I-16)

### 3-(2-methoxy-3-chloro-5-(dichloropropenoxy)phenyl)-5-((5-trifluormethylpyridin-2-yl)-4-(butyl)ether-1-yl)isoxazole

¹H-NMR: δ (CDCl₃)=8.41 (d, 1H, PyH), 7.78 (dd, 1H, PyH), 6.8 (d, 1H, PyH), 7.3 and 7.02 (in each case d, 1H, PhH), 6.58 (s, 1H, isoxazole), 6.15 (t, 1H, CHCCl₂), 4.63 (d, 2H, CH₂CHCCl₂), 4.4 (t, 2H, CH₂OPy), 2.9 (t, 2H, CH₂), 1.9 (m, 4H, two CH₂), 3.68 (s, 3H, OCH₃).

### Example (I-17)

### 3-(2-methoxy-3-chloro-5-(dichloropropenoxy)phenyl)-5-((5-trifluormethylpyridin-2-yl)(methyl)ether)isoxazole

¹H-NMR: δ (CDCl₃)=8.46 (d, 1H, PyH), 7.83 (dd, 1H, Py), 6.93 (d, 1H, Py), 7.33 and 7.05 (in each case d, 1H, PhH), 6.87 (s, 1H, isoxazole), 6.18 (t, 1H, CHCCl₂), 4.62 (d, 2H, CH₂CHCCl₂), 5.6 (s, 2H, CH₂OPy), 3.67 (s, 3H, OCH₃).

### Example (I-18)

### (R/S)-3-(4-methoxy-5-(dichloropropenoxy)phenyl)-5-((3-chloro-5-trifluormethylpyridin-2-yl)(propyl)ether-1-yl)-Δ²-isoxazoline

¹³C-NMR: δ (CDCl₃)=24.9, 31.9, 40.2 (CH₂), 56.0 (O—CH₃), 65.8, 67.4 (CH₂—O), 80.7 (CH), 124.5 (═CCl₂), 125.1 (═CH), 156.0 (C═N—O), 110.9, 111.1 (HC—Ar), 121.0 (HC—Ar), 122.6 (HC—Ar), 147.4, 151.1 (O—C—Ar), 118.7 (Cl—C-Hetar), 135.2 (C-Hetar), 120.7 (F₃C-Hetar), 142.4 (HC-Hetar), 161.2 (O—C-Hetar).

### Example (I-22)

### (R/S)-3-(4-methoxy-5-(dichloropropenoxy)phenyl)-5-((5-trifluormethylpyridin-2-yl)-(butyl)ether-1-yl)Δ²-isoxazoline

¹³C-NMR: δ (CDCl₃)=22.1, 28.7, 35.0, 40.1 (CH₂), 55.9 (O—CH₃), 65.8, 66.5 (CH₂—O), 81.1 (CH), 124.5 (═CCl₂), 125.1 (═CH), 155.9 (C═N—O), 110.8 (HC—Ar), 111.1 (HC—Ar), 111.2 (HC-Hetar), 119.8 (C-Hetar), 120.9 (HC—Ar), 122.6 (HC—Ar), 135.6 (HC-Hetar), 144.9 (HC-Hetar), 147.4 (O—C—Ar), 151.1 (C—Ar), 165.8 (O—C-Hetar).

### Example (I-25)

### (R/S)-3-(2-Chlor-4-methoxy-5-(dichloropropenoxy)phenyl)-5-((3-chloro-5-trifluormethylpyridin-2-yl)-(propyl)ether-1-yl)Δ²-isoxazoline

¹³C-NMR: δ (CDCl₃)=24.9, 31.6, 42.5 (CH₂), 56.2 (O—CH₃), 66.0, 67.4 (CH₂—O), 81.6 (CH), 124.9 (═CCl₂), 124.7 (═CH), 156.2 (C═N—O), 114.2, (HC—Ar), 121.0 (HC—Ar), 113.6 (HC—Ar), 125.6 (Cl—C—Ar), 146.1, 151.1 (O—C—Ar), 118.8 (Cl—C-Hetar), 120.7 (C-Hetar), 123.1 (F₃C-Hetar), 135.2 (HC-Hetar), 142.4 (HC-Hetar), 161.2 (O—C-Hetar).

### Example (I-31)

### (R/S)-3-(2-chloro-4-fluoro-5-(dichlorpropenoxy)phenyl)-5-((5-trifluoromethylpyridin-2-yl)-(propyl)ether-1-yl)Δ²-isoxazoline

¹³C-NMR: δ (CDCl₃)=25.0, 31.6, 42.2 (CH₂), 66.1, 66.3 (CH₂—O), 82.0 (CH), 124.1 (═CH), 124.5 (═CCl₂), 155.8 (C═N—O), 111.2 (HC-Hetar), 116.1 (HC—Ar), 118.7 (HC—Ar), 119.9 (C-Hetar), 121.2 (F₃C-Hetar), 125.0 (Cl—C—Ar), 125.5 (C—Ar), 135.6 (H—C-Hetar), 144.9 (—O—C—Ar), 144.9 (HC-Hetar), 153.0 (F—C—Ar), 165.8 (O—C-Hetar).

### Example (I-62)

(R/S)-3-(2-propoxy-3-chloro-5-(1,1-dichloro-1-propen-3-oxy)-phenyl)-5-((5-trifluoromethylpyridin-2-yl)-3-(propyl)ether-1-yl)-Δ²-isoxazoline: MS (ES+): 567. ¹H-NMR: CDCl₃, δ=8.43 (1H, Py), 7.77 (dd, 1H, Py), 6.80 (d, 1H, Py), 7.02 and 7.12 (in each case d, 1H, PhH), 6.14 (t, 1H, CHCCl₂), 4.62 (d, 2H, CH₂CHCCl₂), 4.8 (m, 1H, CHO (isoxazoline)), 4.42 (m, 2H, CH₂OPy), 3.82 (t, 2H, PhOCH₂), 3.51 and 3.10 (in each case dd, 1H, diastereotopes N═CCH₂ (isoxazoline)), 1.9 (m, 4H, PyOCH₂CH₂CH₂), 1.82 (q, 2H, CH₂CH₃) 1.04 (t, 3H, CH₂CH₃).

### Example (I-63)

### (R/S)-3-(2-butoxy-3-chloro-5-(1,1-dichloro-1-propen-3-oxy)-phenyl)-5-((5-trifluoromethyl-pyridin-2-yl)-3-(propyl)ether-1-yl)-Δ²-isoxazoline

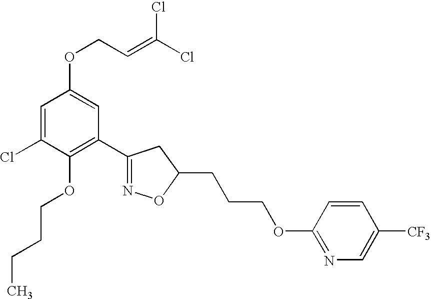

60 mg (0.114 mMol) of (R/S)-3-(2-hydroxy-3-chloro-5-(1,1-dichloro-1-propen-3-oxy)-phenyl)-5-((5-trifluoromethyl-pyridin-2-yl)-3-(propyl)ether-1-yl)-Δ²-isoxazoline, 84 mg (0.457 mMol) of 1-iodobutane and 79 mg (0.571 mMol) of potassium carbonate are stirred in 4 ml of acetone for 18 hours under reflux. The reaction mixture is distributed between water and acetic ether. After the concentration of the organic phase to dryness, the remaining residue is chromatographed over silica gel.

### Example (I-64)

(R/S)-3-(2-isopropoxy-3-chloro-5-(1,1-dichloro-1-propen-3-oxy)-phenyl)-5-((5-trifluoromethylpyridin-2-yl)-3-(propyl)ether-1-yl)-Δ²-isoxazoline: MS (ES+): 567. ¹H-NMR: CDCl₃, δ=8.43 (1H, Py), 7.77 (dd, 1H, Py), 6.80 (d, 1H, Py), 7.02 and 7.06 (in each case d, 1H, PhH), 6.15 (t, 1H, CHCCl₂), 4.62 (d, 2H, CH₂CHCCl₂), 4.8 (m, 1H, CHO (isoxazoline)), 4.42 (m, 2H, CH₂OPy), 4.39 (m, 1H, OCH(CH₃)₂), 3.51 and 3.10 (in each case dd, 1H, diastereotopes N═CCH₂ (isoxazoline)), 1.9 (m, 4H, PyOCH₂CH₂CH₂), 1.27 (pseudo t, 6H, OCH(CH₃)₂).

### Example (I-66)

(R/S)-3-(2-(2-propinyl)-oxy-3-chloro-5-(1,1-dichloro-1-propen-3-oxy)-phenyl)-5-((5-trifluoromethyl-pyridin-2-yl)-3-(propyl)ether-1-yl)-Δ²-isoxazoline: MS (ES+): M 563. ¹H-NMR: CDCl₃, δ=8.43 (1H, Py), 7.77 (dd, 1H, Py), 6.80 (d, 1H, Py), 7.02 and 7.22 (in each case d, 1H, PhH), 6.14 (t, 1H, CHCCl₂), 4.62 (d, 2H, CH₂CHCCl₂), 4.82 (m, 1H, CHO (isoxazoline)), 4.69 (d, 2H, PhOCH₂), 4.42 (m, 2H, CH₂OPy), 3.60 and 3.21 (in each case dd, 1H, diastereotopes N═CCH₂ (isoxazoline)), 2.53 (m, 1H, alkine H), 1.9 (m, 4H, PyOCH₂CH₂CH₂), 1.77 (m, 2H, PhOCH₂CH₂) 1.49 (m, 2H, CH₂CH₃), 0.97 (t, 3H, CH₃).

### Example (I-68)

(R/S)-3-(2-isobutoxy-3-chloro-5-(1,1-dichloro-1-propen-3-oxy)-phenyl)-5-((5-trifluoromethylpyridin-2-yl)-3-(propyl)ether-1-yl)-Δ²-isoxazoline: MS (ES+): 581. ¹H-NMR: CDCl₃, δ=8.43 (1H, Py), 7.77 (dd, 1H, Py), 6.80 (d, 1H, Py), 7.02 and 7.11 (in each case d, 1H, PhH), 6.14 (t, 1H, CHCCl₂), 4.62 (d, 2H, CH₂CHCCl₂), 4.8 (m, 1H, CHO (isoxazoline)), 4.42 (m, 2H, CH₂OPy), 3.62 (m, 2H, PhOCH₂), 3.50 and 3.10 (in each case dd, 1H, diastereotopes N═CCH₂ (isoxazoline)), 1.9 (m, 4H, PyOCH₂CH₂CH₂), 1.04 (m, 6H, CH₃), CH(CH₃)₂ not assigned.

### Example (I-74)

### (R/S)-3-(2-difluoromethyloxy-3-chloro-5-(1,1-dichloro-1-propen-3-oxy)-phenyl)-5-((5-trifluoromethyl-pyridin-2-yl)-3-(propyl)ether-1-yl)-Δ²-isoxazoline

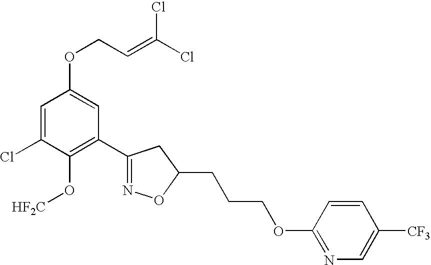

A solution of 60 mg (0.114 mMol) of (R/S)-3-(2-hydroxy-3-chloro-5-(1,1-dichloro-1-propen-3-oxy)-phenyl)-5-((5-trifluormethyl-pyridin-2-yl)-3-(propyl)ether-1-yl)-Δ²-isoxazoline in 0.6 ml of THF at 0° C. is added drop by drop to a suspension of 4 mg (0.167 mMol) of sodium hydride within 10 min. It is stirred for one hour, added to 0.9 ml of DMF and fed into chlorodifluoromethane for 30 min. The reaction mixture is distributed between water and acetic ether. After the concentration of the organic phase to dryness, the remaining residue is chromatographed over silica gel.

One obtains 70 mg (purity 95%, 76% of the theory) of (R/S)-3-(2-difluoromethyloxy-3-chloro-5-(1,1-dichloro-1-propen-3-oxy)-phenyl)-5-((5-trifluoromethyl-pyridin-2-yl)-3-(propyl)ether-1-yl)-Δ²-isoxazoline. MS (ES+): 575. ¹H-NMR: CDCl₃, δ=8.43 (1H, Py), 7.77 (dd, 1H, Py), 6.80 (d, 1H, Py), 7.06 and 7.22 (in each case d, 1H, PhH), 6.52 (t, J=75 Hz, 1H, CH₂), 6.14 (t, 1H, CHCCl₂), 4.65 (d, 2H, CH₂CHCCl₂), 4.85 (m, 1H, CHO (isoxazoline)), 4.42 (m, 2H, CH₂OPy), 3.52 and 3.14 (in each case dd, 1H, diastereotopes N═CCH₂ (isoxazoline)), 1.9 (m, 4H, PyOCH₂CH₂CH₂).

### Example (I-75)

### (R/S)-3-(2-hydroxy-3-chloro-5-(1,1-dichloro-1-propen-3-oxy)-phenyl)-5-((5-trifluoromethylpyridin-2-yl)-3-(propyl)ether-1-yl)-Δ²-isoxazoline

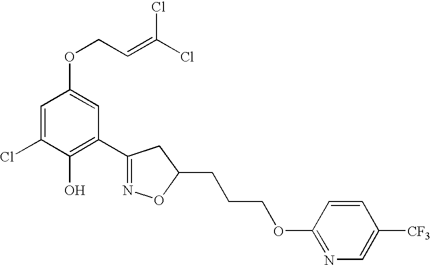

1.0 g (1.45 mMol) of (R/S)-3-(2-(biphenyl-4-ylmethoxy)-3-chloro-5-(1,1-dichloro-1-propen-3-oxy)-phenyl)-5-((5-trifluoromethyl-pyridin-2-yl)-3-(propyl)ether-1-yl)-Δ²-isoxazoline is dissolved in 25 ml of dichloromethane and stirred for 30 min. at room temperature with 0.47 g (2.89 mMol) of iron(III) chloride. 50 ml of water is added and extracted three times with 50 ml of acetic ether in each case. The organic phase is eluted over silica gel and rinsed with acetic ether. After the concentration of the organic phase to dryness, the remaining residue is chromatographed over silica gel.

One obtains 0.42 g (purity 89%, 49% of the theory) of (R/S)-3-(2-hydroxy-3-chloro-5-(1,1-dichloro-1-propen-3-oxy)-phenyl)-5-((5-trifluoromethyl-pyridin-2-yl)-3-(propyl)ether-1-yl)-Δ²-isoxazole. MS (ES+): 525. ¹H-NMR: CDCl₃, δ=10.05 (s, 1H, OH), 8.43 (1H, Py), 7.77 (dd, 1H, Py), 6.80 (d, 1H, Py), 6.64 and 7.03 (in each case d, 1H, PhH), 6.12 (t, 1H, CHCCl₂), 4.62 (d, 2H, CH₂CHCCl₂), 4.85 (m, 1H, CHO (isoxazoline)), 4.42 (m, 2H, CH₂OPy), 3.51 and 3.07 (in each case dd, 1H, diastereotopes N═CCH₂ (isoxazoline)), 1.95 (m, 4H, PyOCH₂CH₂CH₂).

One obtains 52 mg (purity 96%, 75% of the theory) (R/S)-3-(2-butoxy-3-chloro-5-(1,1-dichloro-1-propen-3-oxy)-phenyl)-5-((5-trifluoromethyl-pyridin-2-yl)-3-propyl)ether-1-yl)-Δ²-isoxazoline. MS (ES+): 581, M+Na: 603. ¹H-NMR: CDCl₃, δ=8.43 (1H, Py), 7.77 (dd, 1H, Py), 6.80 (d, 1H, Py), 7.02 and 7.12 (in each case d, 1H, PhH), 6.14 (t, 1H, CHCCl₂), 4.62 (d, 2H, CH₂CHCCl₂), 4.8 (m, 1H, CHO (isoxazoline)), 4.42 (m, 2H, CH₂OPy), 3.86 (t, 2H, PhOCH₂), 3.51 and 3.10 (in each case dd, 1H, diastereotopes N═CCH₂ (isoxazoline)), 1.9 (m, 4H, PyOCH₂CH₂CH₂), 1.77 (m, 2H, PhOCH₂CH₂) 1.49 (m, 2H, CH₂CH₃), 0.97 (t, 3H, CH₃).

### Example (I-76)

### (R/S)-3-(2-acetyloxy-3-chloro-5-(1,1-dichloro-1-propen-3-oxy)-phenyl)-5-((5-trifluoromethylpyridin-2-yl)-3-(propyl)ether-1-yl)-Δ²-isoxazoline

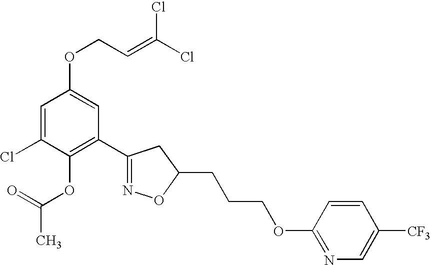

60 mg (0.114 mMol) of (R/S)-3-(2-hydroxy-3-chloro-5-(1,1-dichloro-1-propen-3-oxy)-phenyl)-5-((5-trifluoromethyl-pyridin-2-yl)-3-(propyl)ether-1-yl)-Δ²-isoxazoline are dissolved in 3 ml of pyridine. 23 mg (0.228 mMol) of acetic acid anhydride and a catalytic amount of DMAP are added and the mixture is stirred for two hours at room temperature. The reaction mixture is distributed between water and acetic ether. After the concentration of the organic phase to dryness, the remaining residue is chromatographed over silica gel.

One obtains 62 mg (purity 100%, 95% of the theory) of (R/S)-3-(2-butoxy-3-chloro-5-(1,1-dichloro-1-propen-3-oxy)-phenyl)-5-((5-trifluoromethyl-pyridin-2-yl)-3-(propyl)ether-yl)-Δ²-isoxazoline. MS (ES+): 567. ¹H-NMR: CDCl₃, δ=8.43 (1H, Py), 7.77 (dd, 1H, Py), 6.80 (d, 1H, Py), 6.97 and 7.03 (in each case d, 1H, PhH), 6.14 (t, 1H, CHCCl₂), 4.65 (d, 2H, CH₂CHCCl₂), 4.8 (m, 1H, CHO (isoxazoline)), 4.42 (m, 2H, CH₂OPy), 3.39 and 2.96 (in each case dd, 1H, diastereotopes N═CCH₂ (isoxazoline)), 2.35 (s, 1H, CH₃), 1.9 (m, 4H, PyOCH₂CH₂CH₂).

### Example (I-77)

(R/S)-3-(2-isobutyroxy-3-chloro-5-(1,1-dichloro-1-propen-3-oxy)-phenyl)-5-(5-trifluoromethylpyridin-2-yl)-3-(propyl)ether-1-yl)-Δ²-isoxazoline: MS (ES+): 595. ¹H-NMR: CDCl₃, δ=8.43 (1H, Py), 7.77 (dd, 1H, Py), 6.80 (d, 1H, Py), 6.97 and 7.02 (in each case d, 1H, PhH), 6.14 (t, 1H, CHCCl₂), 4.65 (d, 2H, CH₂CHCCl₂), 4.77 (m, 1H, CHO (isoxazoline)), 4.41 (m, 2H, CH₂OPy), 3.37 and 2.94 (in each case dd, 1H, diastereotopes N═CCH₂ (isoxazoline)), 2.88 (m, 1H, OCH), 1.9 (m, 4H, PyOCH₂CH₂CH₂), 1.34 (d, 6H, CH₃).

### Example (I-78)

(R/S)-3-(2-cyclopropylcarbonyloxy-3-chloro-5-(1,1-dichloro-1-propen-3-oxy)-phenyl)-5-((5-trifluoromethyl-pyridin-2-yl)-3-(propyl)ether-1-yl)-2-isoxazoline: ¹H-NMR: CDCl₃, δ=8.43 (1H, Py), 7.77 (dd, 1H, Py), 6.80 (d, 1H, Py), 7.03 and 7.08 (in each case d, 1H, PhH), 6.14 (t, 1H, CHCCl₂), 4.65 (d, 2H, CH₂CHCCl₂), 4.8 (m, 1H, CHO (isoxazoline)), 4.42 (m, 2H, CH₂OPy), 3.39 and 2.98 (in each case dd, 1H, diastereotopes N═CCH₂ (isoxazoline)), 1.75-2.05 (m, 5H, COCH and PyOCH₂CH₂CH₂), 1.23 (m, 2H, cyPr), 1.07 (m, 2H, cyPr).

### Example (I-79)

(R/S)-3-(2-(3-methyl)-propylcarbonyloxy-3-chloro-5-(1,1-dichloro-1-propen-3-oxy)-phenyl)-5-((5-trifluoromethyl-pyridin-2-yl)-3-(propyl)ether-1-yl)-Δ²-isoxazoline: ¹H-NMR: CDCl₃, δ=8.43 (1H, Py), 7.77 (dd, 1H, Py), 6.80 (d, 1H, Py), 6.98 and 7.04 (in each case d, 1H, PhH), 6.14 (t, 1H, CHCCl₂), 4.65 (d, 2H, CH₂CHCCl₂), 4.8 (m, 1H, CHO (isoxazoline)), 4.42 (m, 2H, CH₂OPy), 3.39 and 2.96 (in each case dd, 1H, diastereotopes N═CCH₂ (isoxazoline)), 2.52 (d, 2H, COCH₂), 2.25 (m, 1H, CH(CH₃)₂), 1.9 (m, 4H, PyOCH₂CH₂CH₂), 1.06 (d, 6H, CH₃).

### Example (I-82)

### (R/S)-3-(2-ethylcarbamoyloxy-3-chloro-5-(1,1-dichloro-1-propen-3-oxy)-phenyl)-5-((5-trifluoromethyl-pyridin-2-yl)-3-(propyl)ether-1-yl)-Δ²-isoxazoline

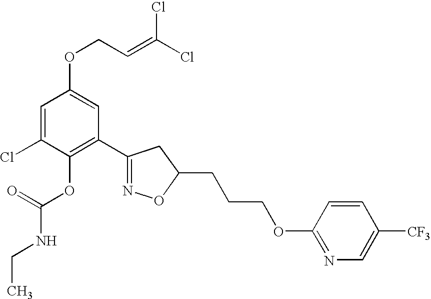

60 mg of 2-chloro-4-(3,3-dichloro-allyloxy)-6-{5-[3-(5-trifluoromethyl-pyridin-2-yloxy)-propyl]-4,5-dihydro-isoxazol-3-yl}-phenol are dissolved in 5 ml of THF. 15 mg (1.3 equivalent) of triethylamine and 9 mg (1.1 equivalent) of ethyl isocyanate are added, and the mixture is stirred overnight at room temperature. The same amounts of triethylamine and ethyl isocyanate are added once again and stirred for two days at room temperature. The reaction mixture is distributed between water and acetic ether. After the concentration of the organic phase to dryness, the remaining residue is chromatographed over silica gel.

One obtains 10 mg (purity 97%, 14% of the theory) as well as 10 mg (purity 63%, 9% of the theory) of (R/S)-3-(2-ethylcarbamoyloxy-3-chloro-5-(1,1-dichloro-1-propen-3-oxy)-phenyl)-5-((5-trifluoromethyl-pyridin-2-yl)-3-(propyl)ether-1-yl)-Δ²-isoxazoline. MS (ES+): 596.

### Example (I-83)

### (R/S)-3-(2-fluoro-5-(1,1-dichloro-1-propen-3-oxy)-phenyl)-5-((5-trifluoromethyl-pyridin-2-yl)-3-(propyl)ether-1-yl)-Δ²-isoxazoline

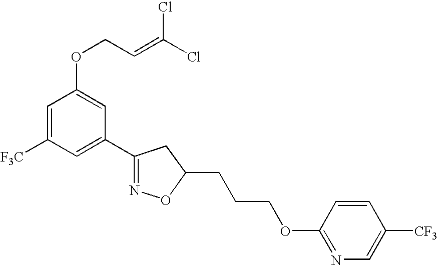

### a) Production of (R/S)-3-(3-fluoro-5-benzyloxy-phenyl)-5-((5-trifluoromethyl-pyridin-2-yl)-3-(propyl)ether-1-yl)-Δ²-isoxazoline

The production takes place analogously to the instructions according to Example (I-1), whereby it is stirred after the addition of NCS for 18 hours at room temperature and after the addition of triethylamine for 18 hours at 80° C., with 370 mg (1.25 mMol) of 3-benzyloxy-5-trifluoromethylbenzaldehyde oxime, 579 mg (2.51 mMol) of 2-pent-4-enyloxy-5-trifluoromethylpyridine, 184 g (1.38 mMol) of NCS, 101 mg (1.38 mMol) of triethylamine, and 15 ml of DMF. After the residue is chromatographed over silica gel, one obtains 220 mg (purity 75%, 25% of the theory) of (R/S)-3-(3-fluoro-5-benzyloxy-phenyl)-5-((5-trifluoromethyl-pyridin-2-yl)-3-(propyl)ether-1-yl)-Δ²-isoxazoline. MS (ES+): 525.

### b) Production of (R/S)-3-(3-fluoro-5-hydroxy-phenyl)-5-((5-trifluoromethyl-pyridin-2-yl)-3-(propyl)ether-1-yl)-Δ²-isoxazoline

220 mg (0.42 mMol) of (R/S)-3-(3-fluoro-5-benzyloxy-phenyl)-5-((5-trifluoromethyl-pyridin-2-yl)-3-propyl)ether-1-yl)-Δ²-isoxazoline are dissolved in 20 ml of ethanol and hydrogenated with 50 mg of palladium on carbon (concentration 10%) and hydrogen over one hour. It is filtered and concentrated to dryness.

One obtains 180 mg (80% purity, 79% of the theory) of (R/S)-3-(3-fluoro-5-hydroxy-phenyl)-5-((5-trifluoromethyl-pyridin-2-yl)-3-(propyl)ether-1-yl)-Δ²-isoxazoline. MS (ES+): 435.

### c) Production of (R/S)-3-(3-fluoro-5-(1,1-dichloro-1-propen-3-oxy)-phenyl)-5-((5-trifluoromethyl-pyridin-2-yl)-3-(propyl)ether-1-yl)-Δ²-isoxazoline

Under a nitrogen atmosphere, 11 mg (0.45 mMol) of sodium hydride is stirred in 15 ml of DMF and added drop by drop into 180 mg (0.41 mMol) of (R/S)-3-(3-fluoro-5-hydroxy-phenyl)-5-((5-trifluoromethyl-pyridin-2-yl)-3-(propyl)ether-1-yl)-Δ²-isoxazoline dissolved in 2 ml DMF. After 20 minutes, 86 mg (0.45 mMol) of dichloropropenyl bromide are added and stirred for 18 hours at room temperature. The reaction mixture is distributed between water and dichloromethane. The organic phase is concentrated to dryness.

One obtains 150 mg (purity 87%, 57% of the theory) of (R/S)-3-(3-fluoro-3-5-(1,1-dichloro-1-propen-3-oxy)-phenyl)-5-((5-trifluoromethyl-pyridin-2-yl)-3-(propyl)ether-1 yl)-Δ²-isoxazoline. MS (ES+): 543. ¹H-NMR: CDCl₃, δ=8.43 (1H, Py), 7.77 (dd, 1H, Py), 6.80 (d, 1H, Py), 7.44(s, 2H, PhH), 7.17 (s, 1H, PhH), 6.16 (t, 1H, CHCCl₂), 4.72 (d, 2H, CH₂CHCCl₂), 4.88 (m, 1H, CHO (isoxazoline)), 4.42 (m, 2H, CH₂OPy), 3.44 and 3.02 (in each case dd, 1H, diastereotopes N═CCH₂ (isoxazoline)), 1.95 (m, 4H, PyOCH₂CH₂CH₂).

### Example (I-85)

(R/S)-3-(3-chloro-5-(1,1-dichloro-1-propen-3-oxy)-phenyl)-5-((3-chloro-5-trifluoromethyl-pyridin-2-yl)-3-(propyl)ether-1-yl)-Δ²-isoxazoline: MS (ES+): 589. ¹H-NMR: CDCl₃, δ=8.32 (1H, Py), 7.84 (d, 1H, Py), 6.94, 7.16 and 7.22 (in each case 1H, PhH), 6.15 (t, 1H, CHCCl₂), 4.66 (d, 2H, CH₂CHCCl₂), 4.87 (m, 1H, CHO (isoxazoline)), 4.51 (m, 2H, CH₂OPy), 3.41 and 2.96 (in each case dd, 1H, diastereotopes N═CCH₂ (isoxazoline)), 1.95 (m, 4H, PyOCH₂CH₂CH₂).

### Example (I-87)

### (R/S)-3-(2-methoxy-3-chloro-5-(1,1-dichloro-1-propen-3-oxy)-phenyl)-5-((5-trifluoromethylpyridin-2-yl)-2-(ethyl)ether-1-yl)-5-methyl-Δ²-isoxazoline

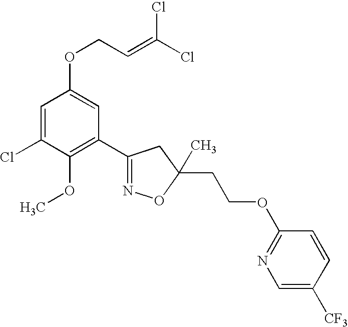

The implementation takes place analogously to Example (I-83a) using approximately 10 equivalents of 2-(3-methyl-but-3-enyloxy)-5-trifluoromethylpyridine. One obtains the product in a yield of 19% MS (ES+): 539.

### Example (I-90)

### (R/S)-3-(2-methoxy-3-chloro-5-(1,1-dichloro-1-propen-3-oxy)-phenyl)-5-((3-chloro-trifluoromethyl-pyridin-2-yl)-2-(ethyl)ether-1-yl)-5-methyl-Δ²-isoxazoline

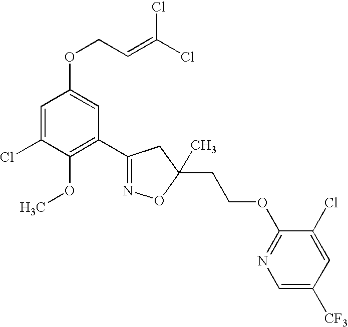

Under nitrogen as a protective gas, 11 mg (0.41 mMol) of sodium hydride are placed in 3 ml of THF, and a solution of 150 mg (0.38 mMol) of (R/S)-3-(2-methoxy-3-chlor-5-(1,1-dichloro-1-propen-3-oxy)-phenyl)-5-hydroxyethyl-5-methyl-Δ²-isoxazoline in 2 ml of THF is added drop by drop while stirring. One stirs for 20 minutes at room temperature, adds 89 mg (0.41 mMol) of 2,3-dichloro-5-trifluoromethylpyridine drop by drop, and allows it to stir for 18 hours. One adds another 11 mg (0.41 mMol) of sodium hydride and stirs for 18 hours. One places the reaction mixture with 50 ml of water, extracts twice with 50 ml of dichloromethane in each case, washes the organic phase once with water and concentrates it to dryness. The residue is purified by means of column chromatography on silica gel.

One obtains 150 mg (purity 95%, 65% of the theory) of (R/S)-3-(2-methoxy-3-chloro-5-(1,1-dichloro-1-propen-3-oxy)-phenyl)-5-((3-chloro-5-trifluoromethyl-pyridin-2-yl)-2-(ethyl)ether-1-yl)-5-methyl-Δ²-isoxazoline. MS (ES+): 573. ¹H-NMR: CDCl₃, δ=8.31 (d, 1H, Py), 7.79 (d, 1H, Py), 7.07 (d, 1H, PhH), 6.98 (d, 1H, PhH), 6.11 (t, 1H, CHCCl₂), 4.58 (d, 2H, CH₂CHCCl₂), 4.63 (m, 2H, CH₂OPy), 3.78 (s, 3H, OCH₃), 3.52 and 3.24 (in each case d, 1H, diastereotopes N═CCH₂ (isoxazoline)), 2.29 (t, 2H, PyOCH₂CH₂), 1.57 (s, 3H, CCH₃).

### Example (I-125)

### 3-[5-(3,3-dichloro-allyloxy)-2-methoxy-phenyl]-5-[3-(2,4-dichloro-phenoxy)-propyl]-[1,2,4]oxadiazole

### a) 5-(3,3-dichloro-allyloxy)-N-hydroxy-2-methoxy-benzamidine

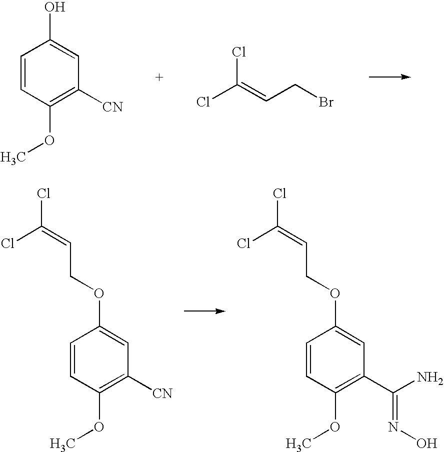

1 g (6.7 mMol) of 5-hydroxy-2-methoxybenzonitrile (Journal of Organic Chemistry (1999), 64(26), 9719-9721), 2.8 g (8.7 mMol) cesium carbonate and 1.27 g (6.7 mMol) of 3-bromo-1,1-dichloropropene are stirred overnight in 20 ml of DMF at 80° C. After filtration, mixed with water and extracted three times with dichloromethane. 585 mg (62% according to LC-MS, 19% of the theory) of 5-(3,3-dichloro-allyoxy)-2-methoxybenzonitrile are obtained. Preparation of the amidoxime: 500 mg (62%, 1.2 mMol) of 5-(3,3-dichloro-allyloxy)-2-methoxybenzonitrile are stirred overnight under reflux together with 535 mg (3.8 mMol) of potassium carbonate and 1.2 g (3.8 mMol) hydroxylamine hydrochloride in 5 ml of ethanol. All is concentrated to dryness, and the residue is absorbed in ethyl acetate and washed with water. According to LC-MS, 5-(3,3-dichloro-allyoxy)-N-hydroxy-2-methoxy-benzamidine is to accrue to 25% initially. Therefore, according to Variant A the mixture is once again caused to react with 93 mg (2.3 mMol) of NaOH and 161 mg (2.3 mMol) of hydroxylamine hydrochloride in 10 ml of ethanol. The solvents are subsequently removed with a rotary evaporator to dryness, and the residue is mixed with water and stirred at room temperature for 10 min. The pH value is subsequently brought to 8 with concentrated ammonia solution, and the precipitated product is isolated. 370 mg (48% according to LC-MS, 51% of the theory) of the 5-(3,3-dichloro-allyoxy)-N-hydroxy-2-methoxy-benzamidine are obtained. MS(ES+)=291.

### b) 3-[5-(3,3-dichloro-allyloxy)-2-methoxy-phenyl]-5-[3-(2,4-dichloro-phenoxy)-propyl]-[1,2,4]oxadiazole

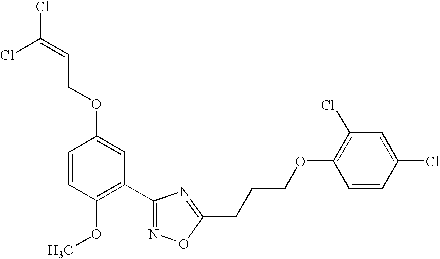

In order to produce 4-(2,4-dichloro-phenoxy)butyric acid chloride, 141 mg (0.56 mMol) of 4-(2,4-dichloro-phenoxy)-butyric acid are mixed with 72 mg (0.56 mMol) of oxalyl chloride and a drop of DMF under nitrogen in 5 ml of absolute dichloromethane (DCM) and stirred at room temperature after complete gas development 15. It is then concentrated to dryness. In a second flask, 150 mg (48%, 0.25 mMol) of 5-(3,3-dichloro-allyloxy)-N-hydroxy-2-methoxy-benzamidine under nitrogen is dissolved in 1 ml of anhydrous pyridine. The 4-(2,4-dichloro-phenoxy)butyric acid chloride previously produced is added and stirred at 90° C. for approximately 24 hours under light nitrogen flow in the open flask. After cooling, water is added and brought to pH<7 with diluted HCl. It is extracted several times with ethyl acetate, the unified organic phases are dried over Na₂SO₄, filtered and concentrated to dryness.

After chromatography over silica gel (dichlormethane:methanol 98:2) and subsequent HPLC separation, 26 mg (21% of the theory) of the 3-[5-(3,3-dichloro-allyloxy)-2-methoxy-phenyl]-5-[3-(2,4-dichloro-phenoxy)-propyl]-[1,2,4]oxadiazole are obtained. MS(ES+)=503. ¹H-NMR: CDCl₃, δ 2.4 (p, 2H, OCH₂CH₂CH₂); 3.2 (t, 2H, OCH₂CH₂CH₂); 3.9 (s, 3H, OCH₃); 4.2 (t, 2H, OCH₂CH₂CH₂); 4.7 (d, CH₂—CH—C═CCl₂); 6.2 (t, 1H, CH₂—CH—C═CCl₂); 6.8 (d, 1H, aryl); 7.0 (m, 2H, aryl); 7.3 (dd, 1H, aryl); 7.4 (dd, 1H, Aryl); 7.5 (dd, 1H, aryl).

### Example (I-126)

### 3-[5-(3,3-dichloro-allyloxy)-2-methoxy-phenyl]-5-[3-(2,4,6-triiodo-phenoxy)-propyl]-[1,2,4]-oxadiazole

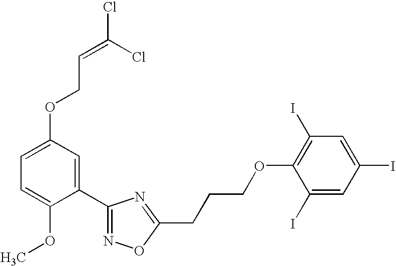

The production takes place analogously to the method described for Example (I-125). MS(ES+)=813.

### Example (I-127)

### 2-(3-{3-[5-(3,3-dichloro-allyloxy)-2-methoxy-phenyl]-[1,2,4]oxadiazol-5-yl}-propoxy)-5-trifluoromethyl-pyridine

### a) 4-(5-trifluoromethyl-pyridin-2-yloxy)-butyric acid ethyl ester

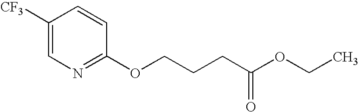

6.57 g (33.7 mMol) of 2-hydroxy-4-trifluoropyridine are added to a 0° C. cold suspension of 0.8 g (33.7 mMol) of NaH in 100 ml of DMF under nitrogen, and stirred 15 minutes at room temperature. It is cooled once again to 0° C. and added drop by drop to 5 g (30.6 mMol) of ethyl-4-bromobutyrate over a period of 15 minutes. It is then stirred for 12 hours at room temperature. It is concentrated to dryness, and the residue is extracted with DCM. The organic phase is washed with water, dried over Na₂SO₄, filtered and concentrated. After chromatography over silica gel (gradient hexane:ethyl acetate 4:1 until ethyl acetate 100%), 1.8 g (20% of the theory) of the 4-(5-trifluoromethyl-pyridin-2-yloxy)-butyric acid ethyl ester is obtained in addition to 7.0 g (80% of the theory) of the 4-(2-oxo-5-trifluoromethyl-2H-pyridin-1-yl)-butyric acid ethyl ester. MS(ES+)=278. ¹H-NMR: CDCl₃, δ 1.3 (t, 3H, CH₃); 2.1 (p, 2H, OCH₂CH₂CH₂CO₂Et); 2.5 (t, 2H, OCH₂CH₂CH₂CO₂Et); 4.15 (q, 2H, OCH₂CH₃); 4.4 (t, OCH₂CH₂CH₂CO₂Et); 6.8 (d, 1H, Py); 7.75 (dd, 1H, Py); 8.4 (s, 1H, Py).

### b) 2-(3-{3-[5-(3,3-dichloro-allyloxy)-2-methoxy-phenyl]-[1,2,4]oxadiazol-5-yl}-propoxy)-5-trifluoromethyl-pyridine

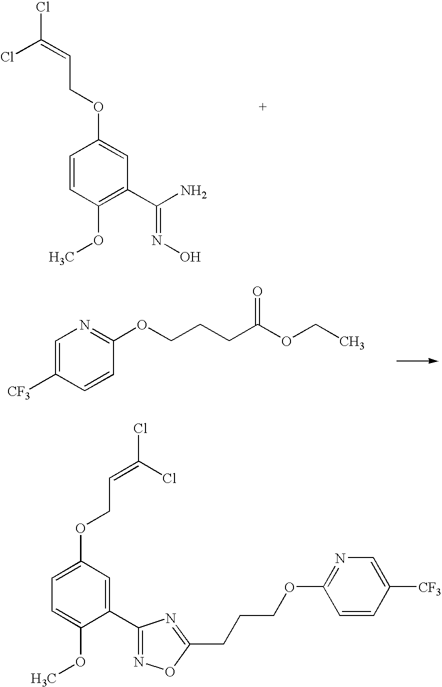

100 mg (48%, 0.16 mMol) 5-(3,3-dichloro-allyloxy)-N-hydroxy-2-methoxy-benzamidine are placed together with molsieb in 5 ml of anhydrous THF and mixed with 9 mg (0.27 mMol) of NaH. After gas development is completed, it is stirred for 20 minutes at room temperature. 190 mg (0.68 mMol) of 4-(5-trifluoromethyl-pyridin-2-yloxy)butyric acid ethyl ester are added and stirred 1 hour under reflux. After filtration, it is concentrated to dryness and chromatographed over silica gel (dichloromethane:methanol 95:5). In addition to 12.4 mg (5% of the theory) of the 2-(3-{3-[5-(3,3-dichloro-allyloxy)-2-methoxy-phenyl]-[1,2,4]oxadiazol-5-yl}-propoxy)-5-trifluoromethylpyridine, 64 mg of the 4-(5-trifluoromethyl-pyridin-2-yloxy) butyric acid ethyl ester are recovered. MS(ES+)=504 (94% according to LC-MS). log P (neutral): 4.93.

### Example (I-128)

### 2-(3-{5-[3-chloro-5-(3,3-dichloro-allyloxy)-2-methoxy-phenyl]-[1,2,4]oxadiazol-3-yl}-propoxy)-5-trifluoromethyl-pyridine

### a) 4-(5-trifluoromethyl-pyridin-2-yloxy)-butyronitrile

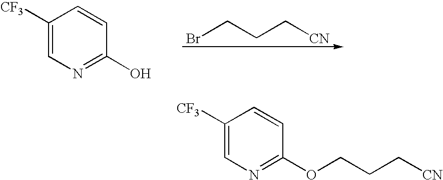

5 g (31 mMol) of 2-hydroxy-4-trifluoropyridine are added to 0° C. cold suspension of 0.77 g (32 mMol) of NaH in 100 ml of dimethylformamide (DMF) under nitrogen stirred for 15 minutes at room temperature. It is once again cooled to 0° C. and mixed with 4-bromo-butanitrile drop by drop within 15 minutes. One allows it to stir for 12 hours at room temperature. After concentration, the residue is absorbed in DCM, washed with water, dried over Na₂SO₄, filtered and concentrated. According to LC-MS, a 1:1.8 mixture of 4-(5-trifluoromethyl-pyridin-2-yloxy)-butyronitrile and 4-(2-oxo-5-trifluoromethyl-2H-pyridin-1-yl)-butyronitrile results. After chromatography over silica gel (dichloromethane:methanol 98:2), one obtains 0.9 g (13% of the theory) of the 4-(5-trifluoromethyl-pyridin-2-uloxy)-butyronitrile. ¹H-NMR: CDCl₃, δ=2.2 (m, 2H, OCH₂CH₂CH₂CN); 2.5 (t, 2H, OCH₂CH₂CH₂CN); 4.5 (t, 2H, OCH₂CH₂CH₂CN); 6.8 (d, 1H, Py); 7.8 (dd, 1H, Py); 8.4 (s, 1H, Py).

### b) N-hydroxy-4-(5-trifluoromethyl-pyridin-2-yloxy)-butyramidine

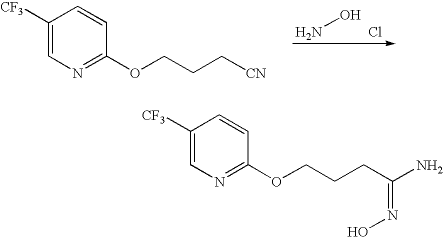

83 mg (2 mMol) of NaOH in 1 ml of water are added to a solution of 145 mg (2 mMol) of hydroxylamine hydrochloride in 10 ml of ethanol (95%). Subsequently, 400 mg (1.7 mMol) of 4-(5-trifluoromethyl-pyridin-2-yloxy)-butyronitrile is added as a solution in 5 ml of ethanol, and everything is stirred overnight under reflux. Addition once again of 72 mg (1 mMol) of hydroxylamine hydrochloride and 41 mg (1 mMol) of sodium hydroxide in 0.5 ml of water. One allows it to stir an additional 3 hours under reflux. Ethanol is removed in the rotary evaporator at 60° C., and approximately 10 ml of water is added to the residue and stirred for 10 minutes at room temperature. by adding concentrated ammonia solution (25% in water), the solution is brought to pH=8, the precipitated product is isolated and subsequently recrystallised from 2 ml of toluene. 160 mg of the N-hydroxy-4-(5-trifluoromethyl-pyridin-2-yloxy)-butyramidine (35% of the theory) are obtained. MS(ES+)=264 (purity: 100% according to LC-MS). ¹H-NMR: CDCl₃, δ=2.1 (m, 2H, OCH₂CH CH₂C═N(OH)NH₂); 2.3 (t, 2H, OCH₂CH CH—C═N(OH)NH₂); 4.4 (t, 2H, OCH₂CH₂CH₂C═N(OH)NH₂); 4.6 (bs, 2H, NH₂); 6.8 (d, 1H, Py); 7.5 (bs, 1H, OH); 7.8 (dd, 1H, Py); 8.4 (s, 1H, Py).

### c) 2-(3-{5-[3-chloro-5-(3,3-dichloro-allyloxy)-2-methoxy-phenyl]-[1,2,4]oxadiazol-3-yl}-propoxy)-5-trifluoromethyl-pyridine

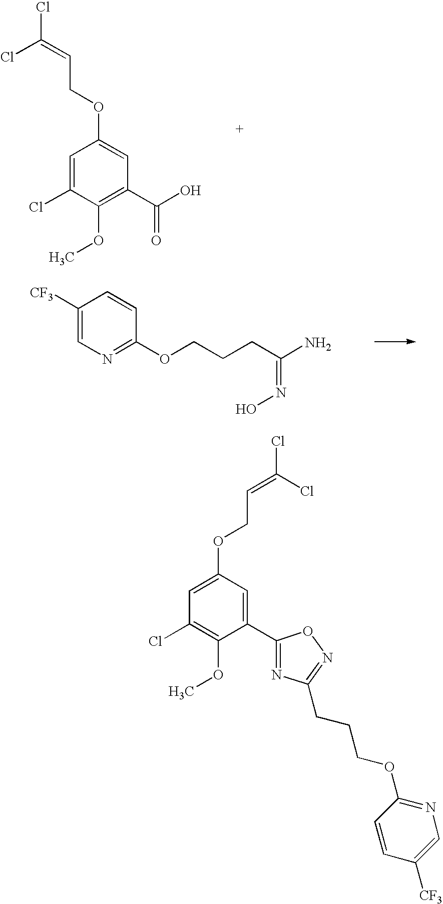

101 mg of (0.32 mMol) 3-chloro-5-(3,3-dichloro-allyloxy)-2-methoxy benzoic acid are dissolved in 5 ml of absolute dichloromethane (DCM) under nitrogen and mixed with 43 mg (0.34 mMol) of oxalyl chloride and a drop of DMF. After gas development is completed, it is stirred for 15 minutes at room temperature and subsequently concentrated to dryness. In a second flask, 103 mg (0.38 mMol) of N-hydroxy-4-(5-5-trifluoromethyl-pyridin-2-yloxy)-butyramidine is dissolved in 1 ml of anhydrous pyridine under nitrogen. The acid chloride is added and stirred for approximately 24 hours at 90° C. in the open flask under light nitrogen flow. One allows it to cool, mixes with approximately 10 ml of water, brings it to a pH<6 with diluted HCl and extracts it several times with ethyl acetate. United organic phases are dried over Na₂SO₄, filtered and concentrated. The raw product is chromatographed over silica gel (hexan:ascetic ether 4:1). One obtains 111 mg (63% of the theory) of the 2-(3-{5-[3-chloro-5-(3,3-dichloro-allyloxy)-2-methoxy-phenyl]-[1,2,4]-oxadiazol-3-yl}-propoxy)-5-trifluoromethyl-pyridine as a colorless solid substance. MS(ES+)=539 (purity: 100% according to LC-MS)

log P: 5.99. ¹H-NMR: CDCl₃, δ=2.3 (m, 2H, CH₂—CH₂—CH₂—O-Py); 3.0 (t, 2H, CH₂—CH₂—CH₂—O-Py); 3.93 (s, 3H, OCH₃); 4.5 (t, 2H, CH₂—CH₂-CH₂—O-Py); 4.6 (d, 2H, J=6.2 Hz, CH₂—CH—C═CCl₂); 6.1 (t, 1H, CH₂—CH—C═CCl₂); 6.8 (d, 1H, Py); 7.2 (dd, 1H, aryl); 7.4 (dd, 1H, aryl); 7.7 (dd, 1H, Py); 8.42 (s, 1H, Py).

### Example (I-129)

### 2-(4-{5-[3-chloro-5-(3,3-dichloro-allyl)-2-methoxy-phenyl]-[1,2,4]oxadiazol-3-yl}-butoxy)-5-trifluoromethyl-pyridine

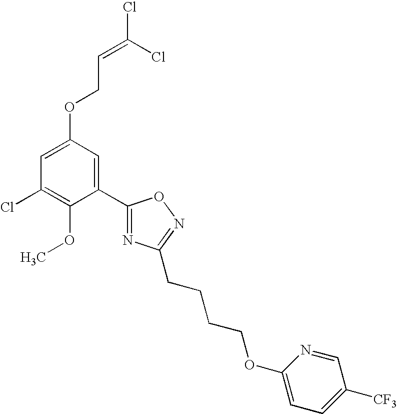

MS(ES+)=552. ¹H-NMR: CDCl₃, δ 1.9-2.1 (m, 4H); 2.9 (t, 2H); 3.9 (s, 3H, OCH₃); 4.4 (t, 2H, PyOCH₂); 4.7 (d, 2H, CH₂—CH—C═CCl₂); 6.1 (t, 1H, CH₂—CH—C═CCl₂); 6.8 (d, 1H, Py); 7.2 (dd, 1H, aryl); 7.4 (dd, 1H, aryl); 7.8 (dd, 1H, Py); 8.4 (s, 1H, Py).

### Example (I-130)

### 3-chloro-2-(4-{5-[3-chloro-5-(3,3-dichloro-allyl)-2-methoxy-phenyl]-[1,2,4]oxadiazol-3-yl}-butoxy)-5-trifluoromethyl-pyridine

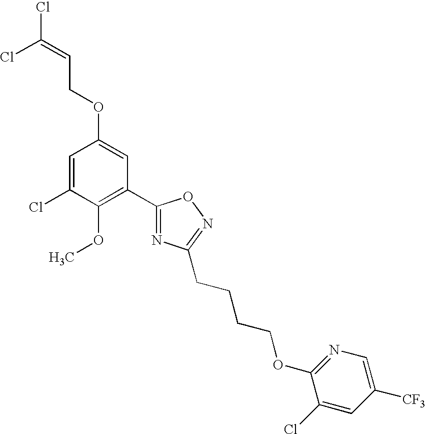

MS(ES+)=586. ¹H-NMR: CDCl₃, δ 1.8-2.1 (m, 4H); 2.9 (t, 2H); 3.9 (s, 3H, OCH₃); 4.5 (t, 2H, PyOCH₂); 4.7 (d, 2H, CH₂—CH—C═CCl₂); 6.1 (t, 1H, CH₂—CH—C═CCl₂); 7.2 (dd, 1H, aryl); 7.5 (dd, 1H, Aryl); 7.8 (dd, 1H, Py); 8.3 (s, 1H, Py).

### Example (I-131)

### 2-(2-{5-[3-chloro-5-(3,3-dichloro-allyloxy)-2-methoxy-phenyl]-[1,2,4]oxadiazol-3-yl}-ethoxy)-5-trifluoromethyl-pyridine

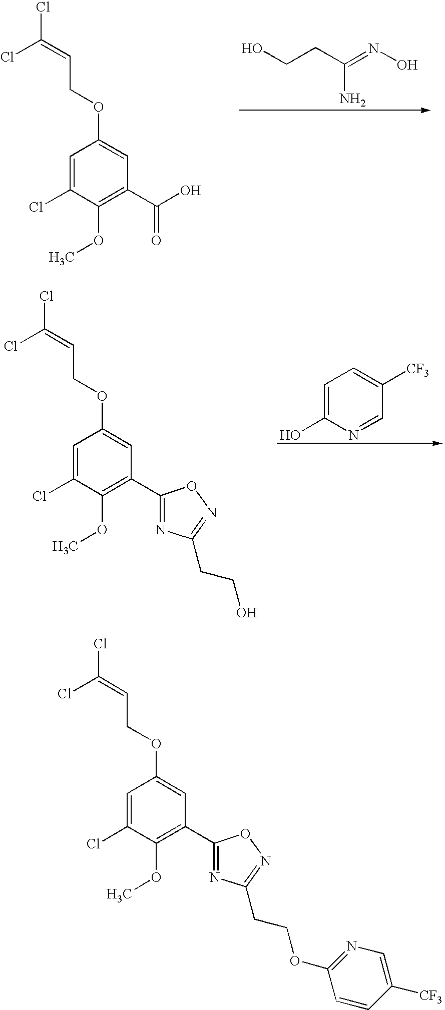

134 mg (0.41 mMol) of O-(7-azabenzotriazol-1-yl)-N,N,N,N-tetramethyluronium PF6 (HATU), 13 mg (0.09 mMol) of 1-hydroxy-1H-benzotriazol hydrate (HOBT) and 82 mg (0.64 mMol) of N,N-diisopropylethylamine (DIPEA) are added to 100 mg (0.32 mMol) of 3-chloro-5-(3,3-dichloro-allyloxy)-2-methoxy-benzoic acid in 5 ml of DMF. One allows it to stir for 15 minutes. 100 mg (0.96 mMol) of 3 N-dihydroxypropionamidine is subsequently added as stirred overnight (at room temperature. Approximately 10 ml of water is added and extracted several times with dichlormethane (DCM), dried over Na₂SO₄, filtered and concentrated to dryness. For dehydration, the residue is absorbed in 5 ml of DMF and heated for 6 hours to 110° C. under a light nitrogen flow. One allows to cool off, dilutes with DCM, washes with water, dries over Na₂SO₄, and concentrates to dryness. Because the product had initially developed at 10% according to LC-MS, the raw product thus obtained was caused to react under the same reaction conditions. 120 mg (purity 21% according to LC-MS) of the 2-{5-[3-chloro-5-(3,3-dichloro-allyloxy)-2-methoxy-phenyl]-[1,2,4]oxadiazol-3-yl}-ethanol is obtained (21% of the theory). 120 mg (21%, 0.06 mMol) of 2-{5-[3-chloro-5-(3,3-dichloro-allyloxy)-2-methoxy-phenyl]-[1,2,4]oxadiazol-3-yl}-ethanol, 57 mg (0.3 mMol) of 2-hydroxy-5-trifluoromethylpyridine and 124 mg (0.4 mMol) of triphenylphosphine are dissolved in 5 ml of absolute THF under argon. 83 mg (0.4 mMol) of diethyl azodicarboxylate (DEAD) are added drop by drop as a solution into 1 ml of THF, and the preparation is stirred overnight at room temperature. After concentration, the raw product is purified by means of preparative HPLC. One obtains 4.6 mg (3% of the theory) of 2-(2-{5-[3-chloro-5-(3,3-dichloro-allyloxy)-2-methoxy-phenyl]-[1,2,4]oxadiazol-3-yl}-ethoxy)-5-trifluoromethyl-pyridine in addition to 7.5 mg of a slightly contaminated fraction of the product. MS(ES+)=525. ¹H-NMR: DMSO, δ=3.3 (m, 2H, CH₂—CH₂—O-Py); 3.8 (s, 3H, OCH₃); 4.8 (m, 4H, CH₂—CH₂-0-Py and CH₂—CH—C═CCl₂); 6.5 (t, 1H, J=6.5 Hz, CH₂—CH—C═CCl₂); 7.0 (d, 1H, Py); 7.4 (dd, 1H, J=3.1 Hz, aryl); 7.5 (dd, 1H, J=3.1 Hz, aryl); 8.1 (dd, 1H, Py); 8.6 (s, 1H, Py).

### Example (I-132)

### 2-{2-[3-chloro-5-(3,3-dichloro-allyloxy)-2-methoxy-phenyl]-4,5-dihydro-oxazol-4-ylmethoxy}-5-trifluoromethyl-pyridine

### a) {2-[3-chloro-5-(3,3-dichloro-allyloxy)-2-methoxy-phenyl]-4,5-dihydro-oxazol-4-yl}-methanol

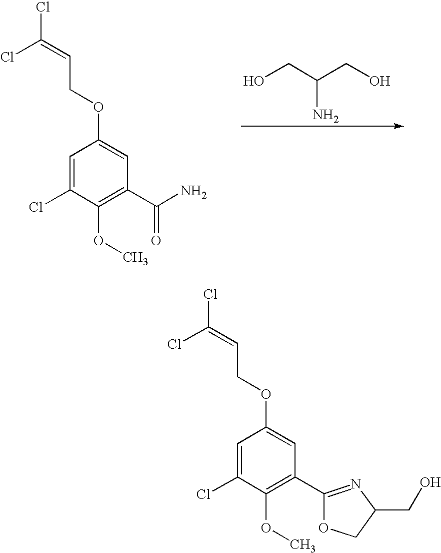

200 mg of 3-chloro-5-(3,3-dichloro-allyloxy)-2-methoxy-benzamide is dissolved in 5 ml of absolute 1,2-ethane dichloride under nitrogen and mixed with 141 mg of triethyl-oxoniumtetrafluoroborate. It is stirred overnight at room temperature, whereby the solid substance slowly goes into solution. 2-aminopropane-1,3-diol is added drop by drop as a solution into 2 ml of dichlorethane, and one allows it to be stirred an additional 48 hours at room temperature. Addition of 10 ml of saturated NaHCO₃ solution. The aqueous phase is extracted several times with dichlormethane (DCM), dried over Na₂SO₄, filtered and concentrated to dryness. The raw product is chromatographed over silica gel (dichloromethane:methanol 95:5). One obtains 103 mg (77% according to LC-MS, 33% of the theory) of the {2-[3-chloro-5-(3,3-dichloro-allyloxy)-2-methoxy-phenyl]-4,5-dihydro-oxazol-4-yl}-methanol. MS(ES+)=366. log P(pH=2.3): 2.05.

### b) 2-{2-[3-chloro-5-(3,3-dichloro-allyloxy)-2-methoxy-phenyl]-4,5-dihydro-oxazol-4-ylmethoxy}-5-trifluoromethyl-pyridine

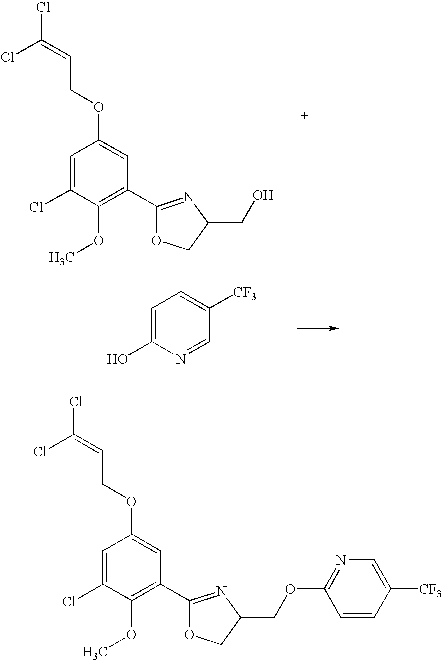

45 mg (0.12 mMol) {2-[3-chloro-5-(3,3-dichloro-allyloxy)-2-methoxy-phenyl]-4,5-dihydro-oxazol-4-yl}-methanol and 22 mg (0.13 mMol) of 2-hydroxy-5-trifluoromethylpyridine is dissolved in 5 ml of absolute DCM under nitrogen together with 38 mg (0.14 mMol) of triphenylphosphine. Diethyl azodicarboxylate (DEAD) is subsequently added drop by drop into 1 ml of absolute DCM. The preparation is stirred overnight at room temperature. After concentration to dryness, the remaining residue is purified by means of preparative HPLC. One obtains 14.5 mg (22% of the theory) of the 2-{2-[3-chloro-5-(3,3-dichloro-allyloxy)-2-methoxy-phenyl]4,5-dihydro-oxazol-4-ylmethoxy}-5-trifluoromethyl-pyridine in addition to 12.2 mg (19% of the theory) of the 1-{2-[3-chloro-5-(3,3-dichloro-allyloxy)-2-methoxy-phenyl]-4,5-dihydro-oxazol-4-ylmethyl}-5-trifluoromethyl-1H-pyridin-2-one. MS(ES+)=510 (purity: 100% according to LC-MS). ¹H-NMR: CDCl₃, δ=3.9 (s, 3H, OCH₃); 4.4 (t, 1H, CH₂-oxzolin); 4.5 (dd, 1H, CH₂OPy); 4.6 (t, 1H, CH₂-oxzolin); 4.66 (m, 3H, CH₂OPy and CH₂—CH—C═CCl₂); 4.7 (m, 1H, CH); 6.1 (t, 1H, CH₂—CH—C═CCl₂); 6.8 (d, 1H, Py); 7.1 (d, 1H, aryl); 7.2 (d, 1H, aryl); 7.8 (dd, 1H, Py); 8.4 (s, 1H, Py).

### Example (I-137)

### 2-(2-{4-[3-chloro-5-(3,3-dichloro-allyloxy)-2-methoxy-phenyl]-thiazol-2-yl}-ethoxy)-5-trifluoromethyl-pyridine

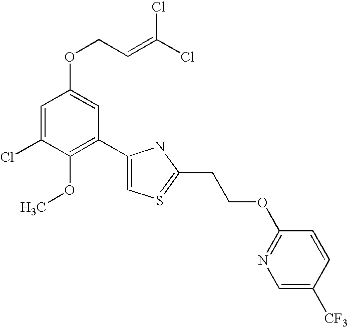

### a) 1-(3-chloro-2,5-dihydroxy-phenyl)-ethanone

The chlorination of 2,5-dihydroxyphenyl-ethanone takes place analogously to the instructions for Example (II-1a) with 15.0 g (89.6 mMol) of 2,5-dihydroxyphenyl-ethanone, 17.1 g (128 mMol) of NCS, and 150 ml of DMF.

One obtains 7.7 g (purity 81%, 33% of the theory) 1-(3-chloro-2,5-dihydroxy-phenyl)-ethanone. MS (ES+): 187.

### b) 1-(3-chloro-2-hydroxy-5-triisopropylsilyloxy-phenyl)-ethanone

The silylation takes place analogously to the instructions for Example (II-5a) with 7.6 g (40.7 mMol) of 1-(3-chloro-2,5-dihydroxy-phenyl)-ethanone, 9.4 g (48.9 mMol) triisopropylsilyl chloride, 5.35 g (52.9 mMol) of triethylamine, and 150 ml of dichlormethane.

One obtains 14.4 g (purity 84%, 86% of the theory) of 1-(3-chloro-2-hydroxy-5-triisopropylsilyoxy-phenyl)-ethanone. MS (ES+): 343.

### c) 1-(3-chloro-2-methoxy-5-triisopropylsilyloxy-phenyl)-ethanone

The methylation takes place analogously to the instructions for Example (I-63) with a reaction time of 2.5 hours with 3.0 g (8.75 mMol) of 1-(3-chloro-2-hydroxy-5-triisopropylsilyloxy-phenyl)ethanone, 1.49 g (10.5 mMol) of methyl iodide, and 1.57 g (11.4 mMol) of potassium carbonate.

One obtains 2.6 g (purity 78%, 65% of the theory) of 1-(3-chloro-2-methoxy-5-triisopropylsilyloxy-phenyl)-ethanone. MS (ES+): 357.

### d) 2-bromo-1-(3-chloro-2-methoxy-5-triisopropylsilyloxy-phenyl)-ethanone

2.6 g (7.28 mMol) of 1-(3-chloro-2-methoxy-5-triisopropylsilyloxy-phenyl)-ethanone are placed in 30 ml of chloroform. 1.4 g (8.74 mMol) of bromine are added drop by drop and stirred for two hours at room temperature. The reaction mixture is distributed between aqueous sodium hydrogen carbonate solution and acetic ether. After concentration of the organic phase to dryness, one obtains 3.17 g (purity 57%, 57% of the theory) of 2-bromo-1-(3-chloro-2-methoxy-5-triisopropylsilyloxy-phenyl)-ethanone. MS (ES+): 437.

### e) [4-(3-chloro-2-methoxy-5-triisopropylsilyloxy-phenyl)-thiazol-2-yl]acetic acid ethyl ester

2.67 g (6.1 mMol) of 2-bromo-1-(3-chloro-2-methoxy-5-triisopropylsilyloxy-phenyl)-ethanone, 0.9 g (6.1 mMol) of thiocarbamoyl acetic acid ethyl ester (CAS No. 13621-50-6) and 1.54 g (18.4 mMol) of 13621-sodium hydrogen carbonate are stirred in 80 ml of ethanol for 4 hours under reflux. The reaction mixture is distributed between water and acetic ether. The organic phase is concentrated to dryness. The residue is purified by means of column chromatography over silica gel.

One obtains 1.24 g (purity 80%, 33% of the theory) of [4-(3-chloro-2-methoxy-5-triisopropylsilanyloxy-phenyl)-thiazol-2-yl]acetic acid ethyl ester. MS (ES+): 484.

### f) 4-(2-methoxy-3-chloro-5-triisopropylsilyloxy-phenyl)-2-hydroxyethyl-thiazole

The reduction takes place analogously to the instructions from Example (II-5c) with 1.42 g (2.9 mMol) of [4-(3-chloro-2-methoxy-5-triisopropylsilanyloxy-phenyl)-thiazol-2-yl]acetic acid ethyl ester, 96 mg (4.4 mMol) of lithium borohydride, and 80 ml of diethylether.

After concentration of the organic phase to dryness, one obtains 1.2 g (purity 68%, 63% of the theory) of 4-(2-methoxy-3-chlor-5-triisopropylsilyloxy-phenyl)-2-hydroxyethylthiazole. MS (ES+): 442.

### g) Production of 2-{2-[4-(3-chloro-2-methoxy-5-triisopropylsilanyloxy-phenyl)-thiazol-2-yl]-ethoxy}-5-trifluoromethyl-pyridine

The production takes place analogously to the instructions according to Example (I-4) with 300 mg (0.68 mMol) of 4-(2-methoxy-3-chloro-5-triisopropylsilyloxy-phenyl)-2-hydroxyethyl-thiazole, 111 mg (0.68 mMol) of 5-trifluoromethyl-2-pyridinol, 356 mg (1.36 mMol) of triphenylphosphane, 236 mg (1.36 mMol) of azodicarboxylic acid diethyl ester and 15 ml of THF.

After the residue was chromatographed over silica gel, one obtains 240 mg (purity 53%, 32% of the theory)₂-{2-[4-(3-chloro-2-methoxy-5-triisopropylsilanyloxy-phenyl)-thiazol-2-yl]-ethoxy}-5-trifluoromethyl-pyridine. MS (ES+): 587.

### h) Production of 3-chloro-4-methoxy-5-{2-[2-(5-trifluoromethyl-pyridin-2-yloxy)-ethyl]-thiazol-4-yl}-phenol

240 mg (purity 53%; 0.22 mMol) of 2-{2-[4-(3-chloro-2-methoxy-5-triisopropylsilanyloxy-phenyl)-thiazol-2-yl]-ethoxy}-5-trifluoromethyl-pyridine are placed in 10 ml of THF at 0° C. 0.51 ml (0.51 mMol, 1M in THF) tetra-n-butylammonium fluoride are added stirred over 18 hours at room temperature. The reaction mixture is distributed between water and acetic ether. The organic phase is concentrated to dryness. The residue is purified by means of column chromatography over silica gel.

One obtains 100 mg (purity 50%, 54% of the theory) of 3-chloro-4-methoxy-5-{2-[2-(5-trifluoromethyl-pyridin-2-yloxy)-ethyl]-thiazol-4-yl}-phenol. MS (ES+): 431.

### i) 2-(2-{4-[3-chloro-5-(3,3-dichloro-allyloxy)-2-methoxy-phenyl]-thiazol-2-yl}-ethoxy)-5-trifluoromethylpyridine

The allylation takes place analogously to the instructions according to Example (I-63) with 90 mg (purity 50%; 0.1 mMol) 3-chloro-4-methoxy-5-{2-[2-(5-trifluoromethyl-pyridin-2-yloxy)-ethyl]-thiazol-4-yl}-phenol, 48 mg (0.25 mMol) of 3-bromo-1,1-dichloropropene, 58 mg (0.42 mMol) of potassium carbonate and 15 ml of acetone. After the residue was chromatographed over silica gel, one obtains 60 mg (purity 91%, 97% of the theory) of 2-(2-{4-[3-chloro-5-(3,3-dichloro-allyloxy)-2-methoxy-phenyl]-thiazol-2-yl}-ethoxy)-5-trifluoromethylpyridine. MS (ES+): 539. ¹H-NMR: CDCl₃, δ=8.45 (1H, Py), 7.78 (dd, 1H, Py), 6.85 (d, 1H, Py), 7.91 (s, 1H, thiazole), 7.63 and 6.91 (in each case d, 1H, PhH), 6.15 (t, 1H, CHCCl₂), 4.67 (d, 2H, CH₂CHCCl₂), 4.81 (t, 2H, CH₂), 3.74 (s, 3H, OCH₃), 3.55 (t, 2H, CH₂).

### Example (I-138)

### 3-(2-(1,1,2,3,3,3-hexafluoro-propoxy)-3-chloro-5-(1,1-dichloro-1-propen-3-oxy)-phenyl)-5-((5-trifluoromethyl-pyridin-2-yl)-3-(propyl)ether 1-yl)-Δ²-isoxazoline

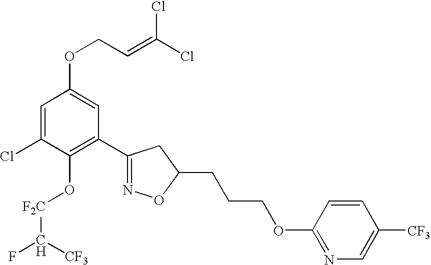

70 mg of (R/S)-3-(2-hydroxy-3-chloro-5-(1,1-dichloro-1-propen-3-oxy)-phenyl)-5-(5-trifluoromethyl-pyridin-2-yl)-3-(propyl)ether-1-yl)-Δ²-isoxazoline are dissolved in 15 ml of THF. 4 mg (0.5 equiv.) of potassium hydroxide are added, and hexafluoropropene is introduced slowly for an hour. The reaction mixture is distributed between water and acetic ether. After the concentration of the organic phase to dryness, the remaining residue is chromatographed over silica gel.

One obtains 10 mg (purity 79%, 9% of the theory) as well as 30 mg (purity 72%, 24% of the theory) of 3-(2-(1,1,2,3,3,3-hexafluoro-propoxy)-3-chloro-5-(1,1-dichloro-1-propen-3-oxy)phenyl)-5-((5-trifluormethyl-pyridin-2-yl)-3-(propyl)ether-1-yl)-Δ²-isoxazoline. MS (ES+): 675.

Starting Substances for the Formula (II):

### Example (II-1)

### 3-chloro-5-(3,3-dichloro-allyloxy)-2-methoxy-benzaldehyde oxime

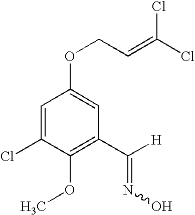

### a) Production of 3-chloro-2,5-dihydroxy-benzoic acid methyl ester

Under nitrogen as a protective gas, 6.75 g (40 mMol) of 2,5-dihydroxy benzoic acid methyl ester is dissolved in 80 mL of anhydrous DMF. While stirring, one adds a total of 6.94 g (5.2 mMol) of N-chloro-succinimide (NCS) in portions at room temperature, whereby the reaction solution slowly turns red. After complete addition, one allows it to stir overnight (at room temperature) for completion of the reaction. The process of the chlorination can be carried about by DC (flow agent n-hexane/acetic ether 1:1), in which one evaluates the reduction of the educt fleck; the preparation is only processed if scarcely any educt is to be seen in the DC (optionally use yet additional NCS). For processing, the preparation is poured into the separating funnel onto 200 mL of water and extracted with a mixture of 200 mL of heptane and 200 mL of acetic acid ethyl ester. The organic phase is washed once again with approximately 100-200 mL of water, and the solvent is subsequently removed (increase bath temperature in the rotary evaporator up to approximately 70° C./15 mbar in order to remove remaining DMF). A brown solid substance (approximately 9 g) remains behind (if no solid substance but rather an oil separates, it must be absorbed again in n-hexane/acetic ether (1:1) and washed with water), which is recrystallised from 200 mL of n-heptane in the presence of approximately 10 mL of acetic acid ethyl ester (85° C. bath temperature in the rotary evaporator, extracted by stirring/crystallised at room temperature), and after drawing off and drying initially yields 2.4 g of a flesh-colored crystallisate. Over the course of an additional crystallisation from the original solution (concentration of the original solution to dryness, recrystallisation of the residue), an additional 2.4 g of product is obtained.

One obtains 4.8 g (59% of the theory) 3-chloro-2,5-dihydroxy-benzoic acid methyl ester. Melting point: 126° C. MS (ES−): 201. ¹H-NMR (300 MHz, CDCl₃): δ (ppm)=3.96 (s, 3H); 4.61 (s, 1H); 7.15 (d, 1H), 7.24 (d, 1H), 10.86 (s, 1H).

### b) Production of 3-chloro-2,5-bis-(3,3-dichloro-allyloxy)-benzoic acid methyl ester

Under nitrogen as a protective gas, 5.4 g (177 mMol) of sodium hydride (80%) is placed in approximately 200 mL of anhydrous DMF, and then a solution of 16.3 g (80.4 mMol) of 3-chloro-2,5-dihydroxy-benzoic acid methyl ester (dissolved in approximately 40 mL of anhydrous DMF) is added drop by drop while stirring. At the same time, hydrogen escapes and the solution then becomes reddish-brown; the carbon content is maintained during the addition by means of a water bath at a temperature of 25-30° C. When the hydrogen formation is completed, one stirs forcefully for another 20 minutes at room temperature, then adds 34 g (173 mMol of 3-bromo-1,1,-dichloropropene (97%) drop by drop within approximately 30 minutes and lets its stir another 1 to 2 hours. For processing, one mixes it with approximately 400 mL of water, extracts the mixture with methylene chloride (2×250 mL), washes the unified organic phases once with water and concentrate it to dryness. A brown oil remains behind that is purified by means of column chromatography on silica gel (conditioning of the column with n-hexane/acetic acid ethyl ester (9:1); elution with 9:1, becoming polar to 1:1). The desired product elutes as rapidly as possible, and the concentration of the corresponding fractions provides a yellow oil that crystallises to a pale yellow solid substance after standing for a long period.

One obtains 23.9 g (71% of the theory) of the 3-chloro-2,5-bis-(3,3-dichloro-allyloxy)-benzoic acid methyl ester. Melting point: 63° C. ¹H-NMR (300 MHz, CDCl₃): δ (ppm)=3.92 (s, 3H); 4.65 (m, 4H); 6.12 (t, 1H); 6.32 (t, 1H); 7.12 (d, 1H), 7.23 (d, 1H).

### c) Production of 3-chloro-5-(3,3-dichloro-allyloxy)-2-hydroxy benzoic acid methyl ester

Under nitrogen as a protective gas, 9.8 g (38.0 mMol) of powdered magnesium bromide etherate is suspended in 200 mL of toluene and heated to approximately 120° C. while stirring vigorously. In the heat while stirring, one adds to this suspension a solution of 10 g (23.8 mmol) of 3-chloro-2,5-bis-(3,3,-dichloro-allyloxy)benzoic acid methyl ester in approximately 50 mL of toluene drop by drop and allows the mixture to stir again 2-4 hours at approximately 120° C. The reaction process in which the disappearance of the educt flecks takes place can be analysed by DC. As soon as no more educt can be detected, one allows the mixture to cool to room temperature and then pours it with approximately 50 mL of concentrated hydrochloric acid into the separating funnel, stirs the phases and adds another approximately 100 mL of water. After separation of the organic phase, one extracts the aqueous phase again twice with approximately 200 mL of toluene and concentrates the united organic phases to dryness. At the same time, the 3-bromo-1,1-dichloropropene generated in the reaction, which has a somewhat pungent and stimulating odor, also finally passes. The residue is recrystallised from methanol (60° C./room temperature), drawn off and the colorless crystallisate dried on the frit. Additional product can be optionally isolated from the original solution by means of a second crystallisation. One obtains 5.56 g (75% of the theory). Melting point: 86° C. MS (ES+): 311. ¹H-NMR (300 MHz, CDCl₃): δ (ppm)=3.98 (s, 3H); 4.60 (d, 2H); 6.12 (t, 1H); 7.20 (d, 1H), 7.27 (d, 1H), 10.94 (s, 1H).

### d) Production of 3-chloro-5-(3,3-dichloro-allyloxy)-2-hydroxy benzoic acid methyl ester

Under nitrogen as a protective gas, 7 g (22.5 mmol) of 3-chloro-5-(3,3-dichloro-allyloxy)-2-hydroxy benzoic acid methyl ester as well as 12.8 g (102 mmol) of dimethylsulfate at room temperature is placed in 150 mL of anhydrous DMF and mixed 13.2 g (95.2 mmol) of anhydrous potassium carbonate while stirring vigorously. Initially, a light yellow suspension develops, and after a few minutes a warm tone develops, whereby the suspension becomes dark yellow. One lets stir another approximately 2 hours at room temperature in approximately 300 mL of water and extracts twice with 400 mL of dichloromethane. After concentration of the united organic phases to dryness, an oily residue remains.

One obtains 7.05 g (96% of the theory) of 3-chloro-5-(3,3-dichloro-allyloxy)-2-methoxy benzoic acid methyl ester. ¹H-NMR (300 MHz, CDCl₃): δ (ppm)=3.89 (s, 3H); 3.95 (s, 3H); 4.63 (d, 2H); 6.12 (t, 1H); 7.10 (d, 1H), 7.21 (d, 1H).

### e) Production of 3-chloro-5-(3,3-dichloro-allyloxy)-2-methoxy benzoic acid

8 g (24.6 mmol) of 3-chloro-5-(3,3-dichloro-allyloxy)-2-methoxy benzoic acid methyl ester are dissolved in approximately 100 mL of methanol and mixed with approximately 40 mL 10% sodium hydroxide solution. The emulsion is heated to about 50° C. (“while stirring”) in the rotary evaporator, and a pale yellow solution results. The process of hydrolysis is controlled from time to time by DC; as soon as the educt can no longer be detected in the DC (typically after 20 minutes), the methanol is extensively distilled, the aqueous solution is transferred into an Erlenmeyer flask and cooled in an ice bath. While stirring vigorously, one now adds concentrated hydrochloric acid up to a clearly acidic reaction, whereby the product precipitates as a colorless solid substance. The precipitate is drawn off, absorbed in methylene chloride, washed with water to remove salts carried along, and the organic phase is concentrated to dryness. A colorless solid substance remains.

One obtains 6.8 g (96% of the theory) of 3-chloro-5-(3,3-dichloro-allyloxy)-2-methoxy benzoic acid methyl ester. Melting point: 103° C. ¹H-NMR (300 MHz, CDCl₃): δ (ppm)=4.02 (s, 3H); 4.66 (d, 2H); 6.14 (t, 1H); 7.20 (d, 1H), 7.53 (d, 1H).

### f) Production of [3-chloro-5-(3,3-dichloro-allyloxy)-2-methoxy-phenyl]-methanol

Under nitrogen as a protective gas, 3.11 g (10 mmol) of 3-chloro-5-(3,3-dichloro-allyloxy)-2-methoxy-benzoic acid is dissolved in approximately 30 mL of anhydrous THF and mixed with 15 mL of a 1 M solution of borane (15 mmol) in THF while stirring. After the hydrogen formation subsides, one allows to stand for approximately 18 hours at room temperature. For processing, one first mixes with approximately 10 mL of water in order to destroy excess borane, then adds approximately 20 mL of dilute sodium hydroxide solution and extracts twice with 100 mL of heptane in each case. After concentration of the organic phase, a pale yellow oil remains.

Man erhält 2.55 g (86% of the theory) [3-chloro-5-(3,3-dichloro-allyloxy)-2-methoxy-phenyl]-methanol. ¹H-NMR (300 MHz, CDCl₃): δ (ppm)=2.16 (t, 1H); 3.86 (s, 3H); 4.61 (d, 2H); 4.71 (d, 2H); 6.14 (t, 1H); 6.86 (s, 2H).

### g) Production of 3-chloro-5-(3,3-dichloro-allyloxy)-2-methoxy-benzaldehyde

Under nitrogen as a protective gas, 2.5 g (8.5 mmol) of [3-chloro-5-(3,3-dichloro-allyloxy)-2-methoxy-phenyl]-methanol is placed in approximately 50 mL of anhydrous dichlormethane and mixed with 2.2 g (10.2 mmol) of pyridine chlorochromate (PCC) while stirring. Shortly after the addition, the reaction solution turns dark brown. One allows the suspension to stir another approximately two hours, then adds approximately 5 mL of isopropanol in order to absorb excess PCC and stirs approximately 10 minutes. For processing, the mixture is filtered using a fluted filter, the residue is rinsed with dichloromethane, the brown filtrate is concentrated to approximately 10 mL and run through a filter column (approximately 150 g of silica gel, “conditioned” with dichloromethane; eluent: dichloromethane).

After concentration of the eluate to dryness, one obtains 2.05 g (82% of the theory) as a colorless solid substance. Melting point: 78° C. ¹H-NMR (300 MHz, CDCl₃): δ (ppm)=3.96 (s, 3H); 4.66 (d, 2H); 6.14 (t, 1H); 7.23 (s, 2H); 10.33 (s, 1H).

### h) Production of 3-chloro-5-(3,3-dichloro-allyloxy)-2-methoxy-benzaldehyde oxime

2.7 g (9.14 mmol) of 3-chloro-5-(3,3-dichloro-allyloxy)-2-methoxy-benzaldehyde, 0.7 g (10 mmol) of 9 hydroxylammonium chloride and 0.82 g (10 mmol) of sodium acetate are suspended in a mixture of 30 mL of ethanol and 15 mL of water und heated to 50° C. for approximately one hour while stirring. Subsequently, the ethanol is removed in the rotary evaporator, and the remaining aqueous suspension is extracted with dichloromethane.

After concentration of the organic phase to dryness, one obtains 2.55 g (90% of the theory) of 3-chloro-5-(3,3-dichloro-allyloxy)-2-methoxy-benzaldehyde oxime as a colorless solid substance. Melting point: 111° C. MS (ES+): 310. ¹H-NMR (400 MHz, CDCl₃): δ (ppm)=3.83 (s, 3H); 4.64 (d, 2H); 6.13 (t, 1H); 6.99 (d, 1H); 7.20 (d, 1H); 7.56 (bs, 1H); 8.37 (s, 1H).

### Example (II-2)

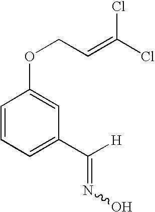

### 3-(3,3-dichloro-allyloxy)-benzaldehyde oxime

500 mg (2.16 mMol) of 3-(3,3-dichloro-allyloxy)benzaldehyde (compare JP-57018658) are dissolved in 15 ml of acetonitrile. 225 mg (3.24 mMol) of hydroxylamine hydrochloride and 0.9 ml (6.48 mMol) of triethylamine are added to this. The reaction mixture is subsequently stirred 3 hours at room temperature (RT) and then stirred with 200 ml of saturated sodium chloride solution. It is extracted twice with 100 ml of dichloromethane in each case, the united organic phases are dried over sodium sulphate and the solvent is evaporated in a vacuum.

One obtains 490 mg (92% of the theory) of 3-(3,3-dichloro-allyloxy)-benzaldehyde oxime, which can be used without further purification for the subsequent reaction.

LC-MS (ES⁺) m/z (%)=246.

The compounds of the general formula (II) listed in Table 2 can also produced analogously to the Examples (II-1) and (II-2) as well as corresponding to the general description of the method according to the invention.

TABLE 2
Examples for the compounds of the formula (II)

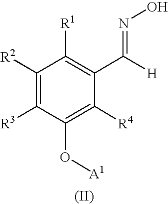

| Ex. no. | A¹ | R¹ | R² | R³ | R⁴ | Physical data |
|---|---|---|---|---|---|---|
| II-3 | 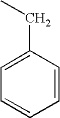 | H | CF₃ | H | H | (see information following this table) |
| II-4 | 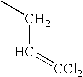 | 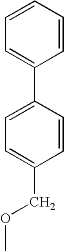 | Cl | H | H | |
| II-5 | 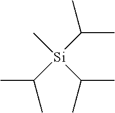 | 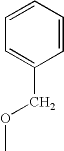 | Cl | H | H | (see information following this table) |
| II-6 | 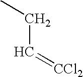 | OC₂H₅ | Cl | H | H | MS (ES+): 324 |
| II-7 | 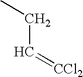 | OCH₃ | H | H | H | MS (ES+): 276 |
| II-8 | 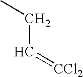 | Cl | H | H | Cl | MS (ES+): 316 |
| II-9 | 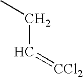 | H | Cl | H | H | MS (ES+): 280 |
| II-10 | 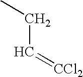 | 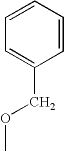 | Cl | H | H | |
| II-11 | 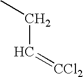 | 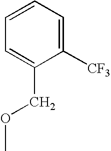 | Cl | H | H | MS (ES+): 454 |
| II-12 | 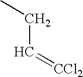 | 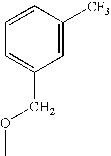 | Cl | H | H | MS (ES+): 454 |
| II-13 | 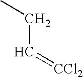 | 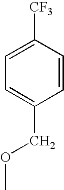 | Cl | H | H | MS (ES+): 454 |
| II-14 | 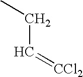 | 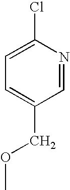 | Cl | H | H | MS (ES+): 423 |
| II-15 | 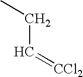 | 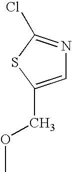 | Cl | H | H | MS (ES+): 429 |
| II-16 | 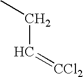 | OCH₃ | Br | H | H | |
| II-17 | 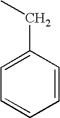 | OCH₃ | Cl | H | H | |
| II-18 | H | OCH₃ | Cl | H | H | |

### Example (II-3)

### 3-trifluoromethyl-5-(3,3-dichloro-allyloxy)benzaldehyde oxime

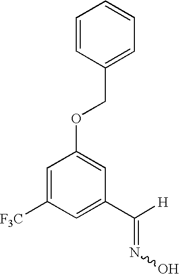

### a) Production of 3-hydroxy-5-trifluoromethyl benzoic acid methyl ester

18 g (66.9 mMol) of 3-bromo-5-trifluoromethyl benzoic acid (CAS No. 328-67-6) and 11.3 g (201 mMol) of potassium hydroxide are added to 150 ml of methanol and stirred for 20 hours in the autoclave at the appropriate pressure (approximately 40 bar). The reaction mixture is added to 100 ml of water and extracted twice with 100 ml of dichloromethane in each case. It is adjusted to pH 1-2 with concentrated HCl and filtered.

One obtains 1.6 g (11% of the theory) of 3-hydroxy-5-trifluoromethyl benzoic acid methyl ester. MS-CI: 221.

### b) Production of 3-benzyloxy-5-trifluoromethyl benzoic acid benzyl ester

990 mg (4.5 mMol) of 3-hydroxy-5-trifluoromethyl benzoic acid methyl ester are stirred with 1.69 g (9.0 mMol) of benzyl bromide and 3.8 g (11.7 mMol) of cesium carbonate in 15 ml of DMF over 18 hours at 80° C. The reaction mixture is distributed between water and acetic ether. The organic phase is concentrated to dryness.

One obtains 1.13 g (65% of the theory) of 3-benzyloxy-5-trifluoromethyl benzoic acid benzyl ester.

### c) Production of (3-benzyloxy-5-trifluoromethyl-phenyl)methanol

The reduction takes place analogously to the instructions from Example (II-5c) with: 880 mg (2.28 mMol) of 3-benzyloxy-5-trifluoromethyl benzoic acid benzyl ester, 74 mg (3.42 mMol) of lithium borohydride, and 30 ml of diethyl ether. After the residue was chromatographed over silica gel, one obtains 400 mg (purity 100%, 62% of the theory) and 300 mg (purity 47%, 22% of the theory) of (3-benzyloxy-5-trifluoromethyl-phenyl)-methanol. ¹H-NMR: CDCl₃, δ=7.4 (m, 5H, PhH), 7.22, 7.18 und 7.14 (in each case 1H, CF₃-PhH), 5.10 (2H, CH₂), 4.71 (2H, CH₂), OH not attached.

### d) Production of 3-benzyloxy-5-trifluoromethyl benzaldehyde

The oxidation takes place analogously to the instructions from Example (II-1) with: 400 mg (1.42 mMol) of (3-benzyloxy-5-trifluoromethyl-phenyl)-methanol, 507 mg (2.35 mMol) of pyridine chlorochromate, and 30 ml of dichlormethane. After concentration of the organic phase to dryness, one obtains 350 g (88% of the theory) of 3-benzyloxy-5-trifluoromethyl benzaldehyde.

### e) Production of 3-Benzyloxy-5-trifluoromethyl benzaldehyde oxime

The formation of the oxime takes place analogously to the instructions from Example (II-2) with a reaction time of 18 hours with: 350 mg (1.25 mMol) of 3-benzyloxy-5-trifluoromethyl benzaldehyde, 130 mg (1.87 mMol) of hydroxylamine hydrochloride, 378 mg (3.75 mMol) of triethylamine, and 20 ml of acetonitrile. After concentration of the organic phase to dryness, one obtains 370 mg (purity 86%, 86% of the theory) of 3-benzyloxy-5-trifluoromethyl-benzaldehyde oxime. MS (ES+): 296.

### Example (II-5)

### 2-benzyloxy-3-chloro-5-triisopropylsilyloxy-benzaldehyde oxime

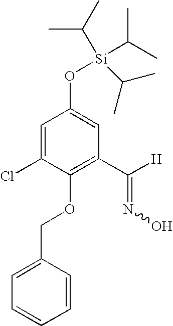

### a) Production of 3-chloro-2-hydroxy-5-triisopropylsilyloxy benzoic acid methyl ester

10.0 g (49 mMol) of 3-chloro-2,5-dihydroxy-benzoic acid methyl ester are dissolved in 200 ml of dichlormethane. 6.48 g (64 mMol) of triethylamine and 11.4 g (59 mMol) of triisopropylsilyl chloride are added drop by drop. The reaction mixture is stirred over 16 hours at room temperature and subsequently distributed between water and acetic ether. The organic phase is concentrated to dryness. One obtains 18.0 g (purity 96%, 97% of the theory) of 3-chloro-2-hydroxy-5-triisopropylsilyloxy benzoic acid methyl ester. MS (ES+): 359.

### b) Production of 2-benzyloxy-3-chloro-5-triisopropylsilyloxy benzoic acid methyl ester

18.0 g (50 mMol) of 3-chloro-2-hydroxy-5-triisopropylsilyloxy benzoic acid methyl ester, 10.3 g (60 mMol) of benzyl bromide and 9.0 g-(65 mMol) of potassium carbonate are stirred in 200 ml of acetonitrile over one hour at room temperature. The reaction mixture is distributed between water and acetic ether. The organic phase is concentrated to dryness. One obtains 22.3 g (purity 75%, 74% of the theory) of 2-benzyloxy-3-chloro-5-triisopropylsilyloxy benzoic acid methyl ester.

MS (ES+): 449.

### c) Production of (2-benzyloxy-3-chloro-5-triisopropylsilyloxy-phenyl)-methanol

300 mg (0.67 mMol) of 2-benzyloxy-3-chloro-5-triisopropylsilyloxy benzoic acid methyl ester are dissolved in 20 ml of diethyl ether. At 0° C., 22 mg (1.0 mMol) of lithium borohydride are added and stirred over 18 hours at room temperature. 5 ml of saturated aqueous ammonium chloride solution and 5 mL of saturated aqueous sodium hydrogen carbonate solution are added. It is extracted with dichloromethane, and the organic phase is concentrated to dryness.

One obtains 280 mg (purity 90%, 89% of the theory) of (2-benzyloxy-3-chloro-5-triisopropylsilyloxy-phenyl)-methanol. MS (ES+): 403. ¹H-NMR: CDCl₃, δ=7.4 (m, 5H, PhH), 6.88 and 6.78 (in each case d, 1H, Cl-PhH), 5.00 (s, 2H, CH₂), 4.50 (d, 2H, CH₂OH), 1.25 (m, 3H, SiCH), 1.1 (18H, CH₃).

### d) Production of 2-benzyloxy-3-chloro-5-triisopropylsilyloxy benzaldehyde

The oxidation takes place analogously to the instructions according to Example (II-1) with: 12.7 g (30 mMol) of (2-benzyloxy-3-chloro-5-triisopropylsilyloxy-phenyl)-methanol, 10.8 g (50 mMol) of pyridine chlorochromate, and 300 ml of dichloromethane.

After concentration of the organic phase to dryness, one obtains 12.2 g (96% of the theory) of 2-benzyloxy-3-chloro-5-triisopropylsilyloxy benzaldehyde. ¹H-NMR: CDCl₃, δ=10.0 (s, 1H, CHO), 7.37 (m, 5H, PhH), 7.22 and 7.16 (in each case d, 1H, Cl-PhH), 5.09 (s, 2H, CH₂), 1.25 (m, 3H, SiCH), 1.1 (18H, CH₃).

### e) Production of 2-benzyloxy-3-chloro-5-triisopropylsilyloxy benzaldehyde oxime

The formation of the oxime takes place analogously to the instructions from Example (II-2) with a reaction time of one hour with: 500 mg (1.19 mMol) of 2-benzyloxy-3-chloro-5-triisopropylsilyloxy benzaldehyde, 124 mg (1.79 mMol) of hydroxylamine hydrochloride, 361 mg (3.58 mMol) of triethylamine, and 20 ml of acetonitrile.

After concentration of the organic phase to dryness, one obtains 490 g (purity 88%, 83% of the theory) of 2-benzyloxy-3-chloro-5-triisopropylsilyloxy benzaldehyde oxime. MS (ES+): 434.

Starting Substances for the Formula (V):

### Example (V-1)

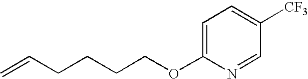

### 2-(n-hex-5-en-1-yl-oxy)-5-trifluoromethyl-pyridine

0.356 g (11.1 mMol) of 75% sodium hydride are stirred in 10 ml of tetrahydrofurane (THF) under protective gas (nitrogen). 1.01 g (10 mMol) of n-hex-5-en-1-ol—dissolved in 2.0 ml of THF—are subsequently added drop by drop at room temperature, and the mixture is stirred 20 minutes. 2.0 g (12 mMol) of 2-chloro-5-trifluoromethylpyridine (T. Haga et al., Heterocycles, 1984, 22(1), p. 117; G. E. Carr et al., J. Chem. Soc., Perkin Trans 1, 1988, p. 921) are subsequently added and the reaction mixture is stirred approximately 16 hours at room temperature. For processing the entire reaction preparation is stirred with 200 ml of water, and extracted three times with 50 ml of dichloromethane in each case. The united organic phases are subsequently washed with water. After the concentration of the organic phase in a vacuum, the remaining residue is chromatographed over silica gel.

One obtains 2.0 g (75% of the theory) of 2-(n-hex-5-en-1-yl-oxy)-5-trifluoromethyl-pyridine. LC-MS (ES+) m/z (%)=246

### Example (V-2)

### 2-pent-4-enyloxy-5-trifluoromethylpyridine

The illustration of 2-pent-4-enyloxy-5-trifluoromethylpyridine takes place corresponding to the Example (III-1) using 4.75 g of penten-5-ol (55.1 mmol), 1.21 g of sodium hydride (60%) (30.3 mmol) and 2-chloro-5-trifluoromethyl-pyridine (27.5 mmol). For processing, the brown suspension obtained with the conversion is mixed with approximately 50 mL of water and extracted with acetic ether/heptane. Remaining after the concentration of the organic phase to dryness is an oil mixture that is purified using chromatography on silica (flow agent heptane/acetic ether 4:1) for the separation of 2-hydroxy-5-trifluoromethyl pyridine.

One obtains 5.15 g (81% of the theory) of 2-pent-4-enyloxy-5-trifluoromethyl pyridine. MS (ES+): 232

### Example (V-3)

### (2-trifluoroethoxypyridin-5-yl)(penten-5-yl)ether

1.1 g (5.7 mmol) of 2-trifluoroethoxy-5-hydroxy-pyridine (produced through the oxidation of 5-(2,2-dimethyl-[1,3,2]dioxaborinan-2-yl)-2-(2,2,2-trifluoroethoxy)-pyridine with hydrogen peroxide in glacial acetic acid; synthesis of 5-(2,2-dimethyl-[1,3,2]dioxaborinan-2-yl)-2-(2,2,2-trifluoroethoxy)-pyridine, known from WO-99/65901), 7.0 g (50 mmol) of potassium carbonate and 1.6 g (10.7 mmol) of 5-bromopentene are suspended or dissolved in approximately 50 mL of DMF while stirring vigorously at room temperature overnight. A green-brown suspension results, which is mixed with approximately 50 mL of water for processing and is extracted twice with 100 mL of dichloromethane in each case. Concentration of the organic phase to dryness provides 1.2 g (80% of the theory) of 2-trifluoroethyoxy-5-pent-4-enyloxy-pyridine as a brown oil; this raw product can be used for further conversions.

### Example (V-4)

### 2-(3-methyl-but-3-enyloxy)-5-trifluoromethyl-pyridine

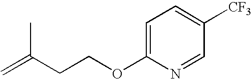

The compound is produced analogously to the instructions according to Example (V-1) with: 267 mg (75%; 8.3 mMol) of sodium hydride, 653 mg (7.6 mMol) of 3-methyl-3-buten-1-ol, 1.5 g (8.3 mMol) of 2-chloro-5-trifluoromethylpyridine, and 12 ml of THF.

After the residue was chromatographed over silica gel, one obtains 1.2 g (purity 76%, 52% of the theory) of 2-(3-methyl-but-3-enyloxy)-5-trifluoromethyl-pyridine. MS(ES+): 323.

Starting Substances for the Formula (VIII):

### Example (VIII-1)

### 2-N,N-dimethylamino-5-(3,3-dichloro-allyloxy)-benzaldehyde

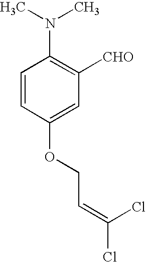

150 mg (0.60 mMol) of 2-fluoro-5-(3,3-dichloro-allyloxy)benzaldehyde are stirred in 10 ml of a mixture of dimethylsulphoxide and water (2.5:1). One subsequently adds 68.7 mg (0.84 mMol) of N,N-dimethyl ammonium chloride and 83.2 mg (0.60 mMol) of potassium carbonate and stirs the reaction mixture approximately 18 hours at 100° C. (also compare methods from: Bioorg. Med. Chem. 9 (2001), p. 677-694). After cooling, the reaction mixture is diluted with 25 ml of water and extracted with methylene chloride. The organic phase is separated, dried and concentrated in a vacuum. The remaining residue is subsequently [purified] by means of column chromatography (eluent: cyclohexane:acetic ether=5:1).

One obtains 25 mg (15% of the theory) of 2-N,N-dimethylamino-5-(3,3-dichloro-allyloxy)-benzaldehyde. C₁₂H₁₃Cl₂NO₂ (274.1). LC-MS (ES⁺) m/z (%) 274.

### Example (VIII-2)

### 2-methylthio-5-(3,3-dichloro-allyloxy)-benzaldehyde

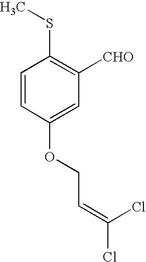

150 mg (0.60 mMol) of 2-fluoro-5-(3,3-dichloro-allyloxy)benzaldehyde are stirred in 10 ml of N,N-dimethylformamide, mixed with 42.2 mg (0.60 mMol) of sodium methanethiolate and allowed to stir approximately 6 hours at a temperature of 65° C. C (also compare method from Chem. 45, 25 (2002), p. 5417). The reaction mixture is subsequently added to water and extracted with methylene chloride. The organic phase is separated, dried and concentrated in a vacuum. The remaining residue is subsequently separated by means of column chromatography (eluent: cyclohexane:acetic ether=4:1).

One obtains 54 mg (32% of the theory) of 2-methylthio-5-(3,3-dichloro-allyloxy)benzaldehyde. C₁₁H₁₀Cl₂O₂S (277.1). LC-MS (ES⁺) m/z (%)=277.

The compounds of the general formula (VIII) listed in the following Table 3 can also produced analogously to the Examples (VIII-1) and (VIII-2) as well as corresponding to the general description of the method according to the invention.

TABLE 3
Starting compounds of the general formula (VIII)

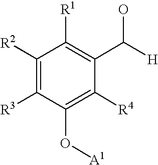

| | | | | | | Physical |
|---|---|---|---|---|---|---|
| Ex. no. | A¹ | R¹ | R² | R³ | R⁴ | data |
| VIII-3 | —CH₂-Ph | —O—CH₃ | Cl | H | H | MS (ES+): |
| | | | | | | 277 |
| VIII-4 | —CH₂—CH═CCl₂ | —O—CH₂-Ph | Cl | H | H | |
| VIII-5 | —CH₂—CH═CCl₂ | —O—CH₂-Ph | Br | H | H | MS (ES+): |
| | | | | | | 341 |
| VIII-6 | —CH₂—CH═CCl₂ | —O—CH₂-(2-Cl-1,3- | Cl | H | H | MS (ES+): |
| | | thiazol-5-yl) | | | | 413 |
| VIII-7 | —CH₂—CH═CCl₂ | —O—CH₂-(2-Cl- | Cl | H | H | MS (ES+): |
| | | pyrid-5-yl) | | | | 408 |
| VIII-8 | —CH₂—CH═CCl₂ | —O—CH₂-(4-CF₃- | Cl | H | H | |
| | | phenyl | | | | |
| VIII-9 | —CH₂—CH═CCl₂ | —O—CH₂-(3-CF₃- | Cl | H | H | MS (ES+): |
| | | phenyl | | | | 439 |
| VIII-10 | —CH₂—CH═CCl₂ | —O—CH₂-(2-CF₃- | Cl | H | H | MS (ES+): |
| | | phenyl | | | | 439 |
| VIII-11 | H | —O—CH₃ | Cl | H | H | MS (ES+): |
| | | | | | | 187 |

Starting Substances of the Formula (IX) (if A¹=H) and the Formula (XI)

TABLE 4
Starting compounds of the general formula (IX) and (XI)

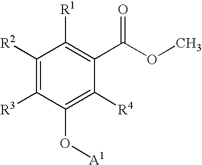

| Ex. | | | | | | Physical |
|---|---|---|---|---|---|---|
| no. | A¹ | R¹ | R² | R³ | R⁴ | data |
| IX-1 | H | —O—CH₂-(2-Cl- | Cl | H | H | MS (ES+): |
| | | pyrid-5-yl) | | | | 328 |
| IX-2 | H | —O—CH₃ | Br | H | H | MS (ES+): |
| | | | | | | 262 |
| IX-3 | H | —OH | Br | H | H | MS (ES+): |
| | | | | | | 247 |
| IX-4 | H | —O—CH₂-Ph | Cl | H | H | |
| IX-5 | H | —O—CH₂-(2-Cl-1,3- | Cl | H | H | MS (ES+): |
| | | thiazol-5-yl) | | | | 334 |
| IX-6 | H | —O—CH₂-(4-CF₃- | Cl | H | H | MS (ES+): |
| | | phenyl | | | | 360 |
| IX-7 | H | —O—CH₂-(3-CF₃- | Cl | H | H | MS (ES+): |
| | | phenyl | | | | 360 |
| IX-8 | H | —O—(CH₂)₂—OCH₃ | Cl | H | H | MS (ES+): |
| | | | | | | 261 |
| XI-1 | —CH₂—CH═CCl₂ | —O—CH₂-(2-Cl- | Cl | H | H | MS (ES+): |
| | | pyrid-5-yl) | | | | 438 |
| XI-2 | —CH₂-(2-Cl-pyrid- | —O—CH₂—(2-Cl- | Cl | H | H | |
| | 5-yl) | pyrid-5-yl) | | | | |
| XI-3 | —CH₂-Ph | —O—CH₃ | Cl | H | H | MS (ES+): |
| | | | | | | 307 |
| XI-4 | —Si(CH₃)2-tBu | —OH | Cl | H | H | MS (ES+): |
| | | | | | | 317 |
| XI-5 | —CH₂-Ph | —O—CH₃ | Br | H | H | MS (ES+): |
| | | | | | | 351 |
| XI-6 | —CH₂—CH═CCl₂ | —O—CH₃ | Br | H | H | MS (ES+): |
| | | | | | | 371 |
| XI-7 | —Si(CH₃)2-tBu | —OH | Br | H | H | MS (ES+): |
| | | | | | | 363 |
| XI-8 | —CH₂—CH═CCl₂ | —O—CH₂-Ph | Cl | H | H | |
| XI-9 | —CH₂—CH═CCl₂ | —O—CH₂-(2-Cl-1,3- | Cl | H | H | MS (ES+): |
| | | thiazol-5-yl) | | | | 443 |
| XI-11 | —CH₂—CH═CCl₂ | —O—CH₂-(4-CF₃- | Cl | H | H | |
| | | phenyl | | | | |
| XI-11 | —CH₂—CH═CCl₂ | —O—CH₂-(3-CF₃- | Cl | H | H | |
| | | phenyl | | | | |
| XI-12 | —CH₂—CH═CCl₂ | —O—CH₂-(2-CF₃- | Cl | H | H | |
| | | phenyl | | | | |

Starting Substances of the Formula (XII):

TABLE 5
Starting compounds of the general formula (XII)

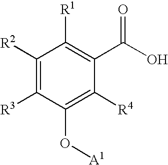

| Ex. | | | | | | Physical |
|---|---|---|---|---|---|---|
| no. | A¹ | R¹ | R² | R³ | R⁴ | data |
| XII-1 | —CH₂-Ph | —O—CH₃ | Cl | H | H | MS (ES+): |
| | | | | | | 293 |
| XII-2 | —H | —O—CH₃ | Cl | H | H | MS (ES+): |
| | | | | | | 203 |
| XII-3 | —CH₂—CH═CCl₂ | —O—CH₃ | Br | H | H | MS (ES+): |
| | | | | | | 356 |
| XII-4 | —CH₂—CH═CCl₂ | —O—CH₂-Ph | Cl | H | H | MS (ES+): |
| | | | | | | 387 |

Starting Substances of the Formula (XIII):

TABLE 6
Starting compounds of the general formula (XIII)

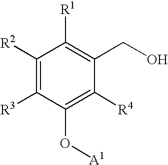

| Ex. | | | | | | Physical |
|---|---|---|---|---|---|---|
| no. | A¹ | R¹ | R² | R³ | R⁴ | data |
| XIII-1 | —CH₂-Ph | —O—CH₃ | Cl | H | H | MS (ES+): |
| | | | | | | 261 |
| | | | | | | (M⁺-H₂O) |
| XIII-2 | —CH₂-Ph | —O—CH₃ | Br | H | H | MS (ES+): |
| | | | | | | 325 |
| | | | | | | (M⁺-H₂O) |
| XIII-3 | —CH₂—CH═CCl₂ | —O—CH₂-Ph | Cl | H | H | MS (ES+): |
| | | | | | | 355 |
| | | | | | | (M⁺-H₂O) |
| XIII-4 | —CH₂—CH═CCl₂ | —O—CH₂-(2-Cl-1,3- | Cl | H | H | MS (ES+): |
| | | thiazol-5-yl) | | | | 415 |
| XIII-5 | —CH₂—CH═CCl₂ | —O—CH₂-(2-Cl- | Cl | H | H | MS (ES+): |
| | | pyrid-5-yl) | | | | 409 |
| XIII-6 | —CH₂—CH═CCl₂ | —O—CH₂-(4-CF₃- | Cl | H | H | MS (ES+): |
| | | phenyl | | | | 424 |
| | | | | | | (M⁺-H₂O) |
| XIII-7 | —CH₂—CH═CCl₂ | —O—CH₂-(3-CF₃- | Cl | H | H | MS (ES+): |
| | | phenyl | | | | 424 |
| | | | | | | (M⁺-H₂O) |
| XIII-8 | —CH₂—CH═CCl₂ | —O—CH₂-(2-CF₃- | Cl | H | H | MS (ES+): |
| | | phenyl | | | | 424 |
| | | | | | | (M⁺-H₂O) |
| XIII-9 | —H | —O—CH₃ | Cl | H | H | MS (ES+): |
| | | | | | | 171 |
| | | | | | | (M⁺-H₂O) |

Additional Starting Substances

### (A-1) 4-(2-methoxy-3-chloro-5-triisopropylsilyloxy-phenyl)-2-hydroxyethyl-thiazole

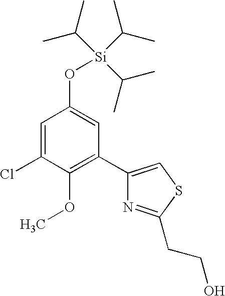

The production of this compound is described in Example (I-37).

### (A-2) (R/S)-3-(2-methoxy-3-chloro-5-hydroxy-phenyl)-5-((5-trifluoromethyl-pyridin-2-yl)-3-(propyl)ether-1-yl)-Δ²-isoxazoline

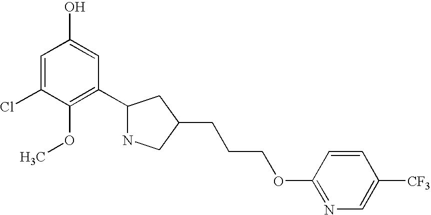

### a) (R/S)-3-(2-methoxy-3-chloro-5-benzyloxy-phenyl)-5-((5-trifluoro-methyl-pyridin-2-yl)-3-(propyl)ether-1-yl)-Δ²-isoxazoline

The compound is produced analogously to Example (I-1), with 1.38 g (4.38 mMol) of 2-methoxy-3-chloro-5-benzyloxyphenyl-benzaldehyd-oxime, 1.2 g (5.20 mMol) of 2-(n-pent-5-en-1-yl-oxy)-5-trifluoromethyl-pyridine, 694.8 mg (5.2 mMol) of N-chloro-succinimide, 718.0 mg (7.1 mMol) of triethylamine, and 81 ml of N,N-dimethylformamide. The reaction preparation is subsequently extracted by shaking with dichloromethane/water, and the separated aqueous phase extracted again with dichloromethane. The united organic phases are dried and concentrated in a vacuum. The remaining residue is chromatographed over silica gel (eluent: cyclohexane/acetone=10:1).

One obtains 592.2 mg (purity 100%, 24% of the theory) of (R/S)-3-(2-methoxy-3-chloro-5-benzyloxy-phenyl)-5-((5-trifluoro-methyl-pyridin-2-yl)-3-(propyl)ether-1-yl)-Δ²-isoxazoline, C₂₆H₂₄ClF₃N₂O₄ (520.9). MS (ES+): 521

### b) (R/S)-3-(2-methoxy-3-chloro-5-hydroxy-phenyl)-5-((5-trifluoro-methyl-pyridin-2-yl)-3-(propyl)ether-1-yl)-Δ²-isoxazoline

924 mg (1.77 mMol) of (R/S)-3-(2-methoxy-3-chloro-5-benzyloxy-phenyl)-5-((5-trifluoro-methyl-pyridin-2-yl)-3-(propyl)ether-1-yl)-Δ²-isoxazoline are stirred in 138.6 ml of ethanol and hydrogenated under normal pressure in the presence of 184.8 mg (1.32 mMol) of palladium(II) hydroxide carbon [20% Pd content] for approximately three hours at room temperature. After the concentration of the entire reaction preparation, the remaining residue is chromatographed over silica gel. (Eluent: cyclohexane/acetone=4:1).

One obtains 730 mg (purity 84%, 80% of the theory) of (R/S)-3-(2-methoxy-3-chloro-5-hydroxyphenyl)-5-((5-trifluoro-methyl-pyridin-2-yl)-3-(propyl)ether-1-yl)-Δ²-isoxazoline, C₁₉H₁₈ClF₃N₂O₄ (430.8). MS (ES+): 431

### (A-3) (R/S)-3-(2-methoxy-3-chloro-4-fluoro-5-hydroxy-phenyl)-5-((3-chloro-5-trifluoromethylpyridin-2-yl)-3-(propyl)ether-1-yl)-Δ²-isoxazoline

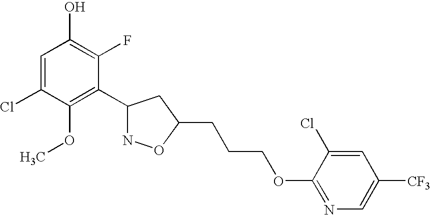

### a) (R/S)-3-(2-methoxy-3-chloro-4-fluoro-5-benzyloxy-phenyl)-5-((3-chloro-5-trifluoro-methyl-pyridin-2-yl)-3-(propyl)ether-1-yl)-Δ²-isoxazoline

500.0 mg (0.09 mMol) of (R/S)-3-(2-methoxy-3-chloro-5-benzyloxy-phenyl)-5-((3-chloro-5-trifluoromethyl-pyridin-2-yl)-3-(propyl)ether-1-yl)-Δ²-isoxazoline are stirred with 318.9 mg (0.09 mMol) of Selectfluor® in 50.0 ml of acetonitrile for approximately 18 hours at 70° C. The reaction preparation is subsequently concentrated in a vacuum and separated by means of preparative HPLC.

One obtains 51.8 mg (10.4% of the theory) of (R/S)-3-(2-methoxy-3-chloro-4-fluoro-5-benzyloxyphenyl)-5-((3-chloro-5-trifluoro-methyl-pyridin-2-yl)-3-(propyl)ether-1-yl)-Δ²-isoxazoline.

¹³C-NMR: δ (CDCl₃, ppm)=24.7, 31.7, 42.9 (CH₂); 62.5 (O—CH₃); 67.4, 72.1 (OCH₂); 117.6, 127.5, 128.4, 128.7, 135.6 (HC—Ar); 123.1 (Cl—C-Py); 143.7 (F—C-Py); 161.2 (O—C—Ar); 120.7 (F₃C—C-Py); 123.1 (CF₃-Py); 115.7 (Het-C—Ar); 123.1 (Cl—C—Ar); 148.1 (C—Ar); 149.9 (C═N—O); 135.2, 142.4 (CH-Py); 161.2 (C-Py). MS (ES+): 573.

### b) (R/S)-3-(2-methoxy-3-chloro-4-fluoro-5-hydroxy-phenyl)-5-((3-chloro-5-trifluoro-methyl-pyridin-2-yl)-3-(propyl)ether-1-yl)-Δ²-isoxazoline

45 mg (0.8 mMol) of (R/S)-3-(2-methoxy-3-chloro-4-fluoro-5-benzyloxy-phenyl)-5-((3-chloro-5-trifluoro-methyl-pyridin-2-yl)-3-(propyl)ether-1-yl)-Δ²-isoxazoline after stirred in 7,5 ml of ethanol and hydrogenated under normal pressure in the presence of 5.0 mg (0.04 mMol) of palladium(II) hydroxide carbon [20% Pd content] for approximately 45 minutes at room temperature. After the concentration of the entire reaction preparation in a vacuum, one obtains 39.1 mg (purity 82%, 85% of the theory) of (R/S)-3-(2-methoxy-3-chloro-4-fluoro-5-hydroxy-phenyl)-5-((3-chloro-5-trifluoro-methyl-pyridin-2-yl)-3-(propyl)ether-1-yl)-Δ²-isoxazoline, which can be used for the subsequent reactions. MS (ES+): 483.

The (R/S)-3-(2-methoxy-3-chloro-4-fluoro-5-hydroxy-phenyl)-5-((5-trifluoro-methyl-pyridin-2-yl)-3-(propyl)ether-1-yl)-Δ²-isoxazoline is produced analogously with 77 mg (0.14 mMol) of (R/S)-3-(2-methoxy-3-chloro-4-fluoro-5-benzyloxy-phenyl)-5-((5-trifluoro-methyl-pyridin-2-yl)-3-(propyl)ether-1-yl)-Δ²-isoxazoline, 10.0 mg (0.04 mMol) of palladium(II) hydroxide carbon [20% Pd content], and 15.0 ml of ethanol. After the concentration of the entire reaction preparation, concentrated in a vacuum, one obtains 63.4 mg (purity 100%, 98.7% of the theory) of (R/S)-3-(2-methoxy-3-chloro-4-fluoro-5-hydroxy-phenyl)-5-((5-trifluoro-methyl-pyridin-2-yl)-3-(propyl)ether-1-yl)-Δ²-isoxazoline, which can be used for the subsequent reactions. MS (ES+): 449.

### (A-4) (R/S)-3-(2-fluoro-5-hydroxy-phenyl)-5-((3-chloro-5-trifluoromethyl-pyridin-2-yl)-3-(propyl) ether-1-yl)-Δ²-isoxazoline

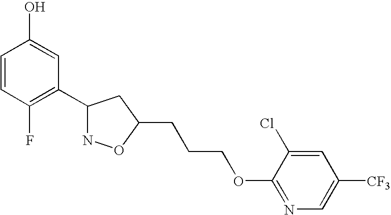

### a) (R/S)-3-(2-fluoro-5-methoxy-phenyl)-5-((3-chloro-5-trifluoro-methyl-pyridin-2-yl)-3-(propyl)ether-1-yl)-Δ²-isoxazoline

The (R/S)-3-(2-fluoro-5-methoxy-phenyl)-5-((3-chloro-5-trifluoro-methyl-pyridin-2-yl)-3-(propyl)ether-1-yl)-Δ²-isoxazoline is produced analogously to Example (I-1) with 250 mg (1.48 mMol) of 2-fluoro-5-methoxy-benzaldehyde oxime, 589 mg (2.22 mMol) of 2-(n-pent-5-en-1-yl-oxy)-3-chloro-5-trifluoromethyl-pyridine, 217.1 mg (1.63 mMol) of N-chloro-succinimide, 154.5 mg (1.63 mMol) of triethylamine, and 15 ml of N,N-dimethylformamide. The reaction preparation is subsequently extracted by shaking with dichloromethane/water, and the separated aqueous phase extracted again with dichloromethane. The united organic phases are dried and concentrated in a vacuum. The remaining residue is chromatographed over silica gel (Eluent: acetic ether/acetone=6:1).

One obtains 361 mg (56% of the theory) of (R/S)-3-(2-fluoro-5-methoxy-phenyl)-5-((3-chloro-5-trifluoro-methyl-pyridin-2-yl)-3-(propyl)ether-1-yl)-Δ²-isoxazoline. MS (ES+): 433.

### b) (R/S)-3-(2-fluoro-5-hydroxy-phenyl)-5-((3-chloro-5-trifluoro-methyl-pyridin-2-yl)-3-(propyl)ether-1-yl)-Δ²-isoxazoline

322 mg (0.74 mMol) of (R/S)-3-(2-fluoro-5-methoxy-phenyl)-5-((3-chloro-5-trifluoro-methyl-pyridin-2-yl)-3-(propyl)ether-1-yl)-Δ²-isoxazoline are stirred in 20 ml of dichloromethane and added drop by drop at −70° C. to 559.1 mg (2.23 mMol) of BBr₃ solution (=2.23 ml) in methylene chloride. The reaction mixture is subsequently stirred for approximately 18 hours at room temperature. For processing, one mixed the reaction mixture with 40 ml of ice/water and stirs an additional hour. The organic phase is subsequently separated and then first washed with saturated NaHCO₃ solution and then with 2M NaOH solution. The inorganic phases are unified, acidified with concentrated hydrochloric acid and extracted with dichloromethane. The organic phase is separated and washed with water. After drying, the united organic phases are concentrated in a vacuum. The remaining residue is chromatographed over silica gel (Eluent: acetic ether/acetone=6:1). One obtains 90 mg (purity 74%, 21% of the theory) of (R/S)-3-(2-fluoro-5-hydroxy-phenyl)-5-((3-chloro-5-trifluoro-methyl-pyridin-2-yl)-3-(propyl)ether-1-yl)-Δ²-isoxazoline, which can be used for subsequent reactions. MS (ES+): 419.

### (A-5) (R/S)-3-(2-N,N-dimethylamino-3-chloro-5-methoxy-phenyl)-5-((3-chloro-5-trifluoromethylpyridin-2-yl)-3-(propyl)ether-1-yl)-Δ²-isoxazoline

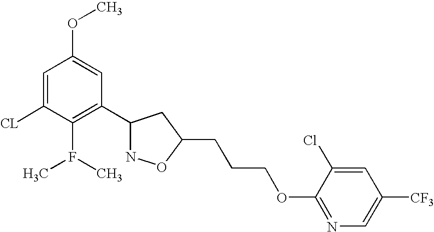

### a) 2-N,N-dimethylamino-3-chloro-5-methoxy-benzaldehyde

2-N,N-dimethylamino-3-chloro-5-methoxy-benzaldehyde: 450 mg (2.5 mMol) of 2-N,N-dimethylamino-5-methoxy-benzaldehyde (production: compare. P. Damhaut et al., Tetrahedron, 53 (16), 5785-5796, 1997) are mixed in portions and under ice cooling (0-10° C.) with 435.8 mg (3.2 mMol) of N-chloro-succinimide in 20 ml of N,N-dimethylformamide and subsequently stirred an additional 18 hours at room temperature. The entire reaction mixture is subsequently added to water and extracted with dichloromethane. The organic phase is dried and concentrated in a vacuum. The remaining residue is chromatographed over silica gel (acetic ether/acetone=5:1).

One obtains 129 mg (purity 87%, 21% of the theory) of 2-N,N-dimethylamino-3-chloro-5-methoxy-benzaldehyde. MS (ES+): 214

### b) 2-N,N-dimethylamino-3-chloro-5-methoxy-benzaldehyde oxime

2-N,N-dimethylamino-3-chloro-5-methoxy-benzaldehyde oxime is produced analogously to Example (II-2) with 130 mg (0.6 mMol) of 2-N,N-dimethylamino-3-chloro-5-methoxy-benzaldehyde, 60 mg (0.91 mMol) of hydroxylamine hydrochloride, 180 mg (1.81 mMol) of triethylamine, and 10 ml of acetonitrile.

One obtains 100 mg (purity 82%, 59% of the theory) of 2-N,N-dimethylamino-3-chloro-5-methoxy-benzaldehyde oxime, which can be used for subsequent reactions. MS (ES+): 229

### c) (R/S)-3-(2-N,N,-dimethylamino-3-chloro-5-methoxy-phenyl)-5-((3-chloro-5-trifluoromethylpyridin-2-yl)-3-(propyl)ether-1-yl)-Δ²-isoxazoline

(R/S)-3-(2-N,N,-dimethylamino-3-chloro-5-methoxy-phenyl)-5-((3-chloro-5-trifluoromethylpyridin-2-yl)-3-(propyl)ether-1-yl)-Δ²-isoxazoline is produced analogously to Example (I-1) with 100 mg (0.44 mMol) of 2-N,N-dimethylamino-3-chloro-5-methoxy-benzaldehyde oxime, 174.3 mg (0.66 mMol) of 2-(n-pent-5-en-1-yl-oxy)-3-chloro-5-trifluoromethyl-pyridine, 64.2 mg (0.48 mMol) of N-chloro-succinimide, 48.6 mg (0.48 mMol) of triethylamine, and 10 ml of N,N-dimethylformamide. The reaction preparation is subsequently extracted by shaking with dichloromethane/water, and the separated aqueous phase extracted again with dichloromethane. The united organic phases are dried and concentrated in a vacuum. The remaining residue is chromatographed over silica gel (cyclohexane/acetone=10:1).

One obtains 10 mg (purity 93%, 4% of the theory) of (R/S)-3-(2-N,N,-dimethylamino-3-chloro-5-methoxy-phenyl)-5-((3-chloro-5-trifluoromethyl-pyridin-2-yl)-3-(propyl)ether-1-yl)-Δ²-isoxazoline, which can be used for subsequent reactions. ¹³C-NMR: δ (CDCl₃, ppm)=24.9, 31.7, 42.5 (CH₂); 42.7 (N—CH₃); 55.8 (O—CH₃); 67.5 (OCH₂); 81.0 (CH); 112.5, 118.6 (HC—Ar); 118.7 (Cl—C-Py); 123.1 (F₃C-Py); 133.1 (C—Ar); 157.1 (O—C—Ar); 120.7 (F₃C—C-Py); 135.2, 142.4 (HC-Py); 136.1 (Cl—C—Ar); 140.8 (C—Ar); 161.2 (C-Py); 157.1 (O—C—Ar). MS (ES+): 494

### (A-6) 2-[3-chloro-5-(3,3-dichloro-allyl)-2-methoxy-phenyl]-4-chloromethyl-oxazol

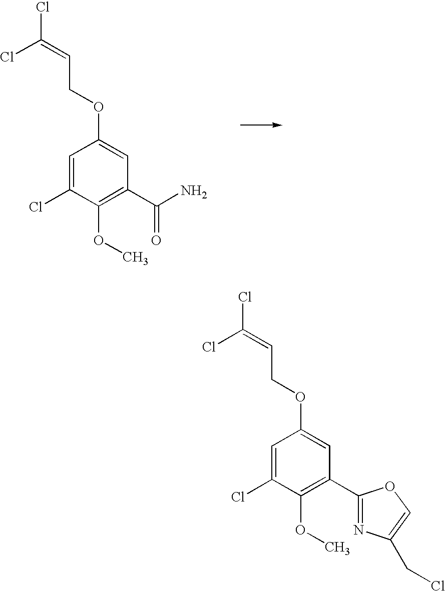

100 mg (0.32 mMol) of 3-chloro-5-(3,3-dichloro-allyloxy)-2-methoxy-benzamide and 82 mg (0.64 Mol) of 1,3-dichloroacetone are stirred together at 150° C. for one hour. Excess 1,3-dichloroacetone is separated by means of chromatography over silica gel (hexane:ethylacetate 4:1).

33.7 mg (61% according to LC-MS, 17% of the theory) of the 2-[3-chloro-5-(3,3-dichloro-allyl)-2-methoxy-phenyl]-4-chloromethyl-oxazole are obtained. MS(ES+)=383.

### APPLICATION EXAMPLES

### Example A

Heliothis armigera Test

Solvent: 7 Parts by weight of dimethylformamide

Emulsifier: 2 Parts by weight of alkyl-aryl polyglycol ether

For the production of a practical active agent preparation, one mixes 1 part by weight of active substance with the specified amount of solvent and emulsifier and dilutes the concentrate to the desired concentration with water containing an emulsifier.

Soy sprouts (Glycine max) are treated through immersion in the active substance preparation of the desired concentration and filled with Heliothis armigera larvae, while the leaves are still moist.

After the desired time the mortality in % is determined. In doing so, 100% means that all larvae were killed; 0% means that no larvae were killed.

With this test, for example, the compound according to production example I-1 shows a mortality of 100% after 7 days at an active substance concentration of 100 ppm.

### Example B

Meloidogyne Test

Solvent: 7 Parts by weight of dimethylformamide

Emulsifier: 2 Parts by weight of alkyl-aryl polyglycol ether

For the production of a practical active agent preparation, one mixes 1 part by weight of active substance with the specified amount of solvent and emulsifier and dilutes the concentrate to the desired concentration with water.

Vessels are filled with sand, active substance solution, Meloidogyne incognita egg/larvae suspension and lettuce seeds. The lettuce seeds germinate and the seedlings grow. The galls grow on the roots.

After the desired time, the nematicidal effect is determined by means of gall formation in %. In doing so, 100% means that no galls were found; 0% means that the number of galls on the treated plants corresponds to the untreated control.

With this test, for example, the compounds according to production examples I-1, I-14, I-28, I-29, I-51 and I-52 already exhibit good effectiveness at an active substance concentration of 20 ppm (Table A):

TABLE A
Meloidogyne test

| Active | Active substance | Effect in % |
|---|---|---|
| substance | concentration in ppm | after 14 days |
| (I-1) | 20 | 80 |
| (I-14) | 20 | 80 |
| (I-28) | 20 | 90 |
| (I-29) | 20 | 90 |
| (I-51) | 20 | 100 |
| (I-52) | 20 | 100 |

### Example C

Plutella Test

Solvent: 7 Parts by weight of dimethylformamide

Emulsifier: 2 Parts by weight of alkyl-aryl polyglycol ether

For the production of a practical active agent preparation, one mixes 1 part by weight of active substance with the specified amount of solvent and emulsifier and dilutes the concentrate to the desired concentration with water containing an emulsifier.

Cabbage leaves (Brassica oleracea) are treated through immersion in the active substance preparation of the desired concentration and filled with cabbage moth larvae (Plutella xylostella), while the leaves are still moist.

After the desired time the mortality in % is determined. In doing so, 100% means that all larvae were killed; 0% means that no larvae were killed.

With this test, for example, the compounds according to production examples I-1 and I-6 exhibit a mortality of 100 after 7 days at an active substance concentration of 100 ppm.

### Example D

Spodoptera exigua Test

Solvent: 7 Parts by weight of dimethylformamide

Emulsifier: 2 Parts by weight of alkyl-aryl polyglycol ether

For the production of a practical active agent preparation, one mixes 1 part by weight of active substance with the specified amount of solvent and emulsifier and dilutes the concentrate to the desired concentration with water containing an emulsifier.

Cabbage leaves (Brassica oleracea) are treated through immersion in the active substance preparation of the desired concentration and filled with fall armyworm larvae (Spodoptera exigua), while the leaves are still moist.

After the desired time the mortality in % is determined. In doing so, 100% means that all larvae were killed; 0% means that no larvae were killed.

With this test, for example, the compounds according to production examples I-1 and I-6 exhibit a mortality of 100 after 7 days at an active substance concentration of 100 ppm.

### Example E

Spodoptera frugiperda Test (Spray Treatment)

Solvent: 78 Parts by weight of acetone
1.5 Parts by weight of dimethylformamide

Emulsifier: 0.5 Parts by weight of alkyl-aryl polyglycol ether

For the production of a practical active agent preparation, one mixes 1 part by weight of active substance with the specified amount of solvent and emulsifier and dilutes the concentrate to the desired concentration with water containing an emulsifier.

Maize leaf slices (Zea mays) are sprayed with an active substance preparation of the desired concentration and filled with fall armyworm larvae (Spodoptera frugiperda) after drying.

After the desired time the effect in % is determined. In doing so, 100% means that all larvae were killed; 0% means that no larvae were killed.

With this test, for example, the compounds according to production examples I-1, I-5, I-6, I-7, I-9, I-10, I-11, I-14, I-15, I-47, I-48 and I-52 exhibit great effectiveness after 7 days at an active substance concentration of 110 ppm (Table B):

TABLE B
Spodoptera frugiperda test

| Active | Active substance | Effect in % |
|---|---|---|
| substance | concentration in ppm | after 7 days |
| (I-1) | 100 | 100 |
| (I-5) | 100 | 10 |
| (I-6) | 100 | 100 |
| (I-7) | 100 | 100 |
| (I-9) | 100 | 100 |
| (I-10) | 100 | 100 |
| (I-11) | 100 | 100 |
| (I-14) | 100 | 100 |
| (I-15) | 100 | 100 |
| (I-52) | 100 | 83 |
| (I-47) | 100 | 100 |
| (I-48) | 100 | 100 |

### Example F

Tetranychus Test (OP-Resistant/Spray Treatment)

Solvent: 78 Parts by weight of acetone
1.5 Parts by weight of dimethylformamide

Emulsifier: 0.5 Parts by weight of alkyl-aryl polyglycol ether

For the production of a practical active agent preparation, one mixes 1 part by weight of active substance with the specified amount of solvent and emulsifier and dilutes the concentrate to the desired concentration with water containing an emulsifier.

Bean leaf slices (Phaseolus vulgaris) that are affected by all stages of the common spider mite (Tetranychus urticae) are sprayed with a active substance preparation of the desired concentration.

After the desired time the effect in % is determined. In doing so, 100% means that all spider mites were killed; 0% means that no spider mites were killed.

With this test, for example, the compounds according to production examples I-1, I-7, I-9, I-10 and I-14 exhibit good effectiveness at an active substance concentration of 100 ppm (Table C):

TABLE C
Tetranychus test

| Active | Active substance | Effect in % |
|---|---|---|
| substance | concentration in ppm | after 7 days |
| (I-1) | 100 | 100 |
| (I-7) | 100 | 80 |
| (I-9) | 100 | 90 |
| (I-10) | 100 | 80 |
| (I-14) | 100 | 90 |

1. Compounds of the formula (I): 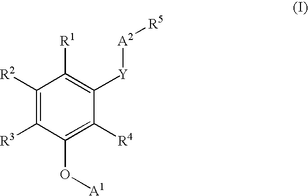
wherein
A¹ stands for one of the groupings —CH₂—CH═CCl₂, —CH₂—CH═CBr₂, —CH₂—CH═CClF, —CH₂—CF═CCl₂, —(CH₂)₂—CH═CF₂, —CH₂—CH═CBrCl, —CH₂—CH═CBrF, —CF═CH—CH═CH₂, —CH₂—CF═CF—CH═CH₂, —CH₂—CH═CClCF₃, —(CH₂)₂—CX₃ and —CH₂—CH═CClCH₃, whereby X stands for halogen,
or stands for one of the groupings 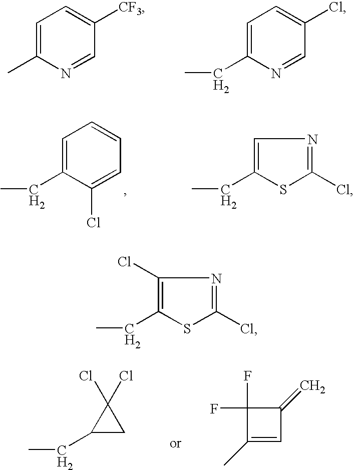
A² in each case stands for straight-chain or branched alkanediyl or alkenediyl with up to 8 carbon atoms in each case, which optionally contain an oxygen atom, a sulphur atom or a grouping selected from SO, SO₂, NH or N(C₁-C₄-alkyl) at the start, at the end or within the carbon chain,
R¹ stands for hydrogen, nitro, hydroxy, amino, cyano, halogen, optionally for alkyl, alkoxy, alkylthio, alkylamino, dialkylamino, alkylcarbonylamino or alkoximinoalkyl with 1 to 10 carbon atoms in the alkyl groups substituted in each case by cyano, halogen, C₁-C₆-alkylsulfinyl, C₁-C₆-alkylsulfonyl or C₁-C₆-alkoxy, for C₁-C₆-alkylcarbonyloxy, for C₁-C₆-alkoxycarbonyloxy, for C₃-C₆-cycloalkoxycarbonyloxy, for C₁-C₆-dialkyaminocarbonyloxy, optionally for aryloxy, arylthio or arylalkyl with 6 or 10 carbon atoms in each case in the aryl groups and optionally 1 to 4 carbon atoms in the alkyl part, substituted in each case by nitro, hydroxy, amino, cyano, halogen, C₁-C₆-alkyl, C₁-C₆-halogen alkyl, C₁-C₆-alkoxy or C₁-C₆-halogenalkoxy, optionally for heterocyclyloxy or heterocyclylthio with up to 10 carbon atoms, up to 4 nitrogen atoms and optionally an oxygen or sulphur atom in each case, substituted in each case by nitro, hydroxy, amino, cyano, halogen, C₁-C₆-alkyl, C₁-C₆-halogenalkyl, C₁-C₆-alkoxy or C₁-C₆-halogenalkoxy, or stands for the grouping —O-A¹, whereby the A¹ has the meaning given above, or stands for the grouping —N(R,R′), whereby R and R′ together stand for straight-chain or branched alkanediyl with up to 8 carbon atoms, which optionally contains an oxygen atom, a sulphur atom or a grouping selected from SO, SO₂, NH or N(C₁-C₄-alkyl) at the beginning of, at the end of or within the carbon chain,
R² stands for hydrogen, nitro, hydroxy, amino, cyano, cyanato, thiocyanato, formyl, halogen, optionally for alkyl, alkoxy, alkylthio, alkylsulfinyl, alkylsulfonyl, alkylamino, dialkylamino or alkylcarbonylamino with 1 to 6 carbon atoms in each case in the alkyl groups, substituted in each case by cyano, halogen or C₁-C₆-alkoxy, for C₁-C₆-alkyl-carbonyl, C₁-C₆-alkoxy-carbonyl, C₁-C₆-alkoximinoformyl, C₁-C₆-alkoximino-acetyl, or for C₂-C₆-alkenyl or C₂-C₆-alkinyl,
R³ stands for hydrogen, nitro, hydroxy, amino, cyano, halogen, optionally for alkyl, alkoxy, alkylthio, alkylamino, dialkylamino or alkylcarbonylamino with 1 to 6 carbon atoms in each case in the alkyl groups, substituted by cyano, halogen or C₁-C₆-alkoxy,
R⁴ stands for hydrogen, nitro, hydroxy, amino, cyano, halogen, optionally for alkyl, alkoxy, alkylthio, alkylamino, dialkylamino or alkylcarbonylamino with 1 to 6 carbon atoms in each case in the alkyl groups, substituted by cyano, halogen or C₁-C₆-alkoxy,
R⁵ stands for hydrogen, for aryl with 6 or 10 carbon atoms in the aryl group optionally substituted in each case by nitro, hydroxy, amino, cyano, halogen, C₁-C₆-alkyl, C₁-C₆-halogenalkyl, C₁-C₆-alkoxy, C₁-C₆-halogenalkoxy, C₁-C₂-alkylendioxy, C₁-C₂-haloalkylendioxy, C₁-C₆-alkylthio, C₁-C₆-halogenalkylthio, C₁-C₆-alkoxyimino-C₁-C₆-alkyl, or for heteroaryl with up to 10 carbon atoms, up to 4 nitrogen atoms and optionally an oxygen or sulphur atom, optionally substituted the same or differently one to three times, whereby the substituents can be selected from the following group:
nitro, hydroxy, amino, cyano, halogen, C₁-C₆-alkyl, C₁-C₆-halogenalkyl, C₁-C₆-alkoxy, C₁-C₆-halogenalkoxy, C₁-C₆-alkylcarbonyl, C₂-C₆-alkoxycarbonyl, C₂-C₆-alkenyl, C₂-C₆-alkenyloxy, C₂-C₆-halogenalkenyl, C₂-C₆-halogenalkenyloxy, C₂-C₆-alkinyl, C₂-C₆-alkinyloxy, C₁-C₂-alkylendioxy, C₁-C₂-haloalkylendioxy, C₁-C₆-alkylthio, C₁-C₆-halogenalkylthio, C₁-C₆-alkoxyimino-C₁-C₆-alkyl and the grouping 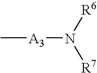
wherein
A³ stands for a single bond, or stands for C₁-C₆-alkanediyl, which is optionally substituted by one to six equivalent or different substituents from the group C₁-C₃-halogenalkyl, C₃-C₈-cycloalkyl and C₃-C₈-cycloalkyl-C₁-C₆-alkyl,
R⁶ stands for hydrogen, cyano, hydroxy, C₁-C₆-alkyl, C₃-C₈-cycloalkyl, C₃-C₈-cycloalkyl-C₁-C₆-alkyl, C₁-C₆-halogenalkyl, C₁-C₆-alkoxy, C₁-C₆-halogenalkoxy, C₂-C₆-alkenyloxy, C₂-C₆-halogenalkenyloxy, C₂-C₆-alkinyloxy, —C(═O)R⁸, —C(═O)R⁸, or optionally for phenyl or benzyl substituted one to five times, the same or differently, in each case in the aryl part by halogen, C₁-C₆-alkyl, C₁-C₆-halogenalkyl, C₁-C₆-alkoxy, C₁-C₆-halogenalkoxy, hydroxy, cyano or nitro,
R⁷ stands for hydrogen, cyano, hydroxy, C₁-C₆-alkyl, C₃-C₈-cycloalkyl, C₃-C₈-cycloalkyl-C₁-C₆-alkyl, C₁-C₆-halogenalkyl, —C(═O)R⁸, —C(═S)R⁸, or for phenyl or benzyl optionally substituted one to five times, the same or differently, in each case in the aryl part by halogen, C₁-C₆-alkyl, C₁-C₆-halogenalkyl, C₁-C₆-alkoxy, C₁-C₆-halogenalkoxy, hydroxy, cyano or nitro, or
R⁷ together with R⁶ stands for C₄-C₈-alkanediyl or C₄-C₈-alkylenediyl optionally substituted in each case one to four times, the same or differently, by C₁-C₆-alkyl, C₃-C₈-cycloalkyl-C₁-C₆-alkyl, C₁-C₆-halogenalkyl, cyano or C₁-C₆-alkylcarbonyl, whereby a CH₂ group can be optionally replaced by O, S or NR⁹, or
R⁷ stands for —C(═O)R⁸ or —C(═S)R⁸, whereby R⁶ und R⁸ then stand together for C₂-C₈-alkanediyl or C₂-C₈-alkylenediyl optionally substituted one to four times, the same or differently, by C₁-C₆-alkyl, C₃-C₈-cycloalkyl-C₁-C₆-alkyl, C₁-C₆-halogenalkyl, cyano or C₁-C₆-alkylcarbonyl, whereby a CH₂ group can be optionally replaced by O, S or NR⁹, or
R⁶ and R⁷ independently from one another stand for —C(═O)R⁸ or —C(═S)R⁸, and both the moieties R⁸ together stand in each case for straight-chain or branched C₂-C₈-alkanediyl or C₂-C₈-alkylenediyl optionally substituted one to four times, the same or differently, by C₁-C₆-alkyl, C₃-C₈-cycloalkyl-C₁-C₆-alkyl, C₁-C₆-halogenalkyl, cyano or C₁-C₆-alkylcarbonyl, and wherein a CH₂ group can be optionally replaced by O, S or NR⁹,
R⁸ stands for C₁-C₆-alkyl, C₁-C₆-halogenalkyl, C₂-C₆-alkenyl, C₂-C₆-halogenalkenyl, C₂-C₆-alkinyl, C₁-C₆-alkoxy, C₁-C₆-halogenalkoxy, C₂-C₆-alkenyloxy, C₂-C₆-halogenalkenyloxy, C₂-C₆-alkinyloxy, C₃-C₆-cycloalkyl, for phenyl or benzyl optionally substituted in each case one to three times, the same or differently, in the aryl part by halogen, cyano, nitro, C₁-C₆-alkyl, C₁-C₆-halogenalkyl, C₁-C₆-alkylcarbonyl, C₂-C₆-alkenyl, C₂-C₆-halogenalkenyl, C₂-C₆-alkynyl, C₁-C₆-alkoxy, C₁-C₆-halogenalkoxy, C₁-C₆-alkoxycarbonyl, C₁-C₃-halogenalkoxycarbonyl or C₂-C₆-halogenalkenyloxy, and
R⁹ stands for hydrogen, C₁-C₆-alkyl, C₁-C₃-halogenalkyl, C₁-C₃-halogenalkylcarbonyl, C₁-C₆-alkoxyalkyl, C₁-C₆-alkylcarbonyl or C₃-C₈-cycloalkyl, and
Y stands for a heterocyclic grouping selected from the following list, connected with the adjacent groupings at two different positions, 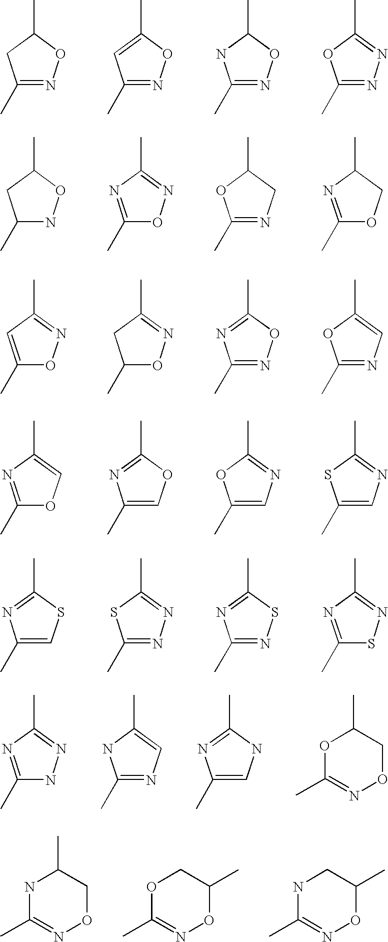
whereby these heterocyclic groupings can be optionally substituted in each case by one or two substituents from the series nitro, hydroxy, amino, cyano, halogen, C₁-C₆ alkyl, C₁-C₆ halogenalkyl, C₁-C₆ alkoxy, C₁-C₆ halogenalkoxy, C₁-C₆ alkylthio, and C₁-C₆ halogenalkythio.

2. Compounds of the formula (I) according to claim 1, characterised in that
A¹ stands for one of the following groupings
—CH₂—CH═CCl₂, —CH₂—CH═CBr₂, —CH₂—CH═CClF, —CH₂—CF═CCl₂, —(CH₂)₂—CH═CF₂, —CH₂—CH═CBrCl, —CH₂—CH═CBrF, —CF═CH—CH═CH₂, —CH₂—CF═CF—CH═CH₂, —CH₂—CH═CClCF₃ and —CH₂—CH═CClCH₃,
or stands for the grouping 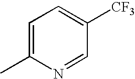
A² stands in each case for straight-chain or branched alkanediyl or alkenediyl with up to 4 carbon atoms in each case, which optionally contains an oxygen atom, a sulphur atom or a grouping selected from SO, SO₂, NH or N(C₁-C₃-alkyl) at the beginning of, at the end of or within the carbon chain,
R¹ stands for hydrogen, nitro, hydroxy, amino, cyano, halogen, for alkyl, alkoxy, alkylthio, alkylamino, dialkylamino, alkylcarbonylamino or alkoximinoalkyl with 1 to 8 carbon atoms in the alkyl groups optionally substituted in each case by cyano, halogen, C₁-C₃-alkylsulphinyl, C₁-C₃-alkylsulphonyl or C₁-C₅-alkoxy, for C₁-C₃-alkylcarbonyloxy, for C₁-C₃-alkoxycarbonyloxy, for C₃-C₅-cycloalkoxycarbonyloxy, for C₁-C₆-dialkyaminocarbonyloxy, optionally for aryloxy, arylthio or aryl-alkyl with 6 or 10 carbon atoms in each case in the aryl groups and optionally 1 to 3 carbon atoms in the alkyl part, for heterocyclooxy or heterocyclylthio with up to 9 carbon atoms, 1 to 4 nitrogen atoms and/or an oxygen or sulphur atom in each case, optionally substituted in each case by nitro, hydroxy, amino, cyano, halogen, C₁-C₅-alkyl, C₁-C₅-halogen alkyl, C₁-C₅-alkoxy or C₁-C₅-halogenalkoxy, or stands for the grouping —O-A¹, whereby A¹ has the meaning given above, or stands for the grouping —N(R,R′), whereby R and R′ together stand for straight-chain or branched alkanediyl with up to 6 carbon atoms, which optionally contains an oxygen atom, a sulphur atom or a grouping selected from SO, SO₂, NH or N(C₁-C₄-alkyl) at the beginning of, at the end of or within the carbon chain,
R² stands for hydrogen, nitro, hydroxy, amino, cyano, cyanato, thiocyanato, formyl, halogen, for alkyl, alkoxy, alkylthio, alkylamino, dialkylamino or alkylcarbonylamino with 1 to 5 carbon atoms in each case in the alkyl groups, optionally substituted in each case by cyano, halogen or C₁-C₅-alkoxy, for C₁-C₅-alkyl-carbonyl, C₁-C₅-alkoxy-carbonyl, C₁-C₅-alkoximinoformyl, C₁-C₅-alkoximino-acetyl, or for C₂-C₅-alkenyl or C₂-C₅-alkinyl,
R³ stands for hydrogen, nitro, halogen, for alkyl, alkoxy, alkylthio or alkylamino with 1 to 5 carbon atoms in each case in the alkyl groups, optionally substituted by cyano, halogen or C₁-C₅-alkoxy,
R⁴ stands for hydrogen, nitro, halogen, for alkyl, alkoxy, alkylthio or alkylamino with 1 to 5 carbon atoms in each case in the alkyl groups, optionally substituted by cyano, halogen or C₁-C₅-alkoxy,
R⁵ stands for hydrogen, for aryl with 6 or 10 carbon atoms in the aryl group optionally substituted in each case by nitro, hydroxy, amino, cyano, halogen, C₁-C₅-alkyl, C₁-C₅-halogenalkyl, C₁-C₅-alkoxy, C₁-C₅-halogenalkoxy, C₁-C₂-alkylendioxy, C₁-C₂-haloalkylendioxy, C₁-C₅-alkylthio, C₁-C₅-halogenalkylthio, C₁-C₅-alkoxyimino-C₁-C₅-alkyl, or for heteroaryl with up to 9 carbon atoms, up to 1 nitrogen atom and optionally an oxygen or sulphur atom, optionally substituted the same or differently one to three times, whereby the substituents can be selected from the following group of substituents:
nitro, hydroxy, amino, cyano, halogen, C₁-C₅-alkyl, C₁-C₅-halogenalkyl, C₁-C₅-alkoxy, C₁-C₅-halogenalkoxy, C₁-C₅-alkylcarbonyl, C₂-C₅-alkoxycarbonyl, C₂-C₅-alkenyl, C₂-C₅-alkenyloxy, C₂-C₅-halogenalkenyl, C₂-C₅-halogenalkenyloxy, C₂-C₅-alkinyl, C₂-C₅-alkinyloxy, C₁-C₅-alkylendioxy, C₁-C₂-haloalkylendioxy, C₁-C₅-alkylthio, C₁-C₅-halogenalkylthio, C₁-C₅-alkoxyimino-C₁-C₅-alkyl and the grouping 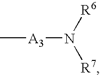
wherein
A³ stands for a single bond, or stands for C₁-C₆-alkanediyl, which is optionally substituted by one to six equivalent or different substituents from the group C₁-C₃-halogenalkyl, C₃-C₈-cycloalkyl and C₃-C₈-cycloalkyl-C₁-C₆-alkyl,
R⁶ stands for hydrogen, cyano, hydroxy, C₁-C₅-alkyl, C₃-C₆-cycloalkyl, C₃-C₆-cycloalkyl-C₁-C₅-alkyl, C₁-C₅-halogenalkyl, C₁-C₅-alkoxy, C₁-C₅-halogenalkoxy, C₂-C₅-alkenyloxy, C₂-C₅-halogenalkenyloxy, C₂-C₅-alkinyloxy, —C(═O)R⁸, —C(═O)R⁸, or for phenyl or benzyl optionally substituted one to five times, the same or differently, in each case in the aryl part by halogen, C₁-C₅-alkyl, C₁-C₅-halogenalkyl, C₁-C₅-alkoxy, C₁-C₅-halogenalkoxy, hydroxy, cyano or nitro,
R⁷ stands for hydrogen, cyano, C₁-C₅-alkyl, C₃-C₆-cycloalkyl, C₃-C₆-cycloalkyl-C₁-C₅-alkyl, C₁-C₅-halogenalkyl, —C(═O)R⁸, —C(═S)R⁸, or for phenyl or benzyl optionally substituted one to five times, the same or differently, in each case in the aryl part by halogen, C₁-C₅-alkyl, C₁-C₅-halogenalkyl, C₁-C₅-alkoxy, C₁-C₅-halogenalkoxy, hydroxy, cyano or nitro,
R⁷ together with R⁶ stands for C₄-C₆-alkanediyl or C₄-C₆-alkylenediyl optionally substituted in each case one to four times, the same or differently, by C₁-C₅-alkyl, C₃-C₆-cycloalkyl-C₁-C₅-alkyl, C₁-C₅-halogenalkyl, cyano or C₁-C₅-alkylcarbonyl, whereby a CH₂ group can be optionally replaced by O, S or NR⁹, or
R⁷ stands for —C(═O)R⁸ or —C(═S)R⁸, whereby R⁶ and R⁸ then stand together for C₂-C₄-alkanediyl or C₂-C₄-alkylenediyl optionally substituted one to four times, the same or differently, by C₁-C₅-alkyl, C₃-C₆-cycloalkyl-C₁-C₅-alkyl, C₁-C₅-halogenalkyl, cyano or C₁-C₅-alkylcarbonyl, whereby a CH₂ group can be optionally replaced by O, S or NR⁹, or
R⁶ and R⁷ independently from one another stand for —C(═O)R⁸ or —C(═S)R⁸, whereby both of the moieties R⁸ together stand in each case for straight-chain or branched C₂-C₄-alkanediyl or C₂-C₄-alkylenediyl optionally substituted one to four times, the same or differently, by C₁-C₅-alkyl, C₃-C₆-cycloalkyl-C₁-C₅-alkyl, C₁-C₅-halogenalkyl, cyano or C₁-C₅-alkylcarbonyl, and wherein a CH₂ group can be optionally replaced by O, S or NR⁹,
R⁸ stands for C₁-C₅-alkyl, C₁-C₅-halogenalkyl, C₂-C₅-alkenyl, C₂-C₅-halogenalkenyl, C₂-C₅-alkinyl, C₁-C₅-alkoxy, C₁-C₅-halogenalkoxy, C₂-C₅-alkenyloxy, C₂-C₅-halogenalkenyloxy, C₂-C₅-alkinyloxy, C₃-C₅-cycloalkyl, for phenyl or benzyl optionally substituted in each case one to three times, the same or differently, in the aryl part by halogen, cyano, nitro, C₁-C₅-alkyl, C₁-C₅-halogenalkyl, C₁-C₅-alkylcarbonyl, C₂-C₅-alkenyl, C₂-C₅-halogenalkenyl, C₂-C₅-alkinyl, C₁-C₅-alkoxy, C₁-C₅-halogenalkoxy, C₁-C₅-alkoxycarbonyl, C₁-C₃-halogenalkoxycarbonyl or C₂-C₅-halogenalkenyloxy,
R⁹ stands for hydrogen, C₁-C₅-alkyl, C₁-C₃-halogenalkyl, C₁-C₃-halogenalkylcarbonyl, C₁-C₅-alkoxyalkyl, C₁-C₅-alkylcarbonyl or C₃-C₆-cycloalkyl, and
Y stands for a heterocyclic grouping selected from the following list, connected with the adjacent groupings at two different positions, 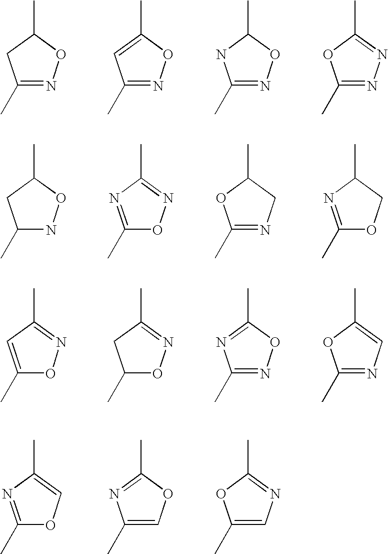
whereby these heterocyclic groupings can be optionally substituted in each case by one or two substituents from the series nitro, hydroxy, amino, cyano, halogen, C₁-C₅ alkyl, C₁-C₅-halogenalkyl, C₁-C₅-alkoxy, C₁-C₅-halogenalkoxy, C₁-C₅-alkylthio, and C₁-C₅-halogenalkythio.

3. Compounds of the formula (I) according to claim 1, characterised in that
A¹ stands for one of the following groupings,
—CH₂—CH═CCl₂, —CH₂—CH═CBr₂, —CH₂—CH═CClF, —CH₂—CH═CBrCl,
A² stands for the following listed alkanediyl groupings
—CH₂—, —CH₂CH₂—, —CH(CH₃)—CH₂—, —CH₂CH(CH₃)—, —CH₂CH₂CH₂—, —CH(CH₃)CH₂CH₂—, —CH₂CH(CH₃)CH₂—, —CH₂CH₂CH(CH₃)—, —CH₂CH₂CH₂CH₂—, —CH₂CH₂CH₂CH₂CH₂—,
which can optionally contain an oxygen atom, a sulphur atom or a grouping selected from SO, SO₂, NH or N(methyl) at the beginning of, at the end of or within the carbon chain in each case,
R¹ stands for hydrogen, nitro, hydroxy, amino, cyano, fluorine, chlorine, bromine, iodine, for methyl, ethyl, n- or i-propyl, n-, i-, s- or t-butyl, methoxy, ethoxy, n- or i-propoxy, n-, i-, s- or t-butoxy, methylthio, ethylthio, n- or i-propylthio, n-, i-, s- or t-butylthio, methylamino, ethylamino, n- or i-propylamino, n-, i-, s- or t-butylamino, dimethylamino, diethylamino, dipropylamino, acetylamino, propionylamino, n- or i-butyroylamino, methoximinomethyl, ethoximinomethyl, methoximinoethyl or ethoximinoethyl optionally substituted in each case by cyano, fluorine, chlorine, methylsulfinyl, methylsulfonyl, methoxy, ethoxy, n- or i-propoxy, for methylcarbonyloxy, ethylcarbonyloxy, n- or i-propylcarbonyloxy, methoxycarbonyloxy, ethoxycarbonyloxy, n- or i-propoxycarbonyloxy, cyclopropoxycarbonyloxy, cyclobutoxycarbonyloxy, cyclopentoxycarbonyloxy, cyclohexoxycarbonyloxy, for phenoxy, naphthyloxy, phenylthio, naphthylthio, benzyl or phenylethyl optionally substituted in each case by nitro, hydroxy, amino, cyano, fluorine, chlorine, bromine, iodine, methyl, ethyl, n- or i-propyl, n-, i-, s- or t-butyl, difluoromethyl, trifluormethyl, chlorodifluoromethyl, fluoroethyl, difluoroethyl, trifluorethyl, chloroethyl, dichloroethyl, trichloroethyl, methoxy, ethoxy, n- or i-propoxy, n-, i-, s- or t-butoxy, fluoromethoxy, difluoromethoxy, trifluoromethoxy, chlorodifluoromethoxy, fluoroethoxy, difluoroethoxy, trifluoroethoxy, chloroethoxy or dichloroethoxy, for heterocyclyloxy or heterocyclylthio with up to 9 carbon atoms, 1 to 4 nitrogen atoms and/or an oxygen or sulphur atom in each case, substituted in each case by nitro, hydroxy, amino, cyano, fluorine, chlorine, bromine, iodine, methyl, ethyl, n- or i-propyl, n-, i-, s- or t-butyl, difluoromethyl, trifluormethyl, chlorodifluoromethyl, fluoroethyl, difluoroethyl, trifluorethyl, chloroethyl, dichloroethyl, trichloroethyl, methoxy, ethoxy, n- or i-propoxy, n-, i-, s- or t-butoxy, fluoromethoxy, difluoromethoxy, trifluoromethoxy, chlorodifluoromethoxy, fluoroethoxy, difluoroethoxy, trifluoroethoxy, chloroethoxy or dichloroethoxy, or for the grouping —O-A¹, whereby A¹ has the meaning provided above, or for the grouping —N(R,R′), whereby R and R′ together with the N atom to which they are connected stand for pyrrolidinyl, piperidinyl, morpholinyl or piperazinyl optionally substituted in each case once or twice by methyl and/or ethyl,
R² stands for hydrogen, nitro, cyano, cyanato, thiocyanato, formyl, fluorine, chlorine, bromine, iodine, for methyl, ethyl, n- or i-propyl, n-, i-, s- or t-butyl, methoxy, ethoxy, n- or i-propoxy, n-, i-, s- or t-butoxy, methylthio, ethylthio, n- or i-propylthio, n-, i-, s- or t-butylthio, methylamino, ethylamino, n- or i-propylamino, n-, i-, s- or t-butylamino, dimethylamino, diethylamino, acetylamino, propionylamino, n- or i-butyroylamino, acetyl, propionyl, n- or i-butyroyl, methoxycarbonyl, ethoxycarbonyl, n- or i-propoxycarbonyl, methoximinoformyl, ethoximinoformyl, methoximinoacetyl or ethoximinoacetyl optionally substituted in each case by cyano, fluorine, chlorine, methoxy, ethoxy, n- or i-propoxy,
R³ stands for hydrogen, nitro, fluorine, chlorine, bromine, iodine, for methyl, ethyl, n- or i-propyl, n-, i-, s- or t-butyl, methoxy, ethoxy, n- or i-propoxy, n-, i-, s- or t-butoxy, methylthio, ethylthio, n- or i-propylthio, n-, i-, s- or t-butylthio, methylamino, ethylamino, n- or i-propylamino, n-, i-, s- or t-butylamino, optionally substituted in each case by cyano, fluorine, chlorine, methoxy, ethoxy, n- or i-propoxy,
R⁴ stands for hydrogen, nitro, fluorine, chlorine, bromine, iodine, for methyl, ethyl, n- or i-propyl, n-, i-, s- or t-butyl, methoxy, ethoxy, n- or i-propoxy, n-, i-, s- or t-butoxy, methylthio, ethylthio, n- or i-propylthio, n-, i-, s- or t-butylthio, methylamino, ethylamino, n- or i-propylamino, n-, i-, s- or t-butylamino, optionally substituted in each case by cyano, fluorine, chlorine, methoxy, ethoxy, n- or i-propoxy,
R⁵ stands for hydrogen, for phenyl or naphthyl substituted by nitro, hydroxy, amino, cyano, fluorine, chlorine, bromine, iodine, methyl, ethyl, n- or i-propyl, n-, i-, s- or t-butyl, difluoromethyl, trifluoromethyl, chlorodifluoromethyl, fluoroethyl, difluoroethyl, trifluoroethyl, chloroethyl, dichloroethyl, trichloroethyl, methoxy, ethoxy, n- or i-propoxy, n-, i-, s- or t-butoxy, fluoromethoxy, difluoromethoxy, trifluoromethoxy, chlorodifluoromethoxy, fluoroethoxy, difluoroethoxy, trifluoroethoxy, chloroethoxy or dichloroethoxy, C₁-C₂-alkylendioxy, C₁-C₂-fluoroalkylendioxy, methylthio, ethylthio, n- or i-propylthio, n-, i-, s- or t-butylthio, difluoromethylthio, trifluoromethylthio, chlorodifluoromethylthio, methoximinomethyl, ethoximinomethyl, methoximinoethyl or ethoximinoethyl, or for optionally substituted heteroaryl from the series furyl, thienyl, pyrrolyl, pyrazolyl, oxazolyl, isoxazolyl, thiazolyl, isothiazolyl, oxadiazolyl, thiadiazolyl, triazolyl, pyridinyl and pyrimidinyl, whereby the substituents can be selected from the following group:
nitro, hydroxy, amino, cyano, fluorine, chlorine, bromine, iodine, methyl, ethyl, n- or i-propyl, n-, i-, s- or t-butyl, difluoromethyl, trifluoromethyl, chlorodifluoromethyl, fluoroethyl, difluoroethyl, trifluoroethyl, chloroethyl, dichloroethyl, trichloroethyl, methoxy, ethoxy, n- or i-propoxy, n-, i-, s- or t-butoxy, fluoromethoxy, difluoromethoxy, trifluoromethoxy, chlorodifluoromethoxy, fluoroethoxy, difluoroethoxy, trifluoroethoxy, chloroethoxy or dichloroethoxy, methylcarbonyl, ethylcarbonyl, n- or i-propylcarbonyl, n-, i-, s- or t-butylcarbonyl, ethoxycarbonyl, n- or i-propoxycarbonyl, n-, i-, s- or t-butoxycarbonyl, ethenyl, 2-propenyl, 1-butenyl, 2-butenyl, 3-butenyl, 1-pentenyl, 2-pentenyl, 3-pentenyl, ethenyloxy, 2-propenyloxy, 1-butenyloxy, 2-butenyloxy, 3-butenyloxy, 1-pentenyloxy, 2-pentenyloxy, 3-pentenyloxy, fluoroethenyl, difluoroethenyl, trifluoroethenyl, chloroethenyl, dichloroethenyl, trichloroethenyl, fluoroethenyloxy, difluoroethenyloxy, trifluoroethenyloxy, chloroethenyloxy, dichloroethenyloxy, trichloroethenyloxy, ethinyl, 1-propinyl, 2-propinyl, 1-butinyl, 2-butinyl, 3-butinyl, 1-pentinyl, 2-pentinyl, 3-pentinyl, C₁-C₂-alkylendioxy, C₁-C₂-fluoroalkylendioxy, methylthio, ethylthio, n- or i-propylthio, n-, i-, s- or t-butylthio, difluoromethylthio, trifluoromethylthio, chlorodifluoromethylthio, methoximinomethyl, ethoximinomethyl, methoximinoethyl or ethoximinoethyl and the grouping 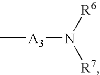
wherein
A³ stands for a single bond or for one of the groups
—CH₂—, —CH₂CH₂—, —CH₂—CH₂—CH₂—, —CH₂—CH₂—CH₂—CH₂—, —CH₂—CH₂—CH₂—CH₂—CH₂—, —CH(CH₃)—, —CH(CH₃)CH₂—CH₂—, —CH(C₂H₅)—, —C(CH₃)₂—, —CH(CH₃)CH₂—, —CH(CH₃)CH(CH₃)— and —CH₂C(CH₃)₂—CH₂—,
which can be optionally substituted with one to four equivalent or different substituents from the group difluoromethyl, trifluoromethyl, chlorodifluoromethyl, fluoroethyl, difluoroethyl, trifluoroethyl, chloroethyl, dichloroethyl, trichloroethyl, cyclopropyl, cyclobutyl, cyclopentyl, cyclohexyl, cyclopropylmethyl, cyclopropylethyl, cyclobutylmethyl, cyclobutylethyl, cyclopentylmethyl, cyclopentylethyl, cyclohexylmethyl and cyclohexylethyl,
R⁶ stands for hydrogen, cyano, hydroxy, methyl, ethyl, n- or i-propyl, n-, i-, s- or t-butyl, cyclopropyl, cyclobutyl, cyclopentyl, cyclohexyl, cyclopropylmethyl, cyclopropylethyl, cyclobutylmethyl, cyclobutylethyl, cyclopentylmethyl, cyclopentylethyl, cyclohexylmethyl and cyclohexylethyl, difluoromethyl, trifluoromethyl, chlorodifluoromethyl, fluoroethyl, difluoroethyl, trifluoroethyl, chloroethyl, dichloroethyl, trichloroethyl, methoxy, ethoxy, n- or i-propoxy, n-, i-, s- or t-butoxy, fluoromethoxy, difluoromethoxy, trifluoromethoxy, chlorodifluoromethoxy, fluoroethoxy, difluoroethoxy, trifluoroethoxy, chloroethoxy or dichloroethoxy, ethenyloxy, 2-propenyloxy, 1-butenyloxy, 2-butenyloxy, 3-butenyloxy, 1-pentenyloxy, 2-pentenyloxy, 3-pentenyloxy, fluoroethenyloxy, difluoroethenyloxy, trifluoroethenyloxy, chloroethenyloxy, dichloroethenyloxy, trichloroethenyloxy, —C(═O)R⁸, —C(═O)R⁸, or for phenyl or benzyl optionally substituted in each case one to five times, the same or differently, in the aryl part by fluorine, chlorine, bromine, methyl, ethyl, n- or i-propyl, n-, i-, s- or t-butyl difluoromethyl, trifluoromethyl, chlorodifluoromethyl, fluoroethyl, difluoroethyl, trifluoroethyl, chloroethyl, dichloroethyl, trichloroethyl, methoxy, ethoxy, n- or i-propoxy, n-, i-, s- or t-butoxy, fluoromethoxy, difluoromethoxy, trifluoromethoxy, chlorodifluoromethoxy, fluoroethoxy, difluoroethoxy, trifluoroethoxy, chloroethoxy or dichloroethoxy, hydroxy, cyano or nitro.
R⁷ particularly preferably stands for hydrogen, cyano, methyl, ethyl, n- or i-propyl, n-, i-, s- or t-butyl, cyclopropyl, cyclobutyl, cyclopentyl, cyclohexyl, cyclopropylmethyl, cyclopropylethyl, cyclobutylmethyl, cyclobutylethyl, cyclopentylmethyl, cyclopentylethyl, cyclohexylmethyl and cyclohexylethyl, difluoromethyl, trifluoromethyl, chlorodifluoromethyl, fluoroethyl, difluoroethyl, trifluoroethyl, chloroethyl, dichloroethyl, trichloroethyl, —C(═O)R⁸, —C(═S)R⁸, or for phenyl or benzyl optionally substituted in each case one to five times, the same or differently, in the aryl part by halogen, methyl, ethyl, n- or i-propyl, n-, i-, s- or t-butyl, difluoromethyl, trifluoromethyl, chlorodifluoromethyl, fluoroethyl, difluoroethyl, trifluoroethyl, chloroethyl, dichloroethyl, trichloroethyl, methoxy, ethoxy, n- or i-propoxy, n-, i-, s- or t-butoxy, fluoromethoxy, difluoromethoxy, trifluoromethoxy, chlorodifluoromethoxy, fluoroethoxy, difluoroethoxy, trifluoroethoxy, chloroethoxy or dichloroethoxy, hydroxy, cyano or nitro, or
R⁷ together with R⁶ stands for alkanediyl or alkylenediyl from the series —CH₂—, —CH₂CH₂—, —CH₂—CH₂—CH₂—, —CH₂—CH₂—CH₂—CH₂—, —CH₂—CH₂—CH₂—CH₂—CH₂—, —CH(CH₃)—, —CH(CH₃)CH₂—CH₂—, —CH(C₂H₅)—, —C(CH₃)₂—, —CH(CH₃)CH₂—, —CH(CH₃)CH(CH₃)—, —CH₂C(CH₃)₂—CH₂—, —CH═CH—, —CH═CH—CH₂—, —CH₂—CH═CH—CH₂—, —CH₂—CH═CH—CH₂—CH₂— and —CH(CH₃)CH═CH—, optionally substituted one to four times, the same or differently, by methyl, ethyl, n- or i-propyl, n-, i-, s- or t-butyl, cyclopropylmethyl, cyclopropylethyl, cyclobutylmethyl, cyclobutylethyl, cyclopentylmethyl, cyclopentylethyl, cyclohexylmethyl and cyclohexylethyl, difluoromethyl, trifluoromethyl, chlorodifluoromethyl, fluoroethyl, difluoroethyl, trifluoroethyl, chloroethyl, dichloroethyl, trichloroethyl, cyano or methylcarbonyl, ethylcarbonyl, n- or i-propylcarbonyl, n-, i-, s- or t-butylcarbonyl, whereby a CH₂ group can be optionally replaced by O, S or NR⁹,
R⁷ stands for —C(═O)R⁸ or —C(═S)R⁸, whereby R⁶ and R⁸ together stand for alkanediyl or alkylenediyl from the series —CH₂—, —CH₂CH₂—, —CH₂—CH₂—CH₂—, —CH₂—CH₂—CH₂—CH₂—, —CH(CH₃)—, —CH(CH₃)CH₂—CH₂—, —CH(C₂H₅)—, —C(CH₃)₂—, —CH(CH₃)CH₂—, —CH(CH₃)CH(CH₃)—, —CH═CH—, —CH═CH—CH₂—, —CH₂—CH═CH—CH₂—, and —CH(CH₃)CH═CH—, optionally substituted in each case one to four times, the same or differently, by methyl, ethyl, n- or i-propyl, n-, i-, s- or t-butyl, cyclopropylmethyl, cyclopropylethyl, cyclobutylmethyl, cyclobutylethyl, cyclopentylmethyl, cyclopentylethyl, cyclohexylmethyl, and cyclohexylethyl, difluoromethyl, trifluoromethyl, chlorodifluoromethyl, fluoroethyl, difluoroethyl, trifluoroethyl, chloroethyl, dichloroethyl, trichloroethyl, cyano or methylcarbonyl, ethylcarbonyl, n- or i-propylcarbonyl, n-, i-, s- or t-butylcarbonyl, and whereby a CH₂ group can be optionally replaced by O, S or NR⁹,
R⁶ and R⁷ stand for —C(═O)R⁸ or —C(═S)R⁸, whereby both of the moieties R⁸ stand for alkanediyl or alkylenediyl from the series —CH₂—, —CH₂CH₂—, —CH₂—CH₂—CH₂—, —CH₂—CH₂—CH₂—CH₂—, —CH(CH₃)—, —CH(CH₃)CH₂—CH₂—, —CH(C₂H₅)—, —C(CH₃)₂—, —CH(CH₃)CH₂—, —CH(CH₃)CH(CH₃)—, —CH═CH—, —CH═CH—CH₂—, —CH₂—CH═CH—CH₂—, and —CH(CH₃)CH═CH—, optionally substituted in each case one to four times, the same or differently, by methyl, ethyl, n- or i-propyl, n-, i-, s- or t-butyl, cyclopropylmethyl, cyclopropylethyl, cyclobutylmethyl, cyclobutylethyl, cyclopentylmethyl, cyclopentylethyl, cyclohexylmethyl and cyclohexylethyl, difluoromethyl, trifluoromethyl, chlorodifluoromethyl, fluoroethyl, difluoroethyl, trifluoroethyl, chloroethyl, dichloroethyl, trichloroethyl, cyano or methylcarbonyl, ethylcarbonyl, n- or i-propylcarbonyl, n-, i-, s- or t-butylcarbonyl, and whereby a CH₂ group can be optionally replaced by O, S or NR⁹,
R⁸ stands for methyl, ethyl, n- or i-propyl, n-, i-, s- or t-butyl, difluoromethyl, trifluoromethyl, chlorodifluoromethyl, fluoroethyl, difluoroethyl, trifluoroethyl, chloroethyl, dichloroethyl, trichloroethyl, ethenyl, 2-propenyl, 1-butenyl, 2-butenyl, 3-butenyl, 1-pentenyl, 2-pentenyl, 3-pentenyl, fluoroethenyl, difluoroethenyl, trifluoroethenyl, chloroethenyl, dichloroethenyl, trichloroethenyl, ethinyl, 1-propinyl, 2-propinyl, 1-butinyl, 2-butinyl, 3-butinyl, 1-pentinyl, 2-pentinyl, 3-pentinyl, methoxy, ethoxy, n- or i-propoxy, n-, i-, s- or t-butoxy, fluoromethoxy, difluoromethoxy, trifluoromethoxy, chlorodifluoromethoxy, fluoroethoxy, difluoroethoxy, trifluoroethoxy, chloroethoxy or dichloroethoxy, ethenyloxy, 2-propenyloxy, 1-butenyloxy, 2-butenyloxy, 3-butenyloxy, 1-pentenyloxy, 2-pentenyloxy, 3-pentenyloxy, fluoroethenyl, difluoroethenyl, trifluoroethenyl, chloroethenyl, dichloroethenyl, trichloroethenyl, ethinyloxy, 1-propinyloxy, 2-propinyloxy, 1-butinyloxy, 2-butinyloxy, 3-butinyloxy, C₃-C₅-cycloalkyl, for phenyl or benzyl optionally substituted in each case one to three times, the same or differently, in the aryl part by halogen, cyano, nitro, methyl, ethyl, n- or i-propyl, n-, i-, s- or t-butyl, difluoromethyl, trifluoromethyl, chlorodifluoromethyl, fluoroethyl, difluoroethyl, trifluoroethyl, chloroethyl, dichloroethyl, trichloroethyl, methylcarbonyl, ethylcarbonyl, n- or i-propylcarbonyl, n-, i-, s- or t-butylcarbonyl, ethenyl, 2-propenyl, 1-butenyl, 2-butenyl, 3-butenyl, 1-pentenyl, 2-pentenyl, 3-pentenyl, fluoroethenyl, difluoroethenyl, trifluoroethenyl, chloroethenyl, dichloroethenyl, trichloroethenyl, ethinyl, 1-propinyl, 2-propinyl, 1-butinyl, 2-butinyl, 3-butinyl, 1-pentinyl, 2-pentinyl, 3-pentinyl, methoxy, ethoxy, n- or i-propoxy, n-, i-, s- or t-butoxy, fluoromethoxy, difluoromethoxy, trifluoromethoxy, chlorodifluoromethoxy, fluoroethoxy, difluoroethoxy, trifluoroethoxy, chloroethoxy or dichloroethoxy, methoxycarbonyl, ethoxycarbonyl, n- or i-propoxycarbonyl, n-, i-, s- or t-butoxycarbonyl, fluoromethoxycarbonyl, difluoromethoxycarbonyl, trifluoromethoxycarbonyl, chlorodifluoromethoxycarbonyl, fluoroethoxycarbonyl, difluoroethoxycarbonyl, trifluoroethoxycarbonyl, chloroethoxycarbonyl or dichloroethoxycarbonyl,
R⁹ stands for hydrogen, methyl, ethyl, n- or i-propyl, n-, i-, s- or t-butyl, difluoromethyl, trifluoromethyl, chlorodifluoromethyl, fluoroethyl, difluoroethyl, trifluoroethyl, chloroethyl, dichloroethyl, trichloroethyl, methoxymethyl, methoxyethyl, ethoxymethyl, ethoxyethyl, n- or i-propoxymethyl, n- or i-propoxyethyl, n-, i-, s- or t-butoxymethyl, n-, i-, s- or t-butoxyethyl, methoxycarbonyl, ethoxycarbonyl, n- or i-propoxycarbonyl, n-, i-, s- or t-butoxycarbonyl, cyclopropyl, cyclobutyl, cyclopentyl or cyclohexyl, and
Y stands for a heterocyclic grouping selected from the following list, connected with the adjacent groupings at two different positions, 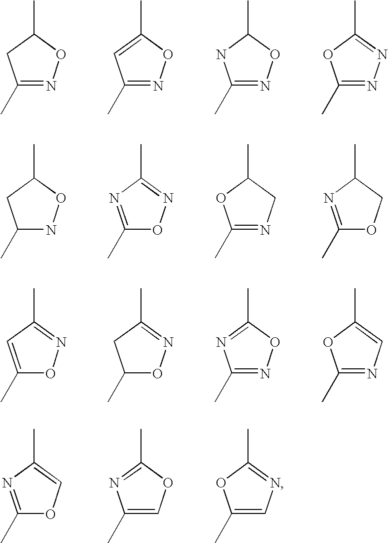
whereby these heterocyclic groupings can be optionally substituted in each case by one or two substituents from the series nitro, hydroxy, amino, cyano, fluorine, chlorine, bromine, methyl, ethyl, n- or i-propyl, n-, i-, s- or t-butyl, difluoromethyl, trifluoromethyl, chlorodifluoromethyl, fluoroethyl, difluoroethyl, trifluoroethyl, chloroethyl, dichloroethyl, trichloroethyl, methoxy, ethoxy, n- or i-propoxy, n-, i-, s- or t-butoxy, fluoromethoxy, difluoromethoxy, trifluoromethoxy, chlorodifluoromethoxy, fluoroethoxy, difluoroethoxy, trifluoroethoxy, chloroethoxy or dichloroethoxy, methylthio, ethylthio, n- or i-propylthio, n-, i-, s- or t-butylthio, difluoromethylthio, trifluoromethylthio or chlorodifluoromethylthio.

4. Compounds of the formula (I) according to claim 1, characterised in that
A¹ stands for the grouping —CH₂—CH═CCl₂,
A² stands for one of the following listed groupings
—CH₂O—, —CH₂CH₂O—, —CH₂CH₂CH₂O—, —CH₂CH₂CH₂CH₂O—,
R¹ stands for hydrogen, nitro, hydroxy, cyano, fluorine, chlorine, bromine, methyl, ethyl, n- or i-propyl, methoxy, ethoxy, n- or i-propoxy, methylthio, ethylthio, n- or i-propylthio, methylamino, ethylamino, n- or i-propylamino, dimethylamino, for phenoxy, phenylthio, benzyl or phenylethyl optionally substituted in each case by nitro, hydroxy, cyano, fluorine, chlorine, bromine, methyl, ethyl, n- or i-propyl, n-, i-, s- or t-butyl, difluoromethyl, trifluoromethyl, chlorodifluoromethyl, fluoroethyl, difluoroethyl, trifluoroethyl, chloroethyl, dichloroethyl, trichloroethyl, methoxy, ethoxy, n- or i-propoxy, fluoromethoxy, difluoromethoxy, trifluoromethoxy, chlorodifluoromethoxy, fluoroethoxy, difluoroethoxy, trifluoroethoxy, chloroethoxy or dichloroethoxy, or for the grouping —O-A¹, whereby A¹ has one of the meanings provided above,
R² stands for hydrogen, cyano, fluorine, chlorine, bromine, methyl, ethyl, methoxy or ethoxy,
R³ stands for hydrogen, cyano, fluorine, chlorine, bromine, methyl, ethyl, methoxy or ethoxy,
R⁴ stands for hydrogen, cyano, fluorine, chlorine or bromine,
R⁵ stands for hydrogen, for phenyl optionally substituted by nitro, cyano, fluorine, chlorine, bromine, methyl, ethyl, n- or i-propyl, difluoromethyl, trifluoromethyl, chlorodifluoromethyl, fluoroethyl, difluoroethyl, trifluoroethyl, chloroethyl, dichloroethyl, trichloroethyl, methoxy, ethoxy, n- or i-propoxy, fluoromethoxy, difluoromethoxy, trifluoromethoxy, chlorodifluoromethoxy, fluoroethoxy, difluoroethoxy, trifluoroethoxy, chloroethoxy or dichloroethoxy, C₁-C₂-alkylendioxy, C₁-C₂-fluoroalkylendioxy, methylthio, ethylthio, n- or i-propylthio, difluoromethylthio, trifluoromethylthio, chlorodifluoromethylthio, methoximinomethyl, ethoximinomethyl, methoximinoethyl or ethoximinoethyl, or for optionally substituted pyridinyl, whereby the substituents can be selected from the following group:
nitro, hydroxy, amino, cyano, fluorine, chlorine, bromine, iodine, methyl, ethyl, n- or i-propyl, n-, i-, s- or t-butyl, difluoromethyl, trifluoromethyl, chlorodifluoromethyl, fluoroethyl, difluoroethyl, trifluoroethyl, chloroethyl, dichloroethyl, trichloroethyl, methoxy, ethoxy, n- or i-propoxy, n-, i-, s- or t-butoxy, fluoromethoxy, difluoromethoxy, trifluoromethoxy, chlorodifluoromethoxy, fluoroethoxy, difluoroethoxy, trifluoroethoxy, chloroethoxy or dichloroethoxy, methylcarbonyl, ethylcarbonyl, n- or i-propylcarbonyl, n-, i-, s- or t-butylcarbonyl, ethoxycarbonyl, n- or i-propoxycarbonyl, n-, i-, s- or t-butoxycarbonyl, ethenyl, 2-propenyl, 1-butenyl, 2-butenyl, 3-butenyl, 1-pentenyl, 2-pentenyl, 3-pentenyl, ethenyloxy, 2-propenyloxy, 1-butenyloxy, 2-butenyloxy, 3-butenyloxy, 1-pentenyloxy, 2-pentenyloxy, 3-pentenyloxy, fluoroethenyl, difluoroethenyl, trifluoroethenyl, chloroethenyl, dichloroethenyl, trichloroethenyl, fluoroethenyloxy, difluoroethenyloxy, trifluoroethenyloxy, chloroethenyloxy, dichloroethenyloxy, trichloroethenyloxy, ethinyl, 1-propinyl, 2-propinyl, 1-butinyl, 2-butinyl, 3-butinyl, 1-pentinyl, 2-pentinyl, 3-pentinyl, C₁-C₂-alkylendioxy, C₁-C₂-fluoroalkylendioxy, methylthio, ethylthio, n- or i-propylthio, n-, i-, s- or t-butylthio, difluoromethylthio, trifluoromethylthio, chlorodifluoromethylthio, methoximinomethyl, ethoximinomethyl, methoximinoethyl or ethoximinoethyl and the grouping 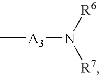
wherein the moieties A³, R⁶ and R⁷ have one of the meanings provided above in claim 1, and
Y stands for the following heterocyclic groupings, 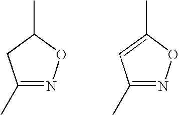
whereby these heterocyclic groupings can be optionally substituted in each case by one or two substituents from the series nitro, hydroxy, cyano, fluorine, chlorine, bromine, methyl, ethyl, n- or i-propyl, difluoromethyl, trifluoromethyl, chlorodifluoromethyl, fluoroethyl, difluoroethyl, trifluoroethyl, chloroethyl, dichloroethyl, trichloroethyl, methoxy, ethoxy, n- or i-propoxy, fluoromethoxy, difluoromethoxy, trifluoromethoxy, chlorodifluoromethoxy, fluoroethoxy, difluoroethoxy, trifluoroethoxy, chloroethoxy or dichloroethoxy, methylthio, ethylthio, n- or i-propylthio, difluoromethylthio, trifluoromethylthio or chlorodifluoromethylthio.

5. Compounds of the formula (I) according to claim 1, characterised in that
R¹ stands for hydrogen, nitro, hydroxy, cyano, fluorine, chlorine, bromine, methyl, ethyl, n- or i-propyl, methoxy, ethoxy, n- or i-propoxy, methylthio, ethylthio, n- or i-propylthio, methylamino, ethylamino, n- or i-propylamino or dimethylamino,
R² stands for hydrogen, cyano, fluorine, chlorine or bromine,
R⁵ stands for hydrogen or for pyridinyl optionally substituted by fluorine, chlorine, bromine, methyl, ethyl, n- or i-propyl, difluoromethyl, trifluoromethyl, chlorodifluoromethyl, fluoroethyl, difluoroethyl, trifluoroethyl, chloroethyl, dichloroethyl, trichloroethyl, methoxy, ethoxy, n- or i-propoxy, fluoromethoxy, difluoromethoxy, trifluoromethoxy, chlorodifluoromethoxy, fluoroethoxy, difluoroethoxy, trifluoroethoxy, chloroethoxy or dichloroethoxy and
Y stands for the following heterocyclic groupings 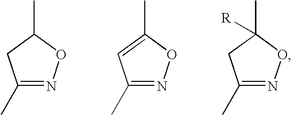
wherein
R stands for C₁-C₄-alkyl.

6. Method for the production of compounds of the formula (I) according to claim 1, characterised in that one mixes compounds of the formula (II) 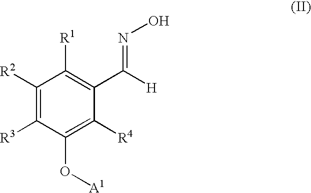
wherein
A¹, R¹, R², R³ and R⁴ have the meaning provided in claim 1,
with halogenation agents, optionally in the presence of one or more diluents,
the compounds of the general formula (III) thus produced 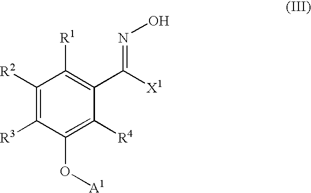
wherein
A¹, R¹, R², R³ and R⁴ have the meaning provided in claim 1,
X¹ stands for halogen,
mixed in situ with one or more acid binding agents,
and the compounds of the formula (IV) thus produced, 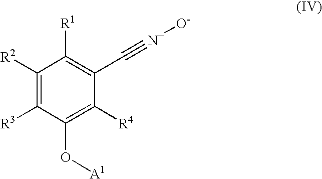
wherein
A¹, R¹, R², R³ and R⁴ have the meaning provided in claim 1,
in situ with alkenes of the general formula (V), 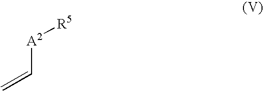
in which
A² and R⁵ have the meaning provided in claim 1 and
the carbon atoms of the olefinic double bond are optionally substituted as provided above for Y,
optionally caused to react in the presence of one or more diluents and optionally in the presence of one or more reaction agents,
and the compounds of the formula (I) thus obtained optionally converted into other compounds of the formula (I) according to traditional methods.

7. Compounds of the formula (II): 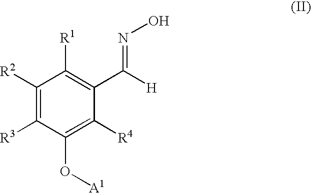
wherein
A¹ stands for one of the groupings —CH₂—CH═CCl₂, —CH₂—CH═CBr₂, —CH₂—CH═CClF, —CH₂—CF═CCl₂, —(CH₂)₂—CH═CF₂, —CH₂—CH═CBrCl, —CH₂—CH═CBrF, —CF═CH—CH═CH₂, —CH₂—CF═CF—CH═CH₂, —CH₂—CH═CClCF₃, —(CH₂)₂CX₃ and —CH₂—CH═CClCH₃, whereby X stands for halogen,
or stands for one of the groupings 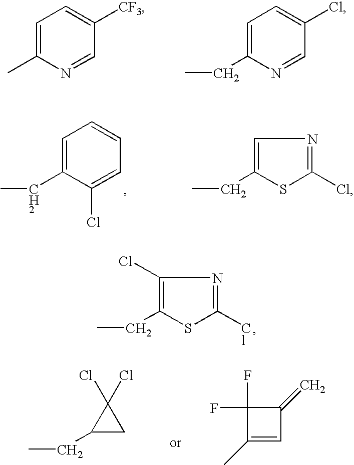
R¹ stands for hydrogen, nitro, hydroxy, amino, cyano, halogen, optionally for alkyl, alkoxy, alkylthio, alkylamino, dialkylamino, alkylcarbonylamino or alkoximinoalkyl with 1 to 10 carbon atoms in the alkyl groups substituted in each case by cyano, halogen, C₁-C₆-alkylsulfinyl, C₁-C₆-alkylsulfonyl or C₁-C₆-alkoxy, for C₁-C₆-alkylcarbonyloxy, for C₁-C₆-alkoxycarbonyloxy, for C₁-C₆-cycloalkoxycarbonyloxy, for C₁-C₆-dialkyaminocarbonyloxy, optionally for aryloxy, arylthio or arylalkyl with 6 or 10 carbon atoms in each case in the aryl groups and optionally 1 to 4 carbon atoms in the alkyl part, substituted in each case by nitro, hydroxy, amino, cyano, halogen, C₁-C₆-alkyl, C₁-C₆-halogen alkyl, C₁-C₆-alkoxy or C₁-C₆-halogenalkoxy, optionally for heterocyclyloxy or heterocyclylthio with up to 10 carbon atoms, up to 4 nitrogen atoms and optionally an oxygen or sulphur atom in each case, substituted in each case by nitro, hydroxy, amino, cyano, halogen, C₁-C₆-alkyl, C₁-C₆-halogenalkyl, C₁-C₆-alkoxy or C₁-C₆-halogenalkoxy, or stands for the grouping —O-A¹, whereby the A¹ has the meaning given above, or stands for the grouping —N(R,R′), whereby R and R′ together stand for straight-chain or branched alkanediyl with up to 8 carbon atoms, which optionally contains an oxygen atom, a sulphur atom or a grouping selected from SO, SO₂, NH or N(C₁-C₄-alkyl) at the beginning of, at the end of or within the carbon chain,
R² stands for hydrogen, nitro, hydroxy, amino, cyano, cyanato, thiocyanato, formyl, halogen, optionally for alkyl, alkoxy, alkylthio, alkylsulfinyl, alkylsulfonyl, alkylamino, dialkylamino or alkylcarbonylamino with 1 to 6 carbon atoms in each case in the alkyl groups, substituted in each case by cyano, halogen or C₁-C₆-alkoxy, for C₁-C₆-alkyl-carbonyl, C₁-C₆-alkoxy-carbonyl, C₁-C₆-alkoximinoformyl, C₁-C₆-alkoximino-acetyl, or for C₂-C₆-alkenyl or C₂-C₆-alkinyl,
R³ stands for hydrogen, nitro, hydroxy, amino, cyano, halogen, optionally for alkyl, alkoxy, alkylthio, alkylamino, dialkylamino or alkylcarbonylamino with 1 to 6 carbon atoms in each case in the alkyl groups, substituted by cyano, halogen or C₁-C₆-alkoxy, and
R⁴ stands for hydrogen, nitro, hydroxy, amino, cyano, halogen, optionally for alkyl, alkoxy, alkylthio, alkylamino, dialkylamino or alkylcarbonylamino with 1 to 6 carbon atoms in each case in the alkyl groups, substituted by cyano, halogen or C₁-C₆-alkoxy.

8. Compounds of the formula (VIII): 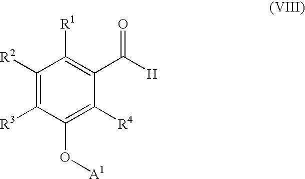
wherein
A¹ stands for one of the groupings —CH₂—CH═CCl₂, —CH₂—CH═CBr₂, —CH₂—CH═CClF, —CH₂—CF═CCl₂, —(CH₂)₂—CH═CF₂, —CH₂—CH═CBrCl, —CH₂—CH═CBrF, —CF═CH—CH═CH₂, —CH₂—CF═CF—CH═CH₂, —CH₂—CH═CClCF₃, —(CH₂)₂—CX₃ and —CH₂—CH═CClCH₃, whereby X stands for halogen,
or stands for one of the groupings 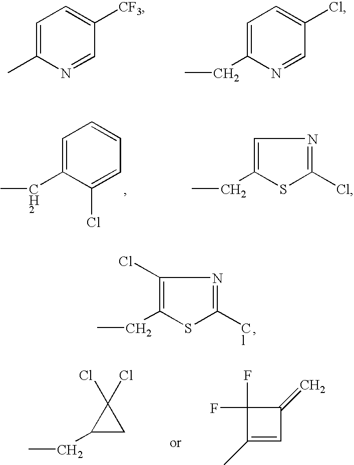
R¹ stands for hydrogen, nitro, hydroxy, amino, cyano, halogen, optionally for alkyl, alkoxy, alkylthio, alkylamino, dialkylamino, alkylcarbonylamino or alkoximinoalkyl with 1 to 10 carbon atoms in the alkyl groups substituted in each case by cyano, halogen, C₁-C₆-alkylsulfinyl, C₁-C₆-alkylsulfonyl or C₁-C₆-alkoxy, for C₁-C₆-alkylcarbonyloxy, for C₁-C₆-alkoxycarbonyloxy, for C₃-C₆-cycloalkoxycarbonyloxy, for C₁-C₆-dialkyaminocarbonyloxy, optionally for aryloxy, arylthio or arylalkyl with 6 or 10 carbon atoms in each case in the aryl groups and optionally 1 to 4 carbon atoms in the alkyl part, substituted in each case by nitro, hydroxy, amino, cyano, halogen, C₁-C₆-alkyl, C₁-C₆-halogen alkyl, C₁-C₆-alkoxy or C₁-C₆-halogenalkoxy, optionally for heterocyclyloxy or heterocyclylthio with up to 10 carbon atoms, up to 4 nitrogen atoms and optionally an oxygen or sulphur atom in each case, substituted in each case by nitro, hydroxy, amino, cyano, halogen, C₁-C₆-alkyl. C₁-C₆-halogenalkyl, C₁-C₆-alkoxy or C₁-C₆-halogenalkoxy, or stands for the grouping —O-A¹, whereby the A¹ has the meaning given above, or stands for the grouping —N(R,R′), whereby R and R′ together stand for straight-chain or branched alkanediyl with up to 8 carbon atoms, which optionally contains an oxygen atom, a sulphur atom or a grouping selected from SO, SO₂, NH or N(C₁-C₄-alkyl) at the beginning of, at the end of or within the carbon chain,
R² stands for hydrogen, nitro, hydroxy, amino, cyano, cyanato, thiocyanato, formyl, halogen, optionally for alkyl, alkoxy, alkylthio, alkylsulfinyl, alkylsulfonyl, alkylamino, dialkylamino or alkylcarbonylamino with 1 to 6 carbon atoms in each case in the alkyl groups, substituted in each case by cyano, halogen or C₁-C₆-alkoxy, for C₁-C₆-alkyl-carbonyl, C₁-C₆-alkoxy-carbonyl, C₁-C₆-alkoximinoformyl, C₁-C₆-alkoximino-acetyl, or for C₂-C₆-alkenyl or C₂-C₆-alkinyl,
R³ stands for hydrogen, nitro, hydroxy, amino, cyano, halogen, optionally for alkyl, alkoxy, alkylthio, alkylamino, dialkylamino or alkylcarbonylamino with 1 to 6 carbon atoms in each case in the alkyl groups, substituted by cyano, halogen or C₁-C₆-alkoxy, and
R⁴ stands for hydrogen, nitro, hydroxy, amino, cyano, halogen, optionally for alkyl, alkoxy, alkylthio, alkylamino, dialkylamino or alkylcarbonylamino with 1 to 6 carbon atoms in each case in the alkyl groups, substituted by cyano, halogen or C₁-C₆-alkoxy,
with the exception of the compound 3-[(3,3-dichloro-2-propenyl)-oxy]benzaldehyde.

9. Compounds of the formula (XI): 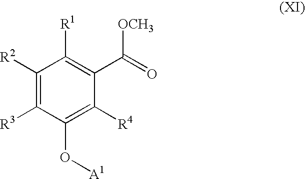
wherein
A¹ stands for one of the groupings —CH₂—CH═CCl₂, —CH₂—CH═CBr₂, —CH₂—CH═CClF, —CH₂—CF═CCl₂, —(CH₂)₂—CH═CF₂, —CH₂—CH═CBrCl, —CH₂—CH═CBrF, —CF═CH—CH═CH₂, —CH₂—CF═CF—CH═CH₂, —CH₂—CH═CClCF₃, —(CH₂)₂—CX₃ and —CH₂—CH═CClCH₃, whereby X stands for halogen,
or stands for one of the groupings 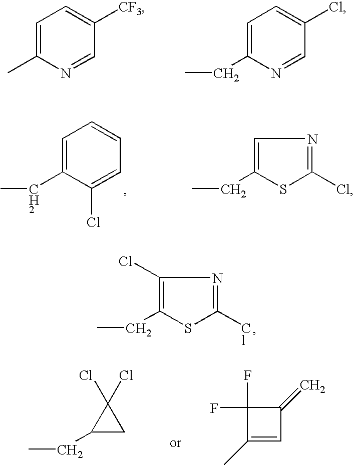
R¹ stands for hydrogen, nitro, hydroxy, amino, cyano, halogen, optionally for alkyl, alkoxy, alkylthio, alkylamino, dialkylamino, alkylcarbonylamino or alkoximinoalkyl with 1 to 10 carbon atoms in the alkyl groups substituted in each case by cyano, halogen, C₁-C₆-alkylsulfinyl, C₁-C₆-alkylsulfonyl or C₁-C₆-alkoxy, for C₁-C₆-alkylcarbonyloxy, for C₁-C₆-alkoxycarbonyloxy, for C₃-C₆-cycloalkoxycarbonyloxy, for C₁-C₆-dialkyaminocarbonyloxy, optionally for aryloxy, arylthio or arylalkyl with 6 or 10 carbon atoms in each case in the aryl groups and optionally 1 to 4 carbon atoms in the alkyl part, substituted in each case by nitro, hydroxy, amino, cyano, halogen, C₁-C₆-alkyl, C₁-C₆-halogen alkyl, C₁-C₆-alkoxy or C₁-C₆-halogenalkoxy, optionally for heterocyclyloxy or heterocyclylthio with up to 10 carbon atoms, up to 4 nitrogen atoms and optionally an oxygen or sulphur atom in each case, substituted in each case by nitro, hydroxy, amino, cyano, halogen, C₁-C₆-alkyl, C₁-C₆-halogenalkyl, C₁-C₆-alkoxy or C₁-C₆-halogenalkoxy, or stands for the grouping —O-A¹, whereby the A¹ has the meaning given above, or stands for the grouping —N(R,R′), whereby R and R′ together stand for straight-chain or branched alkanediyl with up to 8 carbon atoms, which optionally contains an oxygen atom, a sulphur atom or a grouping selected from SO, SO₂, NH or N(C₁-C₄-alkyl) at the beginning of, at the end of or within the carbon chain,
R² stands for hydrogen, nitro, hydroxy, amino, cyano, cyanato, thiocyanato, formyl, halogen, optionally for alkyl, alkoxy, alkylthio, alkylsulfinyl, alkylsulfonyl, alkylamino, dialkylamino or alkylcarbonylamino with 1 to 6 carbon atoms in each case in the alkyl groups, substituted in each case by cyano, halogen or C₁-C₆-alkoxy, for C₁-C₆-alkyl-carbonyl, C₁-C₆-alkoxy-carbonyl, C₁-C₆-alkoximinoformyl, C₁-C₆-alkoximino-acetyl, or for C₂-C₆-alkenyl or C₂-C₆-alkinyl,
R³ stands for hydrogen, nitro, hydroxy, amino, cyano, halogen, optionally for alkyl, alkoxy, alkylthio, alkylamino, dialkylamino or alkylcarbonylamino with 1 to 6 carbon atoms in each case in the alkyl groups, substituted by cyano, halogen or C₁-C₆-alkoxy, and
R⁴ stands for hydrogen, nitro, hydroxy, amino, cyano, halogen, optionally for alkyl, alkoxy, alkylthio, alkylamino, dialkylamino or alkylcarbonylamino with 1 to 6 carbon atoms in each case in the alkyl groups, substituted by cyano, halogen or C₁-C₆-alkoxy.

10. Compounds of the formula (XII): 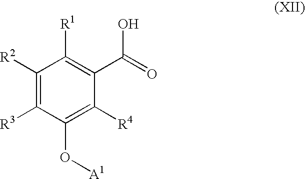
wherein
A¹ stands for one of the groupings —CH₂—CH═CCl₂, —CH₂—CH═CBr₂, —CH₂—CH═CClF, —CH₂—CF═CCl₂, —(CH₂)₂—CH═CF₂, —CH₂—CH═CBrCl, —CH₂—CH═CBrF, —CF═CH—CH═CH₂, —CH₂—CF═CF—CH═CH₂, —CH—CH═CClCF₃, —(CH₂)₂—CX₃ and —CH₂—CH═CClCH₃, whereby X stands for halogen,
or stands for one of the groupings 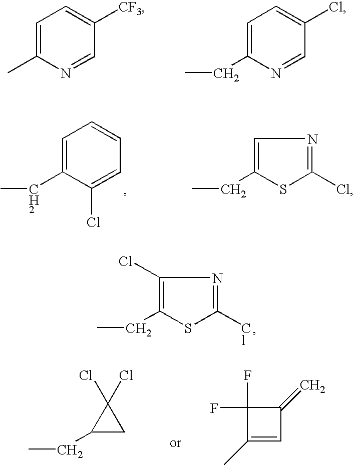
R¹ stands for hydrogen, nitro, hydroxy, amino, cyano, halogen, optionally for alkyl, alkoxy, alkylthio, alkylamino, dialkylamino, alkylcarbonylamino or alkoximinoalkyl with 1 to 10 carbon atoms in the alkyl groups substituted in each case by cyano halogen, C₁-C₆-alkylsulfinyl, C₁-C₆-alkylsulfonyl or C₁-C₆-alkoxy, for C₁-C₆alkylcarbonyloxy, for C₁-C₆-alkoxycarbonyloxy, for C₃-C₃-cycloalkoxycarbonyloxy, for C₁-C₆-dialkyaminocarbonyloxy, optionally for aryloxy, arylthio or arylalkyl with 6 or 10 carbon atoms in each case in the aryl groups and optionally 1 to 4 carbon atoms in the alkyl part, substituted in each case by nitro, hydroxy, amino, cyano, halogen, C₁-C₆-alkyl, C₁-C₆-halogen alkyl, C₁-C₆-alkoxy or C₁-C₆-halogenalkoxy, optionally for heterocyclyloxy or heterocyclylthio with up to 10 carbon atoms, up to 4 nitrogen atoms and optionally an oxygen or sulphur atom in each case, substituted in each case by nitro, hydroxy, amino, cyano, halogen, C₁-C₆-alkyl. C₁-C₆-halogenalkyl, C₁-C₆-alkoxy or C₁-C₆-halogenalkoxy, or stands for the grouping —O-A¹, whereby the A¹ has the meaning given above, or stands for the grouping —N(R,R′), whereby R and R′ together stand for straight-chain or branched alkanediyl with up to 8 carbon atoms, which optionally contains an oxygen atom, a sulphur atom or a grouping selected from SO, SO₂, NH or N(C₁-C₄-alkyl) at the beginning of, at the end of or within the carbon chain,
R² stands for hydrogen, nitro, hydroxy, amino, cyano, cyanato, thiocyanato, formyl, halogen, optionally for alkyl, alkoxy, alkylthio, alkylsulfinyl, alkylsulfonyl, alkylamino, dialkylamino or alkylcarbonylamino with 1 to 6 carbon atoms in each case in the alkyl groups, substituted in each case by cyano, halogen or C₁-C₆-alkoxy, for C₁-C₆-alkyl-carbonyl, C₁-C₆-alkoxy-carbonyl, C₁-C₆-alkoximinoformyl, C₁-C₆-alkoximino-acetyl, or for C₂-C₆-alkenyl or C₂-C₆-alkinyl,
R³ stands for hydrogen, nitro, hydroxy, amino, cyano, halogen, optionally for alkyl, alkoxy, alkylthio, alkylamino, dialkylamino or alkylcarbonylamino with 1 to 6 carbon atoms in each case in the alkyl groups, substituted by cyano, halogen or C₁-C₆-alkoxy, and
R⁴ stands for hydrogen, nitro, hydroxy, amino, cyano, halogen, optionally for alkyl, alkoxy, alkylthio, alkylamino, dialkylamino or alkylcarbonylamino with 1 to 6 carbon atoms in each case in the alkyl groups, substituted by cyano, halogen or C₁-C₆-alkoxy.

11. Compounds of the formula (XIII): 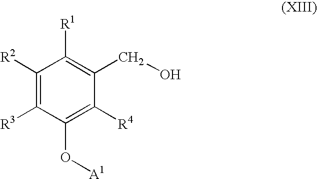
wherein
A¹ stands for one of the groupings —CH₂—CH═CCl₂, —CH₂—CH═CBr₂, —CH₂—CH═CClF, —CH₂—CF═CCl₂, —(CH₂)₂—CH═CF₂, —CH₂—CH═CBrCl, —CH₂—CH═CBrF, —CF═CH—CH═CH₂, —CH₂—CF═CF—CH═CH₂, —CH₂—CH═CClCF₃, —(CH₂)₂—CX₃ and —CH₂—CH═CClCH₃, whereby X stands for halogen,
or stands for one of the groupings 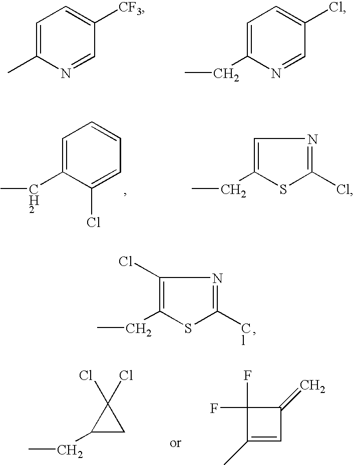
R¹ stands for hydrogen, nitro, hydroxy, amino, cyano, halogen, optionally for alkyl, alkoxy, alkylthio, alkylamino, dialkylamino, alkylcarbonylamino or alkoximinoalkyl with 1 to 10 carbon atoms in the alkyl groups substituted in each case by cyano, halogen, C₁-C₆-alkylsulfinyl, C₁-C₆-alkylsulfonyl or C₁-C₆-alkoxy, for C₁-C₆-alkylcarbonyloxy, for C₁-C₆-alkoxycarbonyloxy, for C₃-C₆-cycloalkoxycarbonyloxy, for C₁-C₆-dialkyaminocarbonyloxy, optionally for aryloxy, arylthio or arylalkyl with 6 or 10 carbon atoms in each case in the aryl groups and optionally 1 to 4 carbon atoms in the alkyl part, substituted in each case by nitro, hydroxy, amino, cyano, halogen, C₁-C₆-alkyl, C₁-C₆-halogen alkyl, C₁-C₆-alkoxy or C₁-C₆-halogenalkoxy, optionally for heterocyclyloxy or heterocyclylthio with up to 10 carbon atoms, up to 4 nitrogen atoms and optionally an oxygen or sulphur atom in each case, substituted in each case by nitro, hydroxy, amino, cyano, halogen, C₁-C₆-alkyl, C₁-C₆-halogenalkyl, C₁-C₆-alkoxy or C₁-C₆-halogenalkoxy, or stands for the grouping —O-A¹, whereby the A¹ has the meaning given above, or stands for the grouping —N(R,R′), whereby R and R′ together stand for straight-chain or branched alkanediyl with up to 8 carbon atoms, which optionally contains an oxygen atom, a sulphur atom or a grouping selected from SO, SO₂, NH or N(C₁-C₄-alkyl) at the beginning of, at the end of or within the carbon chain,
R² stands for hydrogen, nitro, hydroxy, amino, cyano, cyanato, thiocyanato, formyl, halogen, optionally for alkyl, alkoxy, alkylthio, alkylsulfinyl, alkylsulfonyl, alkylamino, dialkylamino or alkylcarbonylamino with 1 to 6 carbon atoms in each case in the alkyl groups, substituted in each case by cyano, halogen or C₁-C₆-alkoxy, for C₁-C₆-alkyl-carbonyl, C₁-C₆-alkoxy-carbonyl, C₁-C₆-alkoximinoformyl, C₁-C₆-alkoximino-acetyl, or for C₂-C₆-alkenyl or C₂-C₆-alkinyl.
R³ stands for hydrogen, nitro, hydroxy, amino, cyano, halogen, optionally for alkyl, alkoxy, alkylthio, alkylamino, dialkylamino or alkylcarbonylamino with 1 to 6 carbon atoms in each case in the alkyl groups, substituted by cyano, halogen or C₁₋₆-alkoxy, and
R⁴ stands for hydrogen, nitro, hydroxy, amino, cyano, halogen, optionally for alkyl, alkoxy, alkylthio, alkylamino, dialkylamino or alkylcarbonylamino with 1 to 6 carbon atoms in each case in the alkyl groups, substituted by cyano, halogen or C₁-C₆-alkoxy.

12. A composition comprising at least one compound of the formula (I) according to claim 1 and traditional extenders or surface-active substances or a combination of one or more extenders and surface-active substances.

13. (canceled)

14. (canceled)

15. A method for combating pests, comprising allowing the composition of claim 1 to act upon the pests and/or their habitat.

16. A method for combating pests, comprising allowing the composition of claim 12 to act upon the pests and/or their habitat.
